SEQUENCE LISTING (185 sequences)

SEQ ID NO: 1 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 5 aa, Artificial Sequence
  HCDR1
SYDMS

SEQ ID NO: 3 - protein, 17 aa, Artificial Sequence
  HCDR2
YISSGGGNTYYPDTVKG

SEQ ID NO: 4 - protein, 16 aa, Artificial Sequence
  HCDR3
EALTMISTRDYYAMDY

SEQ ID NO: 5 - protein, 125 aa, Artificial Sequence
  VH
EVKLVESGGGLVKPGGSLKLSCAASGFAFSSYDMSWVRQTPEKRLEWVAYISSGGGNTYYPDTVKGRFTISRDNAKNTLYLQMSSLKSEDTAMYYCAREALTMISTRDYYAMDYWGQGTSVTVSS

SEQ ID NO: 6 - protein, 15 aa, Artificial Sequence
  LCDR1
RASESVDNYGISFMN

SEQ ID NO: 7 - protein, 7 aa, Artificial Sequence
  LCDR2
AASNQGS

SEQ ID NO: 8 - protein, 9 aa, Artificial Sequence
  LCDR3
QQSKEVPYT

SEQ ID NO: 9 - protein, 112 aa, Artificial Sequence
  VL
DIVMTQPPASLAASLGQRATISCRASESVDNYGISFMNWFQQKPGQPPKLLIYAASNQGSGVPARFSGSGSGTDFSLNIHPMEEDDAAMYFCQQSKEVPYTFGGGTKLEIKR

SEQ ID NO: 10 - protein, 15 aa, Artificial Sequence
  LCDR1
GASESVDNYGISFMD

SEQ ID NO: 11 - protein, 112 aa, Artificial Sequence
  VL
DVLMTQTPASLAVSLGQRATISCGASESVDNYGISFMDWFQQKPGQPPKLLIYAASNQGSGVPARFSGSGSGTDFSLNIHPMEENDAAMYFCQQSKEVPYTFGGGTKLEIKR

SEQ ID NO: 12 - protein, 125 aa, Artificial Sequence
  VH
EVQLVESGGGLVQPGGSLRLSCAASGFTFSSYDMSWVRQAPGKGLEWVSYISSGGGNTYYPDTVKGRFTISRDNAKNSLYLQMNSLRAEDTAVYYCAREALTMISTRDYYAMDYWGQGTLVTVSS

SEQ ID NO: 13 - protein, 112 aa, Artificial Sequence
  VL
DIVLTQSPASLAVSPGQRATITCGASESVDNYGINLIHWYQQKPGQPPKLLIYAASNQGSGVPARFSGSGSGTDFTLTINPVEANDTANYYCQQSKEVPYTFGGGTKLEIKR

SEQ ID NO: 14 - protein, 125 aa, Artificial Sequence
  VH
EVKLVESGGGLVQPGGSLRLSCAASGFAFSSYDMSWVRQAPGKGLEWVAYISSGGGNTYYPDTVKGRFTISRDNAKNSLYLQMNSLRAEDTAVYYCAREALTMISTRDYYAMDYWGQGTLVTVSS

SEQ ID NO: 15 - protein, 112 aa, Artificial Sequence
  VL
DVLMTQSPASLAVSPGQRATISCGASESVDNYGISFMDWFQQKPGQPPKLLIYAASNQGSGVPARFSGSGSGTDFTLTINPMEENDTANYFCQQSKEVPYTFGGGTKLEIKR

SEQ ID NO: 16 - protein, 125 aa, Artificial Sequence
  VH
EVKLVESGGGLVQPGGSLRLSCAASGFAFSSYDMSWVRQAPEKRLEWVAYISSGGGNTYYPDTVKGRFTISRDNAKNSLYLQMNSLRAEDTAVYYCAREALTMISTRDYYAMDYWGQGTLVTVSS

SEQ ID NO: 17 - protein, 112 aa, Artificial Sequence
  VL
DIVLTQSPASLAVSPGQRATITCGASESVDNYGISFMDWFQQKPGQPPKLLIYAASNQGSGVPARFSGSGSGTDFTLTINPVEANDTANYYCQQSKEVPYTFGGGTKLEIKR

SEQ ID NO: 18 - protein, 112 aa, Artificial Sequence
  VL
DIVLTQSPASLAVSPGQRATITCGASESVDNYGISFMDWFQQKPGQPPKLLIYAASNQGSGVPARFSGSGSGTDFTLTINPVEAQDTANYYCQQSKEVPYTFGGGTKLEIKR

SEQ ID NO: 19 - protein, 112 aa, Artificial Sequence
  VL
DIQLTQSPSSLSASVGDRVTITCGASESVDNYGISFMDWYQQKPGKAPKLLIYAASNQGSGVPSRFSGSGSGTDFTLTISSLQPEDFATYYCQQSKEVPYTFGQGTKVEIKR

SEQ ID NO: 20 - protein, 112 aa, Artificial Sequence
  VL
EVVMTQSPATLSVSPGERATLSCGASESVDNYGISFMDWYQQKPGQAPRLLIYAASNQGSGIPARFSGSGSGTEFTLTISSLQSEDFAVYYCQQSKEVPYTFGQGTKVEIKR

SEQ ID NO: 21 - protein, 125 aa, Artificial Sequence
  VH
EVQLVESGGGLVQPGGSLRLSCAASGFAFSSYDMSWVRQAPEKRLEWVAYISSGGGNTYYPDTVKGRFTISRDNAKNTLYLQMNSLRAEDTAVYYCAKEALTMISTRDYYAMDYWGQGTLVTVSS

SEQ ID NO: 22 - protein, 125 aa, Artificial Sequence
  VH
EVQLVESGGGLVQPGGSLRLSCAASGFAFSSYDMSWVRQAPGKGLEWVAYISSGGGNTYYPDTVKGRFTISRDNAKNTLYLQMNSLRAEDTAVYYCAREALTMISTRDYYAMDYWGQGTLVTVSS

SEQ ID NO: 23 - protein, 5 aa, Artificial Sequence
  HCDR1
TYGVH

SEQ ID NO: 24 - protein, 16 aa, Artificial Sequence
  HCDR2
VIWSDGLTTYNSALKS

SEQ ID NO: 25 - protein, 10 aa, Artificial Sequence
  HCDR3
DYTYEYAMDY

SEQ ID NO: 26 - protein, 118 aa, Artificial Sequence
  VH
EVHLKESGPDLVAPSQSLSITCTVSGFSLTTYGVHWVRQPPGKGLEWLVVIWSDGLTTYNSALKSRLSISKDNSKSQVFLKMNSLRTDDTAMYYCARDYTYEYAMDYWGQGTTVTVSS

SEQ ID NO: 27 - protein, 16 aa, Artificial Sequence
  LCDR1
RSSQSLVHSNGNTYLH

SEQ ID NO: 28 - protein, 7 aa, Artificial Sequence
  LCDR2
KVSNRFS

SEQ ID NO: 29 - protein, 9 aa, Artificial Sequence
  LCDR3
SQSTHVPFT

SEQ ID NO: 30 - protein, 113 aa, Artificial Sequence
  VL
DILLTQSPLSLPVSLGDQASISCRSSQSLVHSNGNTYLHWYLQKPGQSPKLLIYKVSNRFSGVPDRFSGSESGTDFTLKISRVEAEDLGVYFCSQSTHVPFTFGSGTKLEIKR

SEQ ID NO: 31 - protein, 5 aa, Artificial Sequence
  HCDR1
DYAMH

SEQ ID NO: 32 - protein, 17 aa, Artificial Sequence
  HCDR2
VISTYSGNTNYNQKFKG

SEQ ID NO: 33 - protein, 6 aa, Artificial Sequence
  HCDR3
GNYGSD

SEQ ID NO: 34 - protein, 115 aa, Artificial Sequence
  VH
QVHVKQSGPELVRPGVSVKISCKGSGYTFTDYAMHWVKQSHAKSLEWIGVISTYSGNTNYNQKFKGKATMTVDKSSSTAYMELARLTSEDSAIYYCARGNYGSDWGQGTLVTVSA

SEQ ID NO: 35 - protein, 17 aa, Artificial Sequence
  LCDR1
KSSQSLLNSSNQKNYLA

SEQ ID NO: 36 - protein, 7 aa, Artificial Sequence
  LCDR2
FASTRES

SEQ ID NO: 37 - protein, 9 aa, Artificial Sequence
  LCDR3
QQHYSTPYT

SEQ ID NO: 38 - protein, 114 aa, Artificial Sequence
  VL
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPYTFGGGTKLELKR

SEQ ID NO: 39 - protein, 115 aa, Artificial Sequence
  VH
QVQLVQSGAEVKKPGASVKVSCKASGYTFTDYAMHWVRQAPGQGLEWMGVISTYSGNTNYNQKFKGRVTMTRDTSISTAYMELSRLRSDDTAVYYCARGNYGSDWGQGTLVTVSS

SEQ ID NO: 40 - protein, 114 aa, Artificial Sequence
  VL
DIVMTQSPDSLAVSLGERATINCKSSQSLLNSSNQKNYLAWYQQKPGQPPKLLIYFASTRESGVPDRFSGSGSGTDFTLTISSLQAEDVAVYYCQQHYSTPYTFGGGTKLEIKR

SEQ ID NO: 41 - protein, 114 aa, Artificial Sequence
  VL
EIVMTQSPGTLSLSPGERATLSCKSSQSLLNSSNQKNYLAWYQQKPGQAPRLLIYFASTRESGIPDRFSGSGSGTDFTLTISRLEPEDFAVYYCQQHYSTPYTFGQGTKVEIKR

SEQ ID NO: 42 - protein, 5 aa, Artificial Sequence
  HCDR1
SYAMS

SEQ ID NO: 43 - protein, 17 aa, Artificial Sequence
  HCDR2
TISSGGSYTYYPDSVKG

SEQ ID NO: 44 - protein, 17 aa, Artificial Sequence
  HCDR2
TISSGGSHTYYPDSAKG

SEQ ID NO: 45 - protein, 8 aa, Artificial Sequence
  HCDR3
QNWDAFVY

SEQ ID NO: 46 - protein, 8 aa, Artificial Sequence
  HCDR3
RNWDAFVY

SEQ ID NO: 47 - protein, 8 aa, Artificial Sequence
  HCDR3
QGWDAFVY

SEQ ID NO: 48 - protein, 8 aa, Artificial Sequence
  HCDR3
QNWRAFVY

SEQ ID NO: 49 - protein, 8 aa, Artificial Sequence
  HCDR3
QNWHAFVY

SEQ ID NO: 50 - protein, 8 aa, Artificial Sequence
  HCDR3
QNWDAFGY

SEQ ID NO: 51 - protein, 8 aa, Artificial Sequence
  HCDR3
QNWDAFVS

SEQ ID NO: 52 - protein, 8 aa, Artificial Sequence
  HCDR3
QNWDVFVY

SEQ ID NO: 53 - protein, 8 aa, Artificial Sequence
  HCDR3
QNWDAFAY

SEQ ID NO: 54 - protein, 8 aa, Artificial Sequence
  HCDR3
QNWDAFVI

SEQ ID NO: 55 - protein, 8 aa, Artificial Sequence
  HCDR3
QNWDAFVA

SEQ ID NO: 56 - protein, 8 aa, Artificial Sequence
  HCDR3
QNWDAFVK

SEQ ID NO: 57 - protein, 117 aa, Artificial Sequence
  VH
EVKLVESGGGLVKPGGSLKLSCAASGFTFSSYAMSWVRQTPEKRLEWVATISSGGSYTYYPDSVKGRFTISRDNAKNTLYLQMSSLRSEDTAMYYCARQNWDAFVYWGQGTLVTASA

SEQ ID NO: 58 - protein, 11 aa, Artificial Sequence
  LCDR1
RAGQSIGTSIH

SEQ ID NO: 59 - protein, 7 aa, Artificial Sequence
  LCDR2
SASESIS

SEQ ID NO: 60 - protein, 9 aa, Artificial Sequence
  LCDR3
QQSNSWPLT

SEQ ID NO: 61 - protein, 108 aa, Artificial Sequence
  VL
DIVLTQSPAILSVSPGERVSFSCRAGQSIGTSIHWYQRRTNGSPRLLIKSASESISGIPSRFSGSGSGTDFTLSINSVESEDIADYYCQQSNSWPLTFGAGTKLEIKR

SEQ ID NO: 62 - protein, 117 aa, Artificial Sequence
  VH
EVQLVESGGGLVKPGGSLRLSCAASGFTFSSYAMSWVRQAPGKGLEWVSTISSGGSYTYYPDSVKGRFTISRDNAKNSLYLQMNSLRAEDTAVYYCARQNWDAFVYWGQGTLVTVSS

SEQ ID NO: 63 - protein, 108 aa, Artificial Sequence
  VL
EIVLTQSPDFQSVTPKEKVTITCRAGQSIGTSIHWYQQKPDQSPKLLIKSASESISGVPSRFSGSGSGTDFTLTINSLEAEDAATYYCQQSNSWPLTFGAGTKLEIKR

SEQ ID NO: 64 - protein, 117 aa, Artificial Sequence
  VH
EVQLVESGGGLVKPGGSLRLSCAASGFTFSSYAMSWVRQAPGKGLEWVSTISSGGSYTYYPDSVKGRFTISRDNAKNSLYLQMNSLRAEDTAVYYCARRNWDAFVYWGQGTLVTVSS

SEQ ID NO: 65 - protein, 117 aa, Artificial Sequence
  VH
EVQLVESGGGLVKPGGSLRLSCAASGFTFSSYAMSWVRQAPGKGLEWVSTISSGGSYTYYPDSVKGRFTISRDNAKNSLYLQMNSLRAEDTAVYYCARQGWDAFVYWGQGTLVTVSS

SEQ ID NO: 66 - protein, 117 aa, Artificial Sequence
  VH
EVQLVESGGGLVKPGGSLRLSCAASGFTFSSYAMSWVRQAPGKGLEWVSTISSGGSYTYYPDSVKGRFTISRDNAKNSLYLQMNSLRAEDTAVYYCARQNWRAFVYWGQGTLVTVSS

SEQ ID NO: 67 - protein, 117 aa, Artificial Sequence
  VH
EVQLVESGGGLVKPGGSLRLSCAASGFTFSSYAMSWVRQAPGKGLEWVSTISSGGSYTYYPDSVKGRFTISRDNAKNSLYLQMNSLRAEDTAVYYCARQNWHAFVYWGQGTLVTVSS

SEQ ID NO: 68 - protein, 117 aa, Artificial Sequence
  VH
EVQLVESGGGLVKPGGSLRLSCAASGFTFSSYAMSWVRQAPGKGLEWVSTISSGGSHTYYPDSAKGRFTISRDNAKNSLYLQMNSLRAEDTAVYYCARQNWDAFGYWGQGTLVTVSS

SEQ ID NO: 69 - protein, 117 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVYWGQGTLVTVSS

SEQ ID NO: 70 - protein, 108 aa, Artificial Sequence
  VL
EIVLTQSPVILSVTPKEKVTFTCRAGQSIGTSIHWYQQRTNQSPKLLIKSASESISGVPSRFSGSGSGTDFTLTINSLEAEDIATYYCQQSNSWPLTFGAGTKLEIKR

SEQ ID NO: 71 - protein, 116 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSGQGTLVTVSS

SEQ ID NO: 72 - protein, 117 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDVFVYWGQGTLVTVSS

SEQ ID NO: 73 - protein, 117 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFAYWGQGTLVTVSS

SEQ ID NO: 74 - protein, 117 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSS

SEQ ID NO: 75 - protein, 108 aa, Artificial Sequence
  VL
EIVLTQSPVILSVTPKEKVTFTCRAGQSIGTSIHWYQQRTDQSPKLLIKSASESISGVPSRFSGSGSGTDFTLTINSLEAEDIATYYCQQSNSWPLTFGAGTKLEIKR

SEQ ID NO: 76 - protein, 108 aa, Artificial Sequence
  VL
DIQMTQSPSSLSASVGDRVTITCRAGQSIGTSIHWYQQKPGKAPKLLIKSASESISGVPSRFSGSGSGTDFTLTISSLQPEDFATYYCQQSNSWPLTFGAGTKLEIKR

SEQ ID NO: 77 - protein, 117 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVIWGQGTLVTVSS

SEQ ID NO: 78 - protein, 117 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVAWGQGTLVTVSS

SEQ ID NO: 79 - protein, 117 aa, Artificial Sequence
  VH
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVKWGQGTLVTVSS

SEQ ID NO: 80 - protein, 108 aa, Artificial Sequence
  VL
EIVLTQSPDFQSVTPKEKVTITCRAGQSIGTSIHWYQRKPDQSPKLLIKSASESISGVPSRFSGSGSGTDFTLTINSLEAEDAATYYCQQSNSWPLTFGAGTKVEIKR

SEQ ID NO: 81 - protein, 5 aa, Artificial Sequence
  HCDR1
SYGVH

SEQ ID NO: 82 - protein, 16 aa, Artificial Sequence
  HCDR2
VIWSDGFTTYNSALKS

SEQ ID NO: 83 - protein, 11 aa, Artificial Sequence
  HCDR3
NDGSYYYAMDY

SEQ ID NO: 84 - protein, 119 aa, Artificial Sequence
  VH
EVKLKESGPGLVAPSQSLSITCTVSGFSLTSYGVHWVRQPPGKGLEWLVVIWSDGFTTYNSALKSRLSISKDNSKSQVFLKMNSLQTDDTAMYYCARNDGSYYYAMDYWGQGTSVTVSS

SEQ ID NO: 85 - protein, 113 aa, Artificial Sequence
  VL
DIELTQPPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPFTFGSGTKLEIKR

SEQ ID NO: 86 - protein, 16 aa, Artificial Sequence
  HCDR2
VIWSDGSTTYNSALKS

SEQ ID NO: 87 - protein, 10 aa, Artificial Sequence
  HCDR3
DYRYEYVMDY

SEQ ID NO: 88 - protein, 118 aa, Artificial Sequence
  VH
QVHVKQSGPDLVAPSQSLSITCTVSGFSLTSYGVHWVRQPPGKGLEWLVVIWSDGSTTYNSALKSRLSISKDNSKSQVFLKMNSLQTDDTAMYYCARDYRYEYVMDYWGQGTSVTVSS

SEQ ID NO: 89 - protein, 9 aa, Artificial Sequence
  LCDR3
SQSIHVPFT

SEQ ID NO: 90 - protein, 113 aa, Artificial Sequence
  VL
DVVMTQTPLSLPVSLGDQASISCRSSQSLVHSNGNTYLHWYLQKPGQSPKVLIYKVSNRFSGVPDRFSGSGSGTDFTLKISRVEAEDLGVYFCSQSIHVPFTFGSGTKLEIKR

SEQ ID NO: 91 - protein, 5 aa, Artificial Sequence
  HCDR1
SNGVH

SEQ ID NO: 92 - protein, 12 aa, Artificial Sequence
  HCDR3
HDGYSYYYAMEY

SEQ ID NO: 93 - protein, 120 aa, Artificial Sequence
  VH
QVQLQQSGPDLVAPSQSLSITCTVSGFSLTSNGVHWVRQPPGKGLEWLVVIWSDGSTTYNSALKSRLSISKDNSKSQVFLKMNSLQTDDTAMYYCARHDGYSYYYAMEYWGQGTTLTVSS

SEQ ID NO: 94 - protein, 9 aa, Artificial Sequence
  LCDR3
SQSTHVPLT

SEQ ID NO: 95 - protein, 113 aa, Artificial Sequence
  VL
DIVMTQVPLSLPVSLGDQASISCRSSQSLVHSNGNTYLHWYLQKPGQSPKLLIYKVSNRFSGVPDRFRGSGSGTDFALKISRVEAEDLGVYFCSQSTHVPLTFGAGTKLEIKR

SEQ ID NO: 96 - protein, 12 aa, Artificial Sequence
  HCDR3
YDNYGYYFAMDY

SEQ ID NO: 97 - protein, 120 aa, Artificial Sequence
  VH
EVKVIESGPDLVAPSQSLSITCTVSGFSLTSYGVHWVRQPPGKGLEWLVVIWSDGSTTYNSALKSRLSISKDNSKSQVFLKMHSLQTDDTAMYYCASYDNYGYYFAMDYWGQGTTLTVSS

SEQ ID NO: 98 - protein, 113 aa, Artificial Sequence
  VL
  misc_feature (111)..(111): Xaa can be any naturally occurring amino acid
DIQLTQSPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLEXKR

SEQ ID NO: 99 - protein, 117 aa, Artificial Sequence
  VH
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSS

SEQ ID NO: 100 - protein, 431 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 667 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 667 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 663 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 104 - protein, 663 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 105 - protein, 664 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 664 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 667 aa, Artificial Sequence; identical to SEQ ID NO: 101
  Heavy chain

SEQ ID NO: 108 - protein, 667 aa, Artificial Sequence; identical to SEQ ID NO: 102
  Heavy chain

SEQ ID NO: 109 - protein, 668 aa, Artificial Sequence
  Heavy chain
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 110 - protein, 668 aa, Artificial Sequence
  Heavy chain
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 111 - protein, 661 aa, Artificial Sequence
  Heavy chain
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 112 - protein, 661 aa, Artificial Sequence
  Heavy chain
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 113 - protein, 668 aa, Artificial Sequence; identical to SEQ ID NO: 109
  Heavy chain

SEQ ID NO: 114 - protein, 668 aa, Artificial Sequence; identical to SEQ ID NO: 110
  Heavy chain

SEQ ID NO: 115 - protein, 661 aa, Artificial Sequence; identical to SEQ ID NO: 111
  Heavy chain

SEQ ID NO: 116 - protein, 661 aa, Artificial Sequence; identical to SEQ ID NO: 112
  Heavy chain

SEQ ID NO: 117 - protein, 447 aa, Artificial Sequence
  Heavy chain
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 447 aa, Artificial Sequence
  Heavy chain
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 447 aa, Artificial Sequence; identical to SEQ ID NO: 117
  Heavy chain

SEQ ID NO: 120 - protein, 447 aa, Artificial Sequence; identical to SEQ ID NO: 118
  Heavy chain

SEQ ID NO: 121-122 - skipped (2 entries)

SEQ ID NO: 123 - protein, 693 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCAASGFAFSSYDMSWVRQAPGKGLEWVAYISSGGGNTYYPDTVKGRFTISRDNAKNTLYLQMNSLRAEDTAVYYCAREALTMISTRDYYAMDYWGQGTLVTVSSGGGGSGGGGSGGGGSDIQLTQSPSSLSASVGDRVTITCGASESVDNYGISFMDWYQQKPGKAPKLLIYAASNQGSGVPSRFSGSGSGTDFTLTISSLQPEDFATYYCQQSKEVPYTFGQGTKVEIKR

SEQ ID NO: 124 - protein, 681 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSGGGGSGGGGSGGGGSEIVLTQSPDFQSVTPKEKVTITCRAGQSIGTSIHWYQRKPDQSPKLLIKSASESISGVPSRFSGSGSGTDFTLTINSLEAEDAATYYCQQSNSWPLTFGAGTKVEIKR

SEQ ID NO: 125 - protein, 681 aa, Artificial Sequence
  Heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSGGGGSGGGGSGGGGSEIVLTQSPDFQSVTPKEKVTITCRAGQSIGTSIHWYQRKPDQSPKLLIKSASESISGVPSRFSGSGSGTDFTLTINSLEAEDAATYYCQQSNSWPLTFGAGTKVEIKR

SEQ ID NO: 126 - protein, 445 aa, Artificial Sequence
  Heavy chain
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 127 - protein, 715 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGGGSGGGGSGGGGSEVKLVESGGGLVQPGGSLRLSCAASGFAFSSYDMSWVRQAPEKRLEWVAYISSGGGNTYYPDTVKGRFTISRDNAKNSLYLQMNSLRAEDTAVYYCAREALTMISTRDYYAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 128 - protein, 716 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGGGSGGGGSGGGGSEVKLVESGGGLVQPGGSLRLSCAASGFAFSSYDMSWVRQAPEKRLEWVAYISSGGGNTYYPDTVKGRFTISRDNAKNSLYLQMNSLRAEDTAVYYCAREALTMISTRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGL

SEQ ID NO: 129 - protein, 707 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGGGSGGGGSGGGGSEVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 130 - protein, 707 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGGGSGGGGSGGGGSEVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 131 - protein, 708 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGGGSGGGGSGGGGSEVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGL

SEQ ID NO: 132 - protein, 708 aa, Artificial Sequence; identical to SEQ ID NO: 131
  Heavy chain

SEQ ID NO: 133 - protein, 666 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 249 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 135 - skipped

SEQ ID NO: 136 - protein, 214 aa, Artificial Sequence
  Light chain
EIVLTQSPVILSVTPKEKVTFTCRAGQSIGTSIHWYQQRTNQSPKLLIKSASESISGVPSRFSGSGSGTDFTLTINSLEAEDIATYYCQQSNSWPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 214 aa, Artificial Sequence
  Light chain
EIVLTQSPDFQSVTPKEKVTITCRAGQSIGTSIHWYQRKPDQSPKLLIKSASESISGVPSRFSGSGSGTDFTLTINSLEAEDAATYYCQQSNSWPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 434 aa, Artificial Sequence
  Light chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEIVLTQSPVILSVTPKEKVTFTCRAGQSIGTSIHWYQQRTNQSPKLLIKSASESISGVPSRFSGSGSGTDFTLTINSLEAEDIATYYCQQSNSWPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 434 aa, Artificial Sequence
  Light chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEIVLTQSPDFQSVTPKEKVTITCRAGQSIGTSIHWYQRKPDQSPKLLIKSASESISGVPSRFSGSGSGTDFTLTINSLEAEDAATYYCQQSNSWPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 219 aa, Artificial Sequence
  Light chain
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 198 aa, Artificial Sequence
  Light chain
DIVLTQSPASLAVSPGQRATITCGASESVDNYGISFMDWFQQKPGQPPKLLIYAASNQGSGVPARFSGSGSGTDFTLTINPVEAQDTANYYCQQSKEVPYTFGGGTKLEIKRTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 214 aa, Artificial Sequence
  Light chain
EIVLTQSPVILSVTPKEKVTFTCRAGQSIGTSIHWYQQRTDQSPKLLIKSASESISGVPSRFSGSGSGTDFTLTINSLEAEDIATYYCQQSNSWPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 20 aa, Artificial Sequence
  linker
NYGGGGSGGGGSGGGGSGNS

SEQ ID NO: 144 - protein, 38 aa, Artificial Sequence
  linker
NYGGGGSGGGGSGGGGSGNYGGGGSGGGGSGGGGSGNS

SEQ ID NO: 145 - protein, 2 aa, Artificial Sequence
  linker
NS

SEQ ID NO: 146 - protein, 8 aa, Artificial Sequence
  linker
GGGGSGNS

SEQ ID NO: 147 - protein, 10 aa, Artificial Sequence
  linker
NYGGGGSGNS

SEQ ID NO: 148 - protein, 13 aa, Artificial Sequence
  linker
GGGGSGGGGSGNS

SEQ ID NO: 149 - protein, 15 aa, Artificial Sequence
  linker
NYGGGGSGGGGSGNS

SEQ ID NO: 150 - protein, 18 aa, Artificial Sequence
  linker
GGGGSGGGGSGGGGSGNS

SEQ ID NO: 151 - protein, 8 aa, Artificial Sequence
  linker
GCPPCPNS

SEQ ID NO: 152 - protein, 5 aa, Artificial Sequence
  linker
GGGGS

SEQ ID NO: 153 - protein, 10 aa, Artificial Sequence
  linker
GGGGSGGGGS

SEQ ID NO: 154 - protein, 15 aa, Artificial Sequence
  linker
GGGGSGGGGSGGGGS

SEQ ID NO: 155 - protein, 16 aa, Artificial Sequence
  linker
SGGGGSGGGGSGGGGS

SEQ ID NO: 156 - protein, 20 aa, Artificial Sequence
  linker
GGGGSGGGGSGGGGSGGGGS

SEQ ID NO: 157 - protein, 21 aa, Artificial Sequence
  linker
QRHNNSSLNTGTQMAGHSPNS

SEQ ID NO: 158 - protein, 16 aa, Artificial Sequence
  linker
SSLNTGTQMAGHSPNS

SEQ ID NO: 159 - protein, 18 aa, Artificial Sequence
  linker
QRHNNSSLNTGTQMAGHS

SEQ ID NO: 160 - protein, 14 aa, Artificial Sequence
  linker
EVQIPLTESYSPNS

SEQ ID NO: 161 - protein, 20 aa, Artificial Sequence
  linker
NSLANQEVQIPLTESYSPNS

SEQ ID NO: 162 - protein, 19 aa, Artificial Sequence
  linker
SGGGGSGGGGSGGGGSPNS

SEQ ID NO: 163 - protein, 19 aa, Artificial Sequence
  linker
SGGGGSGGGGSGGGGSPGS

SEQ ID NO: 164 - protein, 19 aa, Artificial Sequence
  linker
SGGGGSGGGGSGGGGSPAS

SEQ ID NO: 165 - protein, 18 aa, Artificial Sequence
  linker
SGGGGSGGGGSGGGGSPS

SEQ ID NO: 166 - protein, 19 aa, Artificial Sequence
  linker
SGGGGSGGGGSGGGGSPSS

SEQ ID NO: 167 - protein, 205 aa, Artificial Sequence
  aflibercept VEGFR - V1
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 168 - protein, 204 aa, Artificial Sequence
  aflibercept VEGFR - 2
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 169 - protein, 447 aa, Artificial Sequence; identical to SEQ ID NO: 117
  67.2.4A IgG1 HC

SEQ ID NO: 170 - protein, 447 aa, Artificial Sequence; identical to SEQ ID NO: 118
  67.2.4B IgG1 HC

SEQ ID NO: 171 - protein, 443 aa, Artificial Sequence
  67.2.4A IgG2 HC
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 172 - protein, 443 aa, Artificial Sequence
  67.2.4B IgG2 HC
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 173 - protein, 444 aa, Artificial Sequence
  67.2.4A IgG4 HC
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 174 - protein, 444 aa, Artificial Sequence
  67.2.4B IgG4 HC
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 175 - protein, 446 aa, Artificial Sequence
  67.2.4A IgG1 HC - no K
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 446 aa, Artificial Sequence
  67.2.4B IgG1 HC - no K
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 252 aa, Artificial Sequence
  ABM59 scFv
EVQLVESGGGLVQPGGSLRLSCAASGFAFSSYDMSWVRQAPGKGLEWVAYISSGGGNTYYPDTVKGRFTISRDNAKNTLYLQMNSLRAEDTAVYYCAREALTMISTRDYYAMDYWGQGTLVTVSSGGGGSGGGGSGGGGSDIQLTQSPSSLSASVGDRVTITCGASESVDNYGISFMDWYQQKPGKAPKLLIYAASNQGSGVPSRFSGSGSGTDFTLTISSLQPEDFATYYCQQSKEVPYTFGQGTKVEIKR

SEQ ID NO: 178 - protein, 240 aa, Artificial Sequence
  ABM67-A scFv
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSGGGGSGGGGSGGGGSEIVLTQSPDFQSVTPKEKVTITCRAGQSIGTSIHWYQRKPDQSPKLLIKSASESISGVPSRFSGSGSGTDFTLTINSLEAEDAATYYCQQSNSWPLTFGAGTKVEIKR

SEQ ID NO: 179 - protein, 240 aa, Artificial Sequence
  ABM67-B scFv
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSGGGGSGGGGSGGGGSEIVLTQSPDFQSVTPKEKVTITCRAGQSIGTSIHWYQRKPDQSPKLLIKSASESISGVPSRFSGSGSGTDFTLTINSLEAEDAATYYCQQSNSWPLTFGAGTKVEIKR

SEQ ID NO: 180 - protein, 451 aa, Artificial Sequence
  ABM59.6 IgG2 Heavy chain
EVKLVESGGGLVQPGGSLRLSCAASGFAFSSYDMSWVRQAPEKRLEWVAYISSGGGNTYYPDTVKGRFTISRDNAKNSLYLQMNSLRAEDTAVYYCAREALTMISTRDYYAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 181 - protein, 452 aa, Artificial Sequence
  ABM59.6 IgG4 Heavy chain
EVKLVESGGGLVQPGGSLRLSCAASGFAFSSYDMSWVRQAPEKRLEWVAYISSGGGNTYYPDTVKGRFTISRDNAKNSLYLQMNSLRAEDTAVYYCAREALTMISTRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGL

SEQ ID NO: 182 - protein, 443 aa, Artificial Sequence
  ABM67.2.4.2A IgG2 HC
EVQLVESGGGLVKPGGSLRLSCAASGFTFSSYAMSWVRQTPEKRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 183 - protein, 443 aa, Artificial Sequence
  ABM67.2.4.2B IgG2 HC
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 184 - protein, 444 aa, Artificial Sequence
  ABM67.2.4.2A IgG4 HC
EVQLVESGGGLVKPGGSLRLPCAASGFTFSSYAMSWVRQTPERRLEWVATISSGGSYTYYPDSVKGRFTISRDNAKNSLYLQMSSLRAEDTAVYYCARQNWDAFVSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGL

SEQ ID NO: 185 - protein, 444 aa, Artificial Sequence; identical to SEQ ID NO: 184
  ABM67.2.4.2B IgG4 HC

(19) United States
(12) Patent Application Publication
(10) Pub. No.: US 2024/0301048 A1
(43) Pub. Date: Sep. 12, 2024

(54) NEUTRALIZING ANTIBODIES TO IL-17A, FUSION PROTEINS THEREOF, AND USES THEREOF

(71) Applicant: Lanier Biotherapeutics, Inc., Suwanee, GA (US)

(72) Inventors: Daniel WHITE, Suwanee, GA (US); Richard A. SHIMKETS, Commerce, GA (US); Crystal Lyles JACKSON, Jefferson, GA (US); Thomas VINCENT, Athens, GA (US); Yonghua LUO, Suwanee, GA (US)

(21) Appl. No.: 18/260,594

(22) PCT Filed: Jan. 10, 2022
(86) PCT No.: PCT/US2022/011871

§ 371 Date: Jul. 6, 2023

Related U.S. Application Data
(60) Provisional application No. 63/135,184, filed on Jan. 8, 2021.

(51) Int. Cl. C07K 16/24; A61K 9/00; A61K 39/00; A61P 27/02; C07K 16/22

(52) U.S. Cl. CPC C07K 16/244; A61K 9/0019; A61P 27/02; C07K 16/22; C07K 16/241; A61K 2039/505; C07K 2317/24; C07K 2317/31; C07K 2317/565; C07K 2317/622

(57) ABSTRACT

Disclosed are compositions and methods related to anti-IL-17A antibodies and antigen binding fragments thereof. Additionally provided are bi-specific binding proteins comprising the anti-IL-17A binding domains. Additionally provided are methods of use for the compounds and compositions described herein in the treatment of inflammatory diseases and ocular disorders.

### CROSS-REFERENCE TO RELATED APPLICATIONS

This application claims priority to U.S. Provisional Application No. 63/135,184, filed Jan. 8, 2021 the content of which is incorporated herein by reference in its entirety.

### REFERENCE TO THE ELECTRONIC SEQUENCE LISTING

The contents of the text file submitted electronically herewith are incorporated herein by reference in their entirety. A computer readable format copy of the Sequence Listing is provided (filename: LNER-001_01WO_SeqList_ST25.txt, date recorded: Jan. 10, 2022, file size: 324 kilobytes).

### BACKGROUND

Increased levels of IL-17 (i.e., IL-17A) have been associated with several conditions, diseases or disorders including airway inflammation, rheumatoid arthritis (“RA”), osteoarthritis, bone erosion, intraperitoneal abscesses and adhesions, inflammatory bowel disorder (“IBD”), allograft rejection, psoriasis, certain types of cancer, angiogenesis, atherosclerosis, and multiple sclerosis (“MS”) (for a review see Witkowski, et al, Cell. Mol. Life. Sci. 61:567-579, 2004). IL-17A has been further implicated in diseases of the eye, including macular degeneration and diabetic retinopathy. Both IL-17 and IL-17R are up-regulated in the synovial tissue of RA patients. Blocking an IL-17 bioactivity by binding an IL-17 specific antibody or soluble receptor to IL-17 reduces inflammation and bone erosion in various animal arthritis models. (See, e.g., Lubberts et al, Arthritis & Rheumatism, 50:650-659, 2004). Furthermore, IL-17 has IL-10 effects on collagen matrix breakdown and inflammation and joint damage, while IL-17 has synergy with TNF-α to amplify inflammation.

Accordingly, there is a need for compositions that antagonize or neutralize the activity of IL-17 in order to treat disorders, diseases or conditions wherein the presence of IL-17 bioactivity causes or contributes to an undesirable pathological effect or wherein a decrease in IL-17 bioactivity contributes to a desirable therapeutic effect, including inflammatory disorders, cell proliferative and developmental disorders and autoimmune disorders such as RA, MS, psoriasis, IBD, and diseases of the eye.

### SUMMARY

In some embodiments, the present disclosure provides an anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid sequence selected from SEQ ID NO: 2, 23, 31, 42, 81, and 91; (b) the HCDR2 comprises an amino acid sequence selected from SEQ ID NO: 3, 24, 32, 43, 82, and 86; (c) the HCDR3 comprises the amino acid sequence selected from SEQ ID NO: 4, 25, 33, 45, 83, 87, 92, and 96; (d) the LCDR1 comprises the amino acid sequence selected from SEQ ID NO: 6, 10, 27, 35, and 58; (e) the LCDR2 comprises the amino acid sequence selected from SEQ ID NO: 7, 28, 36, and 59; and (f) the LCDR3 comprises the amino acid sequence selected from SEQ ID NO: 8, 29, 37, 60, 89, and 94.

In some embodiments, (a) the HCDR1 comprises the amino acid sequence of SEQ ID NO: 2; (b) the HCDR2 comprises the amino acid sequence of SEQ ID NO: 3; (c) the HCDR3 comprises the amino acid sequence of SEQ ID NO: 4; (d) the LCDR1 comprises the amino acid sequence of SEQ ID NO: 6; (e) the LCDR2 comprises the amino acid sequence of SEQ ID NO: 7; and (f) the LCDR2 comprises the amino acid sequence of SEQ ID NO: 8. In some embodiments, the VH comprises an amino acid sequence that is at least 95%, 96%, 97%, 98%, or 99% identical to SEQ ID NO: 5 and wherein the VL comprises an amino acid sequence that is at least 95%, 96%, 97%, 98%, or 99% identical to SEQ ID NO: 9. In some embodiments, the VH comprises or consists of the amino acid sequence of SEQ ID NO: 5 and wherein the VL comprises or consists of the amino acid sequence of SEQ ID NO: 9.

In some embodiments, (a) the HCDR1 comprises the amino acid sequence of SEQ ID NO: 2; (b) the HCDR2 comprises the amino acid sequence of SEQ ID NO: 3; (c) the HCDR3 comprises the amino acid sequence of SEQ ID NO: 4; (d) the LCDR1 comprises the amino acid sequence of SEQ ID NO: 10; (e) the LCDR2 comprises the amino acid sequence of SEQ ID NO: 7; and (f) the LCDR2 comprises the amino acid sequence of SEQ ID NO: 8. In some embodiments, the VH comprises an amino acid sequence that is at least 95%, 96%, 97%, 98%, or 99% identical to SEQ ID NO: 5 and wherein the VL comprises an amino acid sequence that is at least 95%, 96%, 97%, 98%, or 99% identical to SEQ ID NO: 11. In some embodiments, the VH comprises or consists of the amino acid sequence of SEQ ID NO: 5 and wherein the VL comprises or consists of the amino acid sequence of SEQ ID NO: 11.

In some embodiments, (a) the HCDR1 comprises the amino acid sequence of SEQ ID NO: 23; (b) the HCDR2 comprises the amino acid sequence of SEQ ID NO: 24; (c) the HCDR3 comprises the amino acid sequence of SEQ ID NO: 25; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 29. In some embodiments, the VH comprises an amino acid sequence that is at least 95%, 96%, 97%, 98%, or 99% identical to SEQ ID NO: 26 and wherein the VL comprises an amino acid sequence that is at least 95%, 96%, 97%, 98%, or 99% identical to SEQ ID NO: 30. In some embodiments, the VH comprises or consists of the amino acid sequence of SEQ ID NO: 26 and wherein the VL comprises or consists of the amino acid sequence of SEQ ID NO: 30.

In some embodiments, (a) the HCDR1 comprises the amino acid sequence of SEQ ID NO: 31; (b) the HCDR2 comprises the amino acid sequence of SEQ ID NO: 32; (c) the HCDR3 comprises the amino acid sequence of SEQ ID NO: 33; (d) the LCDR1 comprises the amino acid sequence of SEQ ID NO: 35; (e) the LCDR2 comprises the amino acid sequence of SEQ ID NO: 36; and (f) the LCDR2 comprises the amino acid sequence of SEQ ID NO: 37. In some embodiments, the VH comprises an amino acid sequence that is at least 95%, 96%, 97%, 98%, or 99% identical to SEQ ID NO: 34 and wherein the VL comprises an amino acid sequence that is at least 95%, 96%, 97%, 98%, or 99% identical to SEQ ID NO: 38. In some embodiments, the VH comprises or consists of the amino acid sequence of SEQ ID NO: 34 and wherein the VL comprises or consists of the amino acid sequence of SEQ ID NO: 38.

In some embodiments, (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 45; (d) the LCDR1 comprises the amino acid sequence of SEQ ID NO: 58; (e) the LCDR2 comprises the amino acid sequence of SEQ ID NO: 59; and (f) the LCDR2 comprises the amino acid sequence of SEQ ID NO: 60. In some embodiments, the VH comprises an amino acid sequence that is at least 95%, 96%, 97%, 98%, or 99% identical to SEQ ID NO: 57 and wherein the VL comprises an amino acid sequence that is at least 95%, 96%, 97%, 98%, or 99% identical to SEQ ID NO: 61. In some embodiments, the VH comprises or consists of the amino acid sequence of SEQ ID NO: 57 and wherein the VL comprises or consists of the amino acid sequence of SEQ ID NO: 61.

In some embodiments, (a) the HCDR1 comprises the amino acid sequence of SEQ ID NO: 81; (b) the HCDR2 comprises the amino acid sequence of SEQ ID NO: 82; (c) the HCDR3 comprises the amino acid sequence of SEQ ID NO: 83; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 29. In some embodiments, the VH comprises an amino acid sequence that is at least 95%, 96%, 97%, 98%, or 99% identical to SEQ ID NO: 84 and wherein the VL comprises an amino acid sequence that is at least 95%, 96%, 97%, 98%, or 99% identical to SEQ ID NO: 85. In some embodiments, the VH comprises or consists of the amino acid sequence of SEQ ID NO: 84 and wherein the VL comprises or consists of the amino acid sequence of SEQ ID NO: 85.

In some embodiments, (a) the HCDR1 comprises the amino acid sequence of SEQ ID NO: 81; (b) the HCDR2 comprises the amino acid sequence of SEQ ID NO: 86; (c) the HCDR3 comprises the amino acid sequence of SEQ ID NO: 87; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 89. In some embodiments, the VH comprises an amino acid sequence that is at least 95%, 96%, 97%, 98%, or 99% identical to SEQ ID NO: 85 and wherein the VL comprises an amino acid sequence that is at least 95%, 96%, 97%, 98%, or 99% identical to SEQ ID NO: 88. In some embodiments, the VH comprises or consists of the amino acid sequence of SEQ ID NO: 85 and wherein the VL comprises or consists of the amino acid sequence of SEQ ID NO: 88.

In some embodiments, (a) the HCDR1 comprises the amino acid sequence of SEQ ID NO: 91; (b) the HCDR2 comprises the amino acid sequence of SEQ ID NO: 86; (c) the HCDR3 comprises the amino acid sequence of SEQ ID NO: 92; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 94. In some embodiments, the VH comprises an amino acid sequence that is at least 95%, 96%, 97%, 98%, or 99% identical to SEQ ID NO: 93 and wherein the VL comprises an amino acid sequence that is at least 95%, 96%, 97%, 98%, or 99% identical to SEQ ID NO: 95. In some embodiments, the VH comprises or consists of the amino acid sequence of SEQ ID NO: 93 and wherein the VL comprises or consists of the amino acid sequence of SEQ ID NO: 95.

In some embodiments, (a) the HCDR1 comprises the amino acid sequence of SEQ ID NO: 81; (b) the HCDR2 comprises the amino acid sequence of SEQ ID NO: 86; (c) the HCDR3 comprises the amino acid sequence of SEQ ID NO: 96; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 94. In some embodiments, the VH comprises an amino acid sequence that is at least 95%, 96%, 97%, 98%, or 99% identical to SEQ ID NO: 97 and wherein the VL comprises an amino acid sequence that is at least 95%, 96%, 97%, 98%, or 99% identical to SEQ ID NO: 98. In some embodiments, VH comprises or consists of the amino acid sequence of SEQ ID NO: 97 and wherein the VL comprises or consists of the amino acid sequence of SEQ ID NO: 98.

In some embodiments, the present disclosure provides a humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid sequence selected from SEQ ID NO: 2; (b) the HCDR2 comprises an amino acid sequence selected from SEQ ID NO: 3; (c) the HCDR3 comprises the amino acid sequence selected from SEQ ID NO: 4; (d) the LCDR1 comprises the amino acid sequence selected from SEQ ID NO: 10; (e) the LCDR2 comprises the amino acid sequence selected from SEQ ID NO: 7; and (f) the LCDR3 comprises the amino acid sequence selected from SEQ ID NO: 8. In some embodiments, the VH comprises an amino acid sequence is at least 95%, 96%, 97%, 98%, or 99% identical to one of SEQ ID NOs: 12, 14, 16, 21, and 22 and wherein the VL comprises an amino acid sequence is at least 95%, 96%, 97%, 98%, or 99% identical to one of SEQ ID NOs: 13, 15, 17, 18, 19, and 20.

In some embodiments, (a) the VH comprises an amino acid sequence is at least 95%, 96%, 97%, 98%, or 99% identical SEQ ID NO: 12 and the VL comprises an amino acid sequence is at least 95%, 96%, 97%, 98%, or 99% identical to SEQ ID NO: 13; (b) the VH comprises an amino acid sequence is at least 95%, 96%, 97%, 98%, or 99% identical SEQ ID NO: 14 and the VL comprises an amino acid sequence is at least 95%, 96%, 97%, 98%, or 99% identical to SEQ ID NO: 15; (c) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 15; (d) the VH comprises an amino acid sequence is at least 95%, 96%, 97%, 98%, or 99% identical SEQ ID NO: 14 and the VL comprises an amino acid sequence is at least 95%, 96%, 97%, 98%, or 99% identical to SEQ ID NO: 17; (e) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 17; (f) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 18; (g) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 19; (h) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 20; (i) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 18; (j) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 19; (k) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 20; (1) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 18; (m) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 19; or (n) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 20.

In some embodiments, the VH comprises or consists of one of SEQ ID NOs: 12, 14, 16, 21, and 22 and wherein the VL comprises or consists of one of SEQ ID NOs: 13, 15, 17, 18, 19, and 20. In some embodiments, (a) the VH amino acid sequence comprises or consists of SEQ ID NO: 12 and the VL amino acid sequence comprises or consists of SEQ ID NO: 13; (b) the VH amino acid sequence comprises or consists of SEQ ID NO: 14 and the VL amino acid sequence comprises or consists of SEQ ID NO: 15; (c) the VH amino acid sequence comprises or consists of SEQ ID NO: 16 and the VL amino acid sequence comprises or consists of SEQ ID NO: 15; (d) the VH amino acid sequence comprises or consists of SEQ ID NO: 14 and the VL amino acid sequence comprises or consists of SEQ ID NO: 17; (e) the VH amino acid sequence comprises or consists of SEQ ID NO: 16 and the VL amino acid sequence comprises or consists of SEQ ID NO: 17; (f) the VH amino acid sequence comprises or consists of SEQ ID NO: 16 and the VL amino acid sequence comprises or consists of SEQ ID NO: 18; (g) the VH amino acid sequence comprises or consists of SEQ ID NO: 16 and the VL amino acid sequence comprises or consists of SEQ ID NO: 19; (h) the VH amino acid sequence comprises or consists of SEQ ID NO: 16 and the VL amino acid sequence comprises or consists of SEQ ID NO: 20; (i) the VH amino acid sequence comprises or consists of SEQ ID NO: 21 and the VL amino acid sequence comprises or consists of SEQ ID NO: 18; (j) the VH amino acid sequence comprises or consists of SEQ ID NO: 21 and the VL amino acid sequence comprises or consists of SEQ ID NO: 19; (k) the VH amino acid sequence comprises or consists of SEQ ID NO: 21 and the VL amino acid sequence comprises or consists of SEQ ID NO: 20; (1) the VH amino acid sequence comprises or consists of SEQ ID NO: 22 and the VL amino acid sequence comprises or consists of SEQ ID NO: 18; (m) the VH amino acid sequence comprises or consists of SEQ ID NO: 22 and the VL amino acid sequence comprises or consists of SEQ ID NO: 19; or (n) the VH amino acid sequence comprises or consists of SEQ ID NO: 22 and the VL amino acid sequence comprises or consists of SEQ ID NO: 20.

In some embodiments, the present disclosure provides a humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid sequence selected from SEQ ID NO: 31; (b) the HCDR2 comprises an amino acid sequence selected from SEQ ID NO: 32; (c) the HCDR3 comprises the amino acid sequence selected from SEQ ID NO: 33; (d) the LCDR1 comprises the amino acid sequence selected from SEQ ID NO: 35; (e) the LCDR2 comprises the amino acid sequence selected from SEQ ID NO: 36; and (f) the LCDR3 comprises the amino acid sequence selected from SEQ ID NO: 37.

In some embodiments, the VH comprises an amino acid sequence is at least 95%, 96%, 97%, 98%, or 99% identical to one of SEQ ID NOs: 39 and 34 and wherein the VL comprises an amino acid sequence is at least 95%, 96%, 97%, 98%, or 99% identical to one of SEQ ID NOs: 40 and 41. In some embodiments, (a) the VH comprises an amino acid sequence is at least 95%, 96%, 97%, 98%, or 99% identical SEQ ID NO: 39 and the VL comprises an amino acid sequence is at least 95%, 96%, 97%, 98%, or 99% identical to SEQ ID NO: 40; (b) the VH comprises an amino acid sequence is at least 95%, 96%, 97%, 98%, or 99% identical SEQ ID NO: 39 and the VL comprises an amino acid sequence is at least 95%, 96%, 97%, 98%, or 99% identical to SEQ ID NO: 41; or (c) the VH comprises an amino acid sequence is at least 95%, 96%, 97%, 98%, or 99% identical SEQ ID NO: 34 and the VL comprises an amino acid sequence is at least 95%, 96%, 97%, 98%, or 99% identical to SEQ ID NO: 41.

In some embodiments, the VH comprises or consists of one of SEQ ID NOs: 39 and 34 and wherein the VL comprises or consists of one of SEQ ID NOs: 40 and 41. In some embodiments, (a) the VH amino acid sequence comprises or consists of SEQ ID NO: 39 and the VL amino acid sequence comprises or consists of SEQ ID NO: 40; (b) the VH amino acid sequence comprises or consists of SEQ ID NO: 39 and the VL amino acid sequence comprises or consists of SEQ ID NO: 41; or (c) the VH amino acid sequence comprises or consists of SEQ ID NO: 34 and the VL amino acid sequence comprises or consists of SEQ ID NO: 41.

In some embodiments, the present disclosure provides a humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid of SEQ ID NO: 42; (b) the HCDR2 comprises an amino acid sequence selected from SEQ ID NO: 43 or SEQ ID NO: 44, and (c) the HCDR3 comprises the amino acid sequence selected from any one of SEQ ID NOs: 45-56; (d) the LCDR1 comprises the amino acid sequence selected from SEQ ID NO: 58; (e) the LCDR2 comprises the amino acid sequence selected from SEQ ID NO: 59; and (f) the LCDR3 comprises the amino acid sequence selected from SEQ ID NO: 60.

In some embodiments, the present disclosure provides a humanized anti-IL-17A antibody or antigen-binding fragment thereof, wherein the LCDR1 comprises the amino acid sequence of SEQ ID NO: 58; the LCDR2 comprises the amino acid sequence of SEQ ID NO: 59; and the LCDR3 comprises the amino acid sequence of SEQ ID NO: 60; and wherein: (a) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 45; (b) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 46; (c) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 47; (d) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 48; (e) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 49; (f) the HCDR1 comprises an amino acid sequence selected from SEQ ID NO: 42; the HCDR2 comprises an amino acid sequence selected from SEQ ID NO: 44, and the HCDR3 comprises the amino acid sequence of SEQ ID NO: 50; (g) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 51; (h) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 52; (i) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 53; (j) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 54; (k) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 55; or (1) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 56.

In some embodiments, the VH comprises an amino acid sequence is at least 95%, 96%, 97%, 98%, or 99% identical to one of SEQ ID NOs: 62, 64, 65, 66, 67, 68, 69, 71, 72, 73, 74, 99, 77, 78, or 79 and wherein the VL comprises an amino acid sequence is at least 95%, 96%, 97%, 98%, or 99% identical to one of SEQ ID NOs: 63, 70, 75, 76, or 80. In some embodiments, (a) the VH comprises an amino acid sequence is at least 95%, 96%, 97%, 98%, or 99% identical SEQ ID NO: 62 and the VL comprises an amino acid sequence is at least 95%, 96%, 97%, 98%, or 99% identical to SEQ ID NO: 63; (b) the VH comprises an amino acid sequence is at least 95%, 96%, 97%, 98%, or 99% identical SEQ ID NO: 64 and the VL comprises an amino acid sequence is at least 95%, 96%, 97%, 98%, or 99% identical to SEQ ID NO: 63; (c) the VH comprises an amino acid sequence is at least 95%, 96%, 97%, 98%, or 99% identical SEQ ID NO: 65 and the VL comprises an amino acid sequence is at least 95%, 96%, 97%, 98%, or 99% identical to SEQ ID NO: 63; (d) the VH comprises an amino acid sequence is at least 95%, 96%, 97%, 98%, or 99% identical SEQ ID NO: 66 and the VL comprises an amino acid sequence is at least 95%, 96%, 97%, 98%, or 99% identical to SEQ ID NO: 63; (e) the VH comprises an amino acid sequence is at least 95%, 96%, 97%, 98%, or 99% identical SEQ ID NO: 67 and the VL comprises an amino acid sequence is at least 95%, 96%, 97%, 98%, or 99% identical to SEQ ID NO: 63; (f) the VH comprises an amino acid sequence is at least 95%, 96%, 97%, 98%, or 99% identical SEQ ID NO: 68 and the VL comprises an amino acid sequence is at least 95%, 96%, 97%, 98%, or 99% identical to SEQ ID NO: 63; (g) the VH comprises an amino acid sequence is at least 95%, 96%, 97%, 98%, or 99% identical SEQ ID NO: 69 and the VL comprises an amino acid sequence is at least 95%, 96%, 97%, 98%, or 99% identical to SEQ ID NO: 70; (h) the VH comprises an amino acid sequence is at least 95%, 96%, 97%, 98%, or 99% identical SEQ ID NO: 71 and the VL comprises an amino acid sequence is at least 95%, 96%, 97%, 98%, or 99% identical to SEQ ID NO: 70; (i) the VH comprises an amino acid sequence is at least 95%, 96%, 97%, 98%, or 99% identical SEQ ID NO: 72 and the VL comprises an amino acid sequence is at least 95%, 96%, 97%, 98%, or 99% identical to SEQ ID NO: 70; (j) the VH comprises an amino acid sequence is at least 95%, 96%, 97%, 98%, or 99% identical SEQ ID NO: 73 and the VL comprises an amino acid sequence is at least 95%, 96%, 97%, 98%, or 99% identical to SEQ ID NO: 70; (k) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 70; (1) the VH comprises an amino acid sequence is at least 95%, 96%, 97%, 98%, or 99% identical SEQ ID NO: 99 and the VL comprises an amino acid sequence is at least 95%, 96%, 97%, 98%, or 99% identical to SEQ ID NO: 70; (m) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 75; (n) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 76; (o) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 70; (p) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 75; (q) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 76; (r) the VH comprises an amino acid sequence is at least 95%, 96%, 97%, 98%, or 99% identical SEQ ID NO: 78 and the VL comprises an amino acid sequence is at least 95%, 96%, 97%, 98%, or 99% identical to SEQ ID NO: 70; (s) the VH comprises an amino acid sequence is at least 95%, 96%, 97%, 98%, or 99% identical SEQ ID NO: 79 and the VL comprises an amino acid sequence is at least 95%, 96%, 97%, 98%, or 99% identical to SEQ ID NO: 70; or (t) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 80.

In some embodiments, the VH comprises or consists of one of SEQ ID NOs: 62, 64, 65, 66, 67, 68, 69, 71, 72, 73, 74, 99, 77, 78, or 79 and wherein the VL comprises or consists of one of SEQ ID NOs: 63, 70, 75, 76, or 80. In some embodiments, (a) the VH amino acid sequence comprises or consists of SEQ ID NO: 62 and the VL amino acid sequence comprises or consists of SEQ ID NO: 63; (b) the VH amino acid sequence comprises or consists of SEQ ID NO: 64 and the VL amino acid sequence comprises or consists of SEQ ID NO: 63; (c) the VH amino acid sequence comprises or consists of SEQ ID NO: 65 and the VL amino acid sequence comprises or consists of SEQ ID NO: 63; (d) the VH amino acid sequence comprises or consists of SEQ ID NO: 66 and the VL amino acid sequence comprises or consists of SEQ ID NO: 63; (e) the VH amino acid sequence comprises or consists of SEQ ID NO: 67 and the VL amino acid sequence comprises or consists of SEQ ID NO: 63; (f) the VH amino acid sequence comprises or consists of SEQ ID NO: 68 and the VL amino acid sequence comprises or consists of SEQ ID NO: 63; (g) the VH amino acid sequence comprises or consists of SEQ ID NO: 69 and the VL amino acid sequence comprises or consists of SEQ ID NO: 70; (h) the VH amino acid sequence comprises or consists of SEQ ID NO: 71 and the VL amino acid sequence comprises or consists of SEQ ID NO: 70; (i) the VH amino acid sequence comprises or consists of SEQ ID NO: 72 and the VL amino acid sequence comprises or consists of SEQ ID NO: 70; ( ) the VH amino acid sequence comprises or consists of SEQ ID NO: 73 and the VL amino acid sequence comprises or consists of SEQ ID NO: 70; (k) the VH amino acid sequence comprises or consists of SEQ ID NO: 74 and the VL amino acid sequence comprises or consists of SEQ ID NO: 70; (1) the VH amino acid sequence comprises or consists of SEQ ID NO: 99 and the VL amino acid sequence comprises or consists of SEQ ID NO: 70; (m) the VH amino acid sequence comprises or consists of SEQ ID NO: 74 and the VL amino acid sequence comprises or consists of SEQ ID NO: 75; (n) the VH amino acid sequence comprises or consists of SEQ ID NO: 74 and the VL amino acid sequence comprises or consists of SEQ ID NO: 76; (o) the VH amino acid sequence comprises or consists of SEQ ID NO: 77 and the VL amino acid sequence comprises or consists of SEQ ID NO: 70; (p) the VH amino acid sequence comprises or consists of SEQ ID NO: 77 and the VL amino acid sequence comprises or consists of SEQ ID NO: 75; (q) the VH amino acid sequence comprises or consists of SEQ ID NO: 77 and the VL amino acid sequence comprises or consists of SEQ ID NO: 76; (r) the VH amino acid sequence comprises or consists of SEQ ID NO: 78 and the VL amino acid sequence comprises or consists of SEQ ID NO: 70; (s) the VH amino acid sequence comprises or consists of SEQ ID NO: 79 and the VL amino acid sequence comprises or consists of SEQ ID NO: 70; or (t) the VH amino acid sequence comprises or consists of SEQ ID NO: 74 and the VL amino acid sequence comprises or consists of SEQ ID NO: 80.

In some embodiments, the antigen binding fragment is a single chain variable fragment (scFv). In some embodiments, the scFv comprises an amino acid sequence that is at least 95%, 96%, 97%, 98%, or 99% identical to an amino acid sequence selected from SEQ ID NOs: 177-179. In some embodiments, the scFv comprises or consists of an amino acid sequence selected from SEQ ID NOs: 177-179.

In some embodiments, the present disclosure provides an anti-IL-17A single chain variable fragment (scFv) comprising an amino acid sequence that is at least 95%, 96%, 97%, 98%, or 99% identical to an amino acid sequence selected from SEQ ID NOs: 177-179. In some embodiments, the scFv comprises or consists of an amino acid sequence selected from SEQ ID NOs: 177-179.

In some embodiments, the present disclosure provides a polynucleotide encoding a anti-IL-17A antibody or antigen-binding fragment thereof or a humanized anti-IL-17A antibody or antigen-binding fragment thereof described herein. In some embodiments, the present disclosure provides a polynucleotide encoding the anti-IL-17A scFv described herein. In some embodiments, the present disclosure provides an expression vector comprising a polynucleotide described herein.

In some embodiments, the present disclosure provides host cell comprising a polynucleotide or expression vector described herein. In some embodiments, the present disclosure provides vector comprising a polynucleotide encoding a anti-IL-17A antibody or antigen-binding fragment thereof, a humanized anti-IL-17A antibody or antigen-binding fragment thereof, or a anti-IL-17A scFv described herein. In some embodiments, the vector is an AAV8 or AAV9 vector.

In some embodiments, the present disclosure provides a method of manufacturing the anti-IL-17A antibody or antigen-binding fragment thereof, the humanized anti-IL-17A antibody or antigen-binding fragment thereof, or the anti-IL-17A scFv described herein comprising introducing the expression vector described herein into a host cell.

In some embodiments, the present disclosure provides a bi-specific binding protein comprising a first and a second heavy chain polypeptide and a first and a second light chain polypeptide, wherein the first and second heavy chain polypeptides comprise, from N-terminus to C-terminus, an antigen-binding domain, a linker, and an anti-IL-17A antigen-binding domain. In some embodiments, the anti-IL-17A antigen-binding domain is an anti-IL-17A antibody heavy chain. In some embodiments, the antigen-binding domain is a VEGF-binding domain or a TNFα binding domain.

In some embodiments, the first and second heavy chain polypeptides comprise, from N-terminus to C-terminus, a VEGF antigen-binding domain comprising an amino acid sequence of SEQ ID NO: 167 or 168, a linker, and an anti-IL-17A antibody heavy chain comprising an amino acid sequence selected from SEQ ID NOs: 169-174. In some embodiments, the first and second heavy chain polypeptides comprise an amino acid sequence selected from SEQ ID NOs: 101-108.

In some embodiments, the first and second light chain polypeptides comprise an anti-IL-17A antibody light chain. In some embodiments, the first and second light chain polypeptides comprise the amino acid sequence selected from SEQ ID NO: 136 and 137.

In some embodiments, the first and second heavy chain polypeptide comprising an amino acid sequence selected from the group of SEQ ID NO: 101-108 and the first and second light chain polypeptide comprising an amino acid sequence selected from SEQ ID NO: 136 and 137. In some embodiments, (a) the bi-specific binding protein comprises a first and second heavy chain polypeptide comprising the amino acid sequence of SEQ ID NO: 101 and a first and second light chain polypeptide comprising the amino acid sequence of SEQ ID NO: 136; (b) the bi-specific binding protein comprises a first and second heavy chain polypeptide comprising the amino acid sequence of SEQ ID NO: 102 and a first and second light chain polypeptide comprising the amino acid sequence of SEQ ID NO: 136; (c) the bi-specific binding protein comprises a first and second heavy chain polypeptide comprising the amino acid sequence of SEQ ID NO: 103 and a first and second light chain polypeptide comprising the amino acid sequence of SEQ ID NO: 136; (d) the bi-specific binding protein comprises a first and second heavy chain polypeptide comprising the amino acid sequence of SEQ ID NO: 104 and a first and second light chain polypeptide comprising the amino acid sequence of SEQ ID NO: 136; (e) the bi-specific binding protein comprises a first and second heavy chain polypeptide comprising the amino acid sequence of SEQ ID NO: 105 and a first and second light chain polypeptide comprising the amino acid sequence of SEQ ID NO: 136; (f) the bi-specific binding protein comprises a first and second heavy chain polypeptide comprising the amino acid sequence of SEQ ID NO: 106 and a first and second light chain polypeptide comprising the amino acid sequence of SEQ ID NO: 136; (g) the bi-specific binding protein comprises a first and second heavy chain polypeptide comprising the amino acid sequence of SEQ ID NO: 107 and a first and second light chain polypeptide comprising the amino acid sequence of SEQ ID NO: 137; or (h) the bi-specific binding protein comprises a first and second heavy chain polypeptide comprising the amino acid sequence of SEQ ID NO: 108 and a first and second light chain polypeptide comprising the amino acid sequence of SEQ ID NO: 137.

In some embodiments, the first and second heavy chain polypeptides comprise, from N-terminus to C-terminus, a TNFα antigen-binding domain comprising an amino acid sequence of SEQ ID NO: 134, a linker, and an anti-IL-17A antibody heavy chain comprising an amino acid sequence selected from SEQ ID NOs: 180-185. In some embodiments, the first and second heavy chain polypeptides comprise an amino acid sequence selected from SEQ ID NOs: 127-132. In some embodiments, the first and second light chain polypeptides comprise an anti-IL-17A antibody light chain. In some embodiments, the first and second light chain polypeptides comprise an amino acid sequence selected from SEQ ID NO: 141 and 142.

In some embodiments, the first and second heavy chain polypeptide comprise the amino acid sequence of any one of SEQ ID NO: 127-132 and a first and second light chain polypeptide comprise the amino acid sequence of SEQ ID NO: 141 or 142. In some embodiments, (a) the bi-specific binding protein comprises a first and second heavy chain polypeptide comprising the amino acid sequence of SEQ ID NO: 127 and a first and second light chain polypeptide comprising the amino acid sequence of SEQ ID NO: 141; (b) the bi-specific binding protein comprises a first and second heavy chain polypeptide comprising the amino acid sequence of SEQ ID NO: 128 and a first and second light chain polypeptide comprising the amino acid sequence of SEQ ID NO: 141; (c) the bi-specific binding protein comprises a first and second heavy chain polypeptide comprising the amino acid sequence of SEQ ID NO: 129 and a first and second light chain polypeptide comprising the amino acid sequence of SEQ ID NO: 142; (d) the bi-specific binding protein comprises a first and second heavy chain polypeptide comprising the amino acid sequence of SEQ ID NO: 130 and a first and second light chain polypeptide comprising the amino acid sequence of SEQ ID NO: 142; (e) the bi-specific binding protein comprises a first and second heavy chain polypeptide comprising the amino acid sequence of SEQ ID NO: 131 and a first and second light chain polypeptide comprising the amino acid sequence of SEQ ID NO: 142; or (f) the bi-specific binding protein comprises a first and second heavy chain polypeptide comprising the amino acid sequence of SEQ ID NO: 132 and a first and second light chain polypeptide comprising the amino acid sequence of SEQ ID NO: 142.

In some embodiments, the present disclosure provides a bi-specific binding protein comprising a first and a second heavy chain polypeptide and a first and a second light chain polypeptide, wherein the first and second heavy chain polypeptides comprise, from N-terminus to C-terminus, an anti-IL-17A antigen-binding domain, a linker, and an antigen-binding domain. In some embodiments, the first and second heavy chain polypeptides comprise, from N-terminus to C-terminus, an anti-IL-17A antibody heavy chain, a linker, and an antigen-binding domain. In some embodiments, the antigen-binding domain is a VEGF-binding domain or a TNFα binding domain.

In some embodiments, the first and second heavy chain polypeptides comprise, from N-terminus to C-terminus, an anti-IL-17A antibody heavy chain comprising an amino acid sequence selected from SEQ ID NO: 169, 170, 175, and 176, a linker, and a VEGF antigen-binding domain comprising SEQ ID NO: 167 or 168. In some embodiments, the first and second heavy chain polypeptides comprise an amino acid sequence selected from SEQ ID NO: 109-116. In some embodiments, the first and second light chain polypeptides comprise an anti-IL-17A antibody light chain. In some embodiments, the first and second light chain polypeptides comprise an anti-IL-17A antibody light chain comprising the amino acid sequence of SEQ ID NO: 136 or 137.

In some embodiments, the first and second heavy chain polypeptide comprise an amino acid sequence selected from SEQ ID NO: 109-116 and the first and second light chain polypeptide comprise the amino acid sequence of SEQ ID NO: 136 or 137. In some embodiments, (a) the bi-specific binding protein comprises a first and second heavy chain polypeptide comprising the amino acid sequence of SEQ ID NO: 109 and a first and second light chain polypeptide comprising the amino acid sequence of SEQ ID NO: 136; (b) the bi-specific binding protein comprises a first and second heavy chain polypeptide comprising the amino acid sequence of SEQ ID NO: 110 and a first and second light chain polypeptide comprising the amino acid sequence of SEQ ID NO: 136; (c) the bi-specific binding protein comprises a first and second heavy chain polypeptide comprising the amino acid sequence of SEQ ID NO: 111 and a first and second light chain polypeptide comprising the amino acid sequence of SEQ ID NO: 136; (d) the bi-specific binding protein comprises a first and second heavy chain polypeptide comprising the amino acid sequence of SEQ ID NO: 112 and a first and second light chain polypeptide comprising the amino acid sequence of SEQ ID NO: 136; (e) the bi-specific binding protein comprises a first and second heavy chain polypeptide comprising the amino acid sequence of SEQ ID NO: 113 and a first and second light chain polypeptide comprising the amino acid sequence of SEQ ID NO: 137; (f) the bi-specific binding protein comprises a first and second heavy chain polypeptide comprising the amino acid sequence of SEQ ID NO: 114 and a first and second light chain polypeptide comprising the amino acid sequence of SEQ ID NO: 137; (g) the bi-specific binding protein comprises a first and second heavy chain polypeptide comprising the amino acid sequence of SEQ ID NO: 115 and a first and second light chain polypeptide comprising the amino acid sequence of SEQ ID NO: 137; or (h) the bi-specific binding protein comprises a first and second heavy chain polypeptide comprising the amino acid sequence of SEQ ID NO: 116 and a first and second light chain polypeptide comprising the amino acid sequence of SEQ ID NO: 137.

In some embodiments, the present disclosure provides a bi-specific binding protein comprising a first and a second heavy chain polypeptide and a first and a second light chain polypeptide, wherein the first and second light chains comprise, from N-terminus to C-terminus, an antigen binding domain, a linker, and an anti-IL-17A antigen binding domain. In some embodiments, the first and second heavy chain polypeptides comprise an anti-IL-17A antibody heavy chain. In some embodiments, the first and second heavy chain polypeptides comprise an anti-IL-17A antibody heavy chain comprising an amino acid sequence selected from SEQ ID NO: 117-120. In some embodiments, the anti-IL-17A antigen binding domain is an anti-IL-17A antibody light chain. In some embodiments, the antigen-binding domain is a VEGF-binding domain or a TNFα binding domain.

In some embodiments, the first and second light chains comprise, from N-terminus to C-terminus, a VEGF binding domain comprising SEQ ID NO: 167, a linker, and an anti-IL-17A antibody light chain comprising an amino acid sequence selected from SEQ ID NO: 136 and 137. In some embodiments, the first and second light chain polypeptides comprise an amino acid sequence selected from SEQ ID NO: 138 and 139.

In some embodiments, the first and second heavy chain polypeptides comprise an amino acid sequence selected from SEQ ID NO: 117-120 and the first and second light chain polypeptides comprise an amino acid sequence selected from SEQ ID NO: 138 and 139. In some embodiments, (a) the first and second heavy chain polypeptides comprise an amino acid sequence selected from SEQ ID NO: 117 and the first and second light chain polypeptides comprise an amino acid sequence selected from SEQ ID NO: 138; (b) the first and second heavy chain polypeptides comprise an amino acid sequence selected from SEQ ID NO: 118 and the first and second light chain polypeptides comprise an amino acid sequence selected from SEQ ID NO: 138; (c) the first and second heavy chain polypeptides comprise an amino acid sequence selected from SEQ ID NO: 119 and the first and second light chain polypeptides comprise an amino acid sequence selected from SEQ ID NO: 139; or (d) the first and second heavy chain polypeptides comprise an amino acid sequence selected from SEQ ID NO: 120 and the first and second light chain polypeptides comprise an amino acid sequence selected from SEQ ID NO: 139.

In some embodiments, the present disclosure provides a bi-specific binding protein comprising a first and a second protein monomer, wherein each monomer comprises, from N-terminus to C-terminus, an antigen binding domain, a linker, and an IL-17A binding domain. In some embodiments, the antigen-binding domain is a VEGF-binding domain or a TNFα binding In some embodiments, the first and second monomers comprise, from N-terminus to C-terminus, a VEGF binding domain comprising SEQ ID NO: 100, a linker, and an anti-IL-17A scFv binding domain comprising an amino acid sequence selected from SEQ ID NO: 177-179. In some embodiments, each monomer comprises an amino acid sequence that is at least 90%, 95%, 96%, 97%, 98%, or 99% identical to an amino acid sequence selected from SEQ ID NO: 123-125. In some embodiments, each monomer comprises or consists of an amino acid sequence selected from SEQ ID NO: 123-125. In some embodiments, each monomer comprises an amino acid sequence that is at least 90%, 95%, 96%, 97%, 98%, or 99% identical to SEQ ID NO: 124. In some embodiments, each monomer comprises or consists of SEQ ID NO: 124. In some embodiments, each monomer comprises an amino acid sequence that is at least 90%, 95%, 96%, 97%, 98%, or 99% identical to SEQ ID NO: 125. In some embodiments, each monomer comprises or consists of SEQ ID NO: 125.

In some embodiments, the VEGF antigen-binding domain is a soluble VEGF receptor or an anti-VEGF antibody or antigen-binding fragment thereof. In some embodiments, the soluble VEGF receptor is aflibercept. In some embodiments, the anti-VEGF antibody is selected from bevacizumab, ranibizumab, brolucizumab, and ramucirumab. In some embodiments, the TNFα antigen-binding domain is an anti-TNFα antibody or antigen binding fragment thereof. In some embodiments, the anti-TNFα antibody is selected from adalimumab, infliximab, etanercept, golimumab, and certolizumab.

In some embodiments, the present disclosure provides a polynucleotide encoding a bi-specific binding protein described herein. In some embodiments, the present disclosure provides an expression vector comprising a polynucleotide described herein. In some embodiments, the present disclosure provides a host cell comprising a polynucleotide or an expression vector described herein.

In some embodiments, the present disclosure provides method of manufacturing the bi-specific binding protein described herein, comprising introducing an expression vector into a host cell.

In some embodiments, the present disclosure provides a vector comprising a polynucleotide encoding a the bi-specific binding protein described herein. In some embodiments, the vector is an AAV8 or AAV9 vector.

In some embodiments, the present disclosure provides a method of treating an inflammatory condition in a subject in need thereof, comprising administering an anti-IL-17A antibody or antigen-binding fragment thereof, a humanized anti-IL-17A antibody or antigen-binding fragment thereof, an anti-IL-17A scFv, a bi-specific protein, or a vector described herein, to a subject in need thereof.

In some embodiments, the present disclosure provides a use of an anti-IL-17A antibody or antigen-binding fragment thereof, a humanized anti-IL-17A antibody or antigen-binding fragment thereof, an anti-IL-17A scFv, a bi-specific protein, or a vector described herein for the manufacture of a medicament for treatment of an inflammatory condition in a subject.

In some embodiments, the inflammatory condition is selected from airway inflammation, rheumatoid arthritis, osteoarthritis, bone erosion, intraperitoneal abscesses and adhesions, infectious disease, inflammatory bowel disorder, macular degeneration, allograft rejection, psoriasis, cancer, angiogenesis, atherosclerosis, and multiple sclerosis.

In some embodiments, the present disclosure provides a method of treating an ocular disease in a subject in need thereof, comprising administering an anti-IL-17A antibody or antigen-binding fragment thereof, a humanized anti-IL-17A antibody or antigen-binding fragment thereof, an anti-IL-17A scFv, a bi-specific protein, or a vector described herein, to a subject in need thereof.

In some embodiments, the present disclosure provides a use of an anti-IL-17A antibody or antigen-binding fragment thereof, a humanized anti-IL-17A antibody or antigen-binding fragment thereof, an anti-IL-17A scFv, a bi-specific protein, or a vector described herein for the manufacture of a medicament for treatment of an ocular disease in a subject.

In some embodiments, the ocular disease is selected from macular degeneration, retinitis pigmentosa, and diabetic retinopathy. In some embodiments, the macular degeneration is age-related macular degeneration. In some embodiments, the macular degeneration is wet or dry.

In some embodiments, the present disclosure provides a method of decreasing vascular leakage in the eye in a subject in need thereof, comprising administering an anti-IL-17A antibody or antigen-binding fragment thereof, a humanized anti-IL-17A antibody or antigen-binding fragment thereof, an anti-IL-17A scFv, a bi-specific protein, or a vector described herein.

In some embodiments, the present disclosure provides a use of an anti-IL-17A antibody or antigen-binding fragment thereof, a humanized anti-IL-17A antibody or antigen-binding fragment thereof, an anti-IL-17A scFv, a bi-specific protein, or a vector described herein for the manufacture of a medicament for decreasing vascular leakage in the eye in a subject in need thereof.

In some embodiments, the antibody, antigen-binding fragment thereof, scFv, bi-specific protein, or vector are administered intravenously, intravitreally, subcutaneously, or intramuscularly. In some embodiments, the subject is human.

### BRIEF DESCRIPTION OF THE DRAWINGS

The accompanying drawings, which are incorporate in and constitute a part of this specification, illustrate several embodiments and together with the description illustrate the disclosed compositions and methods.

FIG. 1 shows a sort plot of B cells binding to IL17.

FIG. 2A-FIG. 2B shows the expression levels of monoclonal antibodies in HEK-EXPI cells and denatured gel electrophoresis of antibodies. FIG. 2A shows a human IgG4 positive control. FIG. 2B shows Kaleidoscope pre-stained standard protein marker and lane 1: ABM58; lane 2: ABM59, lane 3: ABM60; lane 4: ABM62; lane 5: ABM64; lane 6: ABM67; lane 7: ABM70; lane 8: ABM72; lane 9: ABM73; lane 10: ABM74; lane 11: ABM79; lane 12: Ixekizumab; and lane 13: Secukinumab.

FIG. 3 Shows a HT-29 cellular potency assay of monoclonal antibodies against IL-17A.

FIG. 4 shows a HT-NIH-3T3 cellular potency assay of monoclonal antibodies against IL-17A.

FIG. 5 shows the Surface plasmon resonance (SPR) of anti-IL17 monoclonal antibodies and calculated affinity of the antibodies.

FIG. 6A-FIG. 6B show the results of VEGF and IL-17A bioactivity assays. FIG. 6A shows the anti-VEGF activity of various bi-specific proteins described herein. FIG. 6B shows the anti-IL-17A activity of various bi-specific proteins described herein.

FIG. 7 provides a schematic of an exemplary anti-VEGF/anti-IL-17A bi-specific protein described herein (653.1).

FIG. 8A-FIG. 8B show fluorescein angiography images from aflibercept (FIG. 8A) and 653.1 (FIG. 8B) treated eyes in an AAA-induced vascular leakage rabbit model.

FIG. 9A-FIG. 9B provides a quantification of the leakage observed in FIG. 8 for very leaky (FIG. 9A) and minimally leaky (FIG. 9B) eyes.

FIG. 10A-FIG. 10D provide summaries of ocular examination scores in treated and untreated eyes (FIG. 10A), anterior and posterior segments (FIG. 10B), and changes in intraocular pressure (FIG. 10C and FIG. 10D) after treatment with aflibercept or 653.1.

FIG. 11A-FIG. 11C provide exemplary schematics of the bi-specific proteins described herein.

### DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide.

The term “antigen-binding fragment” as used herein refers to a polypeptide fragment that contains at least one Complementarity-determining region (CDR) of an immunoglobulin heavy and/or light chain that binds to at least one epitope of the antigen of interest. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind IL-17A. Antigen-binding fragments include proteins that comprise a portion of a full length antibody, generally the antigen binding or variable region thereof, such as Fab, F(ab′)2, Fab′, Fv fragments, minibodies, diabodies, single domain antibody (dAb), single-chain variable fragments (scFv), multispecific antibodies formed from antibody fragments (e.g., bispecific antibodies), and any other modified configuration of the immunoglobulin molecule that comprises an antigen-binding site or fragment of the required specificity.

The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

The term “F(ab)” refers to two of the protein fragments resulting from proteolytic cleavage of IgG molecules by the enzyme papain. Each F(ab) comprises a covalent heterodimer of the VH chain and VL chain and includes an intact antigen-binding site. Each F(ab) is a monovalent antigen-binding fragment. The term “Fab′” refers to a fragment derived from F(ab′)2 and may contain a small portion of Fc. Each Fab′ fragment is a monovalent antigen-binding fragment.

The term “F(ab′)2” refers to a protein fragment of IgG generated by proteolytic cleavage by the enzyme pepsin. Each F(ab′)2 fragment comprises two F(ab′) fragments and is therefore a bivalent antigen-binding fragment.

An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096. In some embodiments, the Fv fragment can be produced by preferential proteolytic cleavage of an IgM, and on rare occasions of an IgG or IgA immunoglobulin molecule. Fv fragments are, however, more commonly derived using recombinant techniques known in the art.

Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The term “diabody” refers to a bispecific antibody in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)).

The term “nanobody” or a “single domain antibody” refers to an antigen-binding fragment consisting of a single monomeric variable antibody domain. The Nanoclone method is a method for generating Nanobodies against a desired target based on automated high-throughput selection of B-cells. (See, WO 2006/079372)

The term “monoclonal antibody” as used herein refers to an antibody obtained from a population of substantially homogeneous antibodies, i.e., the individual antibodies comprising the population are identical except for possible naturally occurring mutations that may be present in minor amounts.

In some embodiments, the term “chimeric antibody” as used herein refers to a monoclonal antibody in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity.

The term “single chain variable fragment” or “scFv” refers to a fusion protein of the variable regions of the heavy (VH) and light chains (VL) of immunoglobulins, connected with a short linker peptide of ten to about 25 amino acids. Huston et al. (1988) Proc. Nat. Acad. Sci. USA 85(16):5879-5883. The linker can connect the N-terminus of the VH with the C-terminus of the VL, or vice versa. A number of methods have been described to discern chemical structures for converting the naturally aggregated—but chemically separated-light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

The term “antigen” refers to a molecule or a portion of a molecule capable of being bound by an antibody or an antigen-binding fragment thereof and additionally capable of being used in an animal to produce antibodies capable of binding to an epitope of that antigen. An antigen may have one or more epitopes.

The term “epitope” refers to a region of an antigen that is bound by an antibody. Epitope determinants can include chemically active surface groupings of molecules such as amino acids, sugar side chains, phosphoryl, or sulfonyl and may have specific three-dimensional structural characteristics, and/or specific charge characteristics.

Herein, the term “specifically binds” refers to the ability of an antibody or antigen-binding fragment thereof to bind a target antigen with a binding affinity (Ka) of at least 10⁵ M⁻¹ while not significantly binding other components or antigens present in a mixture. Reference to an anti-IL-17A antibody herein refers to an antibody or antigen-binding fragment thereof that specifically binds to IL-17A. An antibody specifically binds to an antigen may bind to other peptides or polypeptides with lower affinity as determined by, e.g., radioimmunoassays (RIA), enzyme-linked immunosorbent assays (ELISA), BIAcore, or other assays known in the art. Antibodies that specifically bind to an antigen may be cross-reactive with related antigens.

Binding affinity (Ka) refers to an equilibrium association of a particular interaction expressed in the units of 1/M or M⁻¹. Antibodies or antigen-binding fragments thereof can be classified as “high affinity” antibodies or antigen-binding fragments thereof and “low affinity” antibodies or antigen-binding fragments thereof “High affinity” antibodies or antigen-binding fragments thereof refer to those antibodies or antigen-binding fragments thereof with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” antibodies or antigen-binding fragments thereof refer to those antibodies or antigen-binding fragments thereof with a Ka of up to 107 M⁻¹, up to 10⁶ M⁻¹, up to 10⁵ M⁻¹. Alternatively, affinity can be defined as an equilibrium dissociation constant (Kd) of a particular binding interaction with units of M (e.g., 10⁻⁵ M to 10⁻¹³, or about 500 nM, about 300 nM, about 250 nM, about 200 nM, about 150 nM, about 100 nM, about 50 nM, about 25 nM, about 10 nM, or about 5 nM). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

A “conservative substitution” is recognized in the art as a substitution of one amino acid for another amino acid that has similar properties. Exemplary conservative substitutions are well-known in the art (see, e.g., PCT Application Publication No. WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8).

As used herein, the term “derivative” refers to a modification of one or more amino acid residues of a peptide by chemical or biological means, either with or without an enzyme, e.g., by glycosylation, alkylation, acylation, ester formation, or amide formation.

As used herein, a polypeptide or polynucleotide from which another polypeptide or polynucleotide is derived from is referred to as the “parental” or “reference” polynucleotide or polypeptide. For example, a humanized antibody can be derived from a parental murine antibody.

The term “variant” or “variants” as used herein refers to a polynucleotide or polypeptide with a sequence differing from that of a reference polynucleotide or polypeptide but retaining essential properties of the parental polynucleotide or polypeptide. Generally, variant polynucleotide or polypeptide sequences are overall closely similar, and, in many regions, identical to the parental polynucleotide or polypeptide. For instance, a variant polynucleotide or polypeptide may exhibit at least about 70%, at least about 80%, at least about 90%, at least about 91%, at least about 92%, at least about 93%, at least about 94%, at least about 95%, at least about 96%, at least about 97%, at least about 98% at least about 99%, or at least about 99.5% sequence identity compared to the parental polynucleotide or polypeptide.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

“Fc region” or “Fc domain” refers to a polypeptide sequence corresponding to or derived from the portion of an antibody that is capable of binding to Fc receptors on cells and/or the C1q component of complement, thereby mediating the effector function of an antibody. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc region is a homodimeric protein comprising two polypeptides that are associated by disulfide bonds, and each comprising a hinge region, a CH2 domain, and a CH3 domain. However, more recently the term has been applied to the single chain monomer component consisting of CH3, CH2, and at least a portion of the hinge sufficient to form a disulfide-linked dimer with a second such chain. As such, and depending on the context, use of the terms “Fc region” or “Fc domain” will refer herein to either the dimeric form or the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc domain includes variants of naturally occurring sequences.

The term “immunoglobulin constant region” or “constant region” refers to a peptide or polypeptide sequence that corresponds to or is derived from part or all of one or more constant domains of an immunoglobulin (e.g., CH1, CH2, CH3). In certain embodiments, the constant region does not comprise a CH1 domain. In certain embodiments, the constant domains making up the constant region are human

The terms “light chain variable region” (also referred to as “light chain variable domain” or “VL”) and “heavy chain variable region” (also referred to as “heavy chain variable domain” or “VH”) refer to the variable binding region from an antibody light and heavy chain, respectively. The variable binding regions are made up of discrete, well-defined sub-regions known as “complementarity determining regions” (CDRs) and “framework regions” (FRs).

The term “immunoglobulin light chain constant region” (also referred to as “light chain constant region” or “CL”) is a constant region from an antibody light chain.

The term “immunoglobulin heavy chain constant region” (also referred to as “heavy chain constant region” or “CH”) refers to the constant region from the antibody heavy chain. The CH is further divisible, depending on the antibody isotype into CH1, CH2, and CH3 (IgA, IgD, IgG), or CH1, CH2, CH3, and CH4 domains (IgE, IgM).

As used herein, the term “complementarity determining region” or “CDR” refer to an immunoglobulin (antibody) molecule. There are three CDRs per variable domain: CDR1, CDR2 and CDR3 in the variable domain of the light chain and CDR1, CDR2 and CDR3 in the variable domain of the heavy chain.

As used herein, the term “humanized” refers to an antibody or antigen-binding fragment thereof derived from a non-human species that retains the antigen-binding properties of the original non-human antibody. In some embodiments, the binding fragments of an antibody (e.g., light and heavy chain variable regions, Fab, scFv) are humanized. Non-human antigen-binding fragments can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312). If derived from a non-human source, other regions of the antibody, such as the hinge region and constant region domains, can also be humanized.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The term “polynucleotide” as referred to herein means single-stranded or double-stranded nucleic acid polymers. In certain embodiments, the nucleotides comprising the polynucleotide can be RNA or DNA or a modified form of either type of nucleotide, such as a modified messenger RNA. Said modifications may include, but are not limited to, base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

As used herein, a “polypeptide” or “protein” refers to a single, linear, and contiguous arrangement of covalently linked amino acids. Polypeptides can form one or more intrachain disulfide bonds. The terms polypeptide and protein also encompass embodiments where two polypeptide chains link together in a non-linear fashion, such as via an interchain disulfide bond. Herein, a protein or polypeptide may be an antibody or an antigen-binding fragment of an antibody.

As used herein, the term “transformation,” “transfection,” and “transduction” refer to the transfer of a polynucletide into a cell. As used herein, the term “genetic transformation” refers to the transfer and incorporation of DNA, especially recombinant DNA, into a cell. The transferred nucleic acid can be introduced into a cell via an expression vector.

As used herein, the terms “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual or prevent onset of additional associated diseases. Herein, treatment can refer to a 10%, 20%, 30%, 40%, 50%, 60%, 70%, 80%, 90%, or 100% reduction in the severity of an established infection or a symptom of the infection. For example, a method for treating an inflammatory condition or cancer is considered to be a treatment if there is a 10% reduction in one or more symptoms of the condition or cancer in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the condition or disease or symptoms of the condition or disease. It is understood and herein contemplated that treatments as discussed herein can be prophylactic or therapeutic.

As used in this specification and the appended claims, the singular forms “a,” “an” and “the” include plural references unless the content clearly dictates otherwise.

As used in this specification, the term “and/or” is used in this disclosure to either “and” or “or” unless indicated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Anti-IL-17A Antibodies and Antigen-Binding Fragments Thereof

The present disclosure provides binding antibodies or antigen-binding fragments thereof that specifically bind to IL-17A. Such antibodies are also referred to herein as anti-IL-17A antibodies or antigen binding fragments thereof. In some embodiments, the anti-IL-17A antibodies or antigen binding fragments thereof are humanized.

IL-17A (Gene ID: 3605; NCBI Ref Seq. NP_002181.1; SEQ ID NO: 1) is a 20-30 kD homodimeric glycoprotein produced predominantly by activated CD4+ T cells and functions as a proinflammatory cytokine. IL-17A is secreted by activated T cells at sites of inflammation not in the systemic circulation. IL-17A binds to a type I transmembrane receptor termed IL-17R which is a large ubiquitously expressed protein that demonstrates no significant sequence similarity to other known cytokine receptors.

IL-17A has multiple biologic properties including upregulating adhesion molecules, production of inflammatory molecules, chemokines, antimicrobial peptides, and remodeling proteins, and inducing the production of multiple inflammatory cytokines and chemokines from various cell types including synoviocytes, chondrocytes, fibroblasts, endothelial cells, epithelial cells, keratinocytes, and macrophages. Also, IL-17A induces recruitment of neutrophils to an inflammatory site through induction of chemokine release, stimulates production of prostaglandins and metalloproteinases, and inhibits proteoglycan synthesis. This cytokine also regulates the activities of NF-κB and mitogen-activated protein (MAP) kinases and can stimulate the expression of IL6 and cyclooxygenase-2 (PTGS2/COX-2), as well as enhance the production of nitric oxide (NO). Furthermore, IL-17A plays an important role in the maturation of hematopoietic progenitor cells. It has been demonstrated that IL-17A has signaling roles in different organs and tissues including lung, articular cartilage, bone, brain, hematopoietic cells, kidney, skin, and intestine.

Increased levels of IL-17A have also been associated with several conditions, diseases or disorders including infectious diseases, airway inflammation, rheumatoid arthritis (“RA”), osteoarthritis, bone erosion, intraperitoneal abscesses and adhesions, inflammatory bowel disorder (“IBD”), allograft rejection, psoriasis, certain types of cancer, angiogenesis, atherosclerosis, and multiple sclerosis (“MS”). High levels of this cytokine are associated with several chronic inflammatory diseases including rheumatoid arthritis, psoriasis, and multiple sclerosis. The lung damage induced by the severe acute respiratory syndrome coronavirus 2 (SARS-CoV-2) is to a large extent, a result of the inflammatory response promoted by cytokines such as IL17A. Given its localized distribution at the site of inflammation, IL-17A appears to be a novel target for the treatment of RA and other inflammatory or autoimmune diseases with a potentially greater safety profile than drugs that target the systemic circulation of proinflammatory cytokines such as TNF-α.

The IL-17 family of cytokines presently includes IL-17A, IL-17B, IL-17C, IL-17D, IL-17E and IL-17F. All IL-17 family members have four highly conserved cysteine residues that are involved in the formation of intrachain disulfide linkages and have two or more cysteine residues that may be involved in interchain disulfide linkages. Members of the IL-17 family have no sequence similarity to any other known cytokines.

In some embodiments, the present disclosure provides anti-IL-17A antibodies and antigen binding fragments thereof, as well as bi-specific binding proteins comprising the same. As used herein, the term “antibody” encompasses, but is not limited to, whole immunoglobulin (i.e., an intact antibody) of any class. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with IL-17A such that IL-17 is inhibited from interacting with IL-17RA and/or IL-17RC.

Native antibodies are usually heterotetrameric glycoproteins, composed of two identical light (L) chains and two identical heavy (H) chains. The disclosed anti-IL-17A antibodies, whether monoclonal antibodies, polyclonal antibodies, chimeric antibodies, humanized or human antibodies, as well as antibodies fragments and functional variants can comprise all or a portion of light and heavy chains.

In a complete antibody, typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant (C(H)) domains. Each light chain has a variable domain at one end (V(L)) and a constant(C(L)) domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (1), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used to describe certain domains of the heavy and light chains that differ in sequence among antibodies and are used in the binding and specificity of each particular antibody for its particular antigen. However, the variability is not usually evenly distributed through the variable domains of antibodies. The more highly conserved portions of the variable domains are called the framework (FR). The variable domains of native heavy and light chains each comprise four FR regions, largely adopting a 3-sheet configuration, connected by three complementarity determining regions (CDRs), which form loops connecting, and in some cases forming part of, the 3-sheet structure. The variability is typically concentrated in the CDRs or hypervariable regions both in the light chain and the heavy chain variable domains.

The CDRs in each chain are held together in close proximity by the FR regions and, with the CDRs from the other chain, contribute to the formation of the antigen binding site of antibodies (see Kabat E. A. et al Institutes of Health, Bethesda, Md. (1987)). The CDR regions can be specific for linear epitopes, discontinuous epitopes, or conformational epitopes of proteins or protein fragments, either as present on the protein in its native conformation or, in some cases, as present on the proteins as denatured, e.g., by solubilization in SDS. Epitopes may also consist of posttranslational modifications of proteins.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

The constant domains are not involved directly in binding an antibody to an antigen, but exhibit various effector functions, such as participation of the antibody in antibody-dependent cellular toxicity.

It is understood and herein contemplated that the disclosed CDRs of the heavy chain variable domains in the disclosed anti-IL-17A antibodies or antigen binding fragments thereof can be contiguous or separated by 1, 2, 3, 4, 5, 6, 7,8, 9, 10, 11, 12, 13, 14, 15, 16, 17, 18, 19, 20, 21, 22, 23, 24, 25, 26, 27, 28, 29, 30, 31, 32, 33, 34, 35,36, 37, 38, 39, or 40 amino acids. Thus, disclosed herein are anti-IL-17A antibodies or antigen binding fragments comprising heavy chain variable domains comprising at least two CDRs wherein the first CDR is separated from the second CDR by 10, 11, 12, 13, 14, 15,16, 17, 18, 19, or 20 amino acids. For example, disclosed herein are anti-IL-17A antibodies or antigen binding fragments comprising at least two CDRs wherein the first CDR comprises SEQ ID NO: 2, 23, 31, 42, 81, or 91 and the second CDR comprises SEQ ID NO: 3, 24, 32,43, 44, 82, or 86, and wherein the first CDR and the second CDR are separated by 10, 11, 12, 13, 14, 15,16, 17, 18, 19, or 20 amino acids. Also disclosed herein are anti-IL-17A antibodies or antigen binding fragments comprising three CDRs wherein the first CDR comprises SEQ ID NO: 2, 23, 31, 42, 81, or 91; the second CDR comprises SEQ ID NO: 3, 24, 32,43, 44, 82, or 86; and the third CDR comprises SEQ ID NO: 4, 25, 33, 45, 46, 47, 48, 49, 50, 51, 52, 53, 54, 55, 56, 83, 87, 92, or 96; and wherein the second CDR and the third CDR are separated by 25, 26, 27, 28, 29, 30, 31, 32, 33, 34, 35, 36, 37, 38, 39, or 40 amino acids.

It is understood and herein contemplated that the disclosed CDRs of the light chain variable domains in the disclosed anti-IL-17A antibodies or antigen binding fragments can be contiguous or separated by 1, 2, 3, 4, 5, 6, 7,8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. Thus, disclosed herein are anti-IL-17A antibodies or antigen binding fragments comprising light chain variable domains wherein the light chain variable domain comprises at least two CDRs wherein the first CDR is separated from the second CDR by 10, 11, 12, 13, 14, 15,16, 17, 18, 19, or 20 amino acids. For example, disclosed herein are anti-IL-17A antibodies or antigen binding fragments comprising at least two CDRs wherein the first CDR comprises SEQ ID NO: 6, 10, 27, 35, or 58 and the second CDR comprises SEQ ID NO: 7, 28, 36, or 59, and wherein the first CDR and the second CDR are separated by 10, 11, 12, 13, 14, 15,16, 17, 18, 19, or 20 amino acids. Also disclosed herein are anti-IL-17A antibodies or antigen binding fragments comprising a light chain variable domain wherein the light chain variable domain comprises three CDRs wherein the first CDR comprises SEQ ID NO: 6, 10, 27, 35, or 58; the second CDR comprises SEQ ID NO: 7, 28, 36, or 59; and the third CDR comprises SEQ ID NO: 8, 29, 37, 60, 89, or 94; and wherein the second CDR and the third CDR are separated by 25, 26, 27, 28, 29, 30, 31, 32, 33, 34, 35, 36, 37, 38, 39, or 40 amino acids.

As noted above the disclosed IL-17 binding molecules can also be fragments of antibodies. As antibodies and hybrid antibodies, with dual or multiple antigen or epitope specificities, and fragments, such as F(ab′)2, Fab′, Fab, Fv, sFv, dAb, complementarity determining region (CDR) fragments, single-chain antibodies (scFv), bivalent single-chain antibodies, diabodies, triabodies, tetrabodies, (poly)peptides that contain at least a fragment of an immunoglobulin that is sufficient to confer specific antigen binding to the (poly)peptide, etc., including hybrid fragments. Thus, fragments of the antibodies that retain the ability to bind their specific antigens are provided. For example, fragments of antibodies which maintain IL-17A binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies). Conjugated antibodies or fragments refer to antibodies or fragments that are operatively linked or otherwise physically or functionally associated with an effector moiety or tag, such as inter alia a toxic substance, a radioactive substance, fluorescent substance, a liposome, or an enzyme as described, for example, in U.S. Pat. No. 4,704,692, the contents of which are hereby incorporated by reference.

Regardless of structure, the antigen-binding fragments disclosed herein can bind with the same antigen that is recognized by the intact immunoglobulin. An antigen-binding fragment can comprise a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 35 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least 200 contiguous amino acid residues, or at least contiguous 250 amino acid residues of the amino acid sequence of the binding molecule.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As discussed herein there are numerous variants and derivatives of the antibodies, CDRs, VH, and VL proteins disclosed herein. Protein variants and derivatives are well understood to those of skill in the art and in can involve amino acid sequence modifications. For example, amino acid sequence modifications typically fall into one or more of three classes: substitutional, insertional or deletional variants. As used herein, “insertions” refer to a change in an amino acid or nucleotide sequence resulting in the addition of one or more amino acid or nucleotide residues, respectively, as compared to the parent, often the naturally occurring, molecule. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions, or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions - Exemplary Conservative Substitutions

| Original AA | Conservative Substitution |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table B, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. Conservative amino acid substitutions include the ones in which the amino acid residue is replaced with an amino acid residue having similar structural or chemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cystine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

The replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. In addition, for example, a disclosed conservative derivative of SEQ ID NOs: 1-185 such as the substitution of an isoleucine (I) at for a valine (V). It is understood that for this mutation all of the nucleic acid sequences that encode this particular derivative of the SEQ ID NOs: 1-185 are also disclosed.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

In some embodiments, the present disclosure provides an antibody or antigen-binding fragment that specifically binds to IL-17A and comprises an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein the HCDR1 comprises an amino acid sequence selected from SEQ ID NO: 2, 23, 31, 42, 81, and 91; the HCDR2 comprises an amino acid sequence selected from SEQ ID NO: 3, 24, 32, 43, 44, 82, and 86; the HCDR3 comprises the amino acid sequence of SEQ ID NO: 4, 25, 33, 45, 46, 47, 48, 49, 50, 51, 52, 53, 54, 55, 56, 83, 87, 92, and 96; the LCDR1 comprises the amino acid sequence of SEQ TD NO: 6, 10, 27, 35, and 58; the LCDR2 comprises the amino acid sequence of SEQ TD NO: 7, 28, 26, and 59; and the LCDR3 comprises the amino acid sequence of SEQ ID NO: 8, 29, 37, 60, 89, and 94.

The CDRs of exemplary anti-IL-17A antibodies are shown in Table 3.

TABLE 3
Exemplary anti-IL-17A CDR sequences

| | | | SEQ |
|---|---|---|---|
| Construct | Component | Sequence | ID |
| ABM58 | HCDR1 | SYDMS | 2 |
| ABM58 | HCDR2 | YISSGGGNTYYPDTVKG | 3 |
| ABM58 | HCDR3 | EALTMISTRDYYAMDY | 4 |
| ABM58 | LCDR1 | RASESVDNYGISEMN | 6 |
| ABM58 | LCDR2 | AASNQGS | 7 |
| ABM58 | LCDR3 | QQSKEVPYT | 8 |
| ABM59 | HCDR1 | SYDMS | 2 |
| ABM59 | HCDR2 | YISSGGGNTYYPDTVKG | 3 |
| ABM59 | HCDR3 | EALTMISTRDYYAMDY | 4 |
| ABM59 | LCDR1 | GASESVDNYGISFMD | 10 |
| ABM59 | LCDR2 | AASNOGS | 7 |
| ABM59 | LCDR3 | QQSKEVPYT | 8 |
| ABM60 | HCDR1 | TYGVH | 23 |
| ABM60 | HCDR2 | VIWSDGLTTYNSALKS | 24 |
| ABM60 | HCDR3 | DYTYEYAMDY | 25 |
| ABM60 | LCDR1 | RSSQSLVHSNGNTYLH | 27 |
| ABM60 | LCDR2 | KVSNRFS | 28 |
| ABM60 | LCDR3 | SQSTHVPFT | 29 |
| ABM64 | HCDR1 | DYAMH | 31 |
| ABM64 | HCDR2 | VISTYSGNINYNQKFKG | 32 |
| ABM64 | HCDR3 | GNYGSD | 33 |
| ABM64 | LCDR1 | KSSQSLLNSSNQKNYLA | 35 |
| ABM64 | LCDR2 | FASTRES | 36 |
| ABM64 | LCDR3 | QQHYSTPYT | 37 |
| ABM67 | HCDR1 | SYAMS | 42 |
| ABM67 | HCDR2 | TISSGGSYTYYPDSVKG | 43 |
| ABM67 | HCDR2 - Y57H, | TISSGGSHTYYPDSAKG | 44 |
| | V64A | | |
| ABM67 | HCDR3 | QNWDAFVY | 45 |
| ABM67 | HCDR3 - Q99R | RNWDAFVY | 46 |
| ABM67 | HCDR3 - N100G | QGWDAFVY | 47 |
| ABM67 | HCDR3 - D102R | QNWRAFVY | 48 |
| ABM67 | HCDR3 - D102H | QNWHAFVY | 49 |
| ABM67 | HCDR3 - V105G | QNWDAFGY | 50 |
| ABM67 | HCDR3 - Y106S | QNWDAFVS | 51 |
| ABM67 | HCDR3 - A103V | QNWDVFVY | 52 |
| ABM67 | HCDR3 - V105A | QNWDAFAY | 53 |
| ABM67 | HCDR3 - Y106I | QNWDAFVI | 54 |
| ABM67 | HCDR3 - Y106A | QNWDAFVA | 55 |
| ABM67 | HCDR3 - Y106K | QNWDAFVK | 56 |
| ABM67 | LCDR1 | RAGQSIGTSIH | 58 |
| ABM67 | LCDR2 | SASESIS | 59 |
| ABM67 | LCDR3 | QQSNSWPLT | 60 |
| ABM72 | HCDR1 | SYGVH | 81 |
| ABM72 | HCDR2 | VIWSDGFITYNSALKS | 82 |
| ABM72 | HCDR3 | NDGSYYYAMDY | 83 |
| ABM72 | LCDR1 | RSSQSLVHSNGNTYLH | 27 |
| ABM72 | LCDR2 | KVSNRES | 28 |
| ABM72 | LCDR3 | SQSTHVPFT | 29 |
| ABM73 | HCDR1 | SYGVH | 81 |
| ABM73 | HCDR2 | VIWSDGSTTYNSALKS | 86 |
| ABM73 | HCDR3 | DYRYEYVMDY | 87 |
| ABM73 | LCDR1 | RSSQSLVHSNGNTYLH | 27 |
| ABM73 | LCDR2 | KVSNRES | 28 |
| ABM73 | LCDR3 | SQSIHVPFT | 89 |
| ABM93 | HCDR1 | SNGVH | 91 |
| ABM93 | HCDR2 | VIWSDGSTTYNSALKS | 86 |
| ABM93 | HCDR3 | HDGYSYYYAMEY | 92 |
| ABM93 | LCDR1 | RSSQSLVHSNGNTYLH | 27 |
| ABM93 | LCDR2 | KVSNRFS | 28 |
| ABM93 | LCDR3 | SQSTHVPLT | 94 |
| ABM96 | HCDR1 | SYGVH | 81 |
| ABM96 | HCDR2 | VIWSDGSITYNSALKS | 86 |
| ABM96 | HCDR3 | YDNYGYYFAMDY | 96 |
| ABM96 | LCDR1 | RSSQSLVHSNGNTYIH | 27 |
| ABM96 | LCDR2 | KVSNRES | 28 |
| ABM96 | LCDR3 | SQSTHVPLT | 94 |

In some embodiments, the anti-IL,-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 2; (b) the HCDR2 comprises the amino acid sequence of SEQ ID NO: 3; (c) the HCDR3 comprises the amino acid sequence of SEQ ID NO: 4; (d) the LCDR1 comprises the amino acid sequence of SEQ ID NO: 6; (e) the LCDR2 comprises the amino acid sequence of SEQ ID NO: 7; and (f) the LCDR3 comprises the amino acid sequence of SEQ ID NO: 8. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 2; (b) the HCDR2 consists of the amino acid sequence of SEQ ID NO: 3; (c) the HCDR3 consists of the amino acid sequence of SEQ ID NO: 4; (d) the LCDR1 consists of the amino acid sequence of SEQ ID NO: 6; (e) the LCDR2 consists of the amino acid sequence of SEQ ID NO: 7; and (f) the LCDR3 consists of the amino acid sequence of SEQ ID NO: 8.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 2; (b) the HCDR2 comprises the amino acid sequence of SEQ ID NO: 3; (c) the HCDR3 comprises the amino acid sequence of SEQ ID NO: 4; (d) the LCDR1 comprises the amino acid sequence of SEQ ID NO: 10; (e) the LCDR2 comprises the amino acid sequence of SEQ ID NO: 7; and (f) the LCDR3 comprises the amino acid sequence of SEQ ID NO: 8. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 2; (b) the HCDR2 consists of the amino acid sequence of SEQ ID NO: 3; (c) the HCDR3 consists of the amino acid sequence of SEQ ID NO: 4; (d) the LCDR1 consists of the amino acid sequence of SEQ ID NO: 10; (e) the LCDR2 consists of the amino acid sequence of SEQ ID NO: 7; and (f) the LCDR3 consists of the amino acid sequence of SEQ ID NO: 8.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 23; (b) the HCDR2 comprises the amino acid sequence of SEQ ID NO: 24; (c) the HCDR3 comprises the amino acid sequence of SEQ ID NO: 25; (d) the LCDR1 comprises the amino acid sequence of SEQ ID NO: 27; (e) the LCDR2 comprises the amino acid sequence of SEQ ID NO: 28; and (f) the LCDR3 comprises the amino acid sequence of SEQ ID NO: 29. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 23; (b) the HCDR2 consists of the amino acid sequence of SEQ ID NO: 24; (c) the HCDR3 consists of the amino acid sequence of SEQ ID NO: 25; (d) the LCDR1 consists of the amino acid sequence of SEQ ID NO: 27; (e) the LCDR2 consists of the amino acid sequence of SEQ ID NO: 28; and (f) the LCDR3 consists of the amino acid sequence of SEQ ID NO: 29.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 31; (b) the HCDR2 comprises the amino acid sequence of SEQ ID NO: 32; (c) the HCDR3 comprises the amino acid sequence of SEQ ID NO: 33; (d) the LCDR1 comprises the amino acid sequence of SEQ ID NO: 35; (e) the LCDR2 comprises the amino acid sequence of SEQ ID NO: 36; and (f) the LCDR3 comprises the amino acid sequence of SEQ ID NO: 37. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 31; (b) the HCDR2 consists of the amino acid sequence of SEQ ID NO: 32; (c) the HCDR3 consists of the amino acid sequence of SEQ ID NO: 33; (d) the LCDR1 consists of the amino acid sequence of SEQ ID NO: 35; (e) the LCDR2 consists of the amino acid sequence of SEQ ID NO: 36; and (f) the LCDR3 consists of the amino acid sequence of SEQ ID NO: 37.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 44; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 44; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 51; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 51; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 46; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 46; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 47; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 47; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 48; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 48; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 49; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 49; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 44; (c) the HCDR3 comprises the amino acid sequence of SEQ ID NO: 50; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 44; (c) the HCDR3 consists of the amino acid sequence of SEQ ID NO: 50; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 52; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 52; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 53; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 53; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 54; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 54; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 55; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 55; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 56; (d) the LCDR1 comprises the amino acid sequence of SEQ ID NO: 58; (e) the LCDR2 comprises the amino acid sequence of SEQ ID NO: 59; and (f) the LCDR3 comprises the amino acid sequence of SEQ ID NO: 60. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 42; (b) the HCDR2 consists of the amino acid sequence of SEQ ID NO: 43; (c) the HCDR3 consists of the amino acid sequence of SEQ ID NO: 56; (d) the LCDR1 consists of the amino acid sequence of SEQ ID NO: 58; (e) the LCDR2 consists of the amino acid sequence of SEQ ID NO: 59; and (f) the LCDR3 consists of the amino acid sequence of SEQ ID NO: 60.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 81; (b) the HCDR2 comprises the amino acid sequence of SEQ ID NO: 82; (c) the HCDR3 comprises the amino acid sequence of SEQ ID NO: 83; (d) the LCDR1 comprises the amino acid sequence of SEQ ID NO: 27; (e) the LCDR2 comprises the amino acid sequence of SEQ ID NO: 28; and (f) the LCDR3 comprises the amino acid sequence of SEQ ID NO: 29. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 81; (b) the HCDR2 consists of the amino acid sequence of SEQ ID NO: 82; (c) the HCDR3 consists of the amino acid sequence of SEQ ID NO: 83; (d) the LCDR1 consists of the amino acid sequence of SEQ ID NO: 27; (e) the LCDR2 consists of the amino acid sequence of SEQ ID NO: 28; and (f) the LCDR3 consists of the amino acid sequence of SEQ ID NO: 29.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 81; (b) the HCDR2 comprises the amino acid sequence of SEQ ID NO: 86; (c) the HCDR3 comprises the amino acid sequence of SEQ ID NO: 87; (d) the LCDR1 comprises the amino acid sequence of SEQ ID NO: 27; (e) the LCDR2 comprises the amino acid sequence of SEQ ID NO: 28; and (f) the LCDR3 comprises the amino acid sequence of SEQ ID NO: 89. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 81; (b) the HCDR2 consists of the amino acid sequence of SEQ ID NO: 86; (c) the HCDR3 consists of the amino acid sequence of SEQ ID NO: 87; (d) the LCDR1 consists of the amino acid sequence of SEQ ID NO: 27; (e) the LCDR2 consists of the amino acid sequence of SEQ ID NO: 28; and (f) the LCDR3 consists of the amino acid sequence of SEQ ID NO: 89.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 91; (b) the HCDR2 comprises the amino acid sequence of SEQ ID NO: 86; (c) the HCDR3 comprises the amino acid sequence of SEQ ID NO: 92; (d) the LCDR1 comprises the amino acid sequence of SEQ ID NO: 27; (e) the LCDR2 comprises the amino acid sequence of SEQ ID NO: 28; and (f) the LCDR3 comprises the amino acid sequence of SEQ ID NO: 94. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 91; (b) the HCDR2 consists of the amino acid sequence of SEQ ID NO: 86; (c) the HCDR3 consists of the amino acid sequence of SEQ ID NO: 92; (d) the LCDR1 consists of the amino acid sequence of SEQ ID NO: 27; (e) the LCDR2 consists of the amino acid sequence of SEQ ID NO: 28; and (f) the LCDR3 consists of the amino acid sequence of SEQ ID NO: 94.

In some embodiments, the anti-IL-17A antibody or antigen-binding fragment thereof comprises an HCDR1, HCDR2, HCDR3, LCDR1, LCDR2, and LCDR3, wherein (a) the HCDR1 comprises the amino acid sequence of SEQ ID NO: 81; (b) the HCDR2 comprises the amino acid sequence of SEQ ID NO: 86; (c) the HCDR3 comprises the amino acid sequence of SEQ ID NO: 96; (d) the LCDR1 comprises the amino acid sequence of SEQ ID NO: 27; (e) the LCDR2 comprises the amino acid sequence of SEQ ID NO: 28; and (f) the LCDR3 comprises the amino acid sequence of SEQ ID NO: 94. In some embodiments, the anti-IL-17A antibody or antigen-binding fragment thereof comprises an HCDR1, HCDR2, HCDR3, LCDR1, LCDR2, and LCDR3, wherein (a) the HCDR1 consists of the amino acid sequence of SEQ ID NO: 81; (b) the HCDR2 consists of the amino acid sequence of SEQ ID NO: 86; (c) the HCDR3 consists of the amino acid sequence of SEQ ID NO: 96; (d) the LCDR1 consists of the amino acid sequence of SEQ ID NO: 27; (e) the LCDR2 consists of the amino acid sequence of SEQ ID NO: 28; and (f) the LCDR3 consists of the amino acid sequence of SEQ ID NO: 94.

In some embodiments, the anti-IL-17A antibody or antigen-binding fragment thereof comprises a VH and a VL chain, wherein the VH chain comprises or consists of an amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence selected from the group consisting of SEQ ID NOs: 5, 26, 34, 57, 84, 88, 93, and 97. In some embodiments, the anti-IL-17A antibody or antigen-binding fragment thereof comprises a VH and a VL chain, wherein the VL chain comprises or consists of an amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence selected from the group consisting of SEQ ID NOs: 9, 11, 30, 38, 61, 85, 90, 95, and 98.

In some embodiments, the anti-IL-17A antibody or antigen-binding fragment thereof comprises a VH and a VL chain, wherein the VH chain comprises or consists of an amino acid sequence that is at least about 85%%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence selected from the group consisting of SEQ ID NOs: 5, 26, 34, 57, 84, 88, 93, and 97, and the VL chain comprises or consists of an amino acid sequence that is at least about 85%%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence selected from the group consisting of SEQ ID NOs: 9, 11, 30, 38, 61, 85, 90, 95, and 98.

Exemplary VH and VL sequence of the anti-IL-17A antibodies described herein are provided in Table 4.

TABLE 4
Exemplary parental VH and VL amino acid sequences

| Construct | Component | Sequence | SEQ ID |
|---|---|---|---|
| ABM58 | VH | EVKLVESGGGLVKPGGSLKLSCAASGFAFSSYDMSWVRQTPEK | 5 |
| | | RLEWVAYISSGGGNTYYPDTVKGRFTISRDNAKNTLYLQMSSL | |
| | | KSEDTAMYYCAREALTMISTRDYYAMDYWGQGTSVTVSS | |
| ABM58 | VL | DIVMTQPPASLAASLGQRATISCRASESVDNYGISFMNWFQQK | 9 |
| | | PGQPPKLLIYAASNQGSGVPARFSGSGSGTDFSLNIHPMEEDD | |
| | | AAMYFCQQSKEVPYTFGGGTKLEIKR | |
| ABM59 | VH | EVKLVESGGGLVKPGGSLKLSCAASGFAFSSYDMSWVRQTPEK | 5 |
| | | RLEWVAYISSGGGNTYYPDTVKGRFTISRDNAKNTLYLQMSSL | |
| | | KSEDTAMYYCAREALTMISTRDYYAMDYWGQGTSVTVSS | |
| ABM59 | VL | DVLMTQTPASLAVSLGQRATISCGASESVDNYGISFMDWFQQK | 11 |
| | | PGQPPKLLIYAASNQGSGVPARFSGSGSGTDFSLNIHPMEEND | |
| | | AAMYFCQQSKEVPYTFGGGTKLEIKR | |
| ABM60 | VH | EVHLKESGPDLVAPSQSLSITCTVSGFSLTTYGVHWVRQPPGK | 26 |
| | | GLEWLVVIWSDGLTTYNSALKSRLSISKDNSKSQVFLKMNSLR | |
| | | TDDTAMYYCARDYTYEYAMDYWGQGTTVTVSS | |
| ABM60 | VL | DILLTQSPLSLPVSLGDQASISCRSSQSLVHSNGNTYLHWYLQ | 30 |
| | | KPGQSPKLLIYKVSNRFSGVPDRFSGSESGTDFTLKISRVEAE | |
| | | DLGVYFCSQSTHVPFTFGSGTKLEIKR | |
| ABM64 | VH | QVHVKQSGPELVRPGVSVKISCKGSGYTFTDYAMHWVKQSHAK | 34 |
| | | SLEWIGVISTYSGNTNYNQKFKGKATMTVDKSSSTAYMELARL | |
| | | TSEDSAIYYCARGNYGSDWGQGTLVTVSA | |
| ABM64 | VL | DIVMTQSPSSLAMSVGQKVTMSCKSSQSLLNSSNQKNYLAWYQ | 38 |
| | | QKPGQSPKLLVYFASTRESGVPDRFIGSGSGTDFTLTISSVQA | |
| | | EDLADYFCQQHYSTPYTFGGGTKLELKR | |
| ABM67 | VH | EVKLVESGGGLVKPGGSLKLSCAASGFTFSSYAMSWVRQTPEK | 57 |
| | | RLEWVATISSGGSYTYYPDSVKGRFTISRDNAKNTLYLQMSSL | |
| | | RSEDTAMYYCARQNWDAFVYWGQGTLVTASA | |
| ABM67 | VL | DIVLTQSPAILSVSPGERVSFSCRAGQSIGTSIHWYQRRINGS | 61 |
| | | PRLLIKSASESISGIPSRFSGSGSGTDFTLSINSVESEDIADY | |
| | | YCQQSNSWPLTFGAGTKLEIKR | |
| ABM72 | VH | EVKLKESGPGLVAPSQSLSITCTVSGFSLTSYGVHWVRQPPGK | 84 |
| | | GLEWLVVIWSDGFTTYNSALKSRLSISKDNSKSQVFLKMNSLQ | |
| | | TDDTAMYYCARNDGSYYYAMDYWGQGTSVTVSS | |
| ABM72 | VL | DIELTQPPLSLPVSLGDQASISCRSSQSLVHSNGNTYLHWYLQ | 85 |
| | | KPGQSPKLLIYKVSNRFSGVPDRFSGSGSGTDFTLKISRVEAE | |
| | | DLGVYFCSQSTHVPFTFGSGTKLEIKR | |
| ABM73 | VH | QVHVKQSGPDLVAPSQSLSITCTVSGFSLTSYGVHWVRQPPGK | 88 |
| | | GLEWLVVIWSDGSTTYNSALKSRLSISKDNSKSQVFLKMNSLQ | |
| | | TDDTAMYYCARDYRYEYVMDYWGQGTSVTVSS | |
| ABM73 | VL | DVVMTQTPLSLPVSLGDQASISCRSSQSLVHSNGNTYLHWYLQ | 90 |
| | | KPGQSPKVLIYKVSNRFSGVPDRFSGSGSGTDFTLKISRVEAE | |
| | | DLGVYFCSQSIHVPFTFGSGTKLEIKR | |
| ABM93 | VH | QVQLQQSGPDLVAPSQSLSITCTVSGFSLTSNGVHWVRQPPGK | 93 |
| | | GLEWLVVIWSDGSTTYNSALKSRLSISKDNSKSQVFLKMNSLQ | |
| | | TDDTAMYYCARHDGYSYYYAMEYWGQGTTLTVSS | |
| ABM93 | VL | DIVMTQVPLSLPVSLGDQASISCRSSQSLVHSNGNTYLHWYLQ | 95 |
| | | KPGQSPKLLIYKVSNRFSGVPDRFRGSGSGTDFALKISRVEAE | |
| | | DLGVYFCSQSTHVPLTFGAGTKLEIKR | |
| ABM96 | VH | EVKVIESGPDLVAPSQSLSITCTVSGFSLTSYGVHWVRQPPGK | 97 |
| | | GLEWLVVIWSDGSTTYNSALKSRLSISKDNSKSQVFLKMHSLQ | |
| | | TDDTAMYYCASYDNYGYYFAMDYWGQGTTLTVSS | |
| ABM96 | VL | DIQLTQSPLSLPVSLGDQASISCRSSQSLVHSNGNTYLHWYLQ | 98 |
| | | KPGQSPKLLIYKVSNRFSGVPDRESGSGSGTDFTLKISRVEAE | |
| | | DLGVYFCSQSTHVPLTFGAGTKLEXKR | |

In some embodiments, the present disclosure provides an anti-IL-17A antibody or antigen-fragment thereof referred to as ABM58. In some embodiments, the anti-IL-17A antibody or antigen-fragment thereof comprises a VH amino acid sequence comprising SEQ ID NO: 5 and a VL amino acid sequence comprising SEQ ID NO: 9. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 5 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 9. In some embodiments, the anti-IL-17A antibody or antigen-binding fragment thereof comprises a VH amino acid sequences consisting of SEQ ID NO: 5 and comprises a VL amino acid sequence consisting of SEQ ID NO: 9.

In some embodiments, the present disclosure provides an anti-IL-17A antibody or antigen-fragment thereof referred to as ABM59. In some embodiments, the anti-IL-17A antibody or antigen-fragment thereof comprises a VH amino acid sequence comprising SEQ ID NO: 5 and a VL amino acid sequence comprising SEQ ID NO: 11. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 5 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 11. In some embodiments, the anti-IL-17A antibody or antigen-binding fragment thereof comprises a VH amino acid sequences consisting of SEQ ID NO: 5 and comprises a VL amino acid sequence consisting of SEQ ID NO: 11.

In some embodiments, the present disclosure provides an anti-IL-17A antibody or antigen-fragment thereof referred to as ABM60. In some embodiments, the anti-IL-17A antibody or antigen-fragment thereof comprises a VH amino acid sequence comprising SEQ ID NO: 26 and a VL amino acid sequence comprising SEQ ID NO: 30. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 26 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 30. In some embodiments, the anti-IL-17A antibody or antigen-binding fragment thereof comprises a VH amino acid sequences consisting of SEQ ID NO: 26 and comprises a VL amino acid sequence consisting of SEQ ID NO: 30.

In some embodiments, the present disclosure provides an anti-IL-17A antibody or antigen-fragment thereof referred to as ABM67. In some embodiments, the anti-IL-17A antibody or antigen-fragment thereof comprises a VH amino acid sequence comprising SEQ ID NO: 57 and a VL amino acid sequence comprising SEQ ID NO: 61. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 57 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 61. In some embodiments, the anti-IL-17A antibody or antigen-binding fragment thereof comprises a VH amino acid sequences consisting of SEQ ID NO: 57 and comprises a VL amino acid sequence consisting of SEQ ID NO: 61.

In some embodiments, the present disclosure provides an anti-IL-17A antibody or antigen-fragment thereof referred to as ABM72. In some embodiments, the anti-IL-17A antibody or antigen-fragment thereof comprises a VH amino acid sequence comprising SEQ ID NO: 84 and a VL amino acid sequence comprising SEQ ID NO: 85. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 84 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 85. In some embodiments, the anti-IL-17A antibody or antigen-binding fragment thereof comprises a VH amino acid sequences consisting of SEQ ID NO: 84 and comprises a VL amino acid sequence consisting of SEQ ID NO: 85.

In some embodiments, the present disclosure provides an anti-IL-17A antibody or antigen-fragment thereof referred to as ABM73. In some embodiments, the anti-IL-17A antibody or antigen-fragment thereof comprises a VH amino acid sequence comprising SEQ ID NO: 88 and a VL amino acid sequence comprising SEQ ID NO: 90. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 88 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 90. In some embodiments, the anti-IL-17A antibody or antigen-binding fragment thereof comprises a VH amino acid sequences consisting of SEQ ID NO: 88 and comprises a VL amino acid sequence consisting of SEQ ID NO: 90.

In some embodiments, the present disclosure provides an anti-IL-17A antibody or antigen-fragment thereof referred to as ABM93. In some embodiments, the anti-IL-17A antibody or antigen-fragment thereof comprises a VH amino acid sequence comprising SEQ ID NO: 93 and a VL amino acid sequence comprising SEQ ID NO: 95. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 93 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 95. In some embodiments, the anti-IL-17A antibody or antigen-binding fragment thereof comprises a VH amino acid sequences consisting of SEQ ID NO: 93 and comprises a VL amino acid sequence consisting of SEQ ID NO: 95.

In some embodiments, the present disclosure provides an anti-IL-17A antibody or antigen-fragment thereof referred to as ABM96. In some embodiments, the anti-IL-17A antibody or antigen-fragment thereof comprises a VH amino acid sequence comprising SEQ ID NO: 97 and a VL amino acid sequence comprising SEQ ID NO: 98. In some embodiments, the anti-IL-17A antibody or antigen-binding fragment thereof comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 97 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 98. In some embodiments, the anti-IL-17A antibody or antigen-binding fragment thereof comprises a VH amino acid sequences consisting of SEQ ID NO: 97 and comprises a VL amino acid sequence consisting of SEQ ID NO: 98.

### Humanized Anti-IL-17A Antibodies and Antigen Binding Fragments

In one aspect, the disclosed anti-IL-17A antibodies and antigen binding fragments thereof can be human antibodies or human binding molecules. The term “human”, when applied to the antibodies and antigen binding fragments thereof as defined herein, refers to molecules that are derived from a parental human antibody sequence. When an antibody or antigen binding fragment thereof is derived from a human sequence and subsequently modified, it is still to be considered human as used throughout the specification. In other words, the term human, when applied to antibodies or antigen binding fragments thereof is intended to include antibodies having variable and constant regions derived from human germline immunoglobulin sequences based on variable or constant regions either or not occurring in a human or human lymphocyte or in modified form. Thus, the human binding molecules may include amino acid residues not encoded by human germline immunoglobulin sequences, comprise substitutions and/or deletions (e.g., mutations introduced by for instance random or site-specific mutagenesis in vitro or by somatic mutation in vivo). “Derived from” as used herein refers to the situation that a nucleic acid sequence may be exactly copied from a template, or with minor mutations, such as by error-prone PCR methods, or synthetically made matching the template exactly or with minor modifications.

In some embodiments, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulins. Humanized antibodies include human immunoglobulins (recipient antibody) in which residues from a complementary determining region (CDR) are replaced by residues from a CDR of a non-human species (parental antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the parental CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323,327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “bestfit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

In some aspect, it can be important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

In some embodiments, the anti-IL-17A antibody or antigen-fragment thereof comprises a humanized VH amino acid sequence selected from SEQ ID NO: 12, 14, 16, 21, 22, 39, 62, 64, 65, 66, 67, 68, 69, 71, 72, 73, 74, 99, 77, 78, and 79 and a humanized VL amino acid sequence selected from SEQ ID NO: 13, 15, 17, 18, 19, 20, 40, 41, 63, 70, 75, 76, and 80. Exemplary humanized VH and VL sequences are provided in Table 5.

TABLE 5
Exemplary humanized anti-IL-17A VH and VL sequences

| Parental | | | SEQ |
|---|---|---|---|
| Construct | Component | Sequence | ID |
| ABM59 | 59.1H VH | EVQLVESGGGLVQPGGSLRLSCAASGFTFSSYDMSWVRQAPGK | 12 |
| | | GLEWVSYISSGGGNTYYPDTVKGRFTISRDNAKNSLYLQMNSL | |
| | | RAEDTAVYYCAREALTMISTRDYYAMDYWGQGTLVTVSS | |
| ABM59 | 59.1L VL | DIVLTQSPASLAVSPGQRATITCGASESVDNYGINLIHWYQQK | 13 |
| | | PGQPPKLLIYAASNQGSGVPARFSGSGSGTDFTLTINPVEAND | |
| | | TANYYCQQSKEVPYTFGGGTKLEIKR | |
| ABM59 | 59.2H VH | EVKLVESGGGLVQPGGSLRLSCAASGFAFSSYDMSWVRQAPGK | 14 |
| | | GLEWVAYISSGGGNTYYPDTVKGRFTISRDNAKNSLYLQMNSL | |
| | | RAEDTAVYYCAREALTMISTRDYYAMDYWGQGTLVTVSS | |
| ABM59 | 59.2L VL | DVLMTQSPASLAVSPGQRATISCGASESVDNYGISFMDWFQQK | 15 |
| | | PGQPPKLLIYAASNQGSGVPARFSGSGSGTDFTLTINPMEEND | |
| | | TANYFCQQSKEVPYTFGGGTKLEIKR | |
| ABM59 | 59.3H VH | EVKLVESGGGLVQPGGSLRLSCAASGFAFSSYDMSWVRQAPEK | 16 |
| | | RLEWVAYISSGGGNTYYPDTVKGRFTISRDNAKNSLYLQMNSL | |
| | | RAEDTAVYYCAREALTMISTRDYYAMDYWGQGTLVTVSS | |
| ABM59 | 59.3L VL | DIVLTQSPASLAVSPGQRATITCGASESVDNYGISFMDWFQQK | 17 |
| | | PGQPPKLLIYAASNQGSGVPARFSGSGSGTDFTLTINPVEAND | |
| | | TANYYCQQSKEVPYTFGGGTKLEIKR | |
| ABM59 | 59.4L VL | DIVLTQSPASLAVSPGQRATITCGASESVDNYGISFMDWFQQK | 18 |
| | | PGQPPKLLIYAASNQGSGVPARFSGSGSGTDFTLTINPVEAQD | |
| | | TANYYCQQSKEVPYTFGGGTKLEIKR | |
| ABM59 | 59.5L VL | DIQLTQSPSSLSASVGDRVTITCGASESVDNYGISFMDWYQQK | 19 |
| | | PGKAPKLLIYAASNQGSGVPSRFSGSGSGTDFTLTISSLQPED | |
| | | FATYYCQQSKEVPYTFGqGTKVEIKR | |
| ABM59 | 59.6L VL | EVVMTQSPATLSVSPGERATLSCGASESVDNYGISFMDWYQQK | 20 |
| | | PGQAPRLLIYAASNQGSGIPARFSGSGSGTEFTLTISSLQSED | |
| | | FAVYYCQQSKEVPYTFGqGTKVEIKR | |
| ABM59 | 59.4H VH | EVQLVESGGGLVQPGGSLRLSCAASGFaFSSYDMSWVRQAPEK | 21 |
| | | RLEWVAYISSGGGNTYYPDTVKGRFTISRDNAKNTLYLQMNSL | |
| | | RAEDTAVYYCAKEALTMISTRDYYAMDYWGQGTLVTVSS | |
| ABM59 | 59.5H VH | EVQLVESGGGLVQPGGSLRLSCAASGFaFSSYDMSWVRQAPGK | 22 |
| | | GLEWVAYISSGGGNTYYPDTVKGRFTISRDNAKNtLYLQMNSL | |
| | | RAEDTAVYYCAREALTMISTRDYYAMDYWGQGTLVTVSS | |
| ABM64 | 64.1H VH | QVQLVQSGAEVKKPGASVKVSCKASGYTFTDYAMHWVRQAPGQ | 39 |
| | | GLEWMGVISTYSGNTNYNQKFKGRVTMTRDTSISTAYMELSRL | |
| | | RSDDTAVYYCARGNYGSDWGQGTLVTVSS | |
| ABM64 | 64.1L VL | DIVMTQSPDSLAVSLGERATINCKSSQSLLNSSNQKNYLAWYQ | 40 |
| | | QKPGQPPKLLIYFASTRESGVPDRESGSGSGTDFTLTISSLQA | |
| | | EDVAVYYCQQHYSTPYTFGGGTKLEIKR | |
| ABM64 | 64.3L VL | EIVmTQSPGTLSLSPGERATLSCKSSQSLLNSSNQKNYLAWYQ | 41 |
| | | QKPGQAPRLLIYLAStResGIPDRESGSGSGTDFTLTISRLEP | |
| | | EDFAVYYCQQHYSTPYTFGqGTKvEiKR | |
| ABM67 | 67.1H VH | EVQLVESGGGLVKPGGSLRLSCAASGFTFSSYAMSWVRQAPGK | 62 |
| | | GLEWVSTISSGGSYTYYPDSVKGRFTISRDNAKNSLYLQMNSL | |
| | | RAEDTAVYYCARQNWDAFVYWGQGTLVTVSS | |
| ABM67 | 67.1L VL | EIVLTQSPDFQSVTPKEKVTITCRAGQSIGTSIHWYQQKPDQS | 63 |
| | | PKLLIKSASESISGVPSRFSGSGSGTDFTLTINSLEAEDAATY | |
| | | YCQQSNSWPLTFGAGTKLEIKR | |
| ABM67 | 67.1H (Q99R) | EVQLVESGGGLVKPGGSLRLSCAASGFTFSSYAMSWVRQAPGK | 64 |
| | VH | GLEWVSTISSGGSYTYYPDSVKGRFTISRDNAKNSLYLQMNSL | |
| | | RAEDTAVYYCARRNWDAFVYWGQGTLVTVSS | |
| ABM67 | 67.1H (N100G) | EVQLVESGGGLVKPGGSLRLSCAASGFTFSSYAMSWVRQAPGK | 65 |
| | VH | GLEWVSTISSGGSYTYYPDSVKGRFTISRDNAKNSLYLQMNSL | |
| | | RAEDTAVYYCARQGWDAFVYWGQGTLVTVSS | |
| ABM67 | 67.1H (D102R) | EVQLVESGGGLVKPGGSLRLSCAASGFTFSSYAMSWVRQAPGK | 66 |
| | VH | GLEWVSTISSGGSYTYYPDSVKGRFTISRDNAKNSLYLQMNSL | |
| | | RAEDTAVYYCARQNWRAFVYWGQGTLVTVSS | |
| ABM67 | 67.1H (D102H) | EVQLVESGGGLVKPGGSLRLSCAASGFTFSSYAMSWVRQAPGK | 67 |
| | VH | GLEWVSTISSGGSYTYYPDSVKGRFTISRDNAKNSLYLQMNSL | |
| | | RAEDTAVYYCARQNWHAFVYWGQGTLVTVSS | |
| ABM67 | 67.1H (Y57H, | EVQLVESGGGLVKPGGSLRLSCAASGFTESSYAMSWVRQAPGK | 68 |
| | V64A, V105G) | GLEWVSTISSGGSHTYYPDSAKGRFTISRDNAKNSLYLQMNSL | |
| | VH | RAEDTAVYYCARQNWDAFGYWGQGTLVTVSS | |
| ABM67 | 67.2H VH | EVQLVESGGGLVKPGGSLRLSCAASGFTFSSYAMSWVRQTPEK | 69 |
| | | RLEWVATISSGGSYTYYPDSVKGRFTISRDNAKNSLYLQMSSL | |
| | | RAEDTAVYYCARQNWDAFVYWGQGTLVTVSS | |
| ABM67 | 67.2L VL | EIVLTQSPVILSVTPKEKVTFTCRAGQSIGTSIHWYQQRTNQS | 70 |
| | | PKLLIKSASESISGVPSRFSGSGSGTDFTLTINSLEAEDIATY | |
| | | YCQQSNSWPLTFGAGTKLEIKR | |
| ABM67 | 67.2H (Y106S) | EVQLVESGGGLVKPGGSLRLSCAASGFTFSSYAMSWVRQTPEK | 71 |
| | VH | RLEWVATISSGGSYTYYPDSVKGRFTISRDNAKNSLYLQMSSL | |
| | | RAEDTAVYYCARQNWDAFVSGQGTLVTVSS | |
| ABM67 | 67.2H (A103V) | EVQLVESGGGLVKPGGSLRLSCAASGFTESSYAMSWVRQTPEK | 72 |
| | VH | RLEWVATISSGGSYTYYPDSVKGRFTISRDNAKNSLYLQMSSL | |
| | | RAEDTAVYYCARQNWDVFVYWGQGTLVTVSS | |
| ABM67 | 67.2H (V105A) | EVQLVESGGGLVKPGGSLRLSCAASGFTFSSYAMSWVRQTPEK | 73 |
| | VH | RLEWVATISSGGSYTYYPDSVKGRFTISRDNAKNSLYLQMSSL | |
| | | RAEDTAVYYCARQNWDAFAYWGQGTLVTVSS | |
| ABM67 | 67.2H (Y106S) | EVQLVESGGGLVKPGGSLRLSCAASGFTESSYAMSWVRQTPEK | 74 |
| | VH-A | RLEWVATISSGGSYTYYPDSVKGRFTISRDNAKNSLYLQMSSL | |
| | | RAEDTAVYYCARQNWDAFVSWGQGTLVTVSS | |
| ABM67 | 67.2H (Y106S) | EVQLVESGGGLVKPGGSLRLPCAASGFTFSSYAMSWVRQTPER | 99 |
| | VH-B | RLEWVATISSGGSYTYYPDSVKGRFTISRDNAKNSLYLQMSSL | |
| | | RAEDTAVYYCARQNWDAFVSWGQGTLVTVSS | |
| ABM67 | 67.3L VL | EIVLTQSPVILSVTPKEKVTFTCRAGQSIGTSIHWYQQRTDQS | 75 |
| | | PKLLIKSASESISGVPSRFSGSGSGTDFTLTINSLEAEDIATY | |
| | | YCQQSNSWPLTFGAGTKLEIKR | |
| ABM67 | 67.4L VL | DIQMTQSPSSLSASVGDRVTITCRAGQSIGTSIHWYQQKPGKA | 76 |
| | | PKLLIKSASESISGVPSRFSGSGSGTDFTLTISSLQPEDFATY | |
| | | YCQQSNSWPLTFGAGTKLEIKR | |
| ABM67 | 67.2H (Y106I) | EVQLVESGGGLVKPGGSLRLSCAASGFTFSSYAMSWVRQTPEK | 77 |
| | VH | RLEWVATISSGGSYTYYPDSVKGRFTISRDNAKNSLYLQMSSL | |
| | | RAEDTAVYYCARQNWDAFVIWGQGTLVTVSS | |
| ABM67 | 67.2H (Y106A) | EVQLVESGGGLVKPGGSLRLSCAASGFTFSSYAMSWVRQTPEK | 78 |
| | VH | RLEWVATISSGGSYTYYPDSVKGRFTISRDNAKNSLYLQMSSL | |
| | | RAEDTAVYYCARQNWDAFVAWGQGTLVTVSS | |
| ABM67 | 67.2H (Y106K) | EVQLVESGGGLVKPGGSLRLSCAASGFTFSSYAMSWVRQTPEK | 79 |
| | VH | RLEWVATISSGGSYTYYPDSVKGRFTISRDNAKNSLYLQMSSL | |
| | | RAEDTAVYYCARQNWDAFVKWGQGTLVTVSS | |
| ABM67 | 67.6L VL | EIVLTQSPDFQSVTPKEKVTITCRAGQSIGTSIHWYQRKPDQS | 80 |
| | | PKLLIKSASESISGVPSRFSGSGSGTDFTLTINSLEAEDAATY | |
| | | YCQQSNSWPLTFGAGTKVEIKR | |

In some embodiments, the present disclosure provides an anti-IL-17A antibody or antigen-binding fragment thereof referred to as ABM59.1. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 12 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 13. In some embodiments, the anti-IL-17A antibody or antigen-binding fragment thereof comprises a humanized VH amino acid sequences consisting of SEQ ID NO: 12 and comprises a humanized VL amino acid sequence consisting of SEQ ID NO: 13.

In some embodiments, the present disclosure provides an anti-IL-17A antibody or antigen-binding fragment thereof referred to as ABM59.2. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 14 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 15. In some embodiments, the anti-IL-17A antibody or antigen-binding fragment thereof comprises a humanized VH amino acid sequences consisting of SEQ ID NO: 14 and comprises a humanized VL amino acid sequence consisting of SEQ ID NO: 15.

In some embodiments, the present disclosure provides an anti-IL-17A antibody or antigen-binding fragment thereof referred to as ABM59.3. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 15. In some embodiments, the anti-IL-17A antibody or antigen-binding fragment thereof comprises a humanized VH amino acid sequences consisting of SEQ ID NO: 16 and comprises a humanized VL amino acid sequence consisting of SEQ ID NO: 15.

In some embodiments, the present disclosure provides an anti-IL-17A antibody or antigen-binding fragment thereof referred to as ABM59.4. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 14 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 17. In some embodiments, the anti-IL-17A antibody or antigen-binding fragment thereof comprises a humanized VH amino acid sequences consisting of SEQ ID NO: 14 and comprises a humanized VL amino acid sequence consisting of SEQ ID NO: 17.

In some embodiments, the present disclosure provides an anti-IL-17A antibody or antigen-binding fragment thereof referred to as ABM59.5. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 17. In some embodiments, the anti-IL-17A antibody or antigen-binding fragment thereof comprises a humanized VH amino acid sequences consisting of SEQ ID NO: 16 and comprises a humanized VL amino acid sequence consisting of SEQ ID NO: 17.

In some embodiments, the present disclosure provides an anti-IL-17A antibody or antigen-binding fragment thereof referred to as ABM59.6. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 18. In some embodiments, the anti-IL-17A antibody or antigen-binding fragment thereof comprises a humanized VH amino acid sequences consisting of SEQ ID NO: 16 and comprises a humanized VL amino acid sequence consisting of SEQ ID NO: 18.

In some embodiments, the present disclosure provides an anti-IL-17A antibody or antigen-binding fragment thereof referred to as ABM59.7. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 19. In some embodiments, the anti-IL-17A antibody or antigen-binding fragment thereof comprises a humanized VH amino acid sequences consisting of SEQ ID NO: 16 and comprises a humanized VL amino acid sequence consisting of SEQ ID NO: 19.

In some embodiments, the present disclosure provides an anti-IL-17A antibody or antigen-binding fragment thereof referred to as ABM59.8. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 20. In some embodiments, the anti-IL-17A antibody or antigen-binding fragment thereof comprises a humanized VH amino acid sequences consisting of SEQ ID NO: 16 and comprises a humanized VL amino acid sequence consisting of SEQ ID NO: 20.

In some embodiments, the present disclosure provides an anti-IL-17A antibody or antigen-binding fragment thereof referred to as ABM59.9. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 21 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 18. In some embodiments, the anti-IL-17A antibody or antigen-binding fragment thereof comprises a humanized VH amino acid sequences consisting of SEQ ID NO: 21 and comprises a humanized VL amino acid sequence consisting of SEQ ID NO: 18.

In some embodiments, the present disclosure provides an anti-IL-17A antibody or antigen-binding fragment thereof referred to as ABM59.10. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 21 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 19. In some embodiments, the anti-IL-17A antibody or antigen-binding fragment thereof comprises a humanized VH amino acid sequences consisting of SEQ ID NO: 21 and comprises a humanized VL amino acid sequence consisting of SEQ ID NO: 19.

In some embodiments, the present disclosure provides an anti-IL-17A antibody or antigen-binding fragment thereof referred to as ABM59.11. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 21 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 20. In some embodiments, the anti-IL-17A antibody or antigen-binding fragment thereof comprises a humanized VH amino acid sequences consisting of SEQ ID NO: 21 and comprises a humanized VL amino acid sequence consisting of SEQ ID NO: 20.

In some embodiments, the present disclosure provides an anti-IL-17A antibody or antigen-binding fragment thereof referred to as ABM59.12. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 22 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 18. In some embodiments, the anti-IL-17A antibody or antigen-binding fragment thereof comprises a humanized VH amino acid sequences consisting of SEQ ID NO: 22 and comprises a humanized VL amino acid sequence consisting of SEQ ID NO: 18.

In some embodiments, the present disclosure provides an anti-IL-17A antibody or antigen-binding fragment thereof referred to as ABM59.13. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 22 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 19. In some embodiments, the anti-IL-17A antibody or antigen-binding fragment thereof comprises a humanized VH amino acid sequences consisting of SEQ ID NO: 22 and comprises a humanized VL amino acid sequence consisting of SEQ ID NO: 19.

In some embodiments, the present disclosure provides an anti-IL-17A antibody or antigen-binding fragment thereof referred to as ABM59.14. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 22 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 20. In some embodiments, the anti-IL-17A antibody or antigen-binding fragment thereof comprises a humanized VH amino acid sequences consisting of SEQ ID NO: 22 and comprises a humanized VL amino acid sequence consisting of SEQ ID NO: 20.

In some embodiments, the present disclosure provides an anti-IL-17A antibody or antigen-binding fragment thereof referred to as ABM64.1. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 39 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 40. In some embodiments, the anti-IL-17A antibody or antigen-binding fragment thereof comprises a humanized VH amino acid sequences consisting of SEQ ID NO: 39 and comprises a humanized VL amino acid sequence consisting of SEQ ID NO: 40.

In some embodiments, the present disclosure provides an anti-IL-17A antibody or antigen-binding fragment thereof referred to as ABM64.2. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 39 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 41. In some embodiments, the anti-IL-17A antibody or antigen-binding fragment thereof comprises a humanized VH amino acid sequences consisting of SEQ ID NO: 39 and comprises a humanized VL amino acid sequence consisting of SEQ ID NO: 41.

In some embodiments, the present disclosure provides an anti-IL-17A antibody or antigen-binding fragment thereof referred to as ABM64.3. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 34 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 41. In some embodiments, the anti-IL-17A antibody or antigen-binding fragment thereof comprises a humanized VH amino acid sequences consisting of SEQ ID NO: 34 and comprises a humanized VL amino acid sequence consisting of SEQ ID NO: 41.

In some embodiments, the present disclosure provides an anti-IL-17A antibody or antigen-binding fragment thereof referred to as ABM67.1. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 62 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A antibody or antigen-binding fragment thereof comprises a humanized VH amino acid sequences consisting of SEQ ID NO: 62 and comprises a humanized VL amino acid sequence consisting of SEQ ID NO: 63.

In some embodiments, the present disclosure provides an anti-IL-17A antibody or antigen-binding fragment thereof referred to as ABM67.1.2. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 64 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A antibody or antigen-binding fragment thereof comprises a humanized VH amino acid sequences consisting of SEQ ID NO: 64 and comprises a humanized VL amino acid sequence consisting of SEQ ID NO: 63.

In some embodiments, the present disclosure provides an anti-IL-17A antibody or antigen-binding fragment thereof referred to as ABM67.1.3. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 65 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A antibody or antigen-binding fragment thereof comprises a humanized VH amino acid sequences consisting of SEQ ID NO: 65 and comprises a humanized VL amino acid sequence consisting of SEQ ID NO: 63.

In some embodiments, the present disclosure provides an anti-IL-17A antibody or antigen-binding fragment thereof referred to as ABM67.1.4. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 66 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A antibody or antigen-binding fragment thereof comprises a humanized VH amino acid sequences consisting of SEQ ID NO: 66 and comprises a humanized VL amino acid sequence consisting of SEQ ID NO: 63.

In some embodiments, the present disclosure provides an anti-IL-17A antibody or antigen-binding fragment thereof referred to as ABM67.1.5. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 67 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A antibody or antigen-binding fragment thereof comprises a humanized VH amino acid sequences consisting of SEQ ID NO: 67 and comprises a humanized VL amino acid sequence consisting of SEQ ID NO: 63.

In some embodiments, the present disclosure provides an anti-IL-17A antibody or antigen-binding fragment thereof referred to as ABM67.1.6. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 68 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A antibody or antigen-binding fragment thereof comprises a humanized VH amino acid sequences consisting of SEQ ID NO: 68 and comprises a humanized VL amino acid sequence consisting of SEQ ID NO: 63.

In some embodiments, the present disclosure provides an anti-IL-17A antibody or antigen-binding fragment thereof referred to as ABM67.2. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 69 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 69 and comprises a humanized VL amino acid sequence consisting of SEQ ID NO: 70.

In some embodiments, the present disclosure provides an anti-IL-17A antibody or antigen-binding fragment thereof referred to as ABM67.2.1. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 71 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 71 and comprises a humanized VL amino acid sequence consisting of SEQ ID NO: 70.

In some embodiments, the present disclosure provides an anti-IL-17A antibody or antigen-binding fragment thereof referred to as ABM67.2.2. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 72 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 72 and comprises a humanized VL amino acid sequence consisting of SEQ ID NO: 70.

In some embodiments, the present disclosure provides an anti-IL-17A antibody or antigen-binding fragment thereof referred to as ABM67.2.3. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 73 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 73 and comprises a humanized VL amino acid sequence consisting of SEQ ID NO: 70.

In some embodiments, the present disclosure provides an anti-IL-17A antibody or antigen-binding fragment thereof referred to as ABM67.2.4A. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 74 and comprises a humanized VL amino acid sequence consisting of SEQ ID NO: 70.

In some embodiments, the present disclosure provides an anti-IL-17A antibody or antigen-binding fragment thereof referred to as ABM67.2.4B. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 99 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 99 and comprises a humanized VL amino acid sequence consisting of SEQ ID NO: 70.

In some embodiments, the present disclosure provides an anti-IL-17A antibody or antigen-binding fragment thereof referred to as ABM67.2.4.2 In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 75. In some embodiments, the anti-IL-17A antibody or antigen-binding fragment thereof comprises a humanized VH amino acid sequences consisting of SEQ ID NO: 74 and comprises a humanized VL amino acid sequence consisting of SEQ ID NO: 75.

In some embodiments, the present disclosure provides an anti-IL-17A antibody or antigen-binding fragment thereof referred to as ABM67.2.4.3. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 76. In some embodiments, the anti-IL-17A antibody or antigen-binding fragment thereof comprises a humanized VH amino acid sequences consisting of SEQ ID NO: 74 and comprises a humanized VL amino acid sequence consisting of SEQ ID NO: 76.

In some embodiments, the present disclosure provides an anti-IL-17A antibody or antigen-binding fragment thereof referred to as ABM67.2.5. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 77 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 77 and comprises a humanized VL amino acid sequence consisting of SEQ ID NO: 70.

In some embodiments, the present disclosure provides an anti-IL-17A antibody or antigen-binding fragment thereof referred to as ABM67.2.5.2. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 77 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 75. In some embodiments, the anti-IL-17A antibody or antigen-binding fragment thereof comprises a humanized VH amino acid sequences consisting of SEQ ID NO: 77 and comprises a humanized VL amino acid sequence consisting of SEQ ID NO: 75.

In some embodiments, the present disclosure provides an anti-IL-17A antibody or antigen-binding fragment thereof referred to as ABM67.2.5.3. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 77 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 76. In some embodiments, the anti-IL-17A antibody or antigen-binding fragment thereof comprises a humanized VH amino acid sequences consisting of SEQ ID NO: 77 and comprises a humanized VL amino acid sequence consisting of SEQ ID NO: 76.

In some embodiments, the present disclosure provides an anti-IL-17A antibody or antigen-binding fragment thereof referred to as ABM67.2.6. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 78 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 78 and comprises a humanized VL amino acid sequence consisting of SEQ ID NO: 70.

In some embodiments, the present disclosure provides an anti-IL-17A antibody or antigen-binding fragment thereof referred to as ABM67.2.7. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 79 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A antibody or antigen-binding fragment thereof comprises a humanized VH amino acid sequences consisting of SEQ ID NO: 79 and comprises a humanized VL amino acid sequence consisting of SEQ ID NO: 70.

In some embodiments, the present disclosure provides an anti-IL-17A antibody or antigen-binding fragment thereof referred to as ABM67.2.8. In some embodiments, the anti-IL-17A antibody or antigen-binding fragment thereof comprises a humanized VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a humanized VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 80. In some embodiments, the anti-IL-17A antibody or antigen-binding fragment thereof comprises a humanized VH amino acid sequences consisting of SEQ ID NO: 74 and comprises a humanized VL amino acid sequence consisting of SEQ ID NO: 80.

Exemplary combinations of VH and VL sequences are provided in Table 6 below.

TABLE 6
Exemplary VH and VL combinations

| Construct | VH | SEQ ID | VL | SEQ ID |
|---|---|---|---|---|
| ABM58 | ABM58H | 5 | ABM58L | 9 |
| ABM59 | ABM59H | 5 | ABM59L | 11 |
| ABM59.1 | 59.1H | 12 | 59.1L | 13 |
| ABM59.2 | 59.2H | 14 | 59.2L | 15 |
| ABM59.3 | 59.3H | 16 | 59.2L | 15 |
| ABM59.4 | 59.2H | 14 | 59.3L | 17 |
| ABM59.5 | 59.3H | 16 | 59.3L | 17 |
| ABM59.6 | 59.3H | 16 | 59.4L | 18 |
| ABM59.7 | 59.3H | 16 | 59.5L | 19 |
| ABM59.8 | 59.3H | 16 | 59.6L | 20 |
| ABM59.9 | 59.4H | 21 | 59.4L | 18 |
| ABM59.10 | 59.4H | 21 | 59.5L | 19 |
| ABM59.11 | 59.4H | 21 | 59.6L | 20 |
| ABM59.12 | 59.5H | 22 | 59.4L | 18 |
| ABM59.13 | 59.5H | 22 | 59.5L | 19 |
| ABM59.14 | 59.5H | 22 | 59.6L | 20 |
| ABM60 | ABM60H | 26 | ABM60L | 30 |
| ABM64 | ABM64H | 34 | ABM64L | 38 |
| ABM64.1 | 64.1H | 39 | 64.1L | 40 |
| ABM64.2 | 64.1H | 39 | 64.3L | 41 |
| ABM64.3 | 64.3H | 34 | 64.3L | 41 |
| ABM67 | ABM67H | 57 | ABM67L | 61 |
| ABM67.1 | 67.1H | 62 | 67.1L | 63 |
| ABM67.1.2 | 67.1H (Q99R) | 64 | 67.1L | 63 |
| ABM67.1.3 | 67.1H (N100G) | 65 | 67.1L | 63 |
| ABM67.1.4 | 67.1H (D102R) | 66 | 67.1L | 63 |
| ABM67.1.5 | 67.1H (D102H) | 67 | 67.1L | 63 |
| ABM67.1.6 | 67.1H (Y57H, V64A, V105G) | 68 | 67.1L | 63 |
| ABM67.2 | 67.2H | 69 | 67.2L | 70 |
| ABM67.2.1 | 67.2H (Y106S) | 71 | 67.2L | 70 |
| ABM67.2.2 | 67.2H (A103V) | 72 | 67.2L | 70 |
| ABM67.2.3 | 67.2H (V105A) | 73 | 67.2L | 70 |
| ABM67.2.4A | 67.2H-A (Y106S) | 74 | 67.2L | 70 |
| ABM67.2.4B | 67.2H-B (Y106S) | 99 | 67.2L | 70 |
| ABM67.2.4.2 | 67.2H (Y106S) | 74 | 67.3L | 75 |
| ABM67.2.4.3 | 67.2H (Y106S) | 74 | 67.4L | 76 |
| ABM67.2.5 | 67.2H (Y106I) | 77 | 67.2L | 70 |
| ABM67.2.5.2 | 67.2H (Y106I) | 77 | 67.3L | 75 |
| ABM67.2.5.3 | 67.2H (Y106I) | 77 | 67.4L | 76 |
| ABM67.2.6 | 67.2H (Y106A) | 78 | 67.2L | 70 |
| ABM67.2.7 | 67.2H (Y106K) | 79 | 67.2L | 70 |
| ABM67.2.8 | 67.2H (Y106S) | 74 | 67.6L | 80 |
| ABM72 | ABM72H | 84 | ABM72L | 85 |
| ABM73 | ABM73H | 88 | ABM73L | 90 |
| ABM93 | ABM93H | 93 | ABM93L | 95 |
| ABM96 | ABM96H | 97 | ABM96L | 98 |

Anti-IL-17A scFvs

In some embodiments, the present disclosure provides an anti-IL,-17A scFv comprising a VH amino acid sequence selected from SEQ ID NOs: 5, 12, 14, 16, 21, 22, 26, 34, 39, 57, 62, 64, 65, 66, 67, 68, 69, 71, 72, 73, 74, 99, 77, 78, 79, 84, 88, 93, and 97 and a VL amino acid sequence selected from SEQ ID NOs: 9, 11, 13, 15, 17, 18, 19, 20, 30, 38, 40, 41, 61, 63, 70, 75, 76, 80, 85, 90, 95, and 98.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 5 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 9. In some embodiments, the anti-IL-17A scFv comprises a VH amino acid sequences consisting of SEQ ID NO: 5 and comprises a VL amino acid sequence consisting of SEQ ID NO: 9.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 5 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 11. In some embodiments, the anti-IL-17A scFv comprises a VH amino acid sequences consisting of SEQ ID NO: 5 and comprises a VL amino acid sequence consisting of SEQ ID NO: 11.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 12 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 13. In some embodiments, the anti-IL-17A scFv comprises a VH amino acid sequences consisting of SEQ ID NO: 12 and comprises a VL amino acid sequence consisting of SEQ ID NO: 13.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 14 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 15. In some embodiments, the anti-IL-17A scFv comprises a VH amino acid sequences consisting of SEQ ID NO: 14 and comprises a VL amino acid sequence consisting of SEQ ID NO: 15.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 15. In some embodiments, the anti-IL-17A scFv comprises a VH amino acid sequences consisting of SEQ ID NO: 16 and comprises a VL amino acid sequence consisting of SEQ ID NO: 15.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 14 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 17. In some embodiments, the anti-IL-17A scFv comprises a VH amino acid sequences consisting of SEQ ID NO: 14 and comprises a VL amino acid sequence consisting of SEQ ID NO: 17.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 17. In some embodiments, the anti-IL-17A scFv comprises a VH amino acid sequences consisting of SEQ ID NO: 16 and comprises a VL amino acid sequence consisting of SEQ ID NO: 17.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 18. In some embodiments, the anti-IL-17A scFv comprises a VH amino acid sequences consisting of SEQ ID NO: 16 and comprises a VL amino acid sequence consisting of SEQ ID NO: 18.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 19. In some embodiments, the anti-IL-17A scFv comprises a VH amino acid sequences consisting of SEQ ID NO: 16 and comprises a VL amino acid sequence consisting of SEQ ID NO: 19.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 16 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 20. In some embodiments, the anti-IL-17A scFv comprises a VH amino acid sequences consisting of SEQ ID NO: 16 and comprises a VL amino acid sequence consisting of SEQ ID NO: 20.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 21 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 18. In some embodiments, the anti-IL-17A scFv comprises a VH amino acid sequences consisting of SEQ ID NO: 21 and comprises a VL amino acid sequence consisting of SEQ ID NO: 18.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 21 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 19. In some embodiments, the anti-IL-17A scFv comprises a VH amino acid sequences consisting of SEQ ID NO: 21 and comprises a VL amino acid sequence consisting of SEQ ID NO: 19.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 21 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 20. In some embodiments, the anti-IL-17A scFv comprises a VH amino acid sequences consisting of SEQ ID NO: 21 and comprises a VL amino acid sequence consisting of SEQ ID NO: 20.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 22 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 18. In some embodiments, the anti-IL-17A scFv comprises a VH amino acid sequences consisting of SEQ ID NO: 22 and comprises a VL amino acid sequence consisting of SEQ ID NO: 18.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 22 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 19. In some embodiments, the anti-IL-17A scFv comprises a VH amino acid sequences consisting of SEQ ID NO: 22 and comprises a VL amino acid sequence consisting of SEQ ID NO: 19.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 22 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 20. In some embodiments, the anti-IL-17A scFv comprises a VH amino acid sequences consisting of SEQ ID NO: 22 and comprises a VL amino acid sequence consisting of SEQ ID NO: 20.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 26 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 30. In some embodiments, the anti-IL-17A scFv comprises a VH amino acid sequences consisting of SEQ ID NO: 26 and comprises a VL amino acid sequence consisting of SEQ ID NO: 30.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 34 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 38. In some embodiments, the anti-IL-17A scFv comprises a VH amino acid sequences consisting of SEQ ID NO: 34 and comprises a VL amino acid sequence consisting of SEQ ID NO: 38.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 39 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 40. In some embodiments, the anti-IL-17A scFv comprises a VH amino acid sequences consisting of SEQ ID NO: 39 and comprises a VL amino acid sequence consisting of SEQ ID NO: 40.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 39 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 41. In some embodiments, the anti-IL-17A scFv comprises a VH amino acid sequences consisting of SEQ ID NO: 39 and comprises a VL amino acid sequence consisting of SEQ ID NO: 41.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 34 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 41. In some embodiments, the anti-IL-17A scFv comprises a VH amino acid sequences consisting of SEQ ID NO: 34 and comprises a VL amino acid sequence consisting of SEQ ID NO: 41.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 57 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 61. In some embodiments, the anti-IL-17A scFv comprises a VH amino acid sequences consisting of SEQ ID NO: 57 and comprises a VL amino acid sequence consisting of SEQ ID NO: 61.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 62 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A scFv comprises a VH amino acid sequences consisting of SEQ ID NO: 62 and comprises a VL amino acid sequence consisting of SEQ ID NO: 63.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 64 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A scFv comprises a VH amino acid sequences consisting of SEQ ID NO: 64 and comprises a VL amino acid sequence consisting of SEQ ID NO: 63.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 65 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A scFv comprises a VH amino acid sequences consisting of SEQ ID NO: 65 and comprises a VL amino acid sequence consisting of SEQ ID NO: 63.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 66 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A scFv comprises a VH amino acid sequences consisting of SEQ ID NO: 66 and comprises a VL amino acid sequence consisting of SEQ ID NO: 63.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 67 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A scFv comprises a VH amino acid sequences consisting of SEQ ID NO: 67 and comprises a VL amino acid sequence consisting of SEQ ID NO: 63.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 68 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 63. In some embodiments, the anti-IL-17A scFv comprises a VH amino acid sequences consisting of SEQ ID NO: 68 and comprises a VL amino acid sequence consisting of SEQ ID NO: 63.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 69 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 69 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 71 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 71 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 72 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 72 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 73 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 73 and comprises a VL amino acid sequence consisting of SEQ ID NO: 707.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 74 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 99 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 99 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 75. In some embodiments, the anti-IL-17A scFv comprises a VH amino acid sequences consisting of SEQ ID NO: 74 and comprises a VL amino acid sequence consisting of SEQ ID NO: 75.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 76. In some embodiments, the anti-IL-17A scFv comprises a VH amino acid sequences consisting of SEQ ID NO: 74 and comprises a VL amino acid sequence consisting of SEQ ID NO: 76.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 77 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 77 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 77 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 75. In some embodiments, the anti-IL-17A scFv comprises a VH amino acid sequences consisting of SEQ ID NO: 77 and comprises a VL amino acid sequence consisting of SEQ ID NO: 75.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 77 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 76. In some embodiments, the anti-IL-17A scFv comprises a VH amino acid sequences consisting of SEQ ID NO: 77 and comprises a VL amino acid sequence consisting of SEQ ID NO: 76.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 78 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 78 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 79 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 70. In some embodiments, the anti-IL-17A scFv comprises a VH amino acid sequences consisting of SEQ ID NO: 79 and comprises a VL amino acid sequence consisting of SEQ ID NO: 70.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 74 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 80. In some embodiments, the anti-IL-17A scFv comprises a VH amino acid sequences consisting of SEQ ID NO: 74 and comprises a VL amino acid sequence consisting of SEQ ID NO: 80.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 84 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 85. In some embodiments, the anti-IL-17A scFv comprises a VH amino acid sequences consisting of SEQ ID NO: 84 and comprises a VL amino acid sequence consisting of SEQ ID NO: 85.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 88 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 90. In some embodiments, the anti-IL-17A scFv comprises a VH amino acid sequences consisting of SEQ ID NO: 88 and comprises a VL amino acid sequence consisting of SEQ ID NO: 90.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 93 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 95. In some embodiments, the anti-IL-17A scFv comprises a VH amino acid sequences consisting of SEQ ID NO: 93 and comprises a VL amino acid sequence consisting of SEQ ID NO: 95.

In some embodiments, the anti-IL-17A scFv comprises a VH amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 97 and a VL amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to SEQ ID NO: 98. In some embodiments, the anti-IL-17A scFv comprises a VH amino acid sequences consisting of SEQ ID NO: 97 and comprises a VL amino acid sequence consisting of SEQ ID NO: 98.

In some embodiments, the anti-IL-17A scFv comprises the following structure: VH-linker-VL or VL-linker-VH. As used herein, the term “linker” generally refers to a short polypeptide sequence connecting two sub-domains of a polypeptide. Non-limiting examples of linkers include flexible linkers comprising glycine-serine repeats, and linkers derived from (a) an interdomain region of a transmembrane protein (e.g., a type I transmembrane protein); (b) a stalk region of a type II C-lectin; or (c) an immunoglobulin hinge. In some embodiments, a linker provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids. Exemplary linkers are shown in Table 7.

TABLE 7
Exemplary linkers

| Linker | Amino Acid Sequence | SEQ ID |
|---|---|---|
| STD1 | NYGGGGSGGGGSGGGGSGNS | 143 |
| STD2 | NYGGGGSGGGGSGGGGSGNY | 144 |
| | GGGGSGGGGSGGGGSGNS | |
| H1 | NS | 145 |
| H2 | GGGGSGNS | 146 |
| H3 | NYGGGGSGNS | 147 |
| H4 | GGGGSGGGGSGNS | 148 |
| H5 | NYGGGGSGGGGSGNS | 149 |
| H6 | GGGGSGGGGSGGGGSGNS | 150 |
| H7 | GCPPCPNS | 151 |
| Gly4Ser (G4S) | GGGGS | 152 |
| 2x Gly4Ser (G4S)2 | GGGGSGGGGS | 153 |
| 3x Gly4Ser (G4S)3 | GGGGSGGGGSGGGGS | 154 |
| H105 | SGGGGSGGGGSGGGGS | 155 |
| 4x Gly4Ser (G4S)4 | GGGGSGGGGSGGGGSGGGGS | 156 |
| H75 (NKG2A quadruple | QRHNNSSLNTGTQMAGHSPN | 157 |
| mutant) | S | |
| H83 (NKG2A derived) | SSLNTGTQMAGHSPNS | 158 |
| H106 (NKG2A derived) | QRHNNSSLNTGTQMAGHS | 159 |
| H81 (NKG2D derived) | EVQIPLTESYSPNS | 160 |
| H91 (NKG2D derived) | NSLANQEVQIPLTESYSPNS | 161 |
| H94 | SGGGGSGGGGSGGGGSPNS | 162 |
| H111 | SGGGGSGGGGSGGGGSPGS | 163 |
| H113 | SGGGGSGGGGSGGGGSPAS | 164 |
| H114 | SGGGGSGGGGSGGGGSPS | 165 |
| H115 | SGGGGSGGGGSGGGGSPSS | 166 |

In some embodiments, the anti-IL-17A antibody comprises an antibody light chain and an antibody heavy chain. In some embodiments, the anti-IL-17A antibody is an IgG isotype (e.g. IgG1, IgG2, IgG3, IgG4). In some embodiments, the Fc domain of the anti-IL-17A antibody comprises a wild-type IgG amino acid sequence. Such sequences are known in the art, see e.g. Shields et al., J Biol Chem, (2001) 276:9; 6591-6604.

In some embodiments, the CH2 or CH3 domain of the Fc domain comprises one or more amino acid mutations that alter the function and/or stability of the antibody. For example, in some embodiments, the Fc domain of an anti-IL-17A antibody described herein lacks or has minimal effector functions while retaining the ability to bind some Fc receptors such as the neonatal Fc receptor (FcRn) and retaining a relatively long half-life in vivo. In some embodiments, anti-IL-17A antibodies described do not result in, or substantially reduce the induction of, antibody-dependent cell-mediated cytotoxicity (ADCC), antibody-dependent cell-mediated phagocytosis (ADCP), complement activation, and/or complement-dependent cytotoxicity (CDC). Such mutations are well known in the art, see for example Shields et al., J Biol Chem, (2001) 276:9; 6591-6604; Arduin et al., Mol Immunol (2015) 63:2; 456-463; Vafa et al., Methods (2014) 65:1; 114-126.

In some embodiments, the anti-IL-17A antibodies described are IgG1 isotype antibodies, wherein the IgG1 constant region has a mutation at one or more of the following positions: 228 (S228), 234 (L234), 235 (L235), 237 (G237), 297 (N297), 318 (E318), 320 (K320), 322 (K322), or any combination thereof (numbering according to EU). In some embodiments, the IgG1 Fc domain has an L234A and L235A mutation. In some embodiments, the IgG1 Fc domain has an S228P mutation. As used herein, unless otherwise provided, a position of an amino acid residue in an immunoglobulin molecule is numbered according to EU nomenclature (Ward et al., 1995 Therap. Immunol. 2:77-94). Other numbering systems for amino acid positions within antibodies are known in the art. For example, the IMGT system (Brochet et al, Nucl. Acids Res. (2008) 36, W503-508) and the Kabat numbering system (Kabat, Sequences of Proteins of Immunological Interest, 5^{th} edition, Bethesda, MD: Public Health Service, National Institutes of Health (1991)). Methods and information to convert between one numbering system and the other are known in the art. See, for example, the IMGT Scientific Chart—Correspondence between C numberings, available at imgt.org.

In some embodiments, the anti-IL-17A antibodies described are IgG2 isotype antibodies. the anti-IL-17A antibodies described are IgG3 isotype antibodies. the anti-IL-17A antibodies described are IgG4 isotype antibodies.

In certain embodiments, the anti-IL-17A antibodies and antigen-binding fragments thereof may be prepared using standard molecular biology techniques with regard to selecting antibodies that have a desired specificity. In some embodiments, the anti-IL-17A antibodies and antigen-binding fragments thereof are produced using recombinant DNA technologies. Procedures for the expression and purification of recombinant proteins are well established in the art.

### Bi-Specific Binding Proteins

In some embodiments, the present disclosure provides a bi-specific binding protein comprising an IL-17A binding domain and a second binding domain that specifically binds to a second target antigen.

In some embodiments, the second antigen-binding domain specifically binds to vascular endothelial growth factor (VEGF) or a VEGF receptor. In some embodiments, the VEGF antigen-binding domain is selected from aflibercept (Eylea®), bevacizumab (Avastin®), ranibizumab (Lucentis®), brolucizumab (Beovu®), and ramucirumab (Cyramza®) or an antigen binding fragment thereof. In some embodiments, the VEGF binding domain comprises aflibercept (SEQ ID NO: 100). In some embodiments, the VEGF binding domain comprises the VEGF-R portion of aflibercept (SEQ ID NO: 167). In some embodiments, a VEGF antigen-binding domain is appended to the N-terminus of an anti-IL-17A antibody (e.g., appended to the N-terminus of the heavy chain or the N-terminus of the light chain). In some embodiments, an anti-IL-17A scFv is appended to the N-terminus or the C-terminus of a VEGF binding domain.

In some embodiments, the second antigen-binding domain specifically binds to tumor necrosis factor alpha (TNFα). In some embodiments, the TNFα antigen binding domain is selected from adalimumab (Humira®), infliximab (Remicade®), etanercept (Enbrel®), golimumab (Simponi®), certolizumab (Cimzia®), or an antigen binding fragment thereof. In some embodiments, the TNFα antigen binding domain comprises an adalimumab scFv (SEQ ID NO: 134). In some embodiments, a TNFα antigen-binding domain is appended to the N-terminus of an anti-IL-17A antibody (e.g., appended to the N-terminus of the heavy chain or the N-terminus of the light chain). In some embodiments, an anti-IL-17A scFv is appended to the N-terminus or the C-terminus of a TNFα binding domain.

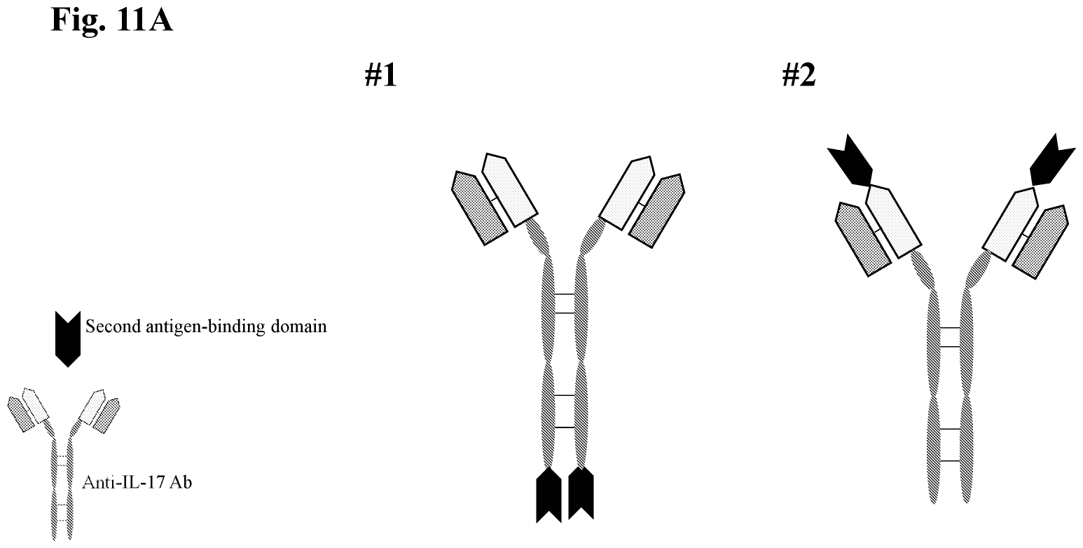

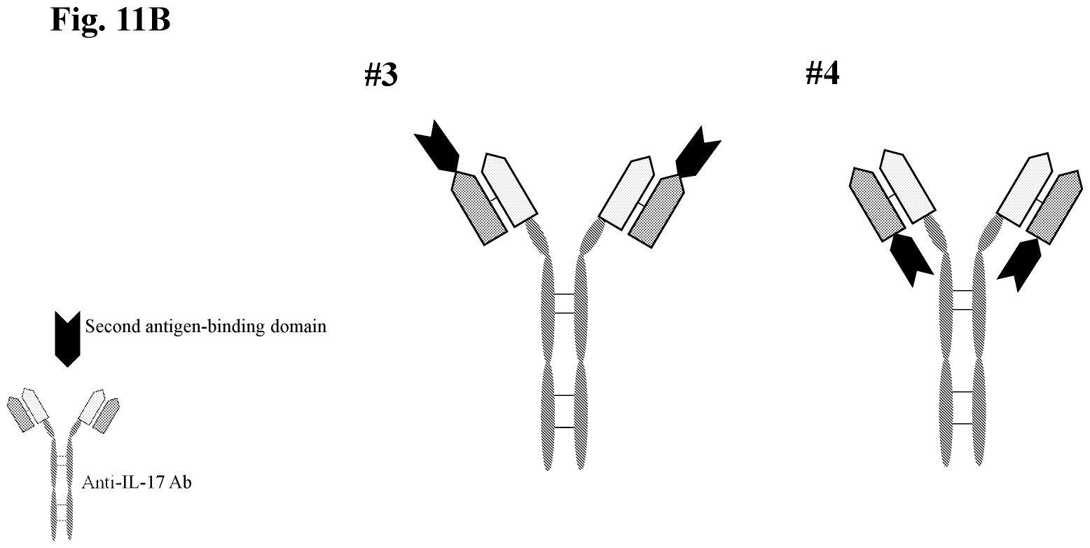

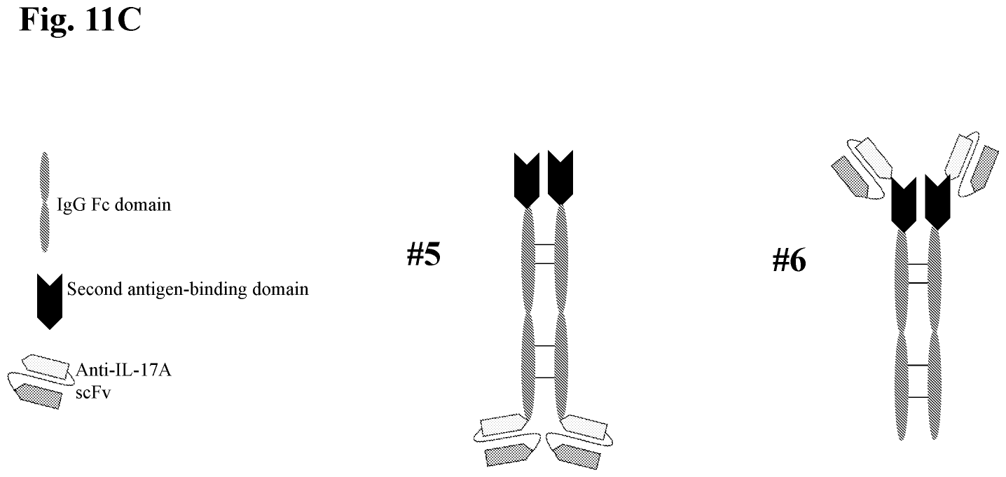

Structures of exemplary bi-specific binding proteins and components thereof of the present disclosure are provided in Tables 8-36 and further exemplified in FIG. 11A-FIG. 11C.

In some embodiments, the present disclosure provides a bi-specific fusion protein referred to herein as 648.1-G1-A. 648.1-G1-A comprises the ABM67.2.4 IgG1 antibody (comprising an IgG1 heavy chain comprising the 67.2.4A VH and a light chain comprising the ABM67.2 VL) with an N-terminal fusion of the VEGF-R portion of aflibercept to the heavy chain. The structure of the heavy chain of 648.1-G1-A comprises:
(a) N′-[aflibercept VEGFR (SEQ ID NO: 167)]-[3×G(4)S]-[67.2.4A IgG1 HC (SEQ ID NO: 169)]-C.

The amino acid sequences of 648.1-G1-A are provided below in Table 8.

TABLE 8
648.1-G1-A Structure and Sequences

| Construct | | SEQ |
|---|---|---|
| component | Amino Acid Sequence | ID |
| aflibercept | SDTGRPFVEMYSEIPEIIHMTEGRELVIPCRVTSPNITVTLKKFPLDTLIPDG | 167 |
| VEGFR | KRIIWDSRKGFIISNATYKEIGLLTCEATVNGHLYKTNYLTHRQTNTIIDVVL | |
| | SPSHGIELSVGEKLVLNCTARTELNVGIDENWEYPSSKHQHKKLVNRDLKTQS | |
| | GSEMKKFLSTLTIDGVTRSDQGLYTCAASSGLMTKKNSTFVRVHEK | |
| 3x G(4)S | GGGGSGGGGSGGGGS | 154 |
| linker | EVQLVESGGGLVKPGGSLRLSCAASGFTFSSYAMSWVRQTPEKRLEWVATISS | 169 |
| 67.2.4A | GGSYTYYPDSVKGRFTISRDNAKNSLYLQMSSLRAEDTAVYYCARQNWDAFVS | |
| IgG1 HC | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVES | |
| | CSVMHEALHNHYTQKSLSLSPGK | |
| Full heavy | SDTGRPFVEMYSEIPEIIHMTEGRELVIPCRVTSPNITVTLKKFPLDTLIPDG | 101 |
| chain fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDENWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLSCAASGFTFSSYAMSWVRQTPEKRL | |
| 67.2.4A HC | EWVATISSGGSYTYYPDSVKGRFTISRDNAKNSLYLQMSSLRAEDTAVYYCAR | |
| | QNWDAFVSWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYICNVNH | |
| | KPSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLTVDKSR | |
| | WQQGNVFSCSVMHEALHNHYTQKSLSLSPGK | |
| Full light | EIVLTQSPVILSVTPKEKVTFTCRAGQSIGTSIHWYQQRTNQSPKLLIKSASE | 136 |
| chain | SISGVPSRFSGSGSGTDFTLTINSLEAEDIATYYCQQSNSWPLTFGAGTKLEI | |
| (comprising | KRTVAAPSVFIFPPSDEQLKSGTASVVCLLNNFYPREAKVQWKVDNALQSGNS | |
| ABM67.2 VL) | QESVTEQDSKDSTYSLSSTLTLSKADYEKHKVYACEVTHQGLSSPVTKSENRG | |
| | EC | |

In some embodiments, the present disclosure provides a bi-specific fusion protein referred to herein as 648.1-G1-B. 648.1-G1-B comprises the ABM67.2.4 IgG1 antibody (comprising an IgG1 heavy chain comprising the 67.2.4B VH and a light chain comprising the ABM67.2 VL) with an N-terminal fusion of the VEGF-R portion of aflibercept to the heavy chain. The structure of the heavy chain of 648.1-G1-B comprises:
(a) N′-[aflibercept VEGFR (SEQ TD NO: 167)]-[3×G(4)S]-[67.2.4B IgG1 HC (SEQ ID NO: 170)]-C′.

The amino acid sequences of 648.1-G1-B are provided below in Table 9.

TABLE 9
648.1-G1-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4B IgG1 HC | EVQLVESGGGLVKPGGSLRLPCAASGFTFSSYAMSWVRQTPERRLEWVATISS | 170 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full heavy chain | SDTGRPFVEMYSEIPEIIHMTEGRELVIPCRVTSPNITVTLKKFPLDTLIPDG | 102 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLPCAASGFTFSSYAMSWVRQTPERRL | |
| 67.2.4B HC | EWVATISSGGSYTYYPDSVKGRFTISRDNAKNSLYLQMSSLRAEDTAVYYCAR | |
| | QNWDAFVSWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYICNVNH | |
| | KPSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLTVDKSR | |
| | WQQGNVFSCSVMHEALHNHYTQKSLSLSPGK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 648.1-G2-A. 648.1-G2-A comprises the ABM67.2.4 IgG2 antibody (comprising an IgG2 heavy chain comprising the 67.2.4A VH and a light chain comprising the ABM67.2 VL) with an N-terminal fusion of the VEGF-R portion of aflibercept to the heavy chain. The structure of the heavy chain of 648.1-G2-A comprises:
(a) N′-[aflibercept VEGFR (SEQ TD NO: 167)]-[3×G(4)S]-[67.2.4A IgG2 HC (SEQ ID NO: 171)]-C′.

The amino acid sequences of 648.1-G2-A are provided below in Table 10.

TABLE 10
648.1-G2-A Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4A IgG2 HC | EVQLVESGGGLVKPGGSLRLSCAASGFTFSSYAMSWVRQTPEKRLEWVATISS | 171 |
| | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSNFGTQTYTCNVDHKPSNTKVD | |
| | KTVERKCCVECPPCPAPPVAGPSVFLFPPKPKDTLMISRTPEVTCVVVDVSHE | |
| | DPEVQFNWYVDGVEVHNAKTKPREEQFNSTFRVVSVLTVVHQDWLNGKEYKCK | |
| | VSNKGLPAPIEKTISKTKGQPREPQVYTLPPSREEMTKNQVSLTCLVKGFYPS | |
| | DISVEWESNGQPENNYKTTPPMLDSDGSFFLYSKLTVDKSRWQQGNVFSCSVM | |
| | HEALHNHYTQKSLSLSPGK | |
| Full heavy chain | SDTGRPFVEMYSEIPEIIHMTEGRELVIPCRVTSPNITVTLKKFPLDTLIPDG | 103 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLSCAASGFTFSSYAMSWVRQTPEKRL | |
| 67.2.4A HC | EWVATISSGGSYTYYPDSVKGRFTISRDNAKNSLYLQMSSLRAEDTAVYYCAR | |
| | QNWDAFVSWGQGTLVTVSSASTKGPSVFPLAPCSRSTSESTAALGCLVKDYFP | |
| | EPVTVSWNSGALTSGVHTFPAVLQSSGLYSLSSVVTVPSSNFGTQTYTCNVDH | |
| | KPSNTKVDKTVERKCCVECPPCPAPPVAGPSVFLFPPKPKDTLMISRTPEVTC | |
| | VVVDVSHEDPEVQFNWYVDGVEVHNAKTKPREEQFNSTFRVVSVLTVVHQDWL | |
| | NGKEYKCKVSNKGLPAPIEKTISKTKGQPREPQVYTLPPSREEMTKNQVSLTC | |
| | LVKGFYPSDISVEWESNGQPENNYKTTPPMLDSDGSFFLYSKLTVDKSRWQQG | |
| | NVFSCSVMHEALHNHYTQKSLSLSPGK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 648.1-G2-B. 648.1-G2-B comprises the ABM67.2.4 IgG2 antibody (comprising an IgG2 heavy chain comprising the 67.2.4B VH and a light chain comprising the ABM67.2 VL) with an N-terminal fusion of the VEGF-R portion of aflibercept to the heavy chain. The structure of the heavy chain of 648.1-G2-B comprises:
(a) N′-[aflibercept VEGFR (SEQ TD NO: 167)]-[3×G(4)S]-[67.2.4B IgG2 HC (SEQ ID NO: 172)]-C′.

The amino acid sequences of 648.1-G2-B are provided below in Table 11.

TABLE 11
648.1-G2-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4B IgG2 HC | EVQLVESGGGLVKPGGSLRLPCAASGFTFSSYAMSWVRQTPERRLEWVATISS | 172 |
| | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSNFGTQTYTCNVDHKPSNTKVD | |
| | KTVERKCCVECPPCPAPPVAGPSVFLFPPKPKDTLMISRTPEVTCVVVDVSHE | |
| | DPEVQFNWYVDGVEVHNAKTKPREEQFNSTFRVVSVLTVVHQDWLNGKEYKCK | |
| | VSNKGLPAPIEKTISKTKGQPREPQVYTLPPSREEMTKNQVSLTCLVKGFYPS | |
| | DISVEWESNGQPENNYKTTPPMLDSDGSFFLYSKLTVDKSRWQQGNVFSCSVM | |
| | HEALHNHYTQKSLSLSPGK | |
| Full heavy chain | SDTGRPFVEMYSEIPEIIHMTEGRELVIPCRVTSPNITVTLKKFPLDTLIPDG | 104 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLPCAASGFTFSSYAMSWVRQTPERRL | |
| 67.2.4B HC | EWVATISSGGSYTYYPDSVKGRFTISRDNAKNSLYLQMSSLRAEDTAVYYCAR | |
| | QNWDAFVSWGQGTLVTVSSASTKGPSVFPLAPCSRSTSESTAALGCLVKDYFP | |
| | EPVTVSWNSGALTSGVHTFPAVLQSSGLYSLSSVVTVPSSNFGTQTYTCNVDH | |
| | KPSNTKVDKTVERKCCVECPPCPAPPVAGPSVFLFPPKPKDTLMISRTPEVTC | |
| | VVVDVSHEDPEVQFNWYVDGVEVHNAKTKPREEQFNSTFRVVSVLTVVHQDWL | |
| | NGKEYKCKVSNKGLPAPIEKTISKTKGQPREPQVYTLPPSREEMTKNQVSLTC | |
| | LVKGFYPSDISVEWESNGQPENNYKTTPPMLDSDGSFFLYSKLTVDKSRWQQG | |
| | NVFSCSVMHEALHNHYTQKSLSLSPGK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 648.1-G4-A. 648.1-G4-A comprises the ABM67.2.4 IgG4 antibody (comprising an IgG2 heavy chain comprising the 67.2.4A VH and a light chain comprising the ABM67.2 VL) with an N-terminal fusion of the VEGF-R portion of aflibercept to the heavy chain. The structure of the heavy chain of 648.1-G4-A comprises:
(a) N′-[aflibercept VEGFR (SEQ TD NO: 167)]-[3×G(4)S]-[67.2.4A IgG4 HC (SEQ ID NO: 173)]-C′.

The amino acid sequences of 648.1-G4-A are provided below in Table 12.

TABLE 12
648.1-G4-A Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGGGGGSGGGGS | 154 |
| 67.2.4A IgG4 HC | EVQLVESGGGLVKPGGSLRLSCAASGFTFSSYAMSWVRQTPEKRLEWVATISS | 173 |
| | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSSLGTKTYTCNVDHKPSNTKVD | |
| | KRVESKYGPPCPSCPAPEFLGGPSVFLFPPKPKDTLMISRTPEVTCVVVDVSQ | |
| | EDPEVQFNWYVDGVEVHNAKTKPREEQFNSTYRVVSVLTVLHQDWLNGKEYKC | |
| | KVSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCSV | |
| | MHEALHNHYTQKSLSLSLGK | |
| Full heavy chain | SDTGRPFVEMYSEIPEIIHMTEGRELVIPCRVTSPNITVTLKKFPLDTLIPDG | 105 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLSCAASGFTFSSYAMSWVRQTPEKRL | |
| 67.2.4A HC | EWVATISSGGSYTYYPDSVKGRFTISRDNAKNSLYLQMSSLRAEDTAVYYCAR | |
| | QNWDAFVSWGQGTLVTVSSASTKGPSVFPLAPCSRSTSESTAALGCLVKDYFP | |
| | EPVTVSWNSGALTSGVHTFPAVLQSSGLYSLSSVVTVPSSSLGTKTYTCNVDH | |
| | KPSNTKVDKRVESKYGPPCPSCPAPEFLGGPSVFLFPPKPKDTLMISRTPEVT | |
| | CVVVDVSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSRWQE | |
| | GNVFSCSVMHEALHNHYTQKSLSLSLGK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 648.1-G4-B. 648.1-G4-B comprises the ABM67.2.4 IgG4 antibody (comprising an IgG2 heavy chain comprising the 67.2.4B VH and a light chain comprising the ABM67.2 VL) with an N-terminal fusion of the VEGF-R portion of aflibercept to the heavy chain. The structure of the heavy chain of 648.1-G4-B comprises:
(a) N′-[aflibercept VEGFR (SEQ TD NO: 167)]-[3×G(4)S]-[67.2.4B IgG4 HC (SEQ ID NO: 174)]-C′.

The amino acid sequences of 648.1-G4-B are provided below in Table 13.

TABLE 13
648.1-G4-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4B IgG4 HC | EVQLVESGGGLVKPGGSLRLPCAASGFTFSSYAMSWVRQTPERRLEWVATISS | 174 |
| | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSSLGTKTYTCNVDHKPSNTKVD | |
| | KRVESKYGPPCPSCPAPEFLGGPSVFLFPPKPKDTLMISRTPEVTCVVVDVSQ | |
| | EDPEVQFNWYVDGVEVHNAKTKPREEQFNSTYRVVSVLTVLHQDWLNGKEYKC | |
| | KVSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCSV | |
| | MHEALHNHYTQKSLSLSLGK | |
| Full heavy chain | SDTGRPFVEMYSEIPEIIHMTEGRELVIPCRVTSPNITVTLKKFPLDTLIPDG | 106 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLPCAASGFTFSSYAMSWVRQTPERRL | |
| 67.2.4B HC | EWVATISSGGSYTYYPDSVKGRFTISRDNAKNSLYLQMSSLRAEDTAVYYCAR | |
| | QNWDAFVSWGQGTLVTVSSASTKGPSVFPLAPCSRSTSESTAALGCLVKDYFP | |
| | EPVTVSWNSGALTSGVHTFPAVLQSSGLYSLSSVVTVPSSSLGTKTYTCNVDH | |
| | KPSNTKVDKRVESKYGPPCPSCPAPEFLGGPSVFLFPPKPKDTLMISRTPEVT | |
| | CVVVDVSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSRWQE | |
| | GNVFSCSVMHEALHNHYTQKSLSLSLGK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 648.2-G1-A. 648.2-G1-A comprises the ABM67.2.8 IgG1 antibody (comprising an IgG1 heavy chain comprising the 67.2.4A VH and a light chain comprising the ABM67.6 VL) with an N-terminal fusion of the VEGF-R portion of aflibercept to the heavy chain. The structure of the heavy chain of 648.2-G1-A comprises:
(a) N′-[aflibercept VEGFR (SEQ TD NO: 167)]-[3×G(4)S]-[67.2.4A IgG1 HC (SEQ ID NO: 169)]-C′.

The amino acid sequences of 648.2-G1-A are provided below in Table 14.

TABLE 14
648.2-G1-A Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4A IgG1 HC | EVQLVESGGGLVKPGGSLRLSCAASGFTFSSYAMSWVRQTPEKRLEWVATISS | 169 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full heavy chain | SDTGRPFVEMYSEIPEIIHMTEGRELVIPCRVTSPNITVTLKKFPLDTLIPDG | 107 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLSCAASGFTFSSYAMSWVRQTPEKRL | |
| 67.2.4A HC | EWVATISSGGSYTYYPDSVKGRFTISRDNAKNSLYLQMSSLRAEDTAVYYCAR | |
| | QNWDAFVSWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYICNVNH | |
| | KPSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLTVDKSR | |
| | WQQGNVFSCSVMHEALHNHYTQKSLSLSPGK | |
| Full light chain | EIVLTQSPDFQSVTPKEKVTITCRAGQSIGTSIHWYQRKPDQSPKLLIKSASE | 137 |
| (comprising | SISGVPSRFSGSGSGTDFTLTINSLEAEDAATYYCQQSNSWPLTFGAGTKVEI | |
| ABM67.6 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 648.2-G1-B. 648.2-G1-B comprises the ABM67.2.8 IgG1 antibody (comprising an IgG1 heavy chain comprising the 67.2.4B VH and a light chain comprising the ABM67.6 VL) with an N-terminal fusion of the VEGF-R portion of aflibercept to the heavy chain. The structure of the heavy chain of 648.2-G1-B comprises:
(a) N′-[aflibercept VEGFR (SEQ TD NO: 167)]-[3×G(4)S]-[67.2.4B IgG1 HC (SEQ ID NO: 170)]-C′.

The amino acid sequences of 648.2-G1-B are provided below in Table 15.

TABLE 15
648.2-G1-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4B IgG1 HC | EVQLVESGGGLVKPGGSLRLPCAASGFTFSSYAMSWVRQTPERRLEWVATISS | 170 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full heavy chain | SDTGRPFVEMYSEIPEIIHMTEGRELVIPCRVTSPNITVTLKKFPLDTLIPDG | 108 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEVQLVESGGGLVKPGGSLRLPCAASGFTFSSYAMSWVRQTPERRL | |
| 67.2.4B HC | EWVATISSGGSYTYYPDSVKGRFTISRDNAKNSLYLQMSSLRAEDTAVYYCAR | |
| | QNWDAFVSWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYICNVNH | |
| | KPSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLTVDKSR | |
| | WQQGNVFSCSVMHEALHNHYTQKSLSLSPGK | |
| Full light chain | EIVLTQSPDFQSVTPKEKVTITCRAGQSIGTSIHWYQRKPDQSPKLLIKSASE | 137 |
| (comprising | SISGVPSRFSGSGSGTDFTLTINSLEAEDAATYYCQQSNSWPLTFGAGTKVEI | |
| ABM67.6 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.1-G1-A. 649.1-G1-A comprises the ABM67.2.4 IgG1 antibody (comprising an IgG1 heavy chain comprising the 67.2.4A VH and a light chain comprising the ABM67.2 VL) with an C-terminal fusion of the VEGF-R portion of aflibercept to the heavy chain. The structure of the heavy chain of 649.1-G1-A comprises:
(a) N′-[67.2.4A IgG1 HC (SEQ TD NO: 169)]-[3×G(4)S]-[aflibercept VEGFR (SEQ TD NO: 167)]-C′.

The amino acid sequences of 649.1-G1-A are provided below in Table 16.

TABLE 16
649.1-G1-A Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4A IgG1 HC | EVQLVESGGGLVKPGGSLRLSCAASGFTFSSYAMSWVRQTPEKRLEWVATISS | 169 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full heavy chain | EVQLVESGGGLVKPGGSLRLSCAASGFTFSSYAMSWVRQTPEKRLEWVATISS | 109 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4A HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGKGGGGGSGGGGSGGGGSSDTGRPFVEMYSEI | |
| | PEIIHMTEGRELVIPCRVTSPNITVTLKKFPLDTLIPDGKRIIWDSRKGFIIS | |
| | NATYKEIGLLTCEATVNGHLYKTNYLTHRQTNTIIDVVLSPSHGIELSVGEKL | |
| | VLNCTARTELNVGIDFNWEYPSSKHQHKKLVNRDLKTQSGSEMKKFLSTLTID | |
| | GVTRSDQGLYTCAASSGLMTKKNSTFVRVHEK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.1-G1-B. 649.1-G1-B comprises the ABM67.2.4 IgG1 antibody (comprising an IgG1 heavy chain comprising the 67.2.4B VH and a light chain comprising the ABM67.2 VL) with a C-terminal fusion of the VEGF-R portion of aflibercept to the heavy chain. The structure of the heavy chain of 649.1-G1-B comprises:
(a) N′-[67.2.4B IgG1 HC (SEQ ID NO: 170)]-[3×G(4)S]-[aflibercept VEGFR (SEQ ID NO: 167)]-C′.

The amino acid sequences of 649.1-G1-B are provided below in Table 17.

TABLE 17
649.1-G1-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| 67.2.4B IgG1 HC | EVQLVESGGGLVKPGGSLRLPCAASGFTFSSYAMSWVRQTPERRLEWVATISS | 170 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full heavy chain | EVQLVESGGGLVKPGGSLRLPCAASGFTFSSYAMSWVRQTPERRLEWVATISS | 110 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4B HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGKGGGGGSGGGGSGGGGSSDTGRPFVEMYSEI | |
| | PEIIHMTEGRELVIPCRVTSPNITVTLKKFPLDTLIPDGKRIIWDSRKGFIIS | |
| | NATYKEIGLLTCEATVNGHLYKTNYLTHRQTNTIIDVVLSPSHGIELSVGEKL | |
| | VLNCTARTELNVGIDFNWEYPSSKHQHKKLVNRDLKTQSGSEMKKFLSTLTID | |
| | GVTRSDQGLYTCAASSGLMTKKNSTFVRVHEK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.2-G1-A. 649.2-G1-A comprises the ABM67.2.4 IgG1 antibody (comprising an IgG1 heavy chain comprising the 67.2.4A VH and a light chain comprising the ABM67.2 VL) with a C-terminal fusion of the VEGF-R portion of aflibercept to the heavy chain. The terminal K on the IgG heavy chain has been removed, and there is a single S at start of VEGFR sequence. The structure of the heavy chain of 649.1-G1-A comprises:
(a) N′-[67.2.4A IgG1 HC (SEQ TD NO: 175)]-[2×G(4)S]-[aflibercept VEGFR (SEQ ID NO: 168)]-C′.

The amino acid sequences of 649.2-G1-A are provided below in Table 18.

TABLE 18
649.2-G1-A Structure and Sequences

| Construct | | |
|---|---|---|
| component | Amino Acid Sequence | SEQ ID |
| aflibercept VEGFR | DTGRPFVEMYSEIPEIIHMTEGRELVIPCRVTSPNITVTLKKFPLDTLIPDGK | 168 |
| | RIIWDSRKGFIISNATYKEIGLLTCEATVNGHLYKTNYLTHRQTNTIIDVVLS | |
| | PSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEK | |
| 2x G(4)S linker | GGGGGSGGGGS | 153 |
| 67.2.4A IgG1 HC | EVQLVESGGGLVKPGGSLRLSCAASGFTFSSYAMSWVRQTPEKRLEWVATISS | 175 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPG | |
| Full heavy chain | EVQLVESGGGLVKPGGSLRLSCAASGFTFSSYAMSWVRQTPEKRLEWVATISS | 111 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4A HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGGGGGGSGGGGSDTGRPFVEMYSEIPEIIHMT | |
| | EGRELVIPCRVTSPNITVTLKKFPLDTLIPDGKRIIWDSRKGFIISNATYKEI | |
| | GLLTCEATVNGHLYKTNYLTHRQTNTIIDVVLSPSHGIELSVGEKLVLNCTAR | |
| | TELNVGIDFNWEYPSSKHQHKKLVNRDLKTQSGSEMKKFLSTLTIDGVTRSDQ | |
| | GLYTCAASSGLMTKKNSTFVRVHEK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.2-G1-B. 649.2-G1-B comprises the ABM67.2.4 IgG1 antibody (comprising an IgG1 heavy chain comprising the 67.2.4B VH and a light chain comprising the ABM67.2 VL) with a C-terminal fusion of the VEGF-R portion of aflibercept to the heavy chain. The terminal K on the IgG heavy chain has been removed, and there is a single S at start of VEGFR sequence. The structure of the heavy chain of 649.1-G1-B comprises:
(a) N′-[67.2.4B IgG1 HC (SEQ ID NO: 176)]-[2×G(4)S]-[aflibercept VEGFR (SEQ TD NO: 168)]-C′.

The amino acid sequences of 649.2-G1-B are provided below in Table 19.

TABLE 19
649.2-G1-B Structure and Sequences

| Construct | | |
|---|---|---|
| component | Amino Acid Sequence | SEQ ID |
| aflibercept VEGFR | DTGRPFVEMYSEIPEIIHMTEGRELVIPCRVTSPNITVTLKKFPLDTLIPDGK | 168 |
| | RIIWDSRKGFIISNATYKEIGLLTCEATVNGHLYKTNYLTHRQTNTIIDVVLS | |
| | PSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEK | |
| 2x G(4)S linker | GGGGGSGGGGS | 153 |
| 67.2.4B IgG1 HC | EVQLVESGGGLVKPGGSLRLPCAASGFTFSSYAMSWVRQTPERRLEWVATISS | 176 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPG | |
| Full heavy chain | EVQLVESGGGLVKPGGSLRLPCAASGFTFSSYAMSWVRQTPERRLEWVATISS | 112 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4B HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGGGGGGSGGGGSDTGRPFVEMYSEIPEIIHMT | |
| | EGRELVIPCRVTSPNITVTLKKFPLDTLIPDGKRIIWDSRKGFIISNATYKEI | |
| | GLLTCEATVNGHLYKTNYLTHRQTNTIIDVVLSPSHGIELSVGEKLVLNCTAR | |
| | TELNVGIDFNWEYPSSKHQHKKLVNRDLKTQSGSEMKKFLSTLTIDGVTRSDQ | |
| | GLYTCAASSGLMTKKNSTFVRVHEK | |
| Full light chain | EIVLTQSPVILSVTPKEKVTFTCRAGQSIGTSIHWYQQRTNQSPKLLIKSASE | 136 |
| (comprising | SISGVPSRFSGSGSGTDFTLTINSLEAEDIATYYCQQSNSWPLTFGAGTKLEI | |
| ABM67.2 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.3-G1-A. 649.3-G1-A comprises the ABM67.2.8 IgG1 antibody (comprising an IgG1 heavy chain comprising the 67.2.4A VH and a light chain comprising the ABM67.6 VL) with a C-terminal fusion of the VEGF-R portion of aflibercept to the heavy chain. The structure of the heavy chain of 649.3-G1-A comprises:
(a) N′-[67.2.4A IgG1 HC (SEQ TD NO: 169)]-[3×G(4)S]-[aflibercept VEGFR (SEQ ID NO: 167)]-C′.

The amino acid sequences of 649.3-G1-A are provided below in Table 20.

TABLE 20
649.3-G1-A Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGGSGGGGS | 154 |
| 67.2.4A IgG1 HC | EVQLVESGGGLVKPGGSLRLSCAASGFTFSSYAMSWVRQTPEKRLEWVATISS | 169 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full heavy chain | EVQLVESGGGLVKPGGSLRLSCAASGFTFSSYAMSWVRQTPEKRLEWVATISS | 113 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4A HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGKGGGGGSGGGGSGGGGSSDTGRPFVEMYSEI | |
| | PEIIHMTEGRELVIPCRVTSPNITVTLKKFPLDTLIPDGKRIIWDSRKGFIIS | |
| | NATYKEIGLLTCEATVNGHLYKTNYLTHRQTNTIIDVVLSPSHGIELSVGEKL | |
| | VLNCTARTELNVGIDFNWEYPSSKHQHKKLVNRDLKTQSGSEMKKFLSTLTID | |
| | GVTRSDQGLYTCAASSGLMTKKNSTFVRVHEK | |
| Full light chain | EIVLTQSPDFQSVTPKEKVTITCRAGQSIGTSIHWYQRKPDQSPKLLIKSASE | 137 |
| (comprising | SISGVPSRFSGSGSGTDFTLTINSLEAEDAATYYCQQSNSWPLTFGAGTKVEI | |
| ABM67.6 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.3-G1-B. 649.3-G1-B comprises the ABM67.2.8 IgG1 antibody (comprising an IgG1 heavy chain comprising the 67.2.4B3 VH and a light chain comprising the ABM67.6 VL) with a C-terminal fusion of the VEGF-R portion of aflibercept to the heavy chain. The structure of the heavy chain of 649.3-G1-B comprises:
(a) N′-[67.2.4B IgG1 HC (SEQ ID NO: 170)]-[3×G(4)S]-[aflibercept VEGFR (SEQ TD NO: 167)]-C′.

The amino acid sequences of 649.3-G1-B are provided below in Table 21.

TABLE 21
649.3-G1-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGGSGGGGSGGGGS | 154 |
| 67.2.4B IgG1 HC | EVQLVESGGGLVKPGGSLRLPCAASGFTFSSYAMSWVRQTPERRLEWVATISS | 170 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full heavy chain | EVQLVESGGGLVKPGGSLRLPCAASGFTFSSYAMSWVRQTPERRLEWVATISS | 114 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4B HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGKGGGGGSGGGGSGGGGSSDTGRPFVEMYSEI | |
| | PEIIHMTEGRELVIPCRVTSPNITVTLKKFPLDTLIPDGKRIIWDSRKGFIIS | |
| | NATYKEIGLLTCEATVNGHLYKTNYLTHRQTNTIIDVVLSPSHGIELSVGEKL | |
| | VLNCTARTELNVGIDFNWEYPSSKHQHKKLVNRDLKTQSGSEMKKFLSTLTID | |
| | GVTRSDQGLYTCAASSGLMTKKNSTFVRVHEK | |
| Full light chain | EIVLTQSPDFQSVTPKEKVTITCRAGQSIGTSIHWYQRKPDQSPKLLIKSASE | 137 |
| (comprising | SISGVPSRFSGSGSGTDFTLTINSLEAEDAATYYCQQSNSWPLTFGAGTKVEI | |
| ABM67.6 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.4-G1-A. 649.4-G1-A comprises the ABM67.2.8 IgG1 antibody (comprising an IgG1 heavy chain comprising the 67.2.4A VH and a light chain comprising the ABM67.6 VL) with a C-terminal fusion of the VEGF-R portion of aflibercept to the heavy chain. The terminal K on the IgG heavy chain has been removed, and there is a single S at start of VEGFR sequence. The structure of the heavy chain of 649.4-G1-A comprises:
(a) N′-[67.2.4A IgG1 HC (SEQ TD NO: 175)]-[2×G(4)S]-[aflibercept VEGFR (SEQ ID NO: 168)]-C′.

The amino acid sequences of 649.4-G1-A are provided below in Table 22.

TABLE 22
649.4-G1-A Structure and Sequences

| Construct | | |
|---|---|---|
| component | Amino Acid Sequence | SEQ ID |
| aflibercept VEGFR | DTGRPFVEMYSEIPEIIHMTEGRELVIPCRVTSPNITVTLKKFPLDTLIPDGK | 168 |
| | RIIWDSRKGFIISNATYKEIGLLTCEATVNGHLYKTNYLTHRQTNTIIDVVLS | |
| | PSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEK | |
| 2x G(4)S linker | GGGGGSGGGGS | 153 |
| 67.2.4A IgG1 HC | EVQLVESGGGLVKPGGSLRLSCAASGFTFSSYAMSWVRQTPEKRLEWVATISS | 175 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVES | |
| | CSVMHEALHNHYTQKSLSLSPG | |
| Full heavy chain | EVQLVESGGGLVKPGGSLRLSCAASGFTFSSYAMSWVRQTPEKRLEWVATISS | 115 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4A HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGGGGGGSGGGGSDTGRPFVEMYSEIPEIIHMT | |
| | EGRELVIPCRVTSPNITVTLKKFPLDTLIPDGKRIIWDSRKGFIISNATYKEI | |
| | GLLTCEATVNGHLYKTNYLTHRQTNTIIDVVLSPSHGIELSVGEKLVLNCTAR | |
| | TELNVGIDFNWEYPSSKHQHKKLVNRDLKTQSGSEMKKFLSTLTIDGVTRSDQ | |
| | GLYTCAASSGLMTKKNSTFVRVHEK | |
| Full light chain | EIVLTQSPDFQSVTPKEKVTITCRAGQSIGTSIHWYQRKPDQSPKLLIKSASE | 137 |
| (comprising | SISGVPSRFSGSGSGTDFTLTINSLEAEDAATYYCQQSNSWPLTFGAGTKVEI | |
| ABM67.6 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 649.4-G1-B. 649.4-G1-B comprises the ABM67.2.8 IgG1 antibody (comprising an IgG1 heavy chain comprising the 67.2.4B VH and a light chain comprising the ABM67.6 VL) with a C-terminal fusion of the VEGF-R portion of aflibercept to the heavy chain. The terminal K on the IgG heavy chain has been removed, and there is a single S at start of VEGFR sequence. The structure of the heavy chain of 649.4-G1-B comprises:
(a) N′-[67.2.4B IgG1 HC (SEQ ID NO: 176)]-[2×G(4)S]-[aflibercept VEGFR (SEQ TD NO: 168)]-C′.

The amino acid sequences of 649.4-G1-B are provided below in Table 23.

TABLE 23
649.4-G1-B Structure and Sequences

| Construct | | |
|---|---|---|
| component | Amino Acid Sequence | SEQ ID |
| aflibercept VEGFR | DTGRPFVEMYSEIPEIIHMTEGRELVIPCRVTSPNITVTLKKFPLDTLIPDGK | 168 |
| | RIIWDSRKGFIISNATYKEIGLLTCEATVNGHLYKTNYLTHRQTNTIIDVVLS | |
| | PSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEK | |
| 2x G(4)S linker | GGGGGSGGGGS | 153 |
| 67.2.4B IgG1 HC | EVQLVESGGGLVKPGGSLRLPCAASGFTFSSYAMSWVRQTPERRLEWVATISS | 176 |
| | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPG | |
| Full heavy chain | EVQLVESGGGLVKPGGSLRLPCAASGFTFSSYAMSWVRQTPERRLEWVATISS | 116 |
| fusion | GGSYTYYPDSVKGRFTISRDNAKNSLYLQMSSLRAEDTAVYYCARQNWDAFVS | |
| aflibercept | WGQGTLVTVSSASTKGPSVFPLAPSSKSTSGGTAALGCLVKDYFPEPVTVSWN | |
| VEGFR | SGALTSGVHTFPAVLQSSGLYSLSSVVTVPSSSLGTQTYICNVNHKPSNTKVD | |
| Linker | KKVEPKSCDKTHTCPPCPAPELLGGPSVFLFPPKPKDTLMISRTPEVTCVVVD | |
| 67.2.4B HC | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVES | |
| | CSVMHEALHNHYTQKSLSLSPGGGGGGSGGGGSDTGRPFVEMYSEIPEIIHMT | |
| | EGRELVIPCRVTSPNITVTLKKFPLDTLIPDGKRIIWDSRKGFIISNATYKEI | |
| | GLLTCEATVNGHLYKTNYLTHRQTNTIIDVVLSPSHGIELSVGEKLVLNCTAR | |
| | TELNVGIDFNWEYPSSKHQHKKLVNRDLKTQSGSEMKKFLSTLTIDGVTRSDQ | |
| | GLYTCAASSGLMTKKNSTFVRVHEK | |
| Full light chain | EIVLTQSPDFQSVTPKEKVTITCRAGQSIGTSIHWYQRKPDQSPKLLIKSASE | 137 |
| (comprising | SISGVPSRFSGSGSGTDFTLTINSLEAEDAATYYCQQSNSWPLTFGAGTKVEI | |
| ABM67.6 VL) | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 651.1-G2-A. 651.1-G2-A comprises the ABM67.2.4 IgG2 antibody (comprising an IgG2 heavy chain comprising the 67.2.4A VH and a light chain comprising the ABM67.2 VL) with an N-terminal fusion of the VEGF-R portion of aflibercept to the light chain. The structure of the light chain of 651.1-G2-A comprises:
(a) N′-[aflibercept VEGFR (SEQ TD NO: 167)]-[3×G(4)S]-[67.2 LC (SEQ ID NO: 136)]-C′.

The amino acid sequences of 651.1-G2-A are provided below in Table 24.

TABLE 24
651.1-G2-A Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| ABM67.2 LC | EIVLTQSPVILSVTPKEKVTFTCRAGQSIGTSIHWYQQRTNQSPKLLIKSASE | 136 |
| | SISGVPSRFSGSGSGTDFTLTINSLEAEDIATYYCQQSNSWPLTFGAGTKLEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| Full heavy chain | EVQLVESGGGLVKPGGSLRLSCAASGFTFSSYAMSWVRQTPEKRLEWVATISS | 117 |
| 67.2.4A IgG2 HC | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full light chain | SDTGRPFVEMYSEIPEIIHMTEGRELVIPCRVTSPNITVTLKKFPLDTLIPDG | 138 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEIVLTQSPVILSVTPKEKVTFTCRAGQSIGTSIHWYQQRTNQSPK | |
| 67.2 LC | LLIKSASESISGVPSRFSGSGSGTDFTLTINSLEAEDIATYYCQQSNSWPLTF | |
| | GAGTKLEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSSP | |
| | VTKSFNRGEC | |

In some embodiments, the present disclosure provides a bi-specific fusion protein referred to herein as 651.1-G2-B. 651.1-G2-B comprises the ABM67.2.4 IgG2 antibody (comprising an IgG2 heavy chain comprising the 67.2.4B VH and a light chain comprising the ABM67.2 VL) with an N-terminal fusion of the VEGF-R portion of aflibercept to the light chain. The structure of the light chain of 651.1-G2-B comprises:
(a) N′-[aflibercept VEGFR (SEQ TD NO: 167)]-[3×G(4)S]—[67.2 LC (SEQ ID NO: 136)]-C′.

The amino acid sequences of 651.1-G2-B are provided below in Table 25.

TABLE 25
651.1-G2-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| ABM67.2 LC | EIVLTQSPVILSVTPKEKVTFTCRAGQSIGTSIHWYQQRTNQSPKLLIKSASE | 136 |
| | SISGVPSRFSGSGSGTDFTLTINSLEAEDIATYYCQQSNSWPLTFGAGTKLEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| Full heavy chain | EVQLVESGGGLVKPGGSLRLPCAASGFTFSSYAMSWVRQTPERRLEWVATISS | 118 |
| 67.2.4B IgG2 HC | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full light chain | SDTGRPFVEMYSEIPEIIHMTEGRELVIPCRVTSPNITVTLKKFPLDTLIPDG | 138 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEIVLTQSPVILSVTPKEKVTFTCRAGQSIGTSIHWYQQRTNQSPK | |
| 67.2 LC | LLIKSASESISGVPSRFSGSGSGTDFTLTINSLEAEDIATYYCQQSNSWPLTF | |
| | GAGTKLEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSSP | |
| | VTKSFNRGEC | |

In some embodiments, the present disclosure provides a bi-specific fusion protein referred to herein as 651.2-G1-A. 651.2-G1-A comprises the ABM67.2.4 IgG1 antibody (comprising an IgG1 heavy chain comprising the 67.2.4A VH and a light chain comprising the ABM67.6 VL) with an N-terminal fusion of the VEGF-R portion of aflibercept to the light chain. The structure of the light chain of 651.2-G1-A comprises:
(a) N′-[aflibercept VEGFR (SEQ TD NO: 167)]-[3×TL(4)S]—[67.6 LC (SEQ ID NO: 137)]-C′.

The amino acid sequences of 651.2-G1-A are provided below in Table 26.

TABLE 26
651.2-G1-A Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| ABM67.6 LC | EIVLTQSPDFQSVTPKEKVTITCRAGQSIGTSIHWYQRKPDQSPKLLIKSASE | 137 |
| | SISGVPSRFSGSGSGTDFTLTINSLEAEDAATYYCQQSNSWPLTFGAGTKVEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| Full heavy chain | EVQLVESGGGLVKPGGSLRLSCAASGFTFSSYAMSWVRQTPEKRLEWVATISS | 119 |
| 67.2.4A IgG1 HC | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVEFS | |
| | CSVMHEALHNHYTQKSLSLSPGK | |
| Full light chain | SDTGRPFVEMYSEIPEIIHMTEGRELVIPCRVTSPNITVTLKKFPLDTLIPDG | 139 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEIVLTQSPDFQSVTPKEKVTITCRAGQSIGTSIHWYQRKPDQSPK | |
| 67.6 LC | LLIKSASESISGVPSRFSGSGSGTDFTLTINSLEAEDAATYYCQQSNSWPLTF | |
| | GAGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSSP | |
| | VTKSFNRGEC | |

In some embodiments, the present disclosure provides a bi-specific fusion protein referred to herein as 651.2-G1-B. 651.2-G1-B comprises the ABM67.2.4 IgG1 antibody (comprising an IgG1 heavy chain comprising the 67.2.4B VH and a light chain comprising the ABM67.6 VL) with an N-terminal fusion of the VEGF-R portion of aflibercept to the light chain. The structure of the light chain of 651.2-G1-B comprises:
(a) N′-[aflibercept VEGFR (SEQ TD NO: 167)]-[3×G(4)S]—[67.6 LC (SEQ ID NO: 137)]-C′.

The amino acid sequences of 651.2-G1-B are provided below in Table 27.

TABLE 27
651.2-G1-B Structure and Sequences

| Construct | | |
|---|---|---|
| component | Amino Acid Sequence | SEQ ID |
| aflibercept VEGFR | SDTGRPFVEMYSEIPEIIHMTEGRELVIPCRVTSPNITVTLKKFPLDTLIPDG | 167 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEK | |
| 3x G(4)S linker | GGGGSGGGGSGGGGS | 154 |
| ABM67.6 LC | EIVLTQSPDFQSVTPKEKVTITCRAGQSIGTSIHWYQRKPDQSPKLLIKSASE | 137 |
| | SISGVPSRFSGSGSGTDFTLTINSLEAEDAATYYCQQSNSWPLTFGAGTKVEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| Full heavy chain | EVQLVESGGGLVKPGGSLRLPCAASGFTFSSYAMSWVRQTPERRLEWVATISS | 120 |
| 67.2.4A IgG1 HC | GGSYTYYPDSVKGRFTISRDNAKNSLYLQMSSLRAEDTAVYYCARQNWDAFVS | |
| | WGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPGK | |
| Full light chain | SDTGRPFVEMYSEIPEIIHMTEGRELVIPCRVTSPNITVTLKKFPLDTLIPDG | 139 |
| fusion | KRIIWDSRKGFIISNATYKEIGLLTCEATVNGHLYKTNYLTHRQTNTIIDVVL | |
| aflibercept | SPSHGIELSVGEKLVLNCTARTELNVGIDFNWEYPSSKHQHKKLVNRDLKTQS | |
| VEGFR | GSEMKKFLSTLTIDGVTRSDQGLYTCAASSGLMTKKNSTFVRVHEKGGGGSGG | |
| Linker | GGSGGGGSEIVLTQSPDFQSVTPKEKVTITCRAGQSIGTSIHWYQRKPDQSPK | |
| 67.6 LC | LLIKSASESISGVPSRFSGSGSGTDFTLTINSLEAEDAATYYCQQSNSWPLTF | |
| | GAGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSSP | |
| | VTKSFNRGEC | |

In some embodiments, the present disclosure provides a bi-specific fusion protein referred to herein as 652.1. 652.1 comprises Aflibercept with a C-terminal fusion of the ABM59 scFv. The structure of 652.1 comprises:
(a) N′-[Aflibercept VEGFR+IgG1 Fc]-[2×G(4)S]—59.5 VH-[3×G(4)S]—59.5 VL-C′

The amino acid sequences of 652.1 are provided below in Table 28.

TABLE 28
652.1 Structure and Sequences

| Construct | | |
|---|---|---|
| component | Amino Acid Sequence | SEQ ID |
| aflibercept | SDTGRPFVEMYSEIPEIIHMTEGRELVIPCRVTSPNITVTLKKFPLDTLIPDG | 100 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEKDKTHTCP | |
| | PCPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQK | |
| | SLSLSPG | |
| 3x G(4)S linker | GGGGSGGGGSGGGGS | 154 |
| 2x G(4)S linker | GGGGSGGGGS | 153 |
| ABM59 scFv | EVQLVESGGGLVQPGGSLRLSCAASGFAFSSYDMSWVRQAPGKGLEWVAYISS | 177 |
| 59.5 VH | GGGNTYYPDTVKGRFTISRDNAKNTLYLQMNSLRAEDTAVYYCAREALTMIST | |
| Linker | RDYYAMDYWGQGTLVTVSSGGGGSGGGGSGGGGSDIQLTQSPSSLSASVGDRV | |
| 59.5 VL | TITCGASESVDNYGISFMDWYQQKPGKAPKLLIYAASNQGSGVPSRFSGSGSG | |
| | TDFTLTISSLQPEDFATYYCQQSKEVPYTFGQGTKVEIKR | |
| Full fusion | SDTGRPFVEMYSEIPEIIHMTEGRELVIPCRVTSPNITVTLKKFPLDTLIPDG | 123 |
| sequence | KRIIWDSRKGFIISNATYKEIGLLTCEATVNGHLYKTNYLTHRQTNTIIDVVL | |
| Aflibercept | SPSHGIELSVGEKLVLNCTARTELNVGIDFNWEYPSSKHQHKKLVNRDLKTQS | |
| Linker | GSEMKKFLSTLTIDGVTRSDQGLYTCAASSGLMTKKNSTFVRVHEKDKTHTCP | |
| ABM59 scFv | PCPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQK | |
| | SLSLSPGGGGGSGGGGSEVQLVESGGGLVQPGGSLRLSCAASGFAFSSYDMSW | |
| | VRQAPGKGLEWVAYISSGGGNTYYPDTVKGRFTISRDNAKNTLYLQMNSLRAE | |
| | DTAVYYCAREALTMISTRDYYAMDYWGQGTLVTVSSGGGGSGGGGSGGGGSDI | |
| | QLTQSPSSLSASVGDRVTITCGASESVDNYGISFMDWYQQKPGKAPKLLIYAA | |
| | SNQGSGVPSRFSGSGSGTDFTLTISSLQPEDFATYYCQQSKEVPYTFGQGTKV | |
| | EIKR | |

In some embodiments, the present disclosure provides a bi-specific fusion protein referred to herein as 653.1-A. 653.1-A comprises Aflibercept with a C-terminal fusion of the ABM67-A scFv. The structure of 653.1-A comprises:
(a) N′-[Aflibercept VEGFR+IgG1 Fc]-[2×G(4)S]—67.2.4A VH-[3×G(4)S]-67.6 VL-C′

The amino acid sequences of 653.1-A are provided below in Table 29.

TABLE 29
653.1-A Structure and Sequences

| Construct | | |
|---|---|---|
| component | Amino Acid Sequence | SEQ ID |
| aflibercept | SDTGRPFVEMYSEIPEIIHMTEGRELVIPCRVTSPNITVTLKKFPLDTLIPDG | 100 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEKDKTHTCP | |
| | PCPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQK | |
| | SLSLSPG | |
| 3x G(4)S linker | GGGGSGGGGSGGGGS | 154 |
| 2x G(4)S linker | GGGGSGGGGS | 153 |
| ABM67-A scFv | EVQLVESGGGLVKPGGSLRLSCAASGFTFSSYAMSWVRQTPEKRLEWVATISS | 178 |
| 67.2.4A VH | GGSYTYYPDSVKGRFTISRDNAKNSLYLQMSSLRAEDTAVYYCARQNWDAFVS | |
| Linker | WGQGTLVTVSSGGGGSGGGGSGGGGSEIVLTQSPDFQSVTPKEKVTITCRAGQ | |
| 67.6 VL | SIGTSIHWYQRKPDQSPKLLIKSASESISGVPSRFSGSGSGTDFTLTINSLEA | |
| | EDAATYYCQQSNSWPLTFGAGTKVEIKR | |
| Full fusion | SDTGRPFVEMYSEIPEIIHMTEGRELVIPCRVTSPNITVTLKKFPLDTLIPDG | 124 |
| sequence | KRIIWDSRKGFIISNATYKEIGLLTCEATVNGHLYKTNYLTHRQTNTIIDVVL | |
| Aflibercept | SPSHGIELSVGEKLVLNCTARTELNVGIDFNWEYPSSKHQHKKLVNRDLKTQS | |
| Linker | GSEMKKFLSTLTIDGVTRSDQGLYTCAASSGLMTKKNSTFVRVHEKDKTHTCP | |
| ABM67-A scFv | PCPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQK | |
| | SLSLSPGGGGGSGGGGSEVQLVESGGGLVKPGGSLRLSCAASGFTFSSYAMSW | |
| | VRQTPEKRLEWVATISSGGSYTYYPDSVKGRFTISRDNAKNSLYLQMSSLRAE | |
| | DTAVYYCARQNWDAFVSWGQGTLVTVSSGGGGSGGGGSGGGGSEIVLTQSPDF | |
| | QSVTPKEKVTITCRAGQSIGTSIHWYQRKPDQSPKLLIKSASESISGVPSRFS | |
| | GSGSGTDFTLTINSLEAEDAATYYCQQSNSWPLTFGAGTKVEIKR | |

In some embodiments, the present disclosure provides a bi-specific fusion protein referred to herein as 653.1-B. 653.1-B comprises Aflibercept with a C-terminal fusion of the ABM67-B scFv. The structure of 653.1-B3 comprises:
(a) N′-[Aflibercept VEGFR+IgG1 FC]-[2×G(4)S]—67.2.4B VH-[3×G(4)S]-67.6 VL-C′

The amino acid sequences of 653.1-B are provided below in Table 30.

TABLE 30
653.1-B Structure and Sequences

| Construct | | |
|---|---|---|
| component | Amino Acid Sequence | SEQ ID |
| aflibercept | SDTGRPFVEMYSEIPEIIHMTEGRELVIPCRVTSPNITVTLKKFPLDTLIPDG | 100 |
| | KRIIWDSRKGFIISNATYKEIGLLTCEATVNGHLYKTNYLTHRQTNTIIDVVL | |
| | SPSHGIELSVGEKLVLNCTARTELNVGIDFNWEYPSSKHQHKKLVNRDLKTQS | |
| | GSEMKKFLSTLTIDGVTRSDQGLYTCAASSGLMTKKNSTFVRVHEKDKTHTCP | |
| | PCPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQK | |
| | SLSLSPG | |
| 3x G(4)S linker | GGGGSGGGGSGGGGS | 154 |
| 2x G(4)S linker | GGGGSGGGGS | 153 |
| ABM67 scFv | EVQLVESGGGLVKPGGSLRLPCAASGFTFSSYAMSWVRQTPERRLEWVATISS | 179 |
| 67.2.4B VH | GGSYTYYPDSVKGRFTISRDNAKNSLYLQMSSLRAEDTAVYYCARQNWDAFVS | |
| Linker | WGQGTLVTVSSGGGGSGGGGSGGGGSEIVLTQSPDFQSVTPKEKVTITCRAGQ | |
| 67.6 VL | SIGTSIHWYQRKPDQSPKLLIKSASESISGVPSRFSGSGSGTDFTLTINSLEA | |
| | EDAATYYCQQSNSWPLTFGAGTKVEIKR | |
| Full fusion | SDTGRPFVEMYSEIPEIIHMTEGRELVIPCRVTSPNITVTLKKFPLDTLIPDG | 125 |
| sequence | KRIIWDSRKGFIISNATYKEIGLLTCEATVNGHLYKTNYLTHRQTNTIIDVVL | |
| Aflibercept | SPSHGIELSVGEKLVLNCTARTELNVGIDFNWEYPSSKHQHKKLVNRDLKTQS | |
| Linker | GSEMKKFLSTLTIDGVTRSDQGLYTCAASSGLMTKKNSTFVRVHEKDKTHTCP | |
| ABM67-B scFv | PCPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQK | |
| | SLSLSPGGGGGSGGGGSEVQLVESGGGLVKPGGSLRLPCAASGFTFSSYAMSW | |
| | VRQTPERRLEWVATISSGGSYTYYPDSVKGRFTISRDNAKNSLYLQMSSLRAE | |
| | DTAVYYCARQNWDAFVSWGQGTLVTVSSGGGGSGGGGSGGGGSEIVLTQSPDF | |
| | QSVTPKEKVTITCRAGQSIGTSIHWYQRKPDQSPKLLIKSASESISGVPSRFS | |
| | GSGSGTDFTLTINSLEAEDAATYYCQQSNSWPLTFGAGTKVEIKR | |

In some embodiments, the present disclosure provides a bi-specific fusion protein referred to herein as 392.1. 392.1 comprises an ABM59.6 IgG2 antibody (comprising an IgG2 heavy chain comprising the ABM59.3 VH and a light chain comprising the ABM59.4 VL) with an N-terminal fusion of the Adalimumab scFv to the heavy chain. The structure of the heavy chain of 392.1 comprises:
(a) N′-[Adalimumab scFv (SEQ ID NO: 134)]-[3×G(4)S]—[ABM59.6 IgG2 HC (SEQ ID NO: 180)]-C′.

The amino acid sequences of 392.1 are provided below in Table 31.

TABLE 31
392.1 Structure and Sequences

| Construct | | |
|---|---|---|
| component | Amino Acid Sequence | SEQ ID |
| Adalimumab | EVQLVESGGGLVQPGRSLRLSCAASGFTFDDYAMHWVRQAPGKGLEWVSAITW | 134 |
| scFv (VH- | NSGHIDYADSVEGRFTISRDNAKNSLYLQMNSLRAEDTAVYYCAKVSYLSTAS | |
| linker-VL) | SLDYWGQGTLVTVSSGGGGSGGGGSGGGGSGGGGSDIQMTQSPSSLSASVGDR | |
| | VTITCRASQGIRNYLAWYQQKPGKAPKLLIYAASTLQSGVPSRFSGSGSGTDF | |
| | ILTISSLQPEDVATYYCQRYNRAPYTFGQGTKVEIKR | |
| 3x G(4)S linker | GGGGSGGGGSGGGGS | 154 |
| 2x G(4)S linker | GGGGSGGGGS | 153 |
| ABM59.6 IgG2 | EVKLVESGGGLVQPGGSLRLSCAASGFAFSSYDMSWVRQAPEKRLEWVAYISS | 180 |
| HC | GGGNTYYPDTVKGRFTISRDNAKNSLYLQMNSLRAEDTAVYYCAREALTMIST | |
| | RDYYAMDYWGQGTLVTVSSASTKGPSVFPLAPCSRSTSESTAALGCLVKDYFP | |
| | EPVTVSWNSGALTSGVHTFPAVLQSSGLYSLSSVVTVPSSNFGTQTYTCNVDH | |
| | KPSNTKVDKTVERKCCVECPPCPAPPVAGPSVFLFPPKPKDTLMISRTPEVTC | |
| | VVVDVSHEDPEVQFNWYVDGVEVHNAKTKPREEQFNSTFRVVSVLTVVHQDWL | |
| | NGKEYKCKVSNKGLPAPIEKTISKTKGQPREPQVYTLPPSREEMTKNQVSLTC | |
| | LVKGFYPSDIAVEWESNGQPENNYKTTPPMLDSDGSFFLYSKLTVDKSRWQQG | |
| | NVFSCSVMHEALHNHYTQKSLSLSPGK | |
| Full heavy chain | EVQLVESGGGLVQPGRSLRLSCAASGFTFDDYAMHWVRQAPGKGLEWVSAITW | 127 |
| fusion sequence | NSGHIDYADSVEGRETISRDNAKNSLYLQMNSLRAEDTAVYYCAKVSYLSTAS | |
| Adalimumab scFv | SLDYWGQGTLVTVSSGGGGSGGGGSGGGGSGGGGSDIQMTQSPSSLSASVGDR | |
| Linker B- | VTITCRASQGIRNYLAWYQQKPGKAPKLLIYAASTLQSGVPSRFSGSGSGTDF | |
| AMB59.6 IgG2 | TLTISSLQPEDVATYYCQRYNRAPYTFGQGTKVEIKRGGGGSGGGGSGGGGSE | |
| HC | VKLVESGGGLVQPGGSLRLSCAASGFAFSSYDMSWVRQAPEKRLEWVAYISSG | |
| | GGNTYYPDTVKGRFTISRDNAKNSLYLQMNSLRAEDTAVYYCAREALTMISTR | |
| | DYYAMDYWGQGTLVTVSSASTKGPSVFPLAPCSRSTSESTAALGCLVKDYFPE | |
| | PVTVSWNSGALTSGVHTFPAVLQSSGLYSLSSVVTVPSSNFGTQTYTCNVDHK | |
| | PSNTKVDKTVERKCCVECPPCPAPPVAGPSVFLFPPKPKDTLMISRTPEVTCV | |
| | VVDVSHEDPEVQFNWYVDGVEVHNAKTKPREEQFNSTFRVVSVLTVVHQDWLN | |
| | GKEYKCKVSNKGLPAPIEKTISKTKGQPREPQVYTLPPSREEMTKNQVSLTCL | |
| | VKGFYPSDIAVEWESNGQPENNYKTTPPMLDSDGSFFLYSKLTVDKSRWQQGN | |
| | VFSCSVMHEALHNHYTQKSLSLSPGK | |
| Light chain | DIVLTQSPASLAVSPGQRATITCGASESVDNYGISFMDWFQQKPGQPPKLLIY | 141 |
| sequence | AASNQGSGVPARFSGSGSGTDFTLTINPVEAQDTANYYCQQSKEVPYTFGGGT | |
| ABM59.4 VL | KLEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTKS | |
| | FNRGEC | |

In some embodiments, the present disclosure provides a bi-specific fusion protein referred to herein as 393.1. 393.1 comprises an ABM59.6 IgG4 antibody (comprising an IgG4 heavy chain comprising the ABM59.3 VH and a light chain comprising the ABM59.4 VL) with an N-terminal fusion of the Adalimumab scFv to the heavy chain. The structure of the heavy chain of 393.1 comprises:
(a) N′-[Adalimumab scFv (SEQ ID NO: 134)]-[3×G(4)S]—[ABM59.6 IgG4 HC (SEQ ID NO: 181)]-C′.

The amino acid sequences of 393.1 are provided below in Table 32.

TABLE 32
393.1 Structure and Sequences

| Construct | | |
|---|---|---|
| component | Amino Acid Sequence | SEQ ID |
| Adalimumab | EVQLVESGGGLVQPGRSLRLSCAASGFTFDDYAMHWVRQAPGKGLEWVSAITW | 134 |
| scFv (VH- | NSGHIDYADSVEGRFTISRDNAKNSLYLQMNSLRAEDTAVYYCAKVSYLSTAS | |
| linker-VL) | SLDYWGQGTLVTVSSGGGGSGGGGSGGGGSGGGGSDIQMTQSPSSLSASVGDR | |
| | VTITCRASQGIRNYLAWYQQKPGKAPKLLIYAASTLQSGVPSRFSGSGSGTDF | |
| | TLTISSLQPEDVATYYCQRYNRAPYTFGQGTKVEIKR | |
| 3x G(4)S linker | GGGGSGGGGSGGGGS | 154 |
| 2x G(4)S linker | GGGGSGGGGS | 153 |
| ABM59.6 IgG4 | EVKLVESGGGLVQPGGSLRLSCAASGFAFSSYDMSWVRQAPEKRLEWVAYISS | 181 |
| HC | GGGNTYYPDTVKGRFTISRDNAKNSLYLQMNSLRAEDTAVYYCAREALTMIST | |
| | RDYYAMDYWGQGTLVTVSSASTKGPSVFPLAPCSRSTSESTAALGCLVKDYFP | |
| | EPVTVSWNSGALTSGVHTFPAVLQSSGLYSLSSVVTVPSSSLGTKTYTCNVDH | |
| | KPSNTKVDKRVESKYGPPCPPCPAPEFLGGPSVFLFPPKPKDTLMISRTPEVT | |
| | CVVVDVSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSRWQE | |
| | GNVFSCSVMHEALHNHYTQKSLSLSLGL | |
| Full heavy chain | EVQLVESGGGLVQPGRSLRLSCAASGFTFDDYAMHWVRQAPGKGLEWVSAITW | 128 |
| fusion sequence | NSGHIDYADSVEGRETISRDNAKNSLYLQMNSLRAEDTAVYYCAKVSYLSTAS | |
| Adalimumab scFv | SLDYWGQGTLVTVSSGGGGSGGGGSGGGGSGGGGSDIQMTQSPSSLSASVGDR | |
| Linker B | VTITCRASQGIRNYLAWYQQKPGKAPKLLIYAASTLQSGVPSRFSGSGSGTDF | |
| AMB59.3 IgG4 | TLTISSLQPEDVATYYCQRYNRAPYTFGQGTKVEIKRGGGGSGGGGSGGGGSE | |
| HC | VKLVESGGGLVQPGGSLRLSCAASGFAFSSYDMSWVRQAPEKRLEWVAYISSG | |
| | GGNTYYPDTVKGRFTISRDNAKNSLYLQMNSLRAEDTAVYYCAREALTMISTR | |
| | DYYAMDYWGQGTLVTVSSASTKGPSVFPLAPCSRSTSESTAALGCLVKDYFPE | |
| | PVTVSWNSGALTSGVHTFPAVLQSSGLYSLSSVVTVPSSSLGTKTYTCNVDHK | |
| | PSNTKVDKRVESKYGPPCPPCPAPEFLGGPSVFLFPPKPKDTLMISRTPEVTC | |
| | VVVDVSQEDPEVQFNWYVDGVEVHNAKTKPREEQFNSTYRVVSVLTVLHQDWL | |
| | NGKEYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRWQEG | |
| | NVFSCSVMHEALHNHYTQKSLSLSLGL | |
| Light chain | DIVLTQSPASLAVSPGQRATITCGASESVDNYGISFMDWFQQKPGQPPKLLIY | 141 |
| sequence | AASNQGSGVPARFSGSGSGTDFTLTINPVEAQDTANYYCQQSKEVPYTFGGGT | |
| ABM59.4 VL | KLEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTKS | |
| | FNRGEC | |

In some embodiments, the present disclosure provides a bi-specific fusion protein referred to herein as 408.1A. 408.1A comprises antibody with N-terminal Adalimumab scFv fusion on HC an ABM67.2.4.2 IgG2 antibody (comprising an IgG2 heavy chain comprising the ABM67.2.4A VH and a light chain comprising the ABM67.3 VL) with an N-terminal fusion of the Adalimumab scFv to the heavy chain. The structure of the heavy chain of 408.1A comprises:
(a) N′-[Adalimumab scFv (SEQ ID NO: 134)]-[3×G(4)S]—[ABM67.2.4.2A IgG2 HC (SEQ ID NO: 182)]-C′.

The amino acid sequences of 408.1A are provided below in Table 33.

TABLE 33
408.1A Structure and Sequences

| Construct | | |
|---|---|---|
| component | Amino Acid Sequence | SEQ ID |
| Adalimumab scFv | EVQLVESGGGLVQPGRSLRLSCAASGFTFDDYAMHWVRQAPGKGLEWVSAITW | 134 |
| (VH-linker-VL) | NSGHIDYADSVEGRFTISRDNAKNSLYLQMNSLRAEDTAVYYCAKVSYLSTAS | |
| | SLDYWGQGTLVTVSSGGGGSGGGGSGGGGSGGGGSDIQMTQSPSSLSASVGDR | |
| | VTITCRASQGIRNYLAWYQQKPGKAPKLLIYAASTLQSGVPSRFSGSGSGTDF | |
| | TLTISSLQPEDVATYYCQRYNRAPYTFGQGTKVEIKR | |
| 3x G(4)S linker | GGGGSGGGGGGGGS | 154 |
| 2x G(4)S linker | GGGGSGGGGS | 153 |
| ABM67.2.4.2A | EVQLVESGGGLVKPGGSLRLSCAASGFTFSSYAMSWVRQTPEKRLEWVATISS | 182 |
| IgG2 HC | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSNFGTQTYTCNVDHKPSNTKVD | |
| | KTVERKCCVECPPCPAPPVAGPSVFLFPPKPKDTLMISRTPEVTCVVVDVSHE | |
| | DPEVQFNWYVDGVEVHNAKTKPREEQFNSTFRVVSVLTVVHQDWLNGKEYKCK | |
| | VSNKGLPAPIEKTISKTKGQPREPQVYTLPPSREEMTKNQVSLTCLVKGFYPS | |
| | DIAVEWESNGQPENNYKTTPPMLDSDGSFFLYSKLTVDKSRWQQGNVFSCSVM | |
| | HEALHNHYTQKSLSLSPGK | |
| Full heavy chain | EVQLVESGGGLVQPGRSLRLSCAASGFTFDDYAMHWVRQAPGKGLEWVSAITW | 129 |
| fusion sequence | NSGHIDYADSVEGRFTISRDNAKNSLYLQMNSLRAEDTAVYYCAKVSYLSTAS | |
| Adalimumab scFv | SLDYWGQGTLVTVSSGGGGSGGGGSGGGGSGGGGSDIQMTQSPSSLSASVGDR | |
| Linker B | VTITCRASQGIRNYLAWYQQKPGKAPKLLIYAASTLQSGVPSRFSGSGSGTDF | |
| ABM67.2.4.2 IgG2 | TLTISSLQPEDVATYYCQRYNRAPYTFGQGTKVEIKRGGGGSGGGGSGGGGSE | |
| HC | VQLVESGGGLVKPGGSLRLSCAASGFTFSSYAMSWVRQTPEKRLEWVATISSG | |
| | GSYTYYPDSVKGRFTISRDNAKNSLYLQMSSLRAEDTAVYYCARQNWDAFVSW | |
| | GQGTLVTVSSASTKGPSVFPLAPCSRSTSESTAALGCLVKDYFPEPVTVSWNS | |
| | GALTSGVHTFPAVLQSSGLYSLSSVVTVPSSNFGTQTYTCNVDHKPSNTKVDK | |
| | TVERKCCVECPPCPAPPVAGPSVFLFPPKPKDTLMISRTPEVTCVVVDVSHED | |
| | PEVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPSD | |
| | IAVEWESNGQPENNYKTTPPMLDSDGSFFLYSKLTVDKSRWQQGNVFSCSVMH | |
| | EALHNHYTQKSLSLSPGK | |
| Light chain | EIVLTQSPVILSVTPKEKVTFTCRAGQSIGTSIHWYQQRTDQSPKLLIKSASE | 142 |
| sequence | SISGVPSRFSGSGSGTDFTLTINSLEAEDIATYYCQQSNSWPLTFGAGTKLEI | |
| ABM67.3 VL | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 408.1B. 408.1B comprises antibody with N-terminal Adalimumab scFv fusion on HC an ABM67.2.4.2 IgG2 antibody (comprising an IgG2 heavy chain comprising the ABM67.2.41B VH and a light chain comprising the ABM67.3 VL) with an N-terminal fusion of the Adalimumab scFv to the heavy chain. The structure of the heavy chain of 408.1B comprises:
(a) N′-[Adalimumab scFv (SEQ ID NO: 134)]-[3×G(4)S]—[ABM67.2.4.2B IgG2 HC (SEQ ID NO: 183)]-C′.

The amino acid sequences of 408.1B are provided below in Table 34.

TABLE 34
408.1B Structure and Sequences

| Construct | | |
|---|---|---|
| component | Amino Acid Sequence | SEQ ID |
| Adalimumab scFv | EVQLVESGGGLVQPGRSLRLSCAASGFTFDDYAMHWVRQAPGKGLEWVSAITW | 134 |
| (VH-linker-VL) | NSGHIDYADSVEGRFTISRDNAKNSLYLQMNSLRAEDTAVYYCAKVSYLSTAS | |
| | SLDYWGQGTLVTVSSGGGGSGGGGSGGGGSGGGGSDIQMTQSPSSLSASVGDR | |
| | VTITCRASQGIRNYLAWYQQKPGKAPKLLIYAASTLQSGVPSRFSGSGSGTDF | |
| | TLTISSLQPEDVATYYCQRYNRAPYTFGQGTKVEIKR | |
| 3x G(4)S linker | GGGGSGGGGSGGGGS | 154 |
| 2x G(4)S linker | GGGGSGGGGS | 153 |
| ABM67.2.4.2B | EVQLVESGGGLVKPGGSLRLPCAASGFTESSYAMSWVRQTPERRLEWVATISS | 183 |
| IgG2 HC | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSNFGTQTYTCNVDHKPSNTKVD | |
| | KTVERKCCVECPPCPAPPVAGPSVFLFPPKPKDTLMISRTPEVTCVVVDVSHE | |
| | DPEVQFNWYVDGVEVHNAKTKPREEQFNSTFRVVSVLTVVHQDWLNGKEYKCK | |
| | VSNKGLPAPIEKTISKTKGQPREPQVYTLPPSREEMTKNQVSLTCLVKGFYPS | |
| | DIAVEWESNGQPENNYKTTPPMLDSDGSFFLYSKLTVDKSRWQQGNVFSCSVM | |
| | HEALHNHYTQKSLSLSPGK | |
| Full heavy chain | EVQLVESGGGLVQPGRSLRLSCAASGFTEDDYAMHWVRQAPGKGLEWVSAITW | 130 |
| fusion sequence | NSGHIDYADSVEGRFTISRDNAKNSLYLQMNSLRAEDTAVYYCAKVSYLSTAS | |
| Adalimumab scFv | SLDYWGQGTLVTVSSGGGGSGGGGSGGGGSGGGGSDIQMTQSPSSLSASVGDR | |
| Linker B | VTITCRASQGIRNYLAWYQQKPGKAPKLLIYAASTLQSGVPSRFSGSGSGTDF | |
| ABM67.2.4.2B | TLTISSLQPEDVATYYCQRYNRAPYTFGQGTKVEIKRGGGGSGGGGSGGGGSE | |
| IgG2 HC | VQLVESGGGLVKPGGSLRLPCAASGFTFSSYAMSWVRQTPERRLEWVATISSG | |
| | GSYTYYPDSVKGRFTISRDNAKNSLYLQMSSLRAEDTAVYYCARQNWDAFVSW | |
| | GQGTLVTVSSASTKGPSVFPLAPCSRSTSESTAALGCLVKDYFPEPVTVSWNS | |
| | GALTSGVHTFPAVLQSSGLYSLSSVVTVPSSNFGTQTYTCNVDHKPSNTKVDK | |
| | TVERKCCVECPPCPAPPVAGPSVFLFPPKPKDTLMISRTPEVTCVVVDVSHED | |
| | PEVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPSD | |
| | IAVEWESNGQPENNYKTTPPMLDSDGSFFLYSKLTVDKSRWQQGNVFSCSVMH | |
| | EALHNHYTQKSLSLSPGK | |
| Light chain | EIVLTQSPVILSVTPKEKVTFTCRAGQSIGTSIHWYQQRTDQSPKLLIKSASE | 142 |
| sequence | SISGVPSRFSGSGSGTDFTLTINSLEAEDIATYYCQQSNSWPLTFGAGTKLEI | |
| ABM67.3 VL | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the present disclosure provides a bi-specific fusion protein referred to herein as 409.1A. 409.1A comprises antibody with N-terminal Adalimumab scFv fusion on HC an ABM67.2.4.2 IgG4 antibody (comprising an IgG4 heavy chain comprising the ABM67.2.4A VH and a light chain comprising the ABM67.3 VL) with an N-terminal fusion of the Adalimumab scFv to the heavy chain. The structure of the heavy chain of 409.1A comprises:
(a) N′-[Adalimumab scFv (SEQ ID NO: 134)]-[3×G(4)S]—[ABM67.2.4.2A IgG4 HC (SEQ ID NO: 184)]-C′.

The amino acid sequences of 409.1A are provided below in Table 35.

TABLE 35
409.1A Structure and Sequences

| Construct | | |
|---|---|---|
| component | Amino Acid Sequence | SEQ ID |
| Adalimumab scFv | EVQLVESGGGLVQPGRSLRLSCAASGFTFDDYAMHWVRQAPGKGLEWVSAITW | 134 |
| (VH-linker-VL) | NSGHIDYADSVEGRFTISRDNAKNSLYLQMNSLRAEDTAVYYCAKVSYLSTAS | |
| | SLDYWGQGTLVTVSSGGGGSGGGGSGGGGSGGGGSDIQMTQSPSSLSASVGDR | |
| | VTITCRASQGIRNYLAWYQQKPGKAPKLLIYAASTLQSGVPSRFSGSGSGTDF | |
| | TLTISSLQPEDVATYYCQRYNRAPYTFGQGTKVEIKR | |
| 3x G(4)S linker | GGGGSGGGGSGGGGS | 154 |
| 2x G(4)S linker | GGGGSGGGGS | 153 |
| ABM67.2.4.2A | EVQLVESGGGLVKPGGSLRLPCAASGFTFSSYAMSWVRQTPERRLEWVATISS | 184 |
| IgG4 HC | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSSLGTKTYTCNVDHKPSNTKVD | |
| | KRVESKYGPPCPPCPAPEFLGGPSVFLFPPKPKDTLMISRTPEVTCVVVDVSQ | |
| | EDPEVQFNWYVDGVEVHNAKTKPREEQFNSTYRVVSVLTVLHQDWLNGKEYKC | |
| | KVSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCSV | |
| | MHEALHNHYTQKSLSLSLGL | |
| Full heavy chain | EVQLVESGGGLVQPGRSLRLSCAASGFTFDDYAMHWVRQAPGKGLEWVSAITW | 131 |
| fusion sequence | NSGHIDYADSVEGRETISRDNAKNSLYLQMNSLRAEDTAVYYCAKVSYLSTAS | |
| Adalimumab scFv | SLDYWGQGTLVTVSSGGGGSGGGGSGGGGSGGGGSDIQMTQSPSSLSASVGDR | |
| Linker B | VTITCRASQGIRNYLAWYQQKPGKAPKLLIYAASTLQSGVPSRFSGSGSGTDE | |
| ABM67.2.4.2 IgG4 | TLTISSLQPEDVATYYCQRYNRAPYTFGQGTKVEIKRGGGGSGGGGSGGGGSE | |
| HC | VQLVESGGGLVKPGGSLRLPCAASGFTFSSYAMSWVRQTPERRLEWVATISSG | |
| | GSYTYYPDSVKGRFTISRDNAKNSLYLQMSSLRAEDTAVYYCARQNWDAFVSW | |
| | GQGTLVTVSSASTKGPSVFPLAPCSRSTSESTAALGCLVKDYFPEPVTVSWNS | |
| | GALTSGVHTFPAVLQSSGLYSLSSVVTVPSSSLGTKTYTCNVDHKPSNTKVDK | |
| | RVESKYGPPCPPCPAPEFLGGPSVFLFPPKPKDTLMISRTPEVTCVVVDVSQE | |
| | DPEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCSVM | |
| | HEALHNHYTQKSLSLSLGL | |
| Light chain | EIVLTQSPVILSVTPKEKVTFTCRAGQSIGTSIHWYQQRTDQSPKLLIKSASE | 142 |
| sequence | SISGVPSRFSGSGSGTDFTLTINSLEAEDIATYYCQQSNSWPLTFGAGTKLEI | |
| ABM67.3 VL | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSENRG | |
| | EC | |

In some embodiments, the present disclosure provides a bi-specific fusion protein referred to herein as 409.1B. 409.1B comprises antibody with N-terminal Adalimumab scFv fusion on HC an ABM67.2.4.2B IgG4 antibody (comprising an IgG4 heavy chain comprising the ABM67.2.41B VH and a light chain comprising the ABM67.3 VL) with an N-terminal fusion of the Adalimumab scFv to the heavy chain. The structure of the heavy chain of 409.1B comprises:
(a) N′-[Adalimumab scFv (SEQ ID NO: 134)]-[3×G(4)S]—[ABM67.2.4.2B IgG4 HC (SEQ ID NO: 185)]-C′.

The amino acid sequences of 409.1B are provided below in Table 36.

TABLE 36
409.1B Structure and Sequences

| Construct | | |
|---|---|---|
| component | Amino Acid Sequence | SEQ ID |
| Adalimumab scFv | EVQLVESGGGLVQPGRSLRLSCAASGFTFDDYAMHWVRQAPGKGLEWVSAITW | 134 |
| (VH-linker-VL) | NSGHIDYADSVEGRFTISRDNAKNSLYLQMNSLRAEDTAVYYCAKVSYLSTAS | |
| | SLDYWGQGTLVTVSSGGGGSGGGGSGGGGSGGGGSDIQMTQSPSSLSASVGDR | |
| | VTITCRASQGIRNYLAWYQQKPGKAPKLLIYAASTLQSGVPSRFSGSGSGTDF | |
| | TLTISSLQPEDVATYYCQRYNRAPYTFGQGTKVEIKR | |
| 3x G(4)S linker | GGGGSGGGGSGGGGS | 154 |
| 2x G(4)S linker | GGGGSGGGGS | 153 |
| ABM67.2.4.2B | EVQLVESGGGLVKPGGSLRLPCAASGFTFSSYAMSWVRQTPERRLEWVATISS | 185 |
| IgG4 HC | GGSYTYYPDSVKGRFTISRDNAKNSLYLQMSSLRAEDTAVYYCARQNWDAFVS | |
| | WGQGTLVTVSSASTKGPSVFPLAPCSRSTSESTAALGCLVKDYFPEPVTVSWN | |
| | SGALTSGVHTFPAVLQSSGLYSLSSVVTVPSSSLGTKTYTCNVDHKPSNTKVD | |
| | KRVESKYGPPCPPCPAPEFLGGPSVFLFPPKPKDTLMISRTPEVTCVVVDVSQ | |
| | EDPEVQFNWYVDGVEVHNAKTKPREEQFNSTYRVVSVLTVLHQDWLNGKEYKC | |
| | KVSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCSV | |
| | MHEALHNHYTQKSLSLSLGL | |
| Full heavy chain | EVQLVESGGGLVQPGRSLRLSCAASGFTFDDYAMHWVRQAPGKGLEWVSAITW | 132 |
| fusion sequence | NSGHIDYADSVEGRETISRDNAKNSLYLQMNSLRAEDTAVYYCAKVSYLSTAS | |
| Adalimumab scFv | SLDYWGQGTLVTVSSGGGGSGGGGSGGGGSGGGGSDIQMTQSPSSLSASVGDR | |
| Linker B | VTITCRASQGIRNYLAWYQQKPGKAPKLLIYAASTLQSGVPSRFSGSGSGTDF | |
| ABM67.2.4.2B | TLTISSLQPEDVATYYCQRYNRAPYTFGQGTKVEIKRGGGGSGGGGSGGGGSE | |
| IgG4 HC | VQLVESGGGLVKPGGSLRLPCAASGFTFSSYAMSWVRQTPERRLEWVATISSG | |
| | GSYTYYPDSVKGRFTISRDNAKNSLYLQMSSLRAEDTAVYYCARQNWDAFVSW | |
| | GQGTLVTVSSASTKGPSVFPLAPCSRSTSESTAALGCLVKDYFPEPVTVSWNS | |
| | GALTSGVHTFPAVLQSSGLYSLSSVVTVPSSSLGTKTYTCNVDHKPSNTKVDK | |
| | RVESKYGPPCPPCPAPEFLGGPSVFLFPPKPKDTLMISRTPEVTCVVVDVSQE | |
| | DPEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCSVM | |
| | HEALHNHYTQKSLSLSLGL | |
| Light chain | EIVLTQSPVILSVTPKEKVTFTCRAGQSIGTSIHWYQQRTDQSPKLLIKSASE | 142 |
| sequence | SISGVPSRFSGSGSGTDFTLTINSLEAEDIATYYCQQSNSWPLTFGAGTKLEI | |
| ABM67.3 VL | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |

In some embodiments, the bi-specific binding proteins of the present disclosure are tetrameric compounds, comprised of dimers of dimers—similar to the structure of naturally occurring antibodies. In such embodiments, the bi-specific binding proteins comprise a first heavy chain polypeptide associated with a first light chain polypeptide and a second heavy chain polypeptide associated with a second light chain polypeptide. These two dimers then form a tetramer via disulfide bonds between the first and second heavy chain polypeptides. See FIG. 11A and FIG. 11B.

In some embodiments, the bi-specific binding proteins of the present disclosure are dimeric proteins comprised of two monomers. In some embodiments, the bi-specific binding proteins comprise a first and a second monomer, each comprising the necessary components to form two antigen-binding domains (e.g., an IL17A binding domain and a second antigen-binding domain). See FIG. 11C.

In some embodiments, the bi-specific binding protein of the present disclosure comprises a first and a second heavy chain polypeptide and a first and a second light chain polypeptide, wherein the first and second heavy chain polypeptides comprise, from N-terminus to C-terminus, an antigen-binding domain, a linker, and an anti-IL-17A antigen-binding domain. In some embodiments, the first and second heavy chain polypeptides comprise, from N-terminus to C-terminus, an antigen-binding domain, a linker, and an anti-IL-17A antibody heavy chain. In some embodiments, the antigen-binding domain is a VEGF-binding domain or a TNFα binding domain.

In some embodiments, the first and second heavy chain polypeptides comprise, from N-terminus to C-terminus, a VEGF antigen-binding domain comprising an amino acid sequence of SEQ ID NO: 167 or 168, a linker, and an anti-IL-17A antibody heavy chain comprising an amino acid sequence selected from SEQ ID NOs: 169-174. In some embodiments, the first and second heavy chain polypeptides comprise an amino acid sequence selected from SEQ ID NOs: 101-108. In some embodiments, the first and second light chain polypeptides comprise an anti-IL-17A antibody light chain. In some embodiments, the first and second light chain polypeptides comprise the amino acid sequence selected from SEQ ID NO: 136 and 137. In some embodiments, the bi-specific binding protein comprises a first and second heavy chain polypeptide comprising an amino acid sequence selected from the group of SEQ ID NO: 101-108 and a first and second light chain polypeptide comprising an amino acid sequence selected from SEQ ID NO: 136 and 137. Exemplary embodiments of this configuration include 648.1-G1-A, 648.1-G1-B, 648.1-G2-A, 648.1-G2-B, 648.1-G4-A, 648.1-G4-B, 648.2-G1-A, and 648.2-G1-B described above.

In some embodiments, the bi-specific binding protein comprises a first and second heavy chain polypeptide comprising the amino acid sequence of SEQ ID NO: 101 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 102 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 103 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 104 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 105 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 106 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 107 and a first and second light chain polypeptide comprising the amino acid sequence of SEQ ID NO: 137. In some embodiments, the bi-specific binding protein comprises a first and second heavy chain polypeptide comprising the amino acid sequence of SEQ ID NO: 108 and a first and second light chain polypeptide comprising the amino acid sequence of SEQ ID NO: 137.

In some embodiments, the first and second heavy chain polypeptides comprise, from N-terminus to C-terminus, a TNFα antigen-binding domain comprising an amino acid sequence of SEQ ID NO: 134, a linker, and an anti-IL-17A antibody heavy chain comprising an amino acid sequence selected from SEQ ID NOs: 180-185. In some embodiments, the first and second heavy chain polypeptides comprise an amino acid sequence selected from SEQ ID NOs: 127-132. In some embodiments, the first and second light chain polypeptides comprise an anti-IL-17A antibody light chain. In some embodiments, the first and second light chain polypeptides comprise the amino acid sequence selected from SEQ ID NO: 141 and 142. In some embodiments, the bi-specific binding protein comprises a first and second heavy chain polypeptide comprising the amino acid sequence of SEQ ID NO: 127-132 and a first and second light chain polypeptide comprising the amino acid sequence of SEQ ID NO: 141 and 142. Exemplary embodiments of this configuration include 392.1, 393.1, 408.1A, 408.1B, 409.1A, and 409.1B described above.

In some embodiments, the bi-specific binding protein comprises a first and second heavy chain polypeptide comprising the amino acid sequence of SEQ ID NO: 127 and a first and second light chain polypeptide comprising the amino acid sequence of SEQ ID NO: 141. In some embodiments, the bi-specific binding protein comprises a first and second heavy chain polypeptide comprising the amino acid sequence of SEQ ID NO: 128 and a first and second light chain polypeptide comprising the amino acid sequence of SEQ ID NO: 141. In some embodiments, the bi-specific binding protein comprises a first and second heavy chain polypeptide comprising the amino acid sequence of SEQ ID NO: 129 and a first and second light chain polypeptide comprising the amino acid sequence of SEQ ID NO: 142. In some embodiments, the bi-specific binding protein comprises a first and second heavy chain polypeptide comprising the amino acid sequence of SEQ ID NO: 130 and a first and second light chain polypeptide comprising the amino acid sequence of SEQ ID NO: 142. In some embodiments, the bi-specific binding protein comprises a first and second heavy chain polypeptide comprising the amino acid sequence of SEQ ID NO: 131 and a first and second light chain polypeptide comprising the amino acid sequence of SEQ ID NO: 142. In some embodiments, the bi-specific binding protein comprises a first and second heavy chain polypeptide comprising the amino acid sequence of SEQ ID NO: 132 and a first and second light chain polypeptide comprising the amino acid sequence of SEQ ID NO: 142.

In some embodiments, the bi-specific binding protein of the present disclosure comprises a first and a second heavy chain polypeptide and a first and a second light chain polypeptide, wherein the first and second heavy chain polypeptides comprise, from N-terminus to C-terminus, an anti-IL-17A antigen-binding domain, a linker, and an antigen-binding domain. In some embodiments, the bi-specific binding protein of the present disclosure comprises a first and a second heavy chain polypeptide and a first and a second light chain polypeptide, wherein the first and second heavy chain polypeptides comprise, from N-terminus to C-terminus, an anti-IL-17A antibody heavy chain, a linker, and an antigen-binding domain. In some embodiments, the antigen-binding domain is a VEGF-binding domain or a TNFα binding domain.

In some embodiments, the first and second heavy chain polypeptides comprise, from N-terminus to C-terminus, an anti-IL-17A antibody heavy chain comprising an amino acid sequence selected from SEQ ID NO: 169, 170, 175, and 176, a linker, and a VEGF antigen-binding domain comprising SEQ ID NO: 167 or 168. In some embodiments, the first and second heavy chain polypeptides comprise an amino acid sequence selected from SEQ ID NO: 109-116. In some embodiments, the first and second light chain polypeptides comprise an anti-IL-17A antibody light chain. In some embodiments, the first and second light chain polypeptides comprise an anti-IL-17A antibody light chain comprising the amino acid sequence of SEQ ID NO: 136 or 137. In some embodiments, the bi-specific binding protein comprises a first and second heavy chain polypeptide comprising an amino acid sequence selected from SEQ ID NO: 109-116 and a first and second light chain polypeptide comprising the amino acid sequence of SEQ ID NO: 136 or 137. Exemplary embodiments of this configuration include 649.1-G1-A, 649.1-G1-B, 649.2-G1-A, 649.2-G1-B, 649.3-G1-A, 649.3-G1-B, 649.4-G1-A, and 649.1-G1-B described above.

In some embodiments, the bi-specific binding protein comprises a first and second heavy chain polypeptide comprising the amino acid sequence of SEQ ID NO: 109 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 110 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 111 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 112 and a first and second light chain polypeptide comprising the amino acid sequence of SEQ ID NO: 136. In some embodiments, the bi-specific binding protein comprises a first and second heavy chain polypeptide comprising the amino acid sequence of SEQ ID NO: 113 and a first and second light chain polypeptide comprising the amino acid sequence of SEQ ID NO: 137. In some embodiments, the bi-specific binding protein comprises a first and second heavy chain polypeptide comprising the amino acid sequence of SEQ ID NO: 114 and a first and second light chain polypeptide comprising the amino acid sequence of SEQ ID NO: 137. In some embodiments, the bi-specific binding protein comprises a first and second heavy chain polypeptide comprising the amino acid sequence of SEQ ID NO: 115 and a first and second light chain polypeptide comprising the amino acid sequence of SEQ ID NO: 137. In some embodiments, the bi-specific binding protein comprises a first and second heavy chain polypeptide comprising the amino acid sequence of SEQ ID NO: 116 and a first and second light chain polypeptide comprising the amino acid sequence of SEQ ID NO: 137.

In some embodiments, the bi-specific binding protein of the present disclosure comprises a first and a second heavy chain polypeptide and a first and a second light chain polypeptide, wherein the first and second heavy chain polypeptides comprise an anti-IL-17A antibody heavy chain. In some embodiments, the first and second light chains comprise, from N-terminus to C-terminus, an antigen binding domain, a linker, and an anti-IL-17A antigen binding domain. In some embodiments, the anti-IL-17A antigen binding domain is an anti-IL-17A antibody light chain. In some embodiments, the antigen-binding domain is a VEGF-binding domain or a TNFα binding domain.

In some embodiments, the bi-specific binding protein of the present disclosure comprises a first and a second heavy chain polypeptide and a first and a second light chain polypeptide, wherein the first and second heavy chain polypeptides comprise an anti-IL-17A antibody heavy chain comprising an amino acid sequence selected from SEQ ID NO: 117-120. In some embodiments, the first and second light chains comprise, from N-terminus to C-terminus, a VEGF binding domain comprising SEQ ID NO: 167, a linker, and an anti-IL-17A antibody light chain comprising an amino acid sequence selected from SEQ ID NO: 136 and 137. In some embodiments, the first and second light chain polypeptides comprise an amino acid sequence selected from SEQ ID NO: 138 and 139. In some embodiments, the first and second heavy chain polypeptides comprise an amino acid sequence selected from SEQ ID NO: 117-120 and the first and second light chain polypeptides comprise an amino acid sequence selected from SEQ ID NO: 138 and 139. Exemplary embodiments of this configuration include 651.1-G2-A, 651.1-G2-B, 651.2-G1A, and 651.2-G1-B described above.

In some embodiments, the first and second heavy chain polypeptides comprise an amino acid sequence selected from SEQ ID NO: 117 and the first and second light chain polypeptides comprise an amino acid sequence selected from SEQ ID NO: 138. In some embodiments, the first and second heavy chain polypeptides comprise an amino acid sequence selected from SEQ ID NO: 118 and the first and second light chain polypeptides comprise an amino acid sequence selected from SEQ ID NO: 138. In some embodiments, the first and second heavy chain polypeptides comprise an amino acid sequence selected from SEQ ID NO: 119 and the first and second light chain polypeptides comprise an amino acid sequence selected from SEQ ID NO: 139. In some embodiments, the first and second heavy chain polypeptides comprise an amino acid sequence selected from SEQ ID NO: 120 and the first and second light chain polypeptides comprise an amino acid sequence selected from SEQ ID NO: 139.

In some embodiments, the bi-specific binding protein of the present disclosure comprises a first and a second protein monomer, wherein each monomer comprises, from N-terminus to C-terminus, an antigen binding domain, a linker, and an IL-17A binding domain. In some embodiments, the antigen-binding domain is a VEGF-binding domain or a TNFα binding domain. In some embodiments, the bi-specific binding protein of the present disclosure comprises a first and a second protein monomer, wherein each monomer comprises, from N-terminus to C-terminus, a VEGF binding domain comprising SEQ ID NO: 100, a linker, and an anti-IL-17A scFv binding domain comprising an amino acid sequence selected from SEQ ID NO: 177-179. In some embodiments, each monomer comprises an amino acid sequence that is at least 90%, 95%, 96%, 97%, 98%, or 99% identical to an amino acid sequence selected from SEQ ID NO: 123-125. In some embodiments, each monomer comprises or consists of an amino acid sequence selected from SEQ ID NO: 123-125. In some embodiments, each monomer comprises an amino acid sequence that is at least 90%, 95%, 96%, 97%, 98%, or 99% identical to an amino acid sequence selected from SEQ ID NO: 124. In some embodiments, each monomer comprises or consists of an amino acid sequence selected from SEQ ID NO: 124. In some embodiments, each monomer comprises an amino acid sequence that is at least 90%, 95%, 96%, 97%, 98%, or 99% identical to an amino acid sequence selected from SEQ ID NO: 125. In some embodiments, each monomer comprises or consists of an amino acid sequence selected from SEQ ID NO: 125.

### Labeled Antibodies and Antigen-Binding Fragments

In one aspect, the disclosed anti-IL-17A antibodies, antigen-binding fragments thereof, and bi-specific binding proteins comprising the same may further comprise a label. As used herein, a label can include a fluorescent dye, a member of a binding pair, such as biotin/streptavidin, a metal (e.g., gold), radioactive substituent, or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts.

Fluorophores are compounds or molecules that luminesce. Typically, fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs-AutoFluorescent Protein-(Quantum Biotechnologies) see sgGFP, sgBFP; Alexa Fluor350™; Alexa Fluor 430™; Alexa Fluor 488™; Alexa Fluor532™; Alexa Fluor 546™; Alexa Fluor 568™; Alexa Fluor 594™; Alexa Fluor 633™; Alexa Fluor 647™; Alexa Fluor660™; Alexa Fluor680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO-1; BOBO-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DUPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow IOGF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFPm (super glow BFP); sgGFP (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1;YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

### Polynucleotides and Methods of Producing Anti-IL-17A Antibodies and Antigen-Binding Fragments

The disclosure also includes polynucleotides (e.g., DNA or RNA) encoding the anti-IL-17A antibodies, antigen-binding fragments thereof, and bi-specific proteins comprising the same of the present disclosure. In some embodiments, the polynucleotides encode a polypeptide that is substantially identical to a polypeptide listed in Tables 3-5 or 7-36. In some embodiments, the polynucleotides encode a polypeptide that is at least about 80%, at least about 90%, at least about 95%, at least about 96%, at least about 97%, at least about 98%, at about least 99%, or at about least 100% identical to a polypeptide listed in Tables 3-5 or 7-36. Polynucleotides of the disclosure also include complementary nucleic acids. In some instances, the sequences will be fully complementary (no mismatches) when aligned. In other instances, there can be up to about a 20% mismatch in the sequences. The polynucleotide sequences provided herein can be exploited using codon optimization, degenerate sequence, silent mutations, and other DNA techniques to optimize expression in a particular host, and the present disclosure encompasses such sequence modifications.

In some embodiments, the polynucleotides of the present disclosure are inserted into a nucleic acid vector. The nucleic acid vector may be a viral vector or a non-viral vector, e.g. a plasmid. Vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. In some embodiments, the vector is a plasmid selected from pXT1, pSG5 (Stratagene), pSVK3, pBPV, pMSG, and pSVLSV40 (Pharmacia). In some embodiments, the vector is a viral vector selected from viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., U.S. Pat. No. 7,078,387; Ali et al., Hum Gene Ther 9:81 86, 1998, Flannery et al,, PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al,, Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Examples of vectors are pClneo vectors (Promega) for expression in mammalian cells; pLenti4/V5-DEST™ pLenti6/V5-DEST™, and pLenti6.2/V5-GW/lacZ (Invitrogen) for lentivirus-mediated gene transfer and expression in mammalian cells.

In some embodiments, the polynucleotide is inserted into a nucleic acid vector and is operably linked to one or more regulatory sequences that control transcription, such as promoters, enhancers, terminators, inducers, or repressors. Exemplary promoters include Non-limiting examples of suitable eukaryotic promoters (promoters functional in a eukaryotic cell) include those from cytomegalovirus (CMV) immediate early, herpes simplex virus (HSV) thymidine kinase, a viral simian virus 40 (SV40) (e.g., early and late SV40), a spleen focus forming virus (SFFV) promoter, long terminal repeats (LTRs) from retrovirus (e.g., a Moloney murine leukemia virus (MoMLV) LTR promoter or a a Rous sarcoma virus (RSV) LTR), a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter, and mouse metallothionein-1.

In some embodiments, the vector is introduced into a host cell for expression of the anti-IL-17A antibody, antigen-binding fragment thereof, and bi-specific protein comprising the same. Accordingly, proteins for use within the present disclosure can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook and Russell, Molecular Cloning: A Laboratory Manual (3rd ed., Cold Spring Harbor Laboratory Press, Cold Spring Harbor, N Y, 2001), and Ausubel et al., Short Protocols in Molecular Biology (4th ed., John Wiley & Sons, 1999).

The gene product encoded by a polynucleotide of the disclosure is expressed in any convenient expression system, including, for example, bacterial, yeast, insect, amphibian, and mammalian systems. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB11 (Hyclone, Logan, UT); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Virginia. Introduction of the DNA construct can use any convenient method, including, e.g. conjugation, bacterial transformation, calcium-precipitated DNA, electroporation, fusion, transfection, infection with viral vectors, biolistics, and the like.

For example, for recombinant expression of an anti-IL-17A antibody, antigen-binding fragment thereof, and bi-specific protein comprising the same as described herein, an expression vector will generally include a nucleic acid segment encoding one or more of the amino acid sequences provided in Tables 3-5 or 7-36, operably linked to a promoter. The expression vector is introduced to a host cell by conventional techniques, and the host cells are then cultured by conventional techniques to produce the encoded polypeptide(s) to produce the corresponding the anti-IL-17A antibodies, antigen-binding fragments thereof, or bi-specific proteins comprising the same.

To direct a recombinant protein into the secretory pathway of a host cell, a secretory signal sequence (also known as a leader sequence) is provided in the expression vector. The secretory signal sequence can be that of the native form of the recombinant protein or can be derived from another secreted protein or synthesized de novo. The secretory signal sequence is operably linked to the polypeptide-encoding DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences can be positioned elsewhere in the DNA sequence of interest (see, e.g., U.S. Pat. Nos. 5,037,743 and 5,143,830). It is understood in the art that secretory signals are cleaved from the mature form of the polypeptides.

Cultured mammalian cells are suitable hosts for production of recombinant polypeptides and proteins of the present disclosure (e.g., anti-IL-17A antibodies, antigen-binding fragments thereof, or bi-specific proteins comprising the same). Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, U.S. Pat. Nos. 4,713,339; 4,784,950; 4,579,821; and 4,656,134.

Transformed or transfected host cells to produce the polypeptides and proteins of the present disclosure (e.g., anti-IL-17A antibodies, antigen-binding fragments thereof, and bi-specific proteins comprising the same) are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins, and minerals. Media can also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

The anti-IL-17A antibodies, antigen-binding fragments thereof, and bi-specific proteins comprising the same of the present disclosure may be purified by conventional protein purification methods, typically by a combination of chromatographic techniques. See generally Affinity Chromatography: Principles & Methods (Pharmacia LKB Biotechnology, Uppsala, Sweden, 1988); Scopes, Protein Purification: Principles and Practice (Springer-Verlag, New York 1994). Proteins comprising an immunoglobulin Fc region can be purified by affinity chromatography on immobilized protein A or protein G. Additional purification steps, such as gel filtration, can be used to obtain the desired level of purity or to provide for desalting, buffer exchange, and the like.

Antibodies may also be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent (e.g., the IL-17A protein) to elicit lymphocytes that produce or are capable of producing antibodies that will specifically bind to the immunizing agent (e.g., the IL-17A protein). Alternatively, the lymphocytes may be immunized in vitro. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of IL-17A expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 Dec.; 17(6):569-76; Kilpatrick K E et al. High-affinity monoclonal antibodies to PED/PEA-15 generated using 5 microg of DNA. Hybridoma. 2000 Aug.; 19(4):297-302, which are incorporated herein by referenced in full for the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of an anti-IL-17 antibody as fusion proteins. The antigen is produced by inserting a gene fragment in-frame between the signal sequence and the mature protein domain of the anti-IL17A antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against IL-17. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art and are described further in the Examples below or in Harlow and Lane Antibodies, A Laboratory Manual Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The term “isolated”, when applied to the anti-IL-17A antibodies and antigen binding fragments thereof as defined herein, refers to antibodies and antigen binding fragments thereof that are substantially free of other proteins or polypeptides, particularly free of other antibodies having different antigenic specificities, and are also substantially free of other cellular or tissue material and/or chemical precursors or other chemicals. For example, when the antibodies and antigen binding fragments thereof are recombinantly produced, they are preferably substantially free of culture medium, and when the binding molecules are produced by chemical synthesis, they are preferably substantially free of chemical precursors or other chemicals, i.e., they are separated from chemical precursors or other chemicals which are involved in the synthesis of the protein. Preferably, substantially free means that the binding molecule will typically comprise about 50%, 60%, 70%, 80% or 90% W/W of a sample, more usually about 95%, and preferably will be over 99% pure.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody (e.g., a bi-specific binding protein described herein) comprising one antigen-binding site having specificity for IL-17A and another antigen-binding site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

Alternatively, the disclosed antibodies can be made utilizing transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

An isolated immunogenically specific antigen-binding fragment of the antibody is also provided. A specific immunogenic antigen-binding fragment of the antibody can be isolated from the whole antibody by chemical or mechanical disruption of the molecule. The purified fragments thus obtained are tested to determine their immunogenicity and specificity by the methods taught herein. Immunoreactive antigen-binding fragment of the antibody, optionally, are synthesized directly. An immunoreactive fragment is defined as an amino acid sequence of at least about two to five consecutive amino acids derived from the antibody amino acid sequence.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, CA). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allows relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with IL-17A. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of the monoclonal antibody or fragment thereof and one or more reagents for detecting binding of the anti-IL-17A antibodies, antigen-binding fragments thereof, and bi-specific proteins comprising the same to the IL-17A molecule. The reagents can include, for example, fluorescent tags, enzymatic tags, or other tags. The reagents can also include secondary or tertiary antibodies or reagents for enzymatic reactions, wherein the enzymatic reactions produce a product that can be visualized.

### Compositions

In some embodiments, the present disclosure provides compositions comprising the anti-IL-17A antibodies or antigen binding fragments thereof, including humanized versions thereof and bi-specific binding proteins comprising the same.

In some embodiments, the present disclosure provides compositions comprising a vector encoding the anti-IL-17A antibodies or antigen binding fragments thereof, including humanized versions thereof and bi-specific binding proteins comprising the same. In some embodiments, the vectors encoding the proteins described herein demonstrate trophism for cells of the eye, including AAV8 and AAV9. In some embodiments, the present disclosure provides a composition comprising a vector encoding the anti-IL-17A antibodies or antigen binding fragments thereof, including humanized versions thereof and bi-specific binding proteins comprising the same, wherein the vector is an AAV8 or an AAV9 vector.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-IL-17 antibody is disclosed and discussed and a number of modifications that can be made to a number of molecules including the anti-IL-17 antibody are discussed, specifically contemplated is each and every combination and permutation of an anti-IL-17 antibody and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The compositions, including antibodies, antigen-binding fragments, bi-specific proteins, and vectors encoding the same, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition maybe administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The compositions may be administered orally, parenterally (e.g., intravenously), by intramuscular injection, by intraperitoneal injection, by intravitreal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

### Therapeutic Uses and Methods

In some embodiments, the present disclosure provides a method of treating an inflammatory disease comprising administering an anti-IL-17A antibody, antigen-binding fragment thereof, or bi-specific protein comprising the same (or a vector encoding any of the forgoing) to a subject in need thereof.

In some embodiments, the disclosed anti-IL-17A antibodies, antigen-binding fragments thereof, or bi-specific binding proteins comprising the same (or a vector encoding any of the forgoing) can be used to treat an inflammatory condition or disease, such as, for example, airway inflammation, rheumatoid arthritis (“RA”), osteoarthritis, bone erosion, intraperitoneal abscesses and adhesions, infectious disease, inflammatory bowel disorder (“IBD”), macular degeneration (including wet and/or dry age-related macular degeneration), allograft rejection, psoriasis, certain types of cancer, angiogenesis, atherosclerosis, and multiple sclerosis (“MS”).

In some embodiments, the present disclosure provides a method of treating an ocular disease comprising administering an anti-IL-17A antibody, antigen-binding fragment thereof, or bi-specific protein comprising the same (or a vector encoding any of the forgoing) to a subject in need thereof. In some embodiments, the ocular disease is macular degeneration (e.g., wet or dry age-related macular degeneration), diabetic retinopathy, or retinitis pigmentosa. In some embodiments, the present disclosure provides a method of treating macular degeneration comprising administering an anti-IL-17A antibody, antigen-binding fragment thereof, or bi-specific protein comprising the same (or a vector encoding any of the forgoing) to a subject in need thereof. In some embodiments, the present disclosure provides a method of treating diabetic retinopathy comprising administering an anti-IL-17A antibody, antigen-binding fragment thereof, or bi-specific protein comprising the same (or a vector encoding any of the forgoing) to a subject in need thereof. In some embodiments, the present disclosure provides a method of treating retinitis pigmentosa comprising administering an anti-IL-17A antibody, antigen-binding fragment thereof, or bi-specific protein comprising the same (or a vector encoding any of the forgoing) to a subject in need thereof.

Dry macular degeneration causes blurred or reduced central vision, due to thinning of the macula. Wet macular degeneration is generally caused by leakage of fluid or blood into the macula. Wet macular degeneration begins as a dry macular degeneration. Symptoms of wet and dry macular degeneration include visual distortions, such as straight lines seeming bent; reduced central vision in one or both eyes; the need for brighter light when reading or doing close-up work; increased difficulty adapting to low light levels, such as when entering a dimly lit restaurant; increased blurriness of printed words; decreased intensity or brightness of colors; difficulty recognizing faces; a well-defined blurry spot or blind spot in your field of vision

Retinitis pigmentosa is a genetic disorder of the eye that involves the progressive loss of rod photoreceptor cells in the back of the eye, followed by loss of cone photoreceptor cells. Diagnosis is typically by an examination of the retina and a finding of dark pigment deposits.

Diabetic retinopathy refers to a medical condition in which damage occurs to the retina due to diabetes mellitus. Chronically high blood sugar from diabetes is associated with damage to the tiny blood vessels in the retina, leading to diabetic retinopathy. Diabetic retinopathy can cause blood vessels in the retina to leak fluid or hemorrhage, distorting vision. In its most advanced stage, new abnormal blood vessels proliferate on the surface of the retina, which can lead to scarring and cell loss in the retina.

In some embodiments, the present disclosure provides methods of decreasing vascular leakage in the eye in a subject in need thereof. A fluorescein angiogram may be used to look for damaged or leaky blood vessels. In this test, a fluorescent dye is injected into the bloodstream, often into an arm vein. Pictures of the retinal blood vessels are taken as the dye reaches the eye. In some embodiments, the present compositions and methods may be used to prevent or lessen the incidence, prevalence, or severity of damaged and/or leaky blood vessels in the eye. In some embodiments, vascular leakage in the eye is reduced by at least 10%, 20%, 30%, 40%, 50%, 60%, 70%, 80%, 90%, or 100% compared to the leakage observed in untreated or control treated eyes.

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

In some embodiments, for treatment methods and uses described herein, the anti-IL-17A antibody, antigen-binding fragment thereof, or bi-specific protein comprising the same (or a vector encoding any of the forgoing) is delivered in a manner consistent with conventional methodologies associated with management of the disease or disorder for which treatment is sought. In accordance with the disclosure herein, a therapeutically effective amount of the protein or polypeptide (or polynucleotide encoding the same) is administered to a subject in need of such treatment for a time and under conditions sufficient to prevent or treat the disease or disorder.

In prophylactic applications, pharmaceutical compositions or medicants comprising the anti-IL-17A antibody, antigen-binding fragment thereof, or bi-specific protein comprising the same (or a vector encoding any of the forgoing) are administered to a patient susceptible to, or otherwise at risk of, a particular disorder in an amount sufficient to eliminate or reduce the risk or delay the onset of the disorder. In therapeutic applications, the anti-IL-17A antibody, antigen-binding fragment thereof, or bi-specific protein comprising the same (or a vector encoding any of the forgoing) is administered to a patient suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

For administration, the anti-IL-17A antibody, antigen-binding fragment thereof, or bi-specific protein comprising the same (or a vector encoding any of the forgoing) may be formulated as a pharmaceutical composition. A pharmaceutical composition may comprise: (i) an anti-IL-17A antibody, antigen-binding fragment thereof, or bi-specific protein comprising the same (or a vector encoding any of the forgoing); and (ii) a pharmaceutically acceptable carrier, diluent, or excipient. A pharmaceutical composition comprising an anti-IL-17A antibody, antigen-binding fragment thereof, or bi-specific protein comprising the same (or a vector encoding any of the forgoing) can be formulated according to known methods to prepare pharmaceutically useful compositions, whereby the therapeutic molecule is combined in a mixture with a pharmaceutically acceptable carrier, diluent, or excipient. Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising a polypeptide or protein described herein may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition comprising the anti-IL-17A antibody, antigen-binding fragment thereof, or bi-specific protein comprising the same (or a vector encoding any of the forgoing) may be administered to a subject in a therapeutically effective amount. According to the methods of the present disclosure, the anti-IL-17A antibody, antigen-binding fragment thereof, or bi-specific protein comprising the same (or a vector encoding any of the forgoing) can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, the anti-IL-17A antibody, antigen-binding fragment thereof, or bi-specific protein comprising the same (or a vector encoding any of the forgoing) can be administered to a subject in a single bolus delivery, via continuous delivery (e.g., continuous transdermal delivery) over an extended time period, or in a repeated administration protocol (e.g., on an hourly, daily, weekly, or monthly basis).

Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy.

Determination of effective dosages in this context is typically based on animal model studies followed up by human clinical trials and is guided by determining effective dosages and administration protocols that significantly reduce the occurrence or severity of the subject disorder in model subjects. Accordingly, a “therapeutically effective amount,” as used herein, refers to an amount of a compound is an amount that achieves the desired biologic or therapeutic effect, namely an amount that prevents, reduces, or ameliorates one or more symptoms of the enumerated diseases being treated or prevented. For example, the therapeutically effective amount of the antibody, or antigen-binding fragment thereof, will depend on the condition to be treated, the severity and course of the condition, whether the antibody is administered for preventive or therapeutic purposes, previous therapy, the patient's clinical history and response to the antibody, the type of antibody, or antigen-binding fragment thereof, used, and the discretion of the attending physician. The anti-IL-17A antibody, antigen-binding fragment thereof, or bi-specific protein comprising the same (or a vector encoding any of the forgoing) is suitably administered to the patent at one time or over a series of treatments and may be administered to the patent at any time from diagnosis onwards. The anti-IL-17A antibody, antigen-binding fragment thereof, or bi-specific protein comprising the same may be administered as the sole treatment or in conjunction with other drugs or therapies useful in treating the condition in question.

In some embodiments, the therapeutically effective amount of the anti-IL-17A antibody, antigen-binding fragment thereof, or bi-specific protein comprising the same (or a vector encoding any of the forgoing) is between about 1 ng/kg body weight/day to about 100 mg/kg body weight/day. In some embodiments, the range of antibody administered is from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day. Dosages within this range can be achieved by single or multiple administrations, including, e.g., multiple administrations per day or daily, weekly, bi-weekly, or monthly administrations. The anti-IL-17A antibody, antigen-binding fragment thereof, or bi-specific protein comprising the same (or a vector encoding any of the forgoing) may be administered, as appropriate or indicated, as a single dose by bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

For administration to a human adult patient, the therapeutically effective amount may be administered in doses in the range of 0.0006 mg to 1000 mg per dose, including but not limited to 0.0006 mg per dose, 0.001 mg per dose, 0.003 mg per dose, 0.006 mg per dose, 0.01 mg per dose, 0.03 mg per dose, 0.06 mg per dose, 0.1 mg per dose, 0.3 mg per dose, 0.6 mg per dose, 1 mg per dose, 3 mg per dose, 6 mg per dose, 10 mg per dose, 30 mg per dose, 60 mg per dose, 100 mg per dose, 300 mg per dose, 600 mg per dose and 1000 mg per dose, and multiple, usually consecutive daily doses may be administered in a course of treatment. The anti-IL-17A antibody, antigen-binding fragment thereof, or bi-specific protein comprising the same (or a vector encoding any of the forgoing) can be administered at different times of the day. In one embodiment the optimal therapeutic dose can be administered in the evening. In another embodiment the optimal therapeutic dose can be administered in the morning. As expected, the dosage will be dependent on the condition, size, age, and condition of the patient.

Dosage of the pharmaceutical composition comprising the anti-IL-17A antibody, antigen-binding fragment thereof, or bi-specific protein comprising the same (or a vector encoding any of the forgoing) can be varied by the attending clinician to maintain a desired concentration at a target site. For example, if an intravenous mode of delivery is selected, local concentration of the agent in the bloodstream at the target tissue can be between about 0.01-50 nM, sometimes between about 1.0 nM and 10, 15, or 25 nM depending on the subject's status and projected measured response. Higher or lower concentrations can be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nM (under standard conditions) would be administered at about twice the dosage of particles with a release rate of 10 nM.

Pharmaceutical compositions comprising the anti-IL-17A antibody, antigen-binding fragment thereof, or bi-specific protein comprising the same (or a vector encoding any of the forgoing) can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

### FURTHER NUMBERED EMBODIMENTS

Further numbered embodiments of the disclosure are provided as follows:
Embodiment 1. An anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid sequence selected from SEQ ID NO: 2, 23, 31, 42, 81, and 91; (b) the HCDR2 comprises an amino acid sequence selected from SEQ ID NO: 3, 24, 32, 43, 82, and 86; (c) the HCDR3 comprises the amino acid sequence selected from SEQ ID NO: 4, 25, 33, 45, 83, 87, 92, and 96; (d) the LCDR1 comprises the amino acid sequence selected from SEQ ID NO: 6, 10, 27, 35, and 58; (e) the LCDR2 comprises the amino acid sequence selected from SEQ ID NO: 7, 28, 36, and 59; and (f) the LCDR3 comprises the amino acid sequence selected from SEQ ID NO: 8, 29, 37, 60, 89, and 94.
Embodiment 2. The anti-IL-17A antibody or antigen-binding fragment thereof of Embodiment 1, wherein (a) the HCDR1 comprises the amino acid sequence of SEQ ID NO: 2; (b) the HCDR2 comprises the amino acid sequence of SEQ ID NO: 3; (c) the HCDR3 comprises the amino acid sequence of SEQ ID NO: 4; (d) the LCDR1 comprises the amino acid sequence of SEQ ID NO: 7; (e) the LCDR2 comprises the amino acid sequence of SEQ ID NO: 8; and (f) the LCDR2 comprises the amino acid sequence of SEQ ID NO: 9.
Embodiment 3. The anti-IL-17A antibody or antigen-binding fragment thereof of Embodiment 1 or Embodiment 2, wherein the VH comprises an amino acid sequence that is at least 95%, 96%, 97%, 98%, or 99% identical to SEQ ID NO: 5 and wherein the VL comprises an amino acid sequence that is at least 95%, 96%, 97%, 98%, or 99% identical to SEQ ID NO: 9.
Embodiment 4. The anti-IL-17A antibody or antigen-binding fragment thereof of Embodiment 3, wherein the VH comprises or consists of the amino acid sequence of SEQ ID NO: 5 and wherein the VL comprises or consists of the amino acid sequence of SEQ ID NO: 9.
Embodiment 5. The anti-IL-17A antibody or antigen-binding fragment thereof of Embodiment 1, wherein (a) the HCDR1 comprises the amino acid sequence of SEQ ID NO: 2; (b) the HCDR2 comprises the amino acid sequence of SEQ ID NO: 3; (c) the HCDR3 comprises the amino acid sequence of SEQ ID NO: 4; (d) the LCDR1 comprises the amino acid sequence of SEQ ID NO: 10; (e) the LCDR2 comprises the amino acid sequence of SEQ ID NO: 7; and (f) the LCDR2 comprises the amino acid sequence of SEQ ID NO: 8.
Embodiment 6. The anti-IL-17A antibody or antigen-binding fragment thereof of Embodiment 1 or Embodiment 5, wherein the VH comprises an amino acid sequence that is at least 95%, 96%, 97%, 98%, or 99% identical to SEQ ID NO: 5 and wherein the VL comprises an amino acid sequence that is at least 95%, 96%, 97%, 98%, or 99% identical to SEQ ID NO: 11.
Embodiment 7. The anti-IL-17A antibody or antigen-binding fragment thereof of Embodiment 6, wherein the VH comprises or consists of the amino acid sequence of SEQ ID NO: 5 and wherein the VL comprises or consists of the amino acid sequence of SEQ ID NO: 11.
Embodiment 8. The anti-IL-17A antibody or antigen-binding fragment thereof of Embodiment 1, wherein (a) the HCDR1 comprises the amino acid sequence of SEQ ID NO: 23; (b) the HCDR2 comprises the amino acid sequence of SEQ ID NO: 24; (c) the HCDR3 comprises the amino acid sequence of SEQ ID NO: 25; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 29.
Embodiment 9. The anti-IL-17A antibody or antigen-binding fragment thereof of Embodiment 1 or Embodiment 8, wherein the VH comprises an amino acid sequence that is at least 95%, 96%, 97%, 98%, or 99% identical to SEQ ID NO: 26 and wherein the VL comprises an amino acid sequence that is at least 95%, 96%, 97%, 98%, or 99% identical to SEQ ID NO: 30.
Embodiment 10. The anti-IL-17A antibody or antigen-binding fragment thereof of Embodiment 9, wherein the VH comprises or consists of the amino acid sequence of SEQ ID NO: 26 and wherein the VL comprises or consists of the amino acid sequence of SEQ ID NO: 30.
Embodiment 11. The anti-IL-17A antibody or antigen-binding fragment thereof of Embodiment 1, wherein (a) the HCDR1 comprises the amino acid sequence of SEQ ID NO: 31; (b) the HCDR2 comprises the amino acid sequence of SEQ ID NO: 32; (c) the HCDR3 comprises the amino acid sequence of SEQ ID NO: 33; (d) the LCDR1 comprises the amino acid sequence of SEQ ID NO: 35; (e) the LCDR2 comprises the amino acid sequence of SEQ ID NO: 36; and (f) the LCDR2 comprises the amino acid sequence of SEQ ID NO: 37.
Embodiment 12. The anti-IL-17A antibody or antigen-binding fragment thereof of Embodiment 1 or Embodiment 11, wherein the VH comprises an amino acid sequence that is at least 95%, 96%, 97%, 98%, or 99% identical to SEQ ID NO: 34 and wherein the VL comprises an amino acid sequence that is at least 95%, 96%, 97%, 98%, or 99% identical to SEQ ID NO: 38.
Embodiment 13. The anti-IL-17A antibody or antigen-binding fragment thereof of Embodiment 12, wherein the VH comprises or consists of the amino acid sequence of SEQ ID NO: 34 and wherein the VL comprises or consists of the amino acid sequence of SEQ ID NO: 38.
Embodiment 14. The anti-IL-17A antibody or antigen-binding fragment thereof of Embodiment 1, wherein (a) the HCDR1 comprises the amino acid sequence of SEQ ID NO: 42; (b) the HCDR2 comprises the amino acid sequence of SEQ ID NO: 43; (c) the HCDR3 comprises the amino acid sequence of SEQ ID NO: 45; (d) the LCDR1 comprises the amino acid sequence of SEQ ID NO: 58; (e) the LCDR2 comprises the amino acid sequence of SEQ ID NO: 59; and (f) the LCDR2 comprises the amino acid sequence of SEQ ID NO: 60.
Embodiment 15. The anti-IL-17A antibody or antigen-binding fragment thereof of Embodiment 1 or Embodiment 14, wherein the VH comprises an amino acid sequence that is at least 95%, 96%, 97%, 98%, or 99% identical to SEQ ID NO: 57 and wherein the VL comprises an amino acid sequence that is at least 95%, 96%, 97%, 98%, or 99% identical to SEQ ID NO: 61.
Embodiment 16. The anti-IL-17A antibody or antigen-binding fragment thereof of Embodiment 15, wherein the VH comprises or consists of the amino acid sequence of SEQ ID NO: 57 and wherein the VL comprises or consists of the amino acid sequence of SEQ ID NO: 61.
Embodiment 17. The anti-IL-17A antibody or antigen-binding fragment thereof of Embodiment 1, wherein (a) the HCDR1 comprises the amino acid sequence of SEQ ID NO: 81; (b) the HCDR2 comprises the amino acid sequence of SEQ ID NO: 82; (c) the HCDR3 comprises the amino acid sequence of SEQ ID NO: 83; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 29.
Embodiment 18. The anti-IL-17A antibody or antigen-binding fragment thereof of Embodiment 1 or Embodiment 17, wherein the VH comprises an amino acid sequence that is at least 95%, 96%, 97%, 98%, or 99% identical to SEQ ID NO: 84 and wherein the VL comprises an amino acid sequence that is at least 95%, 96%, 97%, 98%, or 99% identical to SEQ ID NO: 85.
Embodiment 19. The anti-IL-17A antibody or antigen-binding fragment thereof of Embodiment 18, wherein the VH comprises or consists of the amino acid sequence of SEQ ID NO: 84 and wherein the VL comprises or consists of the amino acid sequence of SEQ ID NO: 85.
Embodiment 20. The anti-IL-17A antibody or antigen-binding fragment thereof of Embodiment 1, wherein (a) the HCDR1 comprises the amino acid sequence of SEQ ID NO: 81; (b) the HCDR2 comprises the amino acid sequence of SEQ ID NO: 86; (c) the HCDR3 comprises the amino acid sequence of SEQ ID NO: 87; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 89.
Embodiment 21. The anti-IL-17A antibody or antigen-binding fragment thereof of Embodiment 1 or Embodiment 20, wherein the VH comprises an amino acid sequence that is at least 95%, 96%, 97%, 98%, or 99% identical to SEQ ID NO: 85 and wherein the VL comprises an amino acid sequence that is at least 95%, 96%, 97%, 98%, or 99% identical to SEQ ID NO: 88.
Embodiment 22. The anti-IL-17A antibody or antigen-binding fragment thereof of Embodiment 21, wherein the VH comprises or consists of the amino acid sequence of SEQ ID NO: 85 and wherein the VL comprises or consists of the amino acid sequence of SEQ ID NO: 88.
Embodiment 23. The anti-IL-17A antibody or antigen-binding fragment thereof of Embodiment 1, wherein (a) the HCDR1 comprises the amino acid sequence of SEQ ID NO: 91; (b) the HCDR2 comprises the amino acid sequence of SEQ ID NO: 86; (c) the HCDR3 comprises the amino acid sequence of SEQ ID NO: 92; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 94.
Embodiment 24. The anti-IL-17A antibody or antigen-binding fragment thereof of Embodiment 1 or Embodiment 23, wherein the VH comprises an amino acid sequence that is at least 95%, 96%, 97%, 98%, or 99% identical to SEQ ID NO: 93 and wherein the VL comprises an amino acid sequence that is at least 95%, 96%, 97%, 98%, or 99% identical to SEQ ID NO: 95.
Embodiment 25. The anti-IL-17A antibody or antigen-binding fragment thereof of Embodiment 24, wherein the VH comprises or consists of the amino acid sequence of SEQ ID NO: 93 and wherein the VL comprises or consists of the amino acid sequence of SEQ ID NO: 95.
Embodiment 26. The anti-IL-17A antibody or antigen-binding fragment thereof of Embodiment 1, wherein (a) the HCDR1 comprises the amino acid sequence of SEQ ID NO: 81; (b) the HCDR2 comprises the amino acid sequence of SEQ ID NO: 86; (c) the HCDR3 comprises the amino acid sequence of SEQ ID NO: 96; (d) the LCDR1 comprises the amino acid sequence of SEQ ID NO: 27; (e) the LCDR2 comprises the amino acid sequence of SEQ ID NO: 28; and (f) the LCDR2 comprises the amino acid sequence of SEQ ID NO: 94.
Embodiment 27. The anti-IL-17A antibody or antigen-binding fragment thereof of Embodiment 1 or Embodiment 26 wherein the VH comprises an amino acid sequence that is at least 95%, 96%, 97%, 98%, or 99% identical to SEQ ID NO: 97 and wherein the VL comprises an amino acid sequence that is at least 95%, 96%, 97%, 98%, or 99% identical to SEQ ID NO: 98.
Embodiment 28. The anti-IL-17A antibody or antigen-binding fragment thereof of Embodiment 27, wherein the VH comprises or consists of the amino acid sequence of SEQ ID NO: 97 and wherein the VL comprises or consists of the amino acid sequence of SEQ ID NO: 98.
Embodiment 29. A humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid sequence selected from SEQ ID NO: 2; (b) the HCDR2 comprises an amino acid sequence selected from SEQ ID NO: 3; (c) the HCDR3 comprises the amino acid sequence selected from SEQ ID NO: 4; (d) the LCDR1 comprises the amino acid sequence selected from SEQ ID NO: 10; (e) the LCDR2 comprises the amino acid sequence selected from SEQ ID NO: 7; and (f) the LCDR3 comprises the amino acid sequence selected from SEQ ID NO: 8.
Embodiment 30. The humanized anti-IL-17A antibody or antigen-binding fragment thereof of Embodiment 29, wherein the VH comprises an amino acid sequence is at least 95%, 96%, 97%, 98%, or 99% identical to one of SEQ ID NOs: 12, 14, 16, 21, and 22 and wherein the VL comprises an amino acid sequence is at least 95%, 96%, 97%, 98%, or 99% identical to one of SEQ ID NOs: 13, 15, 17, 18, 19, and 20.
Embodiment 31. The humanized anti-IL-17A antibody or antigen-binding fragment thereof of Embodiment 30, wherein: (a) the VH comprises an amino acid sequence is at least 95%, 96%, 97%, 98%, or 99% identical SEQ ID NO: 12 and the VL comprises an amino acid sequence is at least 95%, 96%, 97%, 98%, or 99% identical to SEQ ID NO: 13; (b) the VH comprises an amino acid sequence is at least 95%, 96%, 97%, 98%, or 99% identical SEQ ID NO: 14 and the VL comprises an amino acid sequence is at least 95%, 96%, 97%, 98%, or 99% identical to SEQ ID NO: 15; (c) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 15; (d) the VH comprises an amino acid sequence is at least 95%, 96%, 97%, 98%, or 99% identical SEQ ID NO: 14 and the VL comprises an amino acid sequence is at least 95%, 96%, 97%, 98%, or 99% identical to SEQ ID NO: 17; (e) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 17; (f) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 18; (g) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 19; (h) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 20; (i) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 18; (j) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 19; (k) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 20; (1) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 18; (m) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 19; or (n) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 20.
Embodiment 32. The humanized anti-IL-17A antibody or antigen-binding fragment thereof of Embodiment 29, wherein the VH comprises or consists of one of SEQ ID NOs: 12, 14, 16, 21, and 22 and wherein the VL comprises or consists of one of SEQ ID NOs: 13, 15, 17, 18, 19, and 20.
Embodiment 33. The humanized anti-IL-17A antibody or antigen-binding fragment thereof of Embodiment 32 wherein: (a) the VH amino acid sequence comprises or consists of SEQ ID NO: 12 and the VL amino acid sequence comprises or consists of SEQ ID NO: 13; (b) the VH amino acid sequence comprises or consists of SEQ ID NO: 14 and the VL amino acid sequence comprises or consists of SEQ ID NO: 15; (c) the VH amino acid sequence comprises or consists of SEQ ID NO: 16 and the VL amino acid sequence comprises or consists of SEQ ID NO: 15; (d) the VH amino acid sequence comprises or consists of SEQ ID NO: 14 and the VL amino acid sequence comprises or consists of SEQ ID NO: 17; (e) the VH amino acid sequence comprises or consists of SEQ ID NO: 16 and the VL amino acid sequence comprises or consists of SEQ ID NO: 17; (f) the VH amino acid sequence comprises or consists of SEQ ID NO: 16 and the VL amino acid sequence comprises or consists of SEQ ID NO: 18; (g) the VH amino acid sequence comprises or consists of SEQ ID NO: 16 and the VL amino acid sequence comprises or consists of SEQ ID NO: 19; (h) the VH amino acid sequence comprises or consists of SEQ ID NO: 16 and the VL amino acid sequence comprises or consists of SEQ ID NO: 20; (i) the VH amino acid sequence comprises or consists of SEQ ID NO: 21 and the VL amino acid sequence comprises or consists of SEQ ID NO: 18; (j) the VH amino acid sequence comprises or consists of SEQ ID NO: 21 and the VL amino acid sequence comprises or consists of SEQ ID NO: 19; (k) the VH amino acid sequence comprises or consists of SEQ ID NO: 21 and the VL amino acid sequence comprises or consists of SEQ ID NO: 20; (1) the VH amino acid sequence comprises or consists of SEQ ID NO: 22 and the VL amino acid sequence comprises or consists of SEQ ID NO: 18; (m) the VH amino acid sequence comprises or consists of SEQ ID NO: 22 and the VL amino acid sequence comprises or consists of SEQ ID NO: 19; or (n) the VH amino acid sequence comprises or consists of SEQ ID NO: 22 and the VL amino acid sequence comprises or consists of SEQ ID NO: 20.
Embodiment 34. A humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid sequence selected from SEQ ID NO: 31; (b) the HCDR2 comprises an amino acid sequence selected from SEQ ID NO: 32; (c) the HCDR3 comprises the amino acid sequence selected from SEQ ID NO: 33; (d) the LCDR1 comprises the amino acid sequence selected from SEQ ID NO: 35; (e) the LCDR2 comprises the amino acid sequence selected from SEQ ID NO: 36; and (f) the LCDR3 comprises the amino acid sequence selected from SEQ ID NO: 37.
Embodiment 35. The humanized anti-IL-17A antibody or antigen-binding fragment thereof of Embodiment 34, wherein the VH comprises an amino acid sequence is at least 95%, 96%, 97%, 98%, or 99% identical to one of SEQ ID NOs: 39 and 34 and wherein the VL comprises an amino acid sequence is at least 95%, 96%, 97%, 98%, or 99% identical to one of SEQ ID NOs: 40 and 41.
Embodiment 36. The humanized anti-IL-17A antibody or antigen-binding fragment thereof of Embodiment 35, wherein: (a) the VH comprises an amino acid sequence is at least 95%, 96%, 97%, 98%, or 99% identical SEQ ID NO: 39 and the VL comprises an amino acid sequence is at least 95%, 96%, 97%, 98%, or 99% identical to SEQ ID NO: 40; (b) the VH comprises an amino acid sequence is at least 95%, 96%, 97%, 98%, or 99% identical SEQ ID NO: 39 and the VL comprises an amino acid sequence is at least 95%, 96%, 97%, 98%, or 99% identical to SEQ ID NO: 41; or (c) the VH comprises an amino acid sequence is at least 95%, 96%, 97%, 98%, or 99% identical SEQ ID NO: 34 and the VL comprises an amino acid sequence is at least 95%, 96%, 97%, 98%, or 99% identical to SEQ ID NO: 41.
Embodiment 37. The humanized anti-IL-17A antibody or antigen-binding fragment thereof of Embodiment 34, wherein the VH comprises or consists of one of SEQ ID NOs: 39 and 34 and wherein the VL comprises or consists of one of SEQ ID NOs: 40 and 41.
Embodiment 38. The humanized anti-IL-17A antibody or antigen-binding fragment thereof of Embodiment 37, wherein: (a) the VH amino acid sequence comprises or consists of SEQ ID NO: 39 and the VL amino acid sequence comprises or consists of SEQ ID NO: 40; (b) the VH amino acid sequence comprises or consists of SEQ ID NO: 39 and the VL amino acid sequence comprises or consists of SEQ ID NO: 41; or (c) the VH amino acid sequence comprises or consists of SEQ ID NO: 34 and the VL amino acid sequence comprises or consists of SEQ ID NO: 41.
Embodiment 39. A humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein: (a) the HCDR1 comprises an amino acid of SEQ ID NO: 42; (b) the HCDR2 comprises an amino acid sequence selected from SEQ ID NO: 43 or SEQ ID NO: 44, and (c) the HCDR3 comprises the amino acid sequence selected from any one of SEQ ID NOs: 45-56; (d) the LCDR1 comprises the amino acid sequence selected from SEQ ID NO: 58; (e) the LCDR2 comprises the amino acid sequence selected from SEQ ID NO: 59; and (f) the LCDR3 comprises the amino acid sequence selected from SEQ ID NO: 60.
Embodiment 40. The humanized anti-IL-17A antibody or antigen-binding fragment thereof of Embodiment 39, wherein the LCDR1 comprises the amino acid sequence of SEQ ID NO: 58; the LCDR2 comprises the amino acid sequence of SEQ ID NO: 59; and the LCDR3 comprises the amino acid sequence of SEQ ID NO: 60; and wherein: (a) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 45; (b) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 46; (c) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 47; (d) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 48; (e) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 49; (f) the HCDR1 comprises an amino acid sequence selected from SEQ ID NO: 42; the HCDR2 comprises an amino acid sequence selected from SEQ ID NO: 44, and the HCDR3 comprises the amino acid sequence of SEQ ID NO: 50; (g) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 51; (h) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 52; (i) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 53; (j) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 54; (k) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 55; or (1) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 56.
Embodiment 41. The humanized anti-IL-17A antibody or antigen-binding fragment thereof of Embodiment 39, wherein the VH comprises an amino acid sequence is at least 95%, 96%, 97%, 98%, or 99% identical to one of SEQ ID NOs: 62, 64, 65, 66, 67, 68, 69, 71, 72, 73, 74, 99, 77, 78, or 79 and wherein the VL comprises an amino acid sequence is at least 95%, 96%, 97%, 98%, or 99% identical to one of SEQ ID NOs: 63, 70, 75, 76, or 80.
Embodiment 42. The humanized anti-IL-17A antibody or antigen-binding fragment thereof of Embodiment 41, wherein: (a) the VH comprises an amino acid sequence is at least 95%, 96%, 97%, 98%, or 99% identical SEQ ID NO: 62 and the VL comprises an amino acid sequence is at least 95%, 96%, 97%, 98%, or 99% identical to SEQ ID NO: 63; (b) the VH comprises an amino acid sequence is at least 95%, 96%, 97%, 98%, or 99% identical SEQ ID NO: 64 and the VL comprises an amino acid sequence is at least 95%, 96%, 97%, 98%, or 99% identical to SEQ ID NO: 63; (c) the VH comprises an amino acid sequence is at least 95%, 96%, 97%, 98%, or 99% identical SEQ ID NO: 65 and the VL comprises an amino acid sequence is at least 95%, 96%, 97%, 98%, or 99% identical to SEQ ID NO: 63; (d) the VH comprises an amino acid sequence is at least 95%, 96%, 97%, 98%, or 99% identical SEQ ID NO: 66 and the VL comprises an amino acid sequence is at least 95%, 96%, 97%, 98%, or 99% identical to SEQ ID NO: 63; (e) the VH comprises an amino acid sequence is at least 95%, 96%, 97%, 98%, or 99% identical SEQ ID NO: 67 and the VL comprises an amino acid sequence is at least 95%, 96%, 97%, 98%, or 99% identical to SEQ ID NO: 63; (f) the VH comprises an amino acid sequence is at least 95%, 96%, 97%, 98%, or 99% identical SEQ ID NO: 68 and the VL comprises an amino acid sequence is at least 95%, 96%, 97%, 98%, or 99% identical to SEQ ID NO: 63; (g) the VH comprises an amino acid sequence is at least 95%, 96%, 97%, 98%, or 99% identical SEQ ID NO: 69 and the VL comprises an amino acid sequence is at least 95%, 96%, 97%, 98%, or 99% identical to SEQ ID NO: 70; (h) the VH comprises an amino acid sequence is at least 95%, 96%, 97%, 98%, or 99% identical SEQ ID NO: 71 and the VL comprises an amino acid sequence is at least 95%, 96%, 97%, 98%, or 99% identical to SEQ ID NO: 70; (i) the VH comprises an amino acid sequence is at least 95%, 96%, 97%, 98%, or 99% identical SEQ ID NO: 72 and the VL comprises an amino acid sequence is at least 95%, 96%, 97%, 98%, or 99% identical to SEQ ID NO: 70; (j) the VH comprises an amino acid sequence is at least 95%, 96%, 97%, 98%, or 99% identical SEQ ID NO: 73 and the VL comprises an amino acid sequence is at least 95%, 96%, 97%, 98%, or 99% identical to SEQ ID NO: 70; (k) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 70; (1) the VH comprises an amino acid sequence is at least 95%, 96%, 97%, 98%, or 99% identical SEQ ID NO: 99 and the VL comprises an amino acid sequence is at least 95%, 96%, 97%, 98%, or 99% identical to SEQ ID NO: 70; (m) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 75; (n) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 76; (o) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 70; (p) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 75; (q) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 76; (r) the VH comprises an amino acid sequence is at least 95%, 96%, 97%, 98%, or 99% identical SEQ ID NO: 78 and the VL comprises an amino acid sequence is at least 95%, 96%, 97%, 98%, or 99% identical to SEQ ID NO: 70; (s) the VH comprises an amino acid sequence is at least 95%, 96%, 97%, 98%, or 99% identical SEQ ID NO: 79 and the VL comprises an amino acid sequence is at least 95%, 96%, 97%, 98%, or 99% identical to SEQ ID NO: 70; or (t) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 80.
Embodiment 43. The humanized anti-IL-17A antibody or antigen-binding fragment thereof of Embodiment 39, wherein the VH comprises or consists of one of SEQ ID NOs: 62, 64, 65, 66, 67, 68, 69, 71, 72, 73, 74, 99, 77, 78, or 79 and wherein the VL comprises or consists of one of SEQ ID NOs: 63, 70, 75, 76, or 80.
Embodiment 44. The humanized anti-IL-17A antibody or antigen-binding fragment thereof of Embodiment 43, wherein: (a) the VH amino acid sequence comprises or consists of SEQ ID NO: 62 and the VL amino acid sequence comprises or consists of SEQ ID NO: 63; (b) the VH amino acid sequence comprises or consists of SEQ ID NO: 64 and the VL amino acid sequence comprises or consists of SEQ ID NO: 63; (c) the VH amino acid sequence comprises or consists of SEQ ID NO: 65 and the VL amino acid sequence comprises or consists of SEQ ID NO: 63; (d) the VH amino acid sequence comprises or consists of SEQ ID NO: 66 and the VL amino acid sequence comprises or consists of SEQ ID NO: 63; (e) the VH amino acid sequence comprises or consists of SEQ ID NO: 67 and the VL amino acid sequence comprises or consists of SEQ ID NO: 63; (f) the VH amino acid sequence comprises or consists of SEQ ID NO: 68 and the VL amino acid sequence comprises or consists of SEQ ID NO: 63; (g) the VH amino acid sequence comprises or consists of SEQ ID NO: 69 and the VL amino acid sequence comprises or consists of SEQ ID NO: 70; (h) the VH amino acid sequence comprises or consists of SEQ ID NO: 71 and the VL amino acid sequence comprises or consists of SEQ ID NO: 70; (i) the VH amino acid sequence comprises or consists of SEQ ID NO: 72 and the VL amino acid sequence comprises or consists of SEQ ID NO: 70; (j) the VH amino acid sequence comprises or consists of SEQ ID NO: 73 and the VL amino acid sequence comprises or consists of SEQ ID NO: 70; (k) the VH amino acid sequence comprises or consists of SEQ ID NO: 74 and the VL amino acid sequence comprises or consists of SEQ ID NO: 70; (1) the VH amino acid sequence comprises or consists of SEQ ID NO: 99 and the VL amino acid sequence comprises or consists of SEQ ID NO: 70; (m) the VH amino acid sequence comprises or consists of SEQ ID NO: 74 and the VL amino acid sequence comprises or consists of SEQ ID NO: 75; (n) the VH amino acid sequence comprises or consists of SEQ ID NO: 74 and the VL amino acid sequence comprises or consists of SEQ ID NO: 76; (o) the VH amino acid sequence comprises or consists of SEQ ID NO: 77 and the VL amino acid sequence comprises or consists of SEQ ID NO: 70; (p) the VH amino acid sequence comprises or consists of SEQ ID NO: 77 and the VL amino acid sequence comprises or consists of SEQ ID NO: 75; (q) the VH amino acid sequence comprises or consists of SEQ ID NO: 77 and the VL amino acid sequence comprises or consists of SEQ ID NO: 76; (r) the VH amino acid sequence comprises or consists of SEQ ID NO: 78 and the VL amino acid sequence comprises or consists of SEQ ID NO: 70; (s) the VH amino acid sequence comprises or consists of SEQ ID NO: 79 and the VL amino acid sequence comprises or consists of SEQ ID NO: 70; or (t) the VH amino acid sequence comprises or consists of SEQ ID NO: 74 and the VL amino acid sequence comprises or consists of SEQ ID NO: 80.
Embodiment 45. The anti-IL-17A antibody or antigen-binding fragment thereof of any one of Embodiments 1-28 or the humanized anti-IL-17A antibody or antigen-binding fragment thereof of any one of Embodiments 29-44, wherein the antigen binding fragment is a single chain variable fragment (scFv).
Embodiment 46. The anti-IL-17A antibody or antigen-binding fragment thereof or the humanized anti-IL-17A antibody or antigen-binding fragment thereof of Embodiment 45, wherein the scFv comprises an amino acid sequence that is at least 95%, 96%, 97%, 98%, or 99% identical to an amino acid sequence selected from SEQ ID NOs: 177-179.
Embodiment 47. The anti-IL-17A antibody or antigen-binding fragment thereof or the humanized anti-IL-17A antibody or antigen-binding fragment thereof of Embodiment 45, wherein the scFv comprises or consists of an amino acid sequence selected from SEQ ID NOs: 177-179.
Embodiment 48. An anti-IL-17A single chain variable fragment (scFv) comprising an amino acid sequence that is at least 95%, 96%, 97%, 98%, or 99% identical to an amino acid sequence selected from SEQ ID NOs: 177-179.
Embodiment 49. The anti-IL-17A scFv of Embodiment 48, comprising or consisting of an amino acid sequence selected from SEQ ID NOs: 177-179.
Embodiment 50. A polynucleotide encoding the anti-IL-17A antibody or antigen-binding fragment thereof of any one of Embodiments 1-28 or the humanized anti-IL-17A antibody or antigen-binding fragment thereof of any one of Embodiments 29-44.
Embodiment 51. A polynucleotide encoding the anti-IL-17A scFv of any one of Embodiments 48-49.
Embodiment 52. An expression vector comprising the polynucleotide of Embodiment 50 or 51.
Embodiment 53. A host cell comprising the polynucleotide of Embodiment 50 or 51 or the expression vector of Embodiment 52.
Embodiment 54. A vector comprising a polynucleotide encoding the anti-IL-17A antibody or antigen-binding fragment thereof of any one of Embodiments 1-28, the humanized anti-IL-17A antibody or antigen-binding fragment thereof of any one of Embodiments 29-44, or the anti-IL-17A scFv of any one of Embodiments 48-49.
Embodiment 55. The vector of Embodiment 54, wherein the vector is an AAV8 or AAV9 vector.
Embodiment 56. A method of manufacturing the anti-IL-17A antibody or antigen-binding fragment thereof of any one of Embodiments 1-28, the humanized anti-IL-17A antibody or antigen-binding fragment thereof of any one of Embodiments 29-44, or the anti-IL-17A scFv of any one of Embodiments 48-49 comprising introducing the expression vector of Embodiment 52 into a host cell.
Embodiment 57. A bi-specific binding protein comprising a first and a second heavy chain polypeptide and a first and a second light chain polypeptide, wherein the first and second heavy chain polypeptides comprise, from N-terminus to C-terminus, an antigen-binding domain, a linker, and an anti-IL-17A antigen-binding domain.
Embodiment 58. The bi-specific binding protein of Embodiment 57, wherein the anti-IL-17A antigen-binding domain is an anti-IL-17A antibody heavy chain.
Embodiment 59. The bi-specific binding protein of Embodiment 57 or 58, wherein the antigen-binding domain is a VEGF-binding domain or a TNFα binding domain.
Embodiment 60. The bi-specific binding protein of Embodiment 59, wherein the first and second heavy chain polypeptides comprise, from N-terminus to C-terminus, a VEGF antigen-binding domain comprising an amino acid sequence of SEQ ID NO: 167 or 168, a linker, and an anti-IL-17A antibody heavy chain comprising an amino acid sequence selected from SEQ ID NOs: 169-174.
Embodiment 61. The bi-specific binding protein of Embodiment 60, wherein the first and second heavy chain polypeptides comprise an amino acid sequence selected from SEQ ID NOs: 101-108.
Embodiment 62. The bi-specific binding protein of Embodiment 60 or 61, wherein the first and second light chain polypeptides comprise an anti-IL-17A antibody light chain.
Embodiment 63. The bi-specific binding protein of Embodiment 62, wherein the first and second light chain polypeptides comprise the amino acid sequence selected from SEQ ID NO: 136 and 137.
Embodiment 64. The bi-specific binding protein of any one of Embodiments 57-63, wherein the first and second heavy chain polypeptide comprising an amino acid sequence selected from the group of SEQ ID NO: 101-108 and the first and second light chain polypeptide comprising an amino acid sequence selected from SEQ ID NO: 136 and 137.
Embodiment 65. The bi-specific binding protein of Embodiment 64, wherein (a) the bi-specific binding protein comprises a first and second heavy chain polypeptide comprising the amino acid sequence of SEQ ID NO: 101 and a first and second light chain polypeptide comprising the amino acid sequence of SEQ ID NO: 136; (b) the bi-specific binding protein comprises a first and second heavy chain polypeptide comprising the amino acid sequence of SEQ ID NO: 102 and a first and second light chain polypeptide comprising the amino acid sequence of SEQ ID NO: 136; (c) the bi-specific binding protein comprises a first and second heavy chain polypeptide comprising the amino acid sequence of SEQ ID NO: 103 and a first and second light chain polypeptide comprising the amino acid sequence of SEQ ID NO: 136; (d) the bi-specific binding protein comprises a first and second heavy chain polypeptide comprising the amino acid sequence of SEQ ID NO: 104 and a first and second light chain polypeptide comprising the amino acid sequence of SEQ ID NO: 136; (e) the bi-specific binding protein comprises a first and second heavy chain polypeptide comprising the amino acid sequence of SEQ ID NO: 105 and a first and second light chain polypeptide comprising the amino acid sequence of SEQ ID NO: 136; (f) the bi-specific binding protein comprises a first and second heavy chain polypeptide comprising the amino acid sequence of SEQ ID NO: 106 and a first and second light chain polypeptide comprising the amino acid sequence of SEQ ID NO: 136; (g) the bi-specific binding protein comprises a first and second heavy chain polypeptide comprising the amino acid sequence of SEQ ID NO: 107 and a first and second light chain polypeptide comprising the amino acid sequence of SEQ ID NO: 137; or (h) the bi-specific binding protein comprises a first and second heavy chain polypeptide comprising the amino acid sequence of SEQ ID NO: 108 and a first and second light chain polypeptide comprising the amino acid sequence of SEQ ID NO: 137.
Embodiment 66. The bi-specific binding protein of Embodiment 59, wherein the first and second heavy chain polypeptides comprise, from N-terminus to C-terminus, a TNFα antigen-binding domain comprising an amino acid sequence of SEQ ID NO: 134, a linker, and an anti-IL-17A antibody heavy chain comprising an amino acid sequence selected from SEQ ID NOs: 180-185.
Embodiment 67. The bi-specific binding protein of Embodiment 66, wherein the first and second heavy chain polypeptides comprise an amino acid sequence selected from SEQ ID NOs: 127-132.
Embodiment 68. The bi-specific binding protein of Embodiment 66 or 67, wherein the first and second light chain polypeptides comprise an anti-IL-17A antibody light chain.
Embodiment 69. The bi-specific binding protein of Embodiment 68, wherein the first and second light chain polypeptides comprise an amino acid sequence selected from SEQ ID NO: 141 and 142.
Embodiment 70. The bi-specific binding protein of any one of Embodiments 66-69, wherein the first and second heavy chain polypeptide comprise the amino acid sequence of any one of SEQ ID NO: 127-132 and a first and second light chain polypeptide comprise the amino acid sequence of SEQ ID NO: 141 or 142.
Embodiment 71. The bi-specific binding protein of Embodiment 70, wherein (a) the bi-specific binding protein comprises a first and second heavy chain polypeptide comprising the amino acid sequence of SEQ ID NO: 127 and a first and second light chain polypeptide comprising the amino acid sequence of SEQ ID NO: 141; (b) the bi-specific binding protein comprises a first and second heavy chain polypeptide comprising the amino acid sequence of SEQ ID NO: 128 and a first and second light chain polypeptide comprising the amino acid sequence of SEQ ID NO: 141; (c) the bi-specific binding protein comprises a first and second heavy chain polypeptide comprising the amino acid sequence of SEQ ID NO: 129 and a first and second light chain polypeptide comprising the amino acid sequence of SEQ ID NO: 142; (d) the bi-specific binding protein comprises a first and second heavy chain polypeptide comprising the amino acid sequence of SEQ ID NO: 130 and a first and second light chain polypeptide comprising the amino acid sequence of SEQ ID NO: 142; (e) the bi-specific binding protein comprises a first and second heavy chain polypeptide comprising the amino acid sequence of SEQ ID NO: 131 and a first and second light chain polypeptide comprising the amino acid sequence of SEQ ID NO: 142; or (f) the bi-specific binding protein comprises a first and second heavy chain polypeptide comprising the amino acid sequence of SEQ ID NO: 132 and a first and second light chain polypeptide comprising the amino acid sequence of SEQ ID NO: 142.
Embodiment 72. A bi-specific binding protein comprising a first and a second heavy chain polypeptide and a first and a second light chain polypeptide, wherein the first and second heavy chain polypeptides comprise, from N-terminus to C-terminus, an anti-IL-17A antigen-binding domain, a linker, and an antigen-binding domain.
Embodiment 73. The bi-specific binding protein of Embodiment 72, wherein the first and second heavy chain polypeptides comprise, from N-terminus to C-terminus, an anti-IL-17A antibody heavy chain, a linker, and an antigen-binding domain.
Embodiment 74. The bi-specific binding protein of Embodiment 72 or 73, wherein the antigen-binding domain is a VEGF-binding domain or a TNFα binding domain.
Embodiment 75. The bi-specific binding protein of Embodiment 74, wherein the first and second heavy chain polypeptides comprise, from N-terminus to C-terminus, an anti-IL-17A antibody heavy chain comprising an amino acid sequence selected from SEQ ID NO: 169, 170, 175, and 176, a linker, and a VEGF antigen-binding domain comprising SEQ ID NO: 167 or 168.
Embodiment 76. The bi-specific binding protein of Embodiment 75, wherein the first and second heavy chain polypeptides comprise an amino acid sequence selected from SEQ ID NO: 109-116.
Embodiment 77. The bi-specific binding protein of any one of Embodiments 72-76, wherein the first and second light chain polypeptides comprise an anti-IL-17A antibody light chain.
Embodiment 78. The bi-specific binding protein of Embodiment 77, wherein the first and second light chain polypeptides comprise an anti-IL-17A antibody light chain comprising the amino acid sequence of SEQ ID NO: 136 or 137.
Embodiment 79. The bi-specific binding protein of any one of Embodiments 76-78, wherein the first and second heavy chain polypeptide comprise an amino acid sequence selected from SEQ ID NO: 109-116 and the first and second light chain polypeptide comprise the amino acid sequence of SEQ ID NO: 136 or 137.
Embodiment 80. The bi-specific binding protein of Embodiment 79, wherein: (a) the bi-specific binding protein comprises a first and second heavy chain polypeptide comprising the amino acid sequence of SEQ ID NO: 109 and a first and second light chain polypeptide comprising the amino acid sequence of SEQ ID NO: 136; (b) the bi-specific binding protein comprises a first and second heavy chain polypeptide comprising the amino acid sequence of SEQ ID NO: 110 and a first and second light chain polypeptide comprising the amino acid sequence of SEQ ID NO: 136; (c) the bi-specific binding protein comprises a first and second heavy chain polypeptide comprising the amino acid sequence of SEQ ID NO: 111 and a first and second light chain polypeptide comprising the amino acid sequence of SEQ ID NO: 136; (d) the bi-specific binding protein comprises a first and second heavy chain polypeptide comprising the amino acid sequence of SEQ ID NO: 112 and a first and second light chain polypeptide comprising the amino acid sequence of SEQ ID NO: 136; (e) the bi-specific binding protein comprises a first and second heavy chain polypeptide comprising the amino acid sequence of SEQ ID NO: 113 and a first and second light chain polypeptide comprising the amino acid sequence of SEQ ID NO: 137; (f) the bi-specific binding protein comprises a first and second heavy chain polypeptide comprising the amino acid sequence of SEQ ID NO: 114 and a first and second light chain polypeptide comprising the amino acid sequence of SEQ ID NO: 137; (g) the bi-specific binding protein comprises a first and second heavy chain polypeptide comprising the amino acid sequence of SEQ ID NO: 115 and a first and second light chain polypeptide comprising the amino acid sequence of SEQ ID NO: 137; or (h) the bi-specific binding protein comprises a first and second heavy chain polypeptide comprising the amino acid sequence of SEQ ID NO: 116 and a first and second light chain polypeptide comprising the amino acid sequence of SEQ ID NO: 137.
Embodiment 81. A bi-specific binding protein comprising a first and a second heavy chain polypeptide and a first and a second light chain polypeptide, wherein the first and second light chains comprise, from N-terminus to C-terminus, an antigen binding domain, a linker, and an anti-IL-17A antigen binding domain.
Embodiment 82. The bi-specific binding protein of Embodiment 81, wherein the first and second heavy chain polypeptides comprise an anti-IL-17A antibody heavy chain.
Embodiment 83. The bi-specific binding protein of Embodiment 82, wherein the first and second heavy chain polypeptides comprise an anti-IL-17A antibody heavy chain comprising an amino acid sequence selected from SEQ ID NO: 117-120.
Embodiment 84. The bi-specific binding protein of Embodiment 81 or 82, wherein the anti-IL-17A antigen binding domain is an anti-IL-17A antibody light chain.
Embodiment 85. The bi-specific binding protein of any one of Embodiments 81-84, wherein the antigen-binding domain is a VEGF-binding domain or a TNFα binding domain.
Embodiment 86. The bi-specific binding protein of Embodiment 85, wherein the first and second light chains comprise, from N-terminus to C-terminus, a VEGF binding domain comprising SEQ ID NO: 167, a linker, and an anti-IL-17A antibody light chain comprising an amino acid sequence selected from SEQ ID NO: 136 and 137.
Embodiment 87. The bi-specific binding protein of Embodiment 86, wherein the first and second light chain polypeptides comprise an amino acid sequence selected from SEQ ID NO: 138 and 139.
Embodiment 88. The bi-specific binding protein of any one of Embodiments 81-87, wherein the first and second heavy chain polypeptides comprise an amino acid sequence selected from SEQ ID NO: 117-120 and the first and second light chain polypeptides comprise an amino acid sequence selected from SEQ ID NO: 138 and 139.
Embodiment 89. The bi-specific binding protein of Embodiment 88, wherein (a) the first and second heavy chain polypeptides comprise an amino acid sequence selected from SEQ ID NO: 117 and the first and second light chain polypeptides comprise an amino acid sequence selected from SEQ ID NO: 138; (b) the first and second heavy chain polypeptides comprise an amino acid sequence selected from SEQ ID NO: 118 and the first and second light chain polypeptides comprise an amino acid sequence selected from SEQ ID NO: 138; (c) the first and second heavy chain polypeptides comprise an amino acid sequence selected from SEQ ID NO: 119 and the first and second light chain polypeptides comprise an amino acid sequence selected from SEQ ID NO: 139; or (d) the first and second heavy chain polypeptides comprise an amino acid sequence selected from SEQ ID NO: 120 and the first and second light chain polypeptides comprise an amino acid sequence selected from SEQ ID NO: 139.
Embodiment 90. A bi-specific binding protein comprising a first and a second protein monomer, wherein each monomer comprises, from N-terminus to C-terminus, an antigen binding domain, a linker, and an IL-17A binding domain.
Embodiment 91. The bi-specific binding protein of Embodiment 90, wherein the antigen-binding domain is a VEGF-binding domain or a TNFα binding domain.
Embodiment 92. The bi-specific binding protein of Embodiment 91, wherein the first and second monomers comprise, from N-terminus to C-terminus, a VEGF binding domain comprising SEQ ID NO: 100, a linker, and an anti-IL-17A scFv binding domain comprising an amino acid sequence selected from SEQ ID NO: 177-179.
Embodiment 93. The bi-specific binding protein of Embodiment 92 wherein each monomer comprises an amino acid sequence that is at least 90%, 95%, 96%, 97%, 98%, or 99% identical to an amino acid sequence selected from SEQ ID NO: 123-125.
Embodiment 94. The bi-specific binding protein of Embodiment 92, wherein each monomer comprises or consists of an amino acid sequence selected from SEQ ID NO: 123-125.
Embodiment 95. The bi-specific binding protein of Embodiment 92, wherein each monomer comprises an amino acid sequence that is at least 90%, 95%, 96%, 97%, 98%, or 99% identical to SEQ ID NO: 124.
Embodiment 96. The bi-specific binding protein of Embodiment 92, wherein each monomer comprises or consists of SEQ ID NO: 124.
Embodiment 97. The bi-specific binding protein of Embodiment 92, wherein each monomer comprises an amino acid sequence that is at least 90%, 95%, 96%, 97%, 98%, or 99% identical to SEQ ID NO: 125.
Embodiment 98. The bi-specific binding protein of Embodiment 92, wherein each monomer comprises or consists of SEQ ID NO: 125.
Embodiment 99. The bi-specific binding protein of any one of Embodiments 59, 85, or 91, wherein the VEGF antigen-binding domain is a soluble VEGF receptor or an anti-VEGF antibody or antigen-binding fragment thereof.
Embodiment 100. The bi-specific binding protein of Embodiment 99, wherein the soluble VEGF receptor is aflibercept.
Embodiment 101. The bi-specific binding protein of Embodiment 99, wherein the anti-VEGF antibody is selected from bevacizumab, ranibizumab, brolucizumab, and ramucirumab.
Embodiment 102. The bi-specific binding protein of any one of Embodiments 59, 85, or 91, wherein the TNFα antigen-binding domain is an anti-TNFα antibody or antigen binding fragment thereof.
Embodiment 103. The bi-specific binding protein of Embodiment 102, wherein the anti-TNFα antibody is selected from adalimumab, infliximab, etanercept, golimumab, and certolizumab.
Embodiment 104. A polynucleotide encoding the bi-specific binding protein of any one of Embodiments 57-103.
Embodiment 105. An expression vector comprising the polynucleotide of Embodiment 104.
Embodiment 106. A host cell comprising the polynucleotide of Embodiment 104 or the expression vector of Embodiment 105.
Embodiment 107. A method of manufacturing the bi-specific binding protein of any one of Embodiments 57-103, comprising introducing the expression vector of Embodiment 105 into a host cell.
Embodiment 108. A vector comprising a polynucleotide encoding the a the bi-specific binding protein of any one of Embodiments 57-103.
Embodiment 109. The vector of Embodiment 108, wherein the vector is an AAV8 or AAV9 vector.
Embodiment 110. A method of treating an inflammatory condition in a subject in need thereof, comprising administering the anti-IL-17A antibody or antigen-binding fragment thereof of any one of Embodiments 1-28, the humanized anti-IL-17A antibody or antigen-binding fragment thereof of any one of Embodiments 29-44, the anti-IL-17A scFv of any one of Embodiments 48-49, the bi-specific protein of any one of Embodiments 57-103, or the vector of any one of Embodiments 54, 55, 108, or 109, to a subject in need thereof.
Embodiment 111. Use of the anti-IL-17A antibody or antigen-binding fragment thereof of any one of Embodiments 1-28, the humanized anti-IL-17A antibody or antigen-binding fragment thereof of any one of Embodiments 29-44, the anti-TL-17A scFv of any one of Embodiments 48-49, the bi-specific protein of any one of Embodiments 57-103, or the vector of any one of Embodiments 54, 55, 108, or 109 for the manufacture of a medicament for treatment of an inflammatory condition in a subject.
Embodiment 112. The method of Embodiment 110 or use of Embodiment 111, wherein the inflammatory condition is selected from airway inflammation, rheumatoid arthritis, osteoarthritis, bone erosion, intraperitoneal abscesses and adhesions, infectious disease, inflammatory bowel disorder, macular degeneration, allograft rejection, psoriasis, cancer, angiogenesis, atherosclerosis, and multiple sclerosis.
Embodiment 113. A method of treating an ocular disease in a subject in need thereof, comprising administering the anti-IL-17A antibody or antigen-binding fragment thereof of any one of Embodiments 1-28, the humanized anti-IL-17A antibody or antigen-binding fragment thereof of any one of Embodiments 29-44, the anti-IL-17A scFv of any one of Embodiments 48-49, the bi-specific protein of any one of Embodiments 57-103, or the vector of any one of Embodiments 54, 55, 108, or 109, to a subject in need thereof.
Embodiment 114. Use of the anti-IL-17A antibody or antigen-binding fragment thereof of any one of Embodiments 1-28, the humanized anti-IL-17A antibody or antigen-binding fragment thereof of any one of Embodiments 29-44, the anti-TL-17A scFv of any one of Embodiments 48-49, the bi-specific protein of any one of Embodiments 57-103, or the vector of any one of Embodiments 54, 55, 108, or 109 for the manufacture of a medicament for treatment of an ocular disease in a subject.
Embodiment 115. The method of Embodiment 113 or use of Embodiment 114, wherein the ocular disease is selected from macular degeneration, retinitis pigmentosa, and diabetic retinopathy.
Embodiment 116. The method or use of Embodiment 115, wherein the macular degeneration is age-related macular degeneration.
Embodiment 117. The method or use of Embodiment 115 or 116, wherein the macular degeneration is wet or dry.
Embodiment 118. A method of decreasing vascular leakage in the eye in a subject in need thereof, comprising administering the anti-IL-17A antibody or antigen-binding fragment thereof of any one of Embodiments 1-28, the humanized anti-IL-17A antibody or antigen-binding fragment thereof of any one of Embodiments 29-44, the anti-TL-17A scFv of any one of Embodiments 48-49, the bi-specific protein of any one of Embodiments 57-103, or the vector of any one of Embodiments 54, 55, 108, or 109.
Embodiment 119. Use of the anti-IL-17A antibody or antigen-binding fragment thereof of any one of Embodiments 1-28, the humanized anti-IL-17A antibody or antigen-binding fragment thereof of any one of Embodiments 29-44, the anti-IL-17A scFv of any one of Embodiments 48-49, the bi-specific protein of any one of Embodiments 57-103, or the vector of any one of Embodiments 54, 55, 108, or 109 for the manufacture of a medicament for decreasing vascular leakage in the eye in a subject in need thereof.
Embodiment 120. The method or use of any one of Embodiments 110-119, wherein the antibody, antigen-binding fragment thereof, scFv, bi-specific protein, or vector are administered intravenously, intravitreally, subcutaneously, or intramuscularly.
Embodiment 121. The method or use of any one of Embodiments 110-120, wherein the subject is human.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in C or is at ambient temperature, and pressure is at or near atmospheric.

### Example 1: Production of Anti-IL-17A Antibodies

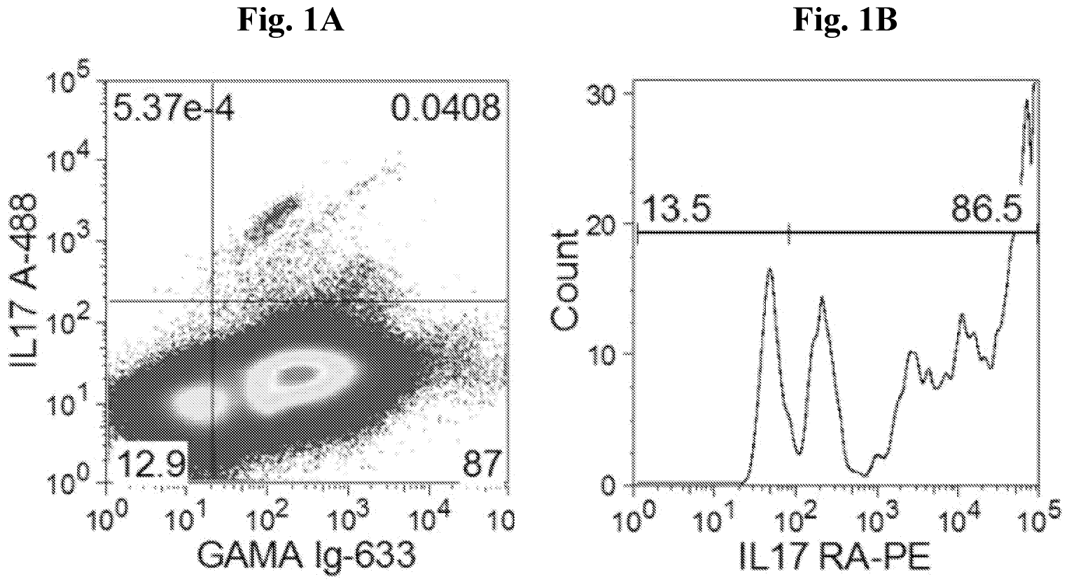

Transgenic mice overexpressing mouse Ig-Alpha, mouse Ig-Beta, and human interleukin 6 were injected intraperitoneally with recombinant human IL-17A (R&D Systems) at 2 week intervals. After a significant immune response was mounted as measured by serum ELISA, the lymph nodes, spleens, and bone marrow cells were harvested. IgM+B cells were removed with magnetic beads, and the remaining cells were sorted for their ability to bind IL-17A and for the ability of the surface antibody to block the binding of IL-17A to IL-17RA using a MoFlo Fluorescence Activated Cell Sorter (FIG. 1).

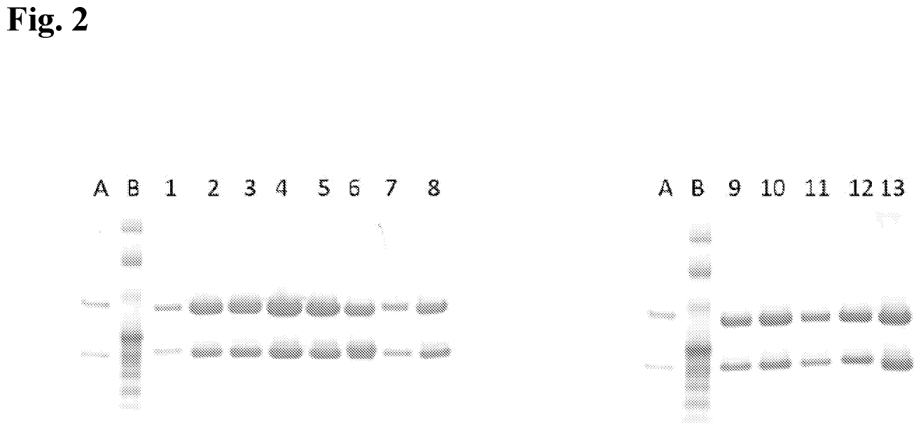

Cells positive for IL-17A binding but negative for IL-17RA binding were sorted into 96-well plates, subjected to single cell RT-PCR to amplify variable regions, and the variable regions were cloned into expression vectors containing either a heavy chain human IgG4 constant region or light chain human IgK constant region. The resulting heavy and light chain clone pairs were transfected into HEK293 cells, and the resulting antibody proteins were purified with protein A resin. Protein titers for each of the ABM antibodies as well as ixekizumab and secukinumab controls are provided in Table 37. Denaturing gels showing the heavy and light chains for each construct are provided in FIG. 2.

TABLE 37
Antibody protein titers

| Antibody | Protein concentration (μg/mL) |
|---|---|
| ABM59 | 59.90 |
| ABM60 | 55.94 |
| ABM64 | 22.26 |
| ABM67 | 48.00 |
| Ixekizumab | 44.01 |
| Secukinumab | 11.23 |

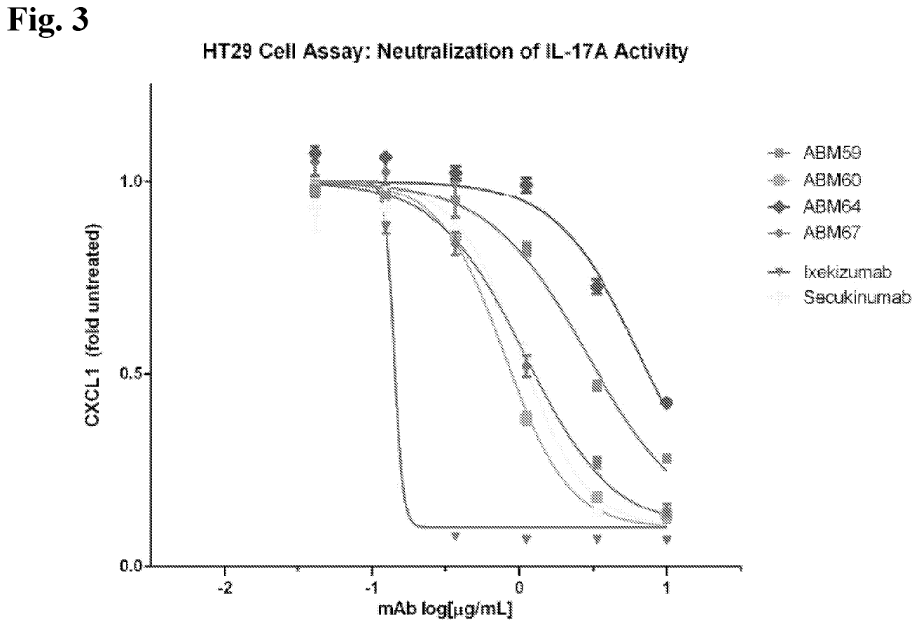

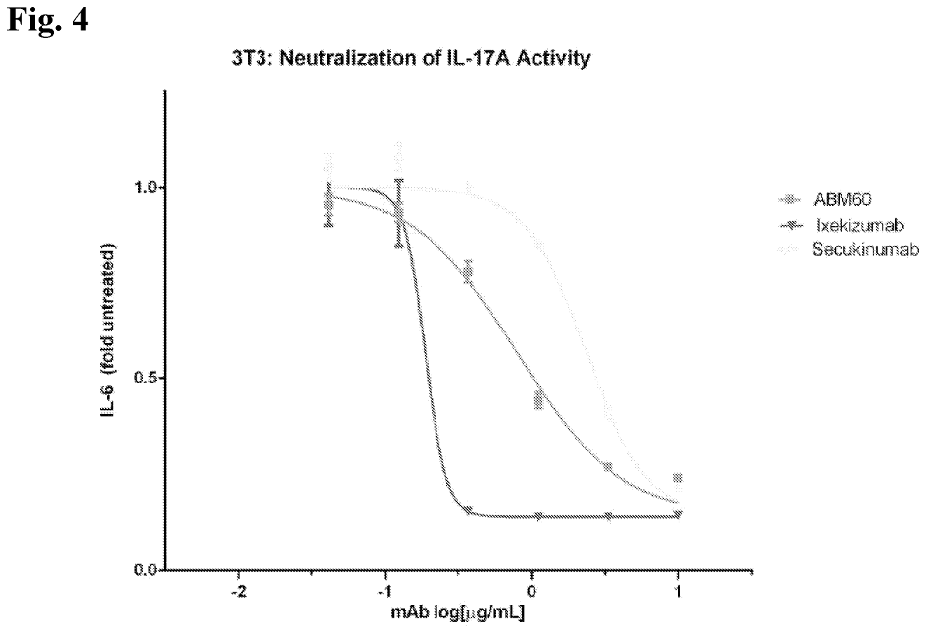

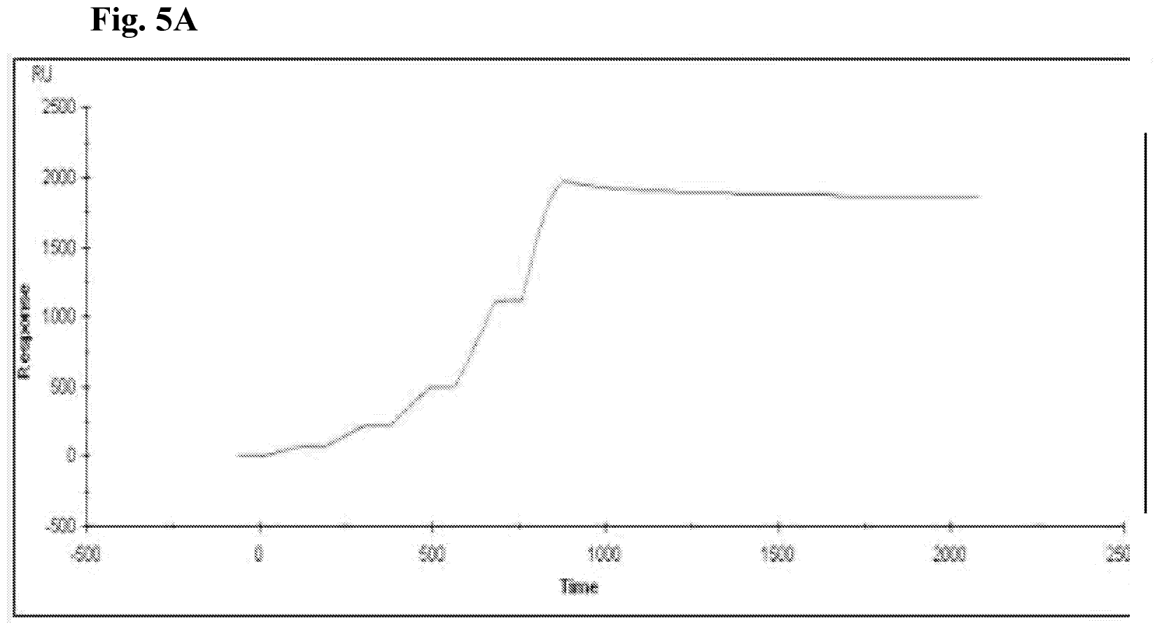

The IL-17 antibodies were then assayed for their ability to neutralize recombinant human IL-17A activity in HT-29 (FIG. 3) or 3T3 (FIG. 4) cells. Their activity was compared to monoclonal antibodies Secukinumab and Ixekizumab.

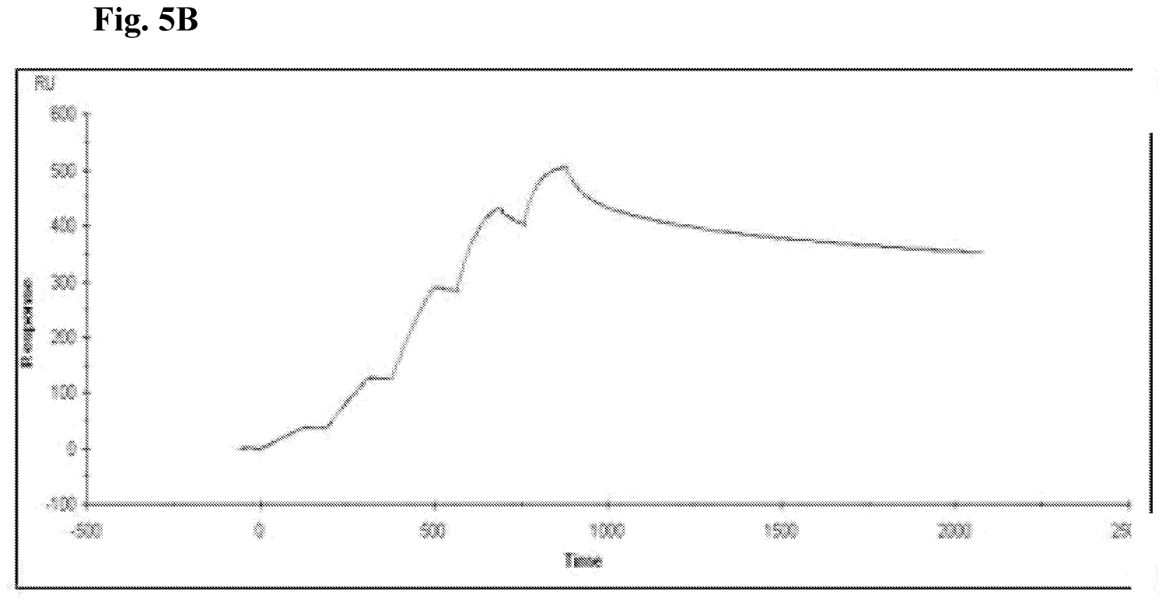

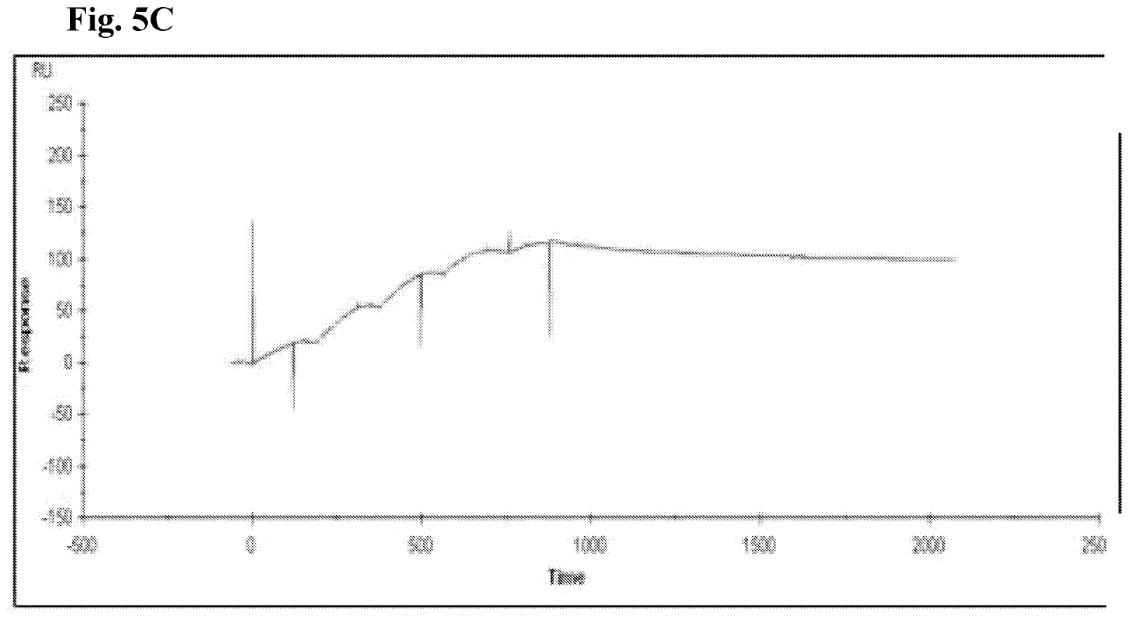

The affinity of the antibodies for IL-17A were measured using Surface Plasma Resonance (SPR) using a Biacore T-100 (GE Healthcare). Briefly, Antibodies were immobilized to carboxymethylated dextran on flow cells 2-4 on CM5 chip (flow cell 1 used as a reference). rhIL17A in HBS-P buffer was run over chip at increasing concentrations (2.5, 5.0, 10, 20, and 40 nM) with a 120 sec injection time for each concentration. After the 5^{th} injection, HBS-P buffer was run through each flow cell and IL17A was allowed to dissociate for 20 minutes. The surface was regenerated with a 30 second exposure of 10 mM Glycine-HCL pH 1.5. Single cycle kinetic analysis was performed on each antibody with the reference cell subtracted. Results are shown in FIG. 5A-FIG. 5C. A summary of the binding characteristic of the antibodies tested is provided in Table 38.

TABLE 38
Antibody binding characteristics

| Clone | Kₒₙ (M⁻¹s⁻¹) | K_{off} (S⁻¹) | K_{D} (pM) |
|---|---|---|---|
| ABM60 | 2.78 × 10⁵ | 5.65 × 10⁻⁵ | 240 |
| ABM67 | 7.13 × 10⁵ | 1.21 × 10⁻⁴ | 169 |
| ABM59 | 1.13 × 10⁶ | 5.70 × 10⁻⁴ | 506 |
| ABM64 | 1.25 × 10⁶ | 3.11 × 10⁻⁴ | 248 |
| Ixekizumab | 2.01 × 10⁶ | 1.39 × 10⁻⁵ | 6.9 |
| Secukinumab | 7.90 × 10⁵ | 1.55 × 10⁻⁴ | 196 |

### Example 2: Generation and Characterization of IL-17A Fusion Proteins

IL-17A antibody variable regions were appended with scFvs of adalimumab or VEGF trap (aflibercept) binding sequences. A description of the fusion proteins generated is provided in Tables 8-36 above.

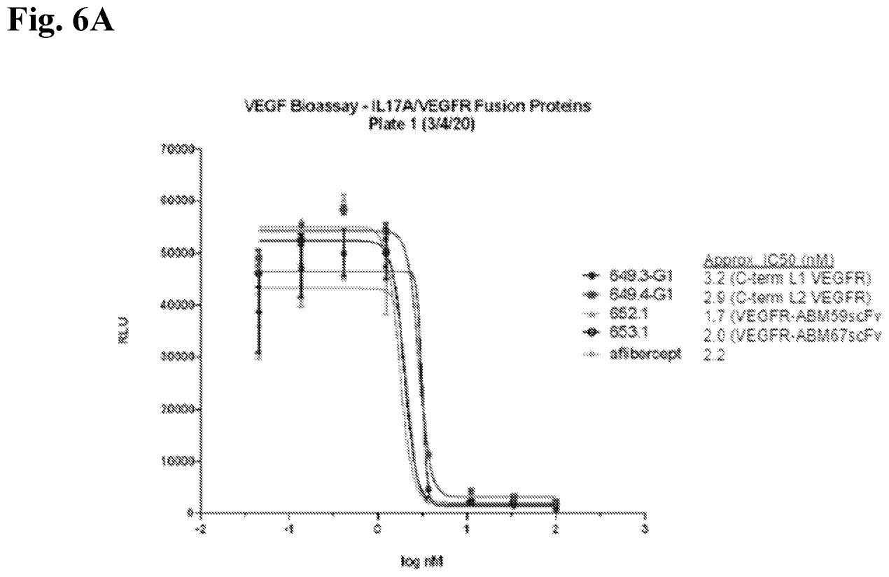

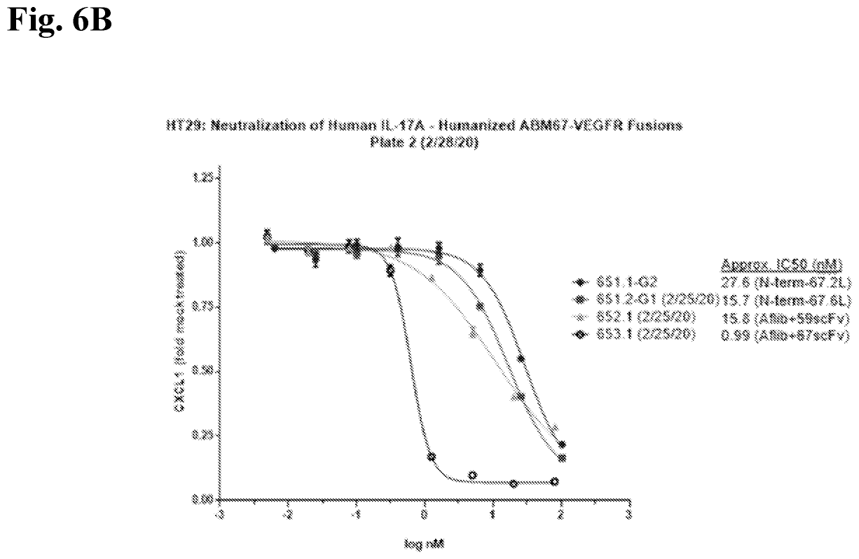

Neutralization of IL-17A activity on HT-29 cells was assayed as above in Example 1. Activity to VEGF was measured in a recombinant cell bioassay (Promega GA2001). Results are shown in Table 39. As shown, 653.1 demonstrates the highest protein yield (244 mg/mL) and the highest potency against IL-17A activity (See FIG. 6B).

TABLE 39

| | | SDS-PAGE | | |
|---|---|---|---|---|
| | Pilot yield (ExpiCHO, | Purity (% Full | IL-17A Bioassay | VEGF Bioassay |
| Fusion | mg/L) | Length) | Potency (IC50, nM) | Potency (IC50, nM) |
| 650 | 72.7, 82.5 | ~85% FL | nt | 2 |
| 648.1-G1 | 58.5, 39.2 | ~80% FL | 47.5 | nt |
| 648.1-G2 | 20.4 | ~80% FL | nt | nt |
| 648.1-G4 | 51.2 | ~70% FL | nt | nt |
| 648.2-G1 | 13.4 | ~75% FL | 8.3, 7.0 | 32.1 |
| 649.1-G1 | 25, 8 | ~75% FL | 13.1, 11.9 | nt |
| 649.2-G1 | 13.8 | ~90% FL | 14.2, 10 | nt |
| 649.3-G1 | 3.7, 69.3 | >90% FL | 5, 10.7, 5.8 | 3.2 |
| 649.4-G1 | 62.15, 73.7 | >90% FL | 16, 14.9, 9.9, 6.6 | 2.9 |
| 651.1-G2 | 33.5 | ~100% FL | 22.6, 27.6 | nt |
| 651.2-G1 | 82.8 | >90% FL | 15.7, 10.9 | 1.2 |
| 652.1 | 83.9 | >95% FL | 15.8, 10.2 | 1.7 |
| 653.1 | 244 | >95% FL | 0.99, 0.76 | 2 |
| ixekizumab | | ~100% FL | 0.39 | nt |

Results of the VEGF bioassay for fusion proteins are shown in FIG. 6A. As shown in FIG. 6, 653.1 demonstrated a potency against VEGF activity that was comparable to aflibercept.

### Example 3

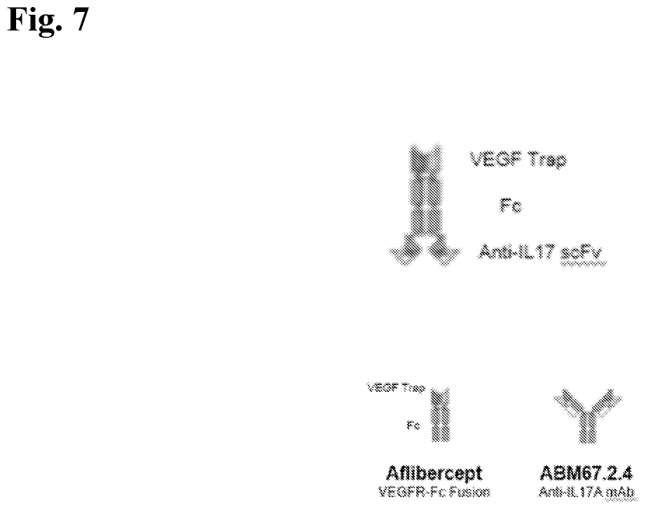

Experiments were performed to test retention of vascular leakage-inhibiting function with an anti-VEGF/anti-IL17A fusion protein, 653.1 compared to the function observed with Aflibercept (Regeneron Tarrytown, NY) in a chronic DL-α-Aminoadipic acid (AAA)-induced vascular leakage model. 653.1 comprises the ABM67 IL-17A scFv binding domain fused to the VEGFR trap binding sequences of Afilbercept (See schematic in FIG. 7).

Dutch Belted rabbits were injected intravitreally (IVT) with 1.0 mg of α-Aminoadipic acid (AAA) (Sigma) in left (OS) eyes two weeks prior to 50 μL IVT administration of Aflibercept (2 mg; Group 1) or 653.1 (2.5 mg; Group 2). Right eyes (OD) remained non-AAA-treated in Group 1 and Group 2. Group 2 OD received 653.1 to assess test article safety. Evaluations at day −2 prior to dose and at day 0 confirmed leakage induction and animals were stratified across groups according to leakage severity. Slit lamp biomicroscopy was used to evaluate ocular inflammatory scores (Hackett-McDonald [HM]) alongside monitoring of intraocular pressure (IOP) on days −2, 1, 3, 7, 14 and 28. Fluorescein angiography and fundoscopy were performed on days −2, 7, 14, 21 and 28. Background-normalized total leakage signal was determined by image analysis to quantify changes in leakage signal over time.

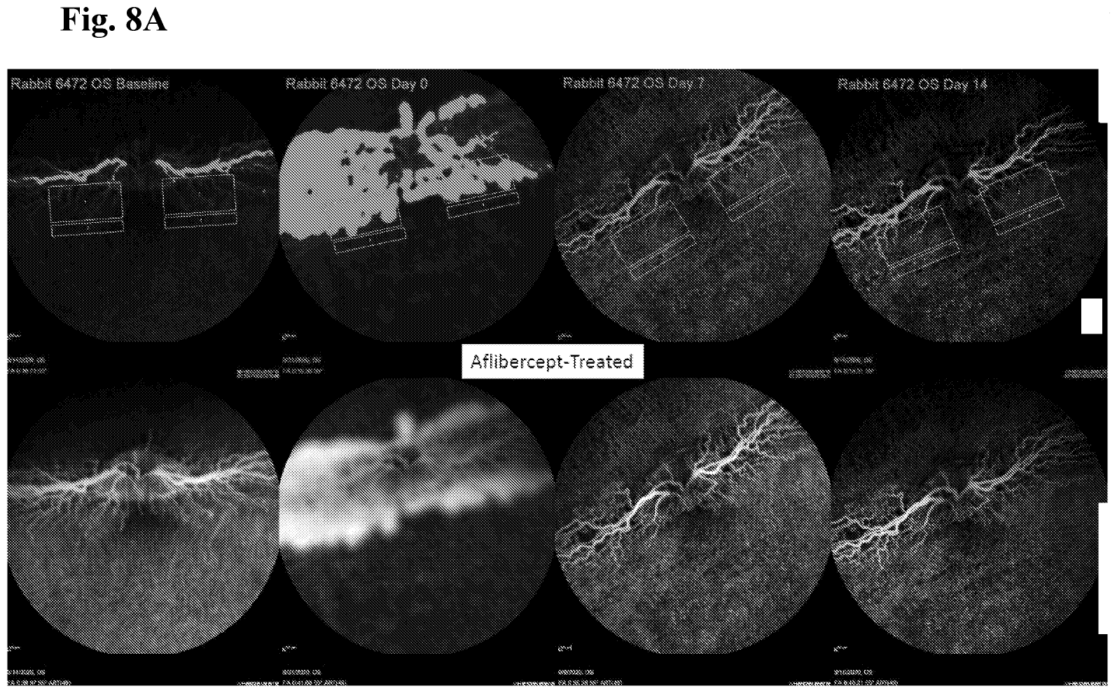

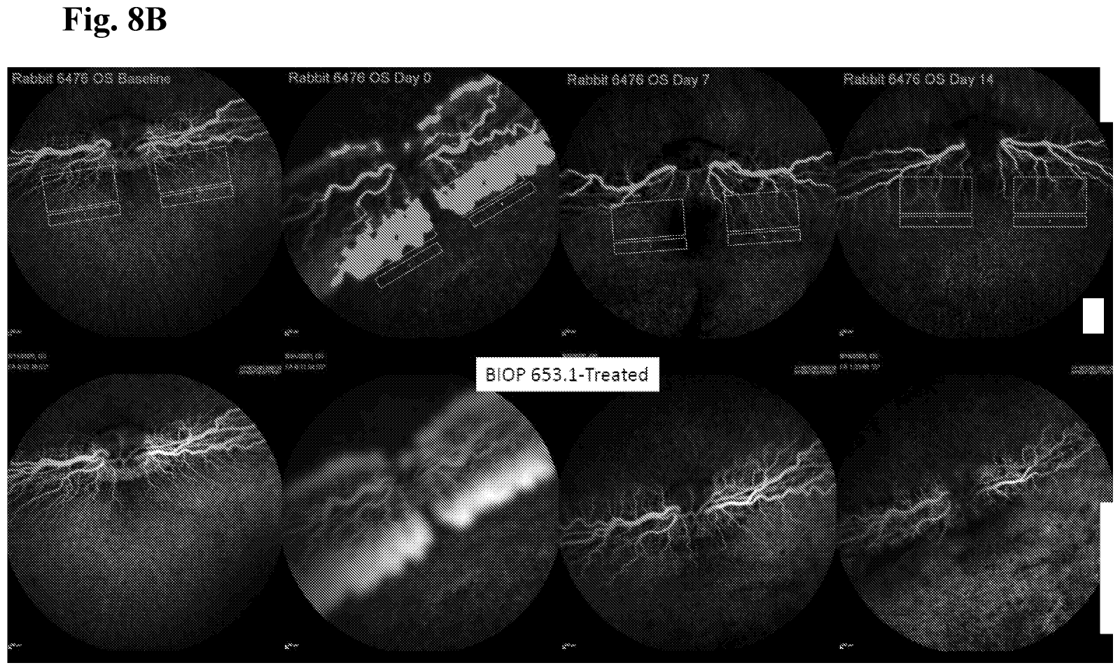

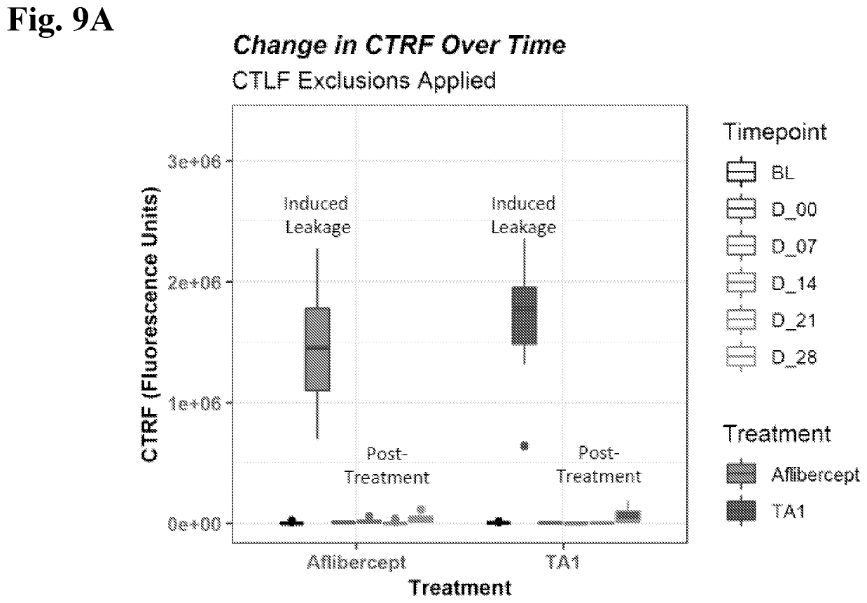

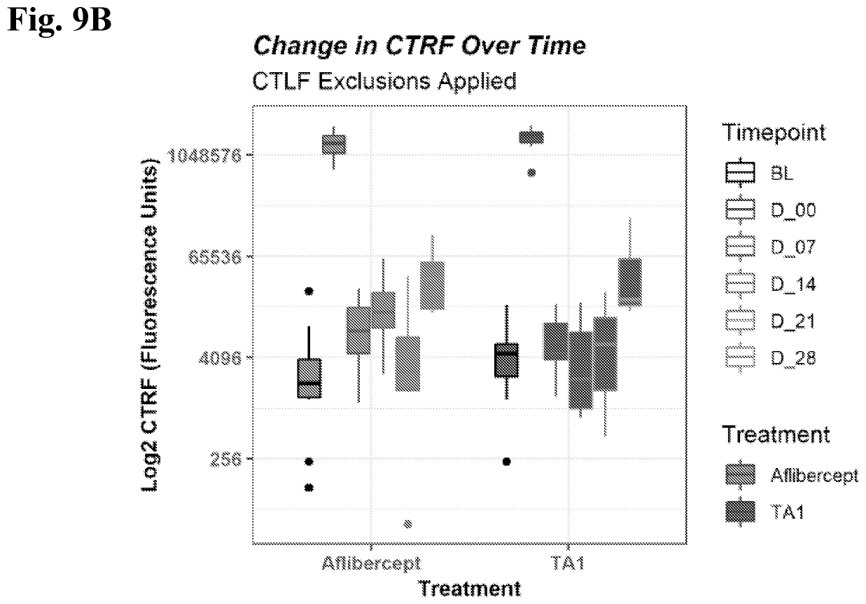

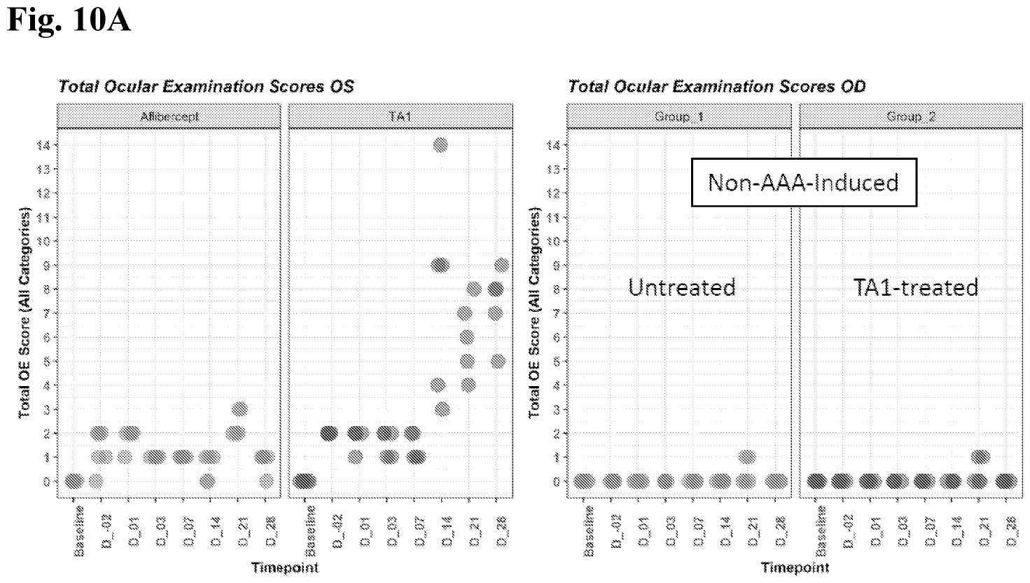

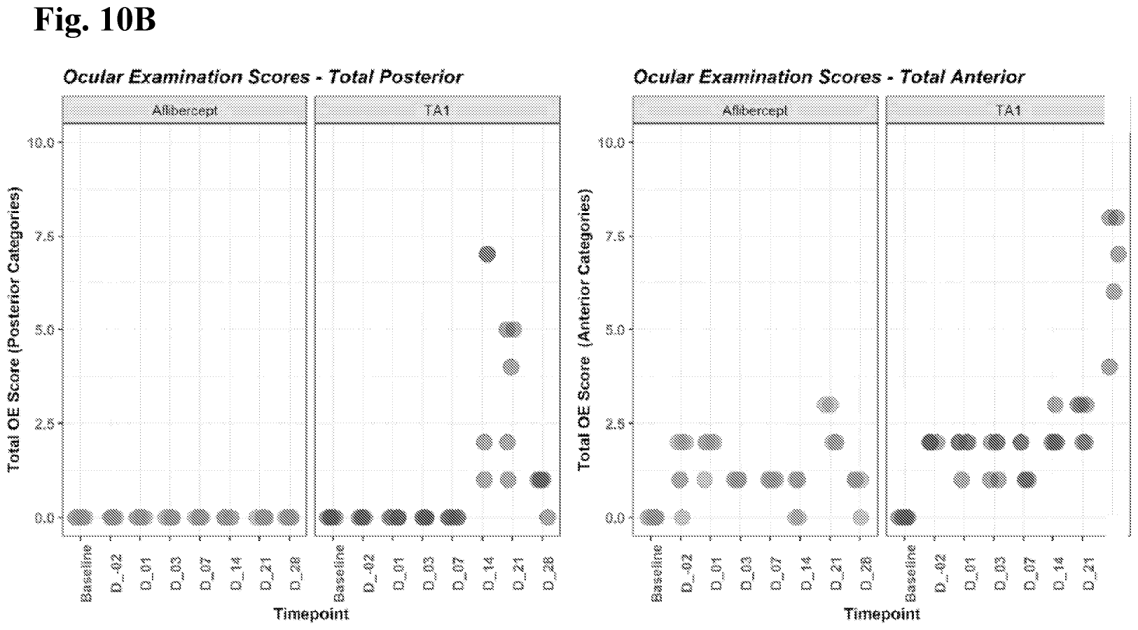

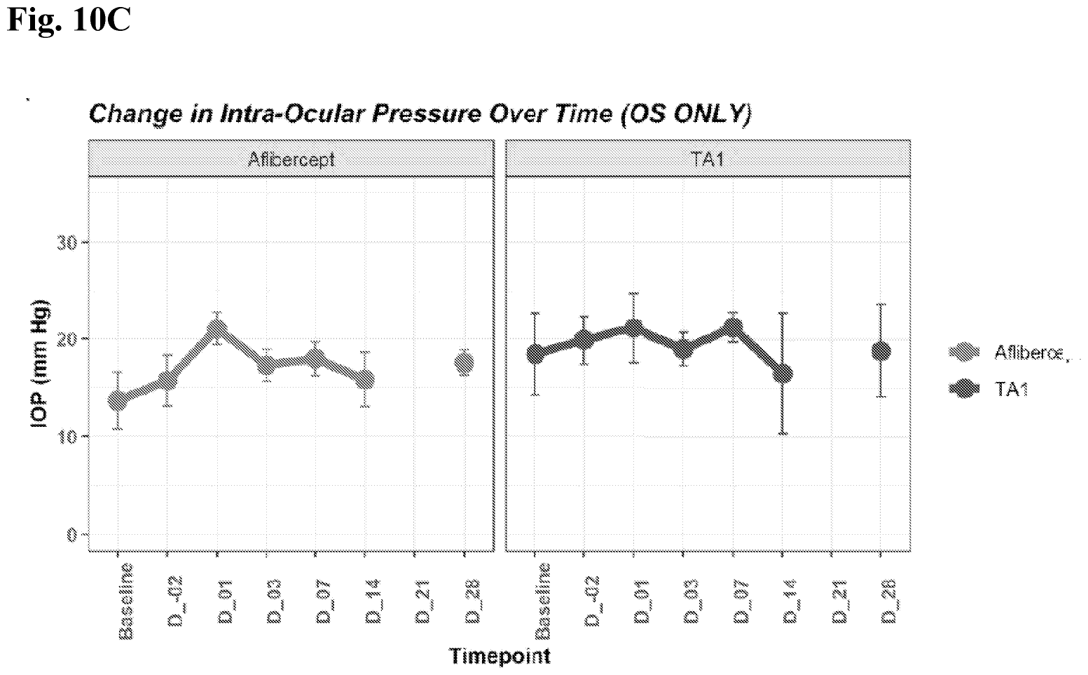

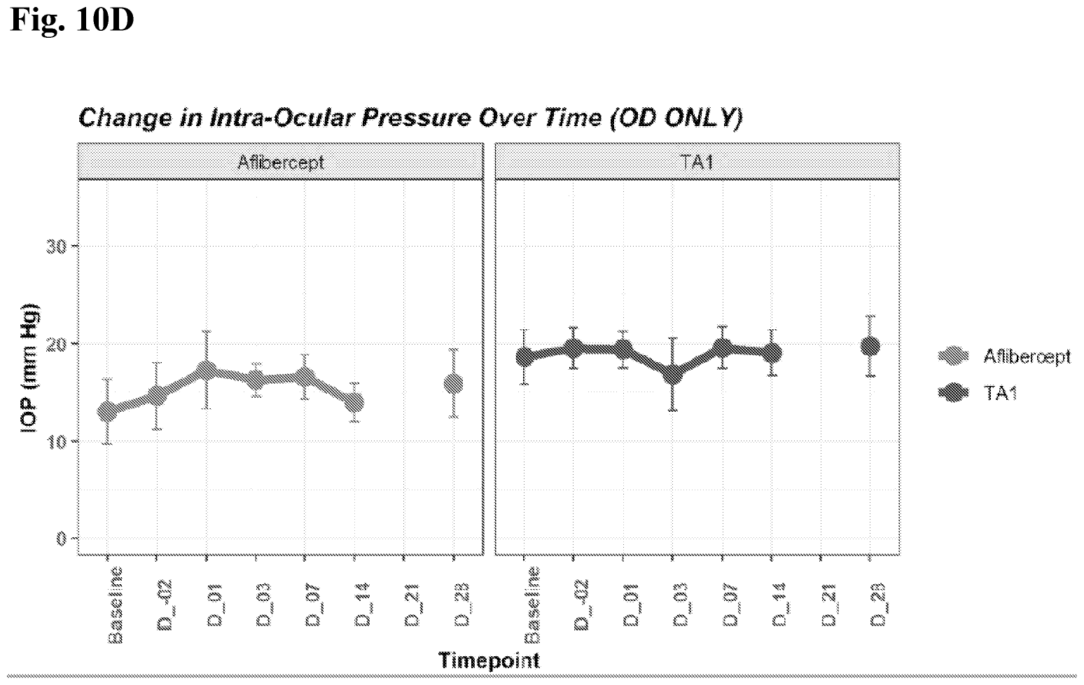

AAA-induced vascular leakage occurred in 10/10 rabbits by day −2. Resolution of leakage was comparable between Aflibercept and 653.1, suggesting that 653.1 retained leakage-inhibiting anti VEGF function (See FIG. 9A, emphasizing the difference observed in very leaky eyes, and FIG. 9B, emphasizing the difference observed between eyes with low or no leakage). Exemplary images comparing the anti-leakage effects of 653.1 and Aflibercept are provided in FIG. 8A and FIG. 8B. By day 14 HM scores were elevated in OS eyes treated with 653.1 compared to Aflibercept-injected OS eyes, and HM scores remained elevated thereafter. See FIG. 10A and FIG. 10B.

Results from IOP measurements and ocular exams were documented. Ocular examinations & IOP results demonstrated that 653.1 showed comparable safety results as Aflibercept and the results in the non-AAA induced eyes provide confidence that the 653.1 treatment itself has low toxicity risk. See FIG. 10C-FIG. 10D.

These results demonstrate inhibition of vascular leakage by 653.1 with efficacy comparable to that of Aflibercept. Future studies with 653.1 will determine half-life and durability of effect.

1. An anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein:
(a) the HCDR1 comprises an amino acid sequence selected from SEQ ID NO: 2, 23, 31, 42, 81, and 91;
(b) the HCDR2 comprises an amino acid sequence selected from SEQ ID NO: 3, 24, 32, 43, 82, and 86;
(c) the HCDR3 comprises the amino acid sequence selected from SEQ ID NO: 4, 25, 33, 45, 83, 87, 92, and 96;
(d) the LCDR1 comprises the amino acid sequence selected from SEQ ID NO: 6, 10, 27, 35, and 58;
(e) the LCDR2 comprises the amino acid sequence selected from SEQ ID NO: 7, 28, 36, and 59; and
(f) the LCDR3 comprises the amino acid sequence selected from SEQ ID NO: 8, 29, 37, 60, 89, and 94.

2. The anti-IL-17A antibody or antigen-binding fragment thereof of claim 1, wherein
(a) the HCDR1 comprises the amino acid sequence of SEQ ID NO: 2;
(b) the HCDR2 comprises the amino acid sequence of SEQ ID NO: 3;
(c) the HCDR3 comprises the amino acid sequence of SEQ ID NO: 4;
(d) the LCDR1 comprises the amino acid sequence of SEQ ID NO: 6;
(e) the LCDR2 comprises the amino acid sequence of SEQ ID NO: 7; and
(f) the LCDR2 comprises the amino acid sequence of SEQ ID NO: 8.

3. The anti-IL-17A antibody or antigen-binding fragment thereof of claim 1 or claim 2, wherein the VH comprises an amino acid sequence that is at least 95%, 96%, 97%, 98%, or 99% identical to SEQ ID NO: 5 and wherein the VL comprises an amino acid sequence that is at least 95%, 96%, 97%, 98%, or 99% identical to SEQ ID NO: 9.

4. The anti-IL-17A antibody or antigen-binding fragment thereof of claim 3, wherein the VH comprises or consists of the amino acid sequence of SEQ ID NO: 5 and wherein the VL comprises or consists of the amino acid sequence of SEQ ID NO: 9.

5. The anti-IL-17A antibody or antigen-binding fragment thereof of claim 1, wherein
(a) the HCDR1 comprises the amino acid sequence of SEQ ID NO: 2;
(b) the HCDR2 comprises the amino acid sequence of SEQ ID NO: 3;
(c) the HCDR3 comprises the amino acid sequence of SEQ ID NO: 4;
(d) the LCDR1 comprises the amino acid sequence of SEQ ID NO: 10;
(e) the LCDR2 comprises the amino acid sequence of SEQ ID NO: 7; and
(f) the LCDR2 comprises the amino acid sequence of SEQ ID NO: 8.

6. The anti-IL-17A antibody or antigen-binding fragment thereof of claim 1 or claim 5, wherein the VH comprises an amino acid sequence that is at least 95%, 96%, 97%, 98%, or 99% identical to SEQ ID NO: 5 and wherein the VL comprises an amino acid sequence that is at least 95%, 96%, 97%, 98%, or 99% identical to SEQ ID NO: 11.

7. The anti-IL-17A antibody or antigen-binding fragment thereof of claim 6, wherein the VH comprises or consists of the amino acid sequence of SEQ ID NO: 5 and wherein the VL comprises or consists of the amino acid sequence of SEQ ID NO: 11.

8. The anti-IL-17A antibody or antigen-binding fragment thereof of claim 1, wherein
(a) the HCDR1 comprises the amino acid sequence of SEQ ID NO: 23;
(b) the HCDR2 comprises the amino acid sequence of SEQ ID NO: 24;
(c) the HCDR3 comprises the amino acid sequence of SEQ ID NO: 25;
(d) the LCDR1 comprises the amino acid sequence of SEQ ID NO: 27;
(e) the LCDR2 comprises the amino acid sequence of SEQ ID NO: 28; and
(f) the LCDR2 comprises the amino acid sequence of SEQ ID NO: 29.

9. The anti-IL-17A antibody or antigen-binding fragment thereof of claim 1 or claim 8, wherein the VH comprises an amino acid sequence that is at least 95%, 96%, 97%, 98%, or 99% identical to SEQ ID NO: 26 and wherein the VL comprises an amino acid sequence that is at least 95%, 96%, 97%, 98%, or 99% identical to SEQ ID NO: 30.

10. The anti-IL-17A antibody or antigen-binding fragment thereof of claim 9, wherein the VH comprises or consists of the amino acid sequence of SEQ ID NO: 26 and wherein the VL comprises or consists of the amino acid sequence of SEQ ID NO: 30.

11. The anti-IL-17A antibody or antigen-binding fragment thereof of claim 1, wherein
(a) the HCDR1 comprises the amino acid sequence of SEQ ID NO: 31;
(b) the HCDR2 comprises the amino acid sequence of SEQ ID NO: 32;
(c) the HCDR3 comprises the amino acid sequence of SEQ ID NO: 33;
(d) the LCDR1 comprises the amino acid sequence of SEQ ID NO: 35;
(e) the LCDR2 comprises the amino acid sequence of SEQ ID NO: 36; and
(f) the LCDR2 comprises the amino acid sequence of SEQ ID NO: 37.

12. The anti-IL-17A antibody or antigen-binding fragment thereof of claim 1 or claim 11, wherein the VH comprises an amino acid sequence that is at least 95%, 96%, 97%, 98%, or 99% identical to SEQ ID NO: 34 and wherein the VL comprises an amino acid sequence that is at least 95%, 96%, 97%, 98%, or 99% identical to SEQ ID NO: 38.

13. The anti-IL-17A antibody or antigen-binding fragment thereof of claim 12, wherein the VH comprises or consists of the amino acid sequence of SEQ ID NO: 34 and wherein the VL comprises or consists of the amino acid sequence of SEQ ID NO: 38.

14. The anti-IL-17A antibody or antigen-binding fragment thereof of claim 1, wherein
(a) the HCDR1 comprises the amino acid sequence of SEQ ID NO: 42;
(b) the HCDR2 comprises the amino acid sequence of SEQ ID NO: 43;
(c) the HCDR3 comprises the amino acid sequence of SEQ ID NO: 45;
(d) the LCDR1 comprises the amino acid sequence of SEQ ID NO: 58;
(e) the LCDR2 comprises the amino acid sequence of SEQ ID NO: 59; and
(f) the LCDR2 comprises the amino acid sequence of SEQ ID NO: 60.

15. The anti-IL-17A antibody or antigen-binding fragment thereof of claim 1 or claim 14, wherein the VH comprises an amino acid sequence that is at least 95%, 96%, 97%, 98%, or 99% identical to SEQ ID NO: 57 and wherein the VL comprises an amino acid sequence that is at least 95%, 96%, 97%, 98%, or 99% identical to SEQ ID NO: 61.

16. The anti-IL-17A antibody or antigen-binding fragment thereof of claim 15, wherein the VH comprises or consists of the amino acid sequence of SEQ ID NO: 57 and wherein the VL comprises or consists of the amino acid sequence of SEQ ID NO: 61.

17. The anti-IL-17A antibody or antigen-binding fragment thereof of claim 1, wherein
(a) the HCDR1 comprises the amino acid sequence of SEQ ID NO: 81;
(b) the HCDR2 comprises the amino acid sequence of SEQ ID NO: 82;
(c) the HCDR3 comprises the amino acid sequence of SEQ ID NO: 83;
(d) the LCDR1 comprises the amino acid sequence of SEQ ID NO: 27;
(e) the LCDR2 comprises the amino acid sequence of SEQ ID NO: 28; and
(f) the LCDR2 comprises the amino acid sequence of SEQ ID NO: 29.

18. The anti-IL-17A antibody or antigen-binding fragment thereof of claim 1 or claim 17, wherein the VH comprises an amino acid sequence that is at least 95%, 96%, 97%, 98%, or 99% identical to SEQ ID NO: 84 and wherein the VL comprises an amino acid sequence that is at least 95%, 96%, 97%, 98%, or 99% identical to SEQ ID NO: 85.

19. The anti-IL-17A antibody or antigen-binding fragment thereof of claim 18, wherein the VH comprises or consists of the amino acid sequence of SEQ ID NO: 84 and wherein the VL comprises or consists of the amino acid sequence of SEQ ID NO: 85.

20. The anti-IL-17A antibody or antigen-binding fragment thereof of claim 1, wherein
(a) the HCDR1 comprises the amino acid sequence of SEQ ID NO: 81;
(b) the HCDR2 comprises the amino acid sequence of SEQ ID NO: 86;
(c) the HCDR3 comprises the amino acid sequence of SEQ ID NO: 87;
(d) the LCDR1 comprises the amino acid sequence of SEQ ID NO: 27;
(e) the LCDR2 comprises the amino acid sequence of SEQ ID NO: 28; and
(f) the LCDR2 comprises the amino acid sequence of SEQ ID NO: 89.

21. The anti-IL-17A antibody or antigen-binding fragment thereof of claim 1 or claim 20, wherein the VH comprises an amino acid sequence that is at least 95%, 96%, 97%, 98%, or 99% identical to SEQ ID NO: 85 and wherein the VL comprises an amino acid sequence that is at least 95%, 96%, 97%, 98%, or 99% identical to SEQ ID NO: 88.

22. The anti-IL-17A antibody or antigen-binding fragment thereof of claim 21, wherein the VH comprises or consists of the amino acid sequence of SEQ ID NO: 85 and wherein the VL comprises or consists of the amino acid sequence of SEQ ID NO: 88.

23. The anti-IL-17A antibody or antigen-binding fragment thereof of claim 1, wherein
(a) the HCDR1 comprises the amino acid sequence of SEQ ID NO: 91;
(b) the HCDR2 comprises the amino acid sequence of SEQ ID NO: 86;
(c) the HCDR3 comprises the amino acid sequence of SEQ ID NO: 92;
(d) the LCDR1 comprises the amino acid sequence of SEQ ID NO: 27;
(e) the LCDR2 comprises the amino acid sequence of SEQ ID NO: 28; and
(f) the LCDR2 comprises the amino acid sequence of SEQ ID NO: 94.

24. The anti-IL-17A antibody or antigen-binding fragment thereof of claim 1 or claim 23, wherein the VH comprises an amino acid sequence that is at least 95%, 96%, 97%, 98%, or 99% identical to SEQ ID NO: 93 and wherein the VL comprises an amino acid sequence that is at least 95%, 96%, 97%, 98%, or 99% identical to SEQ ID NO: 95.

25. The anti-IL-17A antibody or antigen-binding fragment thereof of claim 24, wherein the VH comprises or consists of the amino acid sequence of SEQ ID NO: 93 and wherein the VL comprises or consists of the amino acid sequence of SEQ ID NO: 95.

26. The anti-IL-17A antibody or antigen-binding fragment thereof of claim 1, wherein
(a) the HCDR1 comprises the amino acid sequence of SEQ ID NO: 81;
(b) the HCDR2 comprises the amino acid sequence of SEQ ID NO: 86;
(c) the HCDR3 comprises the amino acid sequence of SEQ ID NO: 96;
(d) the LCDR1 comprises the amino acid sequence of SEQ ID NO: 27;
(e) the LCDR2 comprises the amino acid sequence of SEQ ID NO: 28; and
(f) the LCDR2 comprises the amino acid sequence of SEQ ID NO: 94.

27. The anti-IL-17A antibody or antigen-binding fragment thereof of claim 1 or claim 26 wherein the VH comprises an amino acid sequence that is at least 95%, 96%, 97%, 98%, or 99% identical to SEQ ID NO: 97 and wherein the VL comprises an amino acid sequence that is at least 95%, 96%, 97%, 98%, or 99% identical to SEQ ID NO: 98.

28. The anti-IL-17A antibody or antigen-binding fragment thereof of claim 27, wherein the VH comprises or consists of the amino acid sequence of SEQ ID NO: 97 and wherein the VL comprises or consists of the amino acid sequence of SEQ ID NO: 98.

29. A humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein:
(a) the HCDR1 comprises an amino acid sequence selected from SEQ ID NO: 2;
(b) the HCDR2 comprises an amino acid sequence selected from SEQ ID NO: 3, and
(c) the HCDR3 comprises the amino acid sequence selected from SEQ ID NO: 4;
(d) the LCDR1 comprises the amino acid sequence selected from SEQ ID NO: 10;
(e) the LCDR2 comprises the amino acid sequence selected from SEQ ID NO: 7; and
(f) the LCDR3 comprises the amino acid sequence selected from SEQ ID NO: 8.

30. The humanized anti-IL-17A antibody or antigen-binding fragment thereof of claim 29, wherein the VH comprises an amino acid sequence is at least 95%, 96%, 97%, 98%, or 99% identical to one of SEQ ID NOs: 12, 14, 16, 21, and 22 and wherein the VL comprises an amino acid sequence is at least 95%, 96%, 97%, 98%, or 99% identical to one of SEQ ID NOs: 13, 15, 17, 18, 19, and 20.

31. The humanized anti-IL-17A antibody or antigen-binding fragment thereof of claim 30, wherein:
(a) the VH comprises an amino acid sequence is at least 95%, 96%, 97%, 98%, or 99% identical SEQ ID NO: 12 and the VL comprises an amino acid sequence is at least 95%, 96%, 97%, 98%, or 99% identical to SEQ ID NO: 13;
(b) the VH comprises an amino acid sequence is at least 95%, 96%, 97%, 98%, or 99% identical SEQ ID NO: 14 and the VL comprises an amino acid sequence is at least 95%, 96%, 97%, 98%, or 99% identical to SEQ ID NO: 15;
(c) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 15;
(d) the VH comprises an amino acid sequence is at least 95%, 96%, 97%, 98%, or 99% identical SEQ ID NO: 14 and the VL comprises an amino acid sequence is at least 95%, 96%, 97%, 98%, or 99% identical to SEQ ID NO: 17;
(e) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 17;
(f) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 18;
(g) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 19;
(h) the VH comprises an amino acid sequence is at least 95%, 96%, 97%, 98%, or 99% identical SEQ ID NO: 16 and the VL comprises an amino acid sequence is at least 95%, 96%, 97%, 98%, or 99% identical to SEQ ID NO: 20;
(i) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 18;
(j) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 19;
(k) the VH comprises an amino acid sequence is at least 95%, 96%, 97%, 98%, or 99% identical SEQ ID NO: 21 and the VL comprises an amino acid sequence is at least 95%, 96%, 97%, 98%, or 99% identical to SEQ ID NO: 20;
(1) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 18;
(m) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 19; or
(n) the VH comprises an amino acid sequence is at least 95%, 96%, 97%, 98%, or 99% identical SEQ ID NO: 22 and the VL comprises an amino acid sequence is at least 95%, 96%, 97%, 98%, or 99% identical to SEQ ID NO: 20.

32. The humanized anti-IL-17A antibody or antigen-binding fragment thereof of claim 29, wherein the VH comprises or consists of one of SEQ ID NOs: 12, 14, 16, 21, and 22 and wherein the VL comprises or consists of one of SEQ ID NOs: 13, 15, 17, 18, 19, and 20.

33. The humanized anti-IL-17A antibody or antigen-binding fragment thereof of claim 32 wherein:
(a) the VH amino acid sequence comprises or consists of SEQ ID NO: 12 and the VL amino acid sequence comprises or consists of SEQ ID NO: 13;
(b) the VH amino acid sequence comprises or consists of SEQ ID NO: 14 and the VL amino acid sequence comprises or consists of SEQ ID NO: 15;
(c) the VH amino acid sequence comprises or consists of SEQ ID NO: 16 and the VL amino acid sequence comprises or consists of SEQ ID NO: 15;
(d) the VH amino acid sequence comprises or consists of SEQ ID NO: 14 and the VL amino acid sequence comprises or consists of SEQ ID NO: 17;
(e) the VH amino acid sequence comprises or consists of SEQ ID NO: 16 and the VL amino acid sequence comprises or consists of SEQ ID NO: 17;
(f) the VH amino acid sequence comprises or consists of SEQ ID NO: 16 and the VL amino acid sequence comprises or consists of SEQ ID NO: 18;
(g) the VH amino acid sequence comprises or consists of SEQ ID NO: 16 and the VL amino acid sequence comprises or consists of SEQ ID NO: 19;
(h) the VH amino acid sequence comprises or consists of SEQ ID NO: 16 and the VL amino acid sequence comprises or consists of SEQ ID NO: 20;
(i) the VH amino acid sequence comprises or consists of SEQ ID NO: 21 and the VL amino acid sequence comprises or consists of SEQ ID NO: 18;
(j) the VH amino acid sequence comprises or consists of SEQ ID NO: 21 and the VL amino acid sequence comprises or consists of SEQ ID NO: 19;
(k) the VH amino acid sequence comprises or consists of SEQ ID NO: 21 and the VL amino acid sequence comprises or consists of SEQ ID NO: 20;
(l) the VH amino acid sequence comprises or consists of SEQ ID NO: 22 and the VL amino acid sequence comprises or consists of SEQ ID NO: 18;
(m) the VH amino acid sequence comprises or consists of SEQ ID NO: 22 and the VL amino acid sequence comprises or consists of SEQ ID NO: 19; or
(n) the VH amino acid sequence comprises or consists of SEQ ID NO: 22 and the VL amino acid sequence comprises or consists of SEQ ID NO: 20.

34. A humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein:
(a) the HCDR1 comprises an amino acid sequence selected from SEQ ID NO: 31;
(b) the HCDR2 comprises an amino acid sequence selected from SEQ ID NO: 32, and
(c) the HCDR3 comprises the amino acid sequence selected from SEQ ID NO: 33;
(d) the LCDR1 comprises the amino acid sequence selected from SEQ ID NO: 35;
(e) the LCDR2 comprises the amino acid sequence selected from SEQ ID NO: 36; and
(f) the LCDR3 comprises the amino acid sequence selected from SEQ ID NO: 37.

35. The humanized anti-IL-17A antibody or antigen-binding fragment thereof of claim 34, wherein the VH comprises an amino acid sequence is at least 95%, 96%, 97%, 98%, or 99% identical to one of SEQ ID NOs: 39 and 34 and wherein the VL comprises an amino acid sequence is at least 95%, 96%, 97%, 98%, or 99% identical to one of SEQ ID NOs: 40 and 41.

36. The humanized anti-IL-17A antibody or antigen-binding fragment thereof of claim 35, wherein:
(a) the VH comprises an amino acid sequence is at least 95%, 96%, 97%, 98%, or 99% identical SEQ ID NO: 39 and the VL comprises an amino acid sequence is at least 95%, 96%, 97%, 98%, or 99% identical to SEQ ID NO: 40;
(b) the VH comprises an amino acid sequence is at least 95%, 96%, 97%, 98%, or 99% identical SEQ ID NO: 39 and the VL comprises an amino acid sequence is at least 95%, 96%, 97%, 98%, or 99% identical to SEQ ID NO: 41; or
(c) the VH comprises an amino acid sequence is at least 95%, 96%, 97%, 98%, or 99% identical SEQ ID NO: 34 and the VL comprises an amino acid sequence is at least 95%, 96%, 97%, 98%, or 99% identical to SEQ ID NO: 41.

37. The humanized anti-IL-17A antibody or antigen-binding fragment thereof of claim 34, wherein the VH comprises or consists of one of SEQ ID NOs: 39 and 34 and wherein the VL comprises or consists of one of SEQ ID NOs: 40 and 41.

38. The humanized anti-IL-17A antibody or antigen-binding fragment thereof of claim 37, wherein:
(a) the VH amino acid sequence comprises or consists of SEQ ID NO: 39 and the VL amino acid sequence comprises or consists of SEQ ID NO: 40;
(b) the VH amino acid sequence comprises or consists of SEQ ID NO: 39 and the VL amino acid sequence comprises or consists of SEQ ID NO: 41; or
(c) the VH amino acid sequence comprises or consists of SEQ ID NO: 34 and the VL amino acid sequence comprises or consists of SEQ ID NO: 41.

39. A humanized anti-IL-17A antibody or antigen-binding fragment thereof comprising an immunoglobulin heavy chain variable region (VH) comprising a heavy chain complementarity determining region (CDR) 1 (HCDR1), a heavy chain CDR2 (HCDR2), and a heavy chain CDR3 (HCDR3), and an immunoglobulin light chain variable region (VL) comprising a light chain complementarity determining region (CDR) 1 (LCDR1), a light chain CDR2 (LCDR2), and a light chain CDR3 (LCDR3), wherein:
(a) the HCDR1 comprises an amino acid of SEQ ID NO: 42;
(b) the HCDR2 comprises an amino acid sequence selected from SEQ ID NO: 43 or SEQ ID NO: 44, and
(c) the HCDR3 comprises the amino acid sequence selected from any one of SEQ ID NOs: 45-56;
(d) the LCDR1 comprises the amino acid sequence selected from SEQ ID NO: 58;
(e) the LCDR2 comprises the amino acid sequence selected from SEQ ID NO: 59; and
(f) the LCDR3 comprises the amino acid sequence selected from SEQ ID NO: 60.

40. The humanized anti-IL-17A antibody or antigen-binding fragment thereof of claim 39, wherein the LCDR1 comprises the amino acid sequence of SEQ ID NO: 58; the LCDR2 comprises the amino acid sequence of SEQ ID NO: 59; and the LCDR3 comprises the amino acid sequence of SEQ ID NO: 60; and wherein:
(a) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 45;
(b) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 46;
(c) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 47;
(d) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 48;
(e) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 49;
(f) the HCDR1 comprises an amino acid sequence selected from SEQ ID NO: 42; the HCDR2 comprises an amino acid sequence selected from SEQ ID NO: 44, and the HCDR3 comprises the amino acid sequence of SEQ ID NO: 50;
(g) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 51;
(h) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 52;
(i) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 53;
(j) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 54;
(k) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 55; or
(l) the HCDR1 comprises an amino acid sequence selected from SEQ ID NO: 42; the HCDR2 comprises an amino acid sequence selected from SEQ ID NO: 43, and the HCDR3 comprises the amino acid sequence of SEQ ID NO: 56.

41. The humanized anti-IL-17A antibody or antigen-binding fragment thereof of claim 39, wherein the VH comprises an amino acid sequence is at least 95%, 96%, 97%, 98%, or 99% identical to one of SEQ ID NOs: 62, 64, 65, 66, 67, 68, 69, 71, 72, 73, 74, 99, 77, 78, or 79 and wherein the VL comprises an amino acid sequence is at least 95%, 96%, 97%, 98%, or 99% identical to one of SEQ ID NOs: 63, 70, 75, 76, or 80.

42. The humanized anti-IL-17A antibody or antigen-binding fragment thereof of claim 41, wherein:
(a) the VH comprises an amino acid sequence is at least 95%, 96%, 97%, 98%, or 99% identical SEQ ID NO: 62 and the VL comprises an amino acid sequence is at least 95%, 96%, 97%, 98%, or 99% identical to SEQ ID NO: 63;
(b) the VH comprises an amino acid sequence is at least 95%, 96%, 97%, 98%, or 99% identical SEQ ID NO: 64 and the VL comprises an amino acid sequence is at least 95%, 96%, 97%, 98%, or 99% identical to SEQ ID NO: 63;
(c) the VH comprises an amino acid sequence is at least 95%, 96%, 97%, 98%, or 99% identical SEQ ID NO: 65 and the VL comprises an amino acid sequence is at least 95%, 96%, 97%, 98%, or 99% identical to SEQ ID NO: 63;
(d) the VH comprises an amino acid sequence is at least 95%, 96%, 97%, 98%, or 99% identical SEQ ID NO: 66 and the VL comprises an amino acid sequence is at least 95%, 96%, 97%, 98%, or 99% identical to SEQ ID NO: 63;
(e) the VH comprises an amino acid sequence is at least 95%, 96%, 97%, 98%, or 99% identical SEQ ID NO: 67 and the VL comprises an amino acid sequence is at least 95%, 96%, 97%, 98%, or 99% identical to SEQ ID NO: 63;
(f) the VH comprises an amino acid sequence is at least 95%, 96%, 97%, 98%, or 99% identical SEQ ID NO: 68 and the VL comprises an amino acid sequence is at least 95%, 96%, 97%, 98%, or 99% identical to SEQ ID NO: 63;
(g) the VH comprises an amino acid sequence is at least 95%, 96%, 97%, 98%, or 99% identical SEQ ID NO: 69 and the VL comprises an amino acid sequence is at least 95%, 96%, 97%, 98%, or 99% identical to SEQ ID NO: 70;
(h) the VH comprises an amino acid sequence is at least 95%, 96%, 97%, 98%, or 99% identical SEQ ID NO: 71 and the VL comprises an amino acid sequence is at least 95%, 96%, 97%, 98%, or 99% identical to SEQ ID NO: 70;
(i) the VH comprises an amino acid sequence is at least 95%, 96%, 97%, 98%, or 99% identical SEQ ID NO: 72 and the VL comprises an amino acid sequence is at least 95%, 96%, 97%, 98%, or 99% identical to SEQ ID NO: 70;
(j) the VH comprises an amino acid sequence is at least 95%, 96%, 97%, 98%, or 99% identical SEQ ID NO: 73 and the VL comprises an amino acid sequence is at least 95%, 96%, 97%, 98%, or 99% identical to SEQ ID NO: 70;
(k) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 70;
(l) the VH comprises an amino acid sequence is at least 95%, 96%, 97%, 98%, or 99% identical SEQ ID NO: 99 and the VL comprises an amino acid sequence is at least 95%, 96%, 97%, 98%, or 99% identical to SEQ ID NO: 70;
(m) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 75;
(n) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 76;
(o) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 70;
(p) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 75;
(q) the VH comprises an amino acid sequence is at least 95%, 96%, 97%, 98%, or 99% identical SEQ ID NO: 77 and the VL comprises an amino acid sequence is at least 95%, 96%, 97%, 98%, or 99% identical to SEQ ID NO: 76;
(r) the VH comprises an amino acid sequence is at least 95%, 96%, 97%, 98%, or 99% identical SEQ ID NO: 78 and the VL comprises an amino acid sequence is at least 95%, 96%, 97%, 98%, or 99% identical to SEQ ID NO: 70;
(s) the VH comprises an amino acid sequence is at least 95%, 96%, 97%, 98%, or 99% identical SEQ ID NO: 79 and the VL comprises an amino acid sequence is at least 95%, 96%, 97%, 98%, or 99% identical to SEQ ID NO: 70; or
(t) the VH comprises an amino acid sequence is at least 95%, 96%, 97%, 98%, or 99% identical SEQ ID NO: 74 and the VL comprises an amino acid sequence is at least 95%, 96%, 97%, 98%, or 99% identical to SEQ ID NO: 80.

43. The humanized anti-IL-17A antibody or antigen-binding fragment thereof of claim 39, wherein the VH comprises or consists of one of SEQ ID NOs: 62, 64, 65, 66, 67, 68, 69, 71, 72, 73, 74, 99, 77, 78, or 79 and wherein the VL comprises or consists of one of SEQ ID NOs: 63, 70, 75, 76, or 80.

44. The humanized anti-IL-17A antibody or antigen-binding fragment thereof of claim 43, wherein:
(a) the VH amino acid sequence comprises or consists of SEQ ID NO: 62 and the VL amino acid sequence comprises or consists of SEQ ID NO: 63;
(b) the VH amino acid sequence comprises or consists of SEQ ID NO: 64 and the VL amino acid sequence comprises or consists of SEQ ID NO: 63;
(c) the VH amino acid sequence comprises or consists of SEQ ID NO: 65 and the VL amino acid sequence comprises or consists of SEQ ID NO: 63;
(d) the VH amino acid sequence comprises or consists of SEQ ID NO: 66 and the VL amino acid sequence comprises or consists of SEQ ID NO: 63;
(e) the VH amino acid sequence comprises or consists of SEQ ID NO: 67 and the VL amino acid sequence comprises or consists of SEQ ID NO: 63;
(f) the VH amino acid sequence comprises or consists of SEQ ID NO: 68 and the VL amino acid sequence comprises or consists of SEQ ID NO: 63;
(g) the VH amino acid sequence comprises or consists of SEQ ID NO: 69 and the VL amino acid sequence comprises or consists of SEQ ID NO: 70;
(h) the VH amino acid sequence comprises or consists of SEQ ID NO: 71 and the VL amino acid sequence comprises or consists of SEQ ID NO: 70;
(i) the VH amino acid sequence comprises or consists of SEQ ID NO: 72 and the VL amino acid sequence comprises or consists of SEQ ID NO: 70;
(j) the VH amino acid sequence comprises or consists of SEQ ID NO: 73 and the VL amino acid sequence comprises or consists of SEQ ID NO: 70;
(k) the VH amino acid sequence comprises or consists of SEQ ID NO: 74 and the VL amino acid sequence comprises or consists of SEQ ID NO: 70;
(l) the VH amino acid sequence comprises or consists of SEQ ID NO: 99 and the VL amino acid sequence comprises or consists of SEQ ID NO: 70;
(m) the VH amino acid sequence comprises or consists of SEQ ID NO: 74 and the VL amino acid sequence comprises or consists of SEQ ID NO: 75;
(n) the VH amino acid sequence comprises or consists of SEQ ID NO: 74 and the VL amino acid sequence comprises or consists of SEQ ID NO: 76;
(o) the VH amino acid sequence comprises or consists of SEQ ID NO: 77 and the VL amino acid sequence comprises or consists of SEQ ID NO: 70;
(p) the VH amino acid sequence comprises or consists of SEQ ID NO: 77 and the VL amino acid sequence comprises or consists of SEQ ID NO: 75;
(q) the VH amino acid sequence comprises or consists of SEQ ID NO: 77 and the VL amino acid sequence comprises or consists of SEQ ID NO: 76;
(r) the VH amino acid sequence comprises or consists of SEQ ID NO: 78 and the VL amino acid sequence comprises or consists of SEQ ID NO: 70;
(s) the VH amino acid sequence comprises or consists of SEQ ID NO: 79 and the VL amino acid sequence comprises or consists of SEQ ID NO: 70; or
(t) the VH amino acid sequence comprises or consists of SEQ ID NO: 74 and the VL amino acid sequence comprises or consists of SEQ ID NO: 80.

45. The anti-IL-17A antibody or antigen-binding fragment thereof of any one of claims 1-28 or the humanized anti-IL-17A antibody or antigen-binding fragment thereof of any one of claims 29-44, wherein the antigen binding fragment is a single chain variable fragment (scFv).

46. The anti-IL-17A antibody or antigen-binding fragment thereof or the humanized anti-IL-17A antibody or antigen-binding fragment thereof of claim 45, wherein the scFv comprises an amino acid sequence that is at least 95%, 96%, 97%, 98%, or 99% identical to an amino acid sequence selected from SEQ ID NOs: 177-179.

47. The anti-IL-17A antibody or antigen-binding fragment thereof or the humanized anti-IL-17A antibody or antigen-binding fragment thereof of claim 45, wherein the scFv comprises or consists of an amino acid sequence selected from SEQ ID NOs: 177-179.

48. An anti-IL-17A single chain variable fragment (scFv) comprising an amino acid sequence that is at least 95%, 96%, 97%, 98%, or 99% identical to an amino acid sequence selected from SEQ ID NOs: 177-179.

49. The anti-IL-17A scFv of claim 48, comprising or consisting of an amino acid sequence selected from SEQ ID NOs: 177-179.

50. A polynucleotide encoding the anti-IL-17A antibody or antigen-binding fragment thereof of any one of claims 1-28 or the humanized anti-IL-17A antibody or antigen-binding fragment thereof of any one of claims 29-44.

51. A polynucleotide encoding the anti-IL-17A scFv of any one of claims 48-49.

52. An expression vector comprising the polynucleotide of claim 50 or 51.

53. A host cell comprising the polynucleotide of claim 50 or 51 or the expression vector of claim 52.

54. A vector comprising a polynucleotide encoding the anti-IL-17A antibody or antigen-binding fragment thereof of any one of claims 1-28, the humanized anti-IL-17A antibody or antigen-binding fragment thereof of any one of claims 29-44, or the anti-IL-17A scFv of any one of claims 48-49.

55. The vector of claim 54, wherein the vector is an AAV8 or AAV9 vector.

56. A method of manufacturing the anti-IL-17A antibody or antigen-binding fragment thereof of any one of claims 1-28, the humanized anti-IL-17A antibody or antigen-binding fragment thereof of any one of claims 29-44, or the anti-IL-17A scFv of any one of claims 48-49 comprising introducing the expression vector of claim 52 into a host cell.

57. A bi-specific binding protein comprising a first and a second heavy chain polypeptide and a first and a second light chain polypeptide, wherein the first and second heavy chain polypeptides comprise, from N-terminus to C-terminus, an antigen-binding domain, a linker, and an anti-IL-17A antigen-binding domain.

58. The bi-specific binding protein of claim 57, wherein the anti-IL-17A antigen-binding domain is an anti-IL-17A antibody heavy chain.

59. The bi-specific binding protein of claim 57 or 58, wherein the antigen-binding domain is a VEGF-binding domain or a TNFα binding domain.

60. The bi-specific binding protein of claim 59, wherein the first and second heavy chain polypeptides comprise, from N-terminus to C-terminus, a VEGF antigen-binding domain comprising an amino acid sequence of SEQ ID NO: 167 or 168, a linker, and an anti-IL-17A antibody heavy chain comprising an amino acid sequence selected from SEQ ID NOs: 169-174.

61. The bi-specific binding protein of claim 60, wherein the first and second heavy chain polypeptides comprise an amino acid sequence selected from SEQ ID NOs: 101-108.

62. The bi-specific binding protein of claim 60 or 61, wherein the first and second light chain polypeptides comprise an anti-IL-17A antibody light chain.

63. The bi-specific binding protein of claim 62, wherein the first and second light chain polypeptides comprise the amino acid sequence selected from SEQ ID NO: 136 and 137.

64. The bi-specific binding protein of any one of claims 57-63, wherein the first and second heavy chain polypeptide comprising an amino acid sequence selected from the group of SEQ ID NO: 101-108 and the first and second light chain polypeptide comprising an amino acid sequence selected from SEQ ID NO: 136 and 137.

65. The bi-specific binding protein of claim 64, wherein
(a) the bi-specific binding protein comprises a first and second heavy chain polypeptide comprising the amino acid sequence of SEQ ID NO: 101 and a first and second light chain polypeptide comprising the amino acid sequence of SEQ ID NO: 136;
(b) the bi-specific binding protein comprises a first and second heavy chain polypeptide comprising the amino acid sequence of SEQ ID NO: 102 and a first and second light chain polypeptide comprising the amino acid sequence of SEQ ID NO: 136;
(c) the bi-specific binding protein comprises a first and second heavy chain polypeptide comprising the amino acid sequence of SEQ ID NO: 103 and a first and second light chain polypeptide comprising the amino acid sequence of SEQ ID NO: 136;
(d) the bi-specific binding protein comprises a first and second heavy chain polypeptide comprising the amino acid sequence of SEQ ID NO: 104 and a first and second light chain polypeptide comprising the amino acid sequence of SEQ ID NO: 136;
(e) the bi-specific binding protein comprises a first and second heavy chain polypeptide comprising the amino acid sequence of SEQ ID NO: 105 and a first and second light chain polypeptide comprising the amino acid sequence of SEQ ID NO: 136;
(f) the bi-specific binding protein comprises a first and second heavy chain polypeptide comprising the amino acid sequence of SEQ ID NO: 106 and a first and second light chain polypeptide comprising the amino acid sequence of SEQ ID NO: 136;
(g) the bi-specific binding protein comprises a first and second heavy chain polypeptide comprising the amino acid sequence of SEQ ID NO: 107 and a first and second light chain polypeptide comprising the amino acid sequence of SEQ ID NO: 137; or
(h) the bi-specific binding protein comprises a first and second heavy chain polypeptide comprising the amino acid sequence of SEQ ID NO: 108 and a first and second light chain polypeptide comprising the amino acid sequence of SEQ ID NO: 137.

66. The bi-specific binding protein of claim 59, wherein the first and second heavy chain polypeptides comprise, from N-terminus to C-terminus, a TNFα antigen-binding domain comprising an amino acid sequence of SEQ ID NO: 134, a linker, and an anti-IL-17A antibody heavy chain comprising an amino acid sequence selected from SEQ ID NOs: 180-185.

67. The bi-specific binding protein of claim 66, wherein the first and second heavy chain polypeptides comprise an amino acid sequence selected from SEQ ID NOs: 127-132.

68. The bi-specific binding protein of claim 66 or 67, wherein the first and second light chain polypeptides comprise an anti-IL-17A antibody light chain.

69. The bi-specific binding protein of claim 68, wherein the first and second light chain polypeptides comprise an amino acid sequence selected from SEQ ID NO: 141 and 142.

70. The bi-specific binding protein of any one of claims 66-69, wherein the first and second heavy chain polypeptide comprise the amino acid sequence of any one of SEQ ID NO: 127-132 and a first and second light chain polypeptide comprise the amino acid sequence of SEQ ID NO: 141 or 142.

71. The bi-specific binding protein of claim 70, wherein
(a) the bi-specific binding protein comprises a first and second heavy chain polypeptide comprising the amino acid sequence of SEQ ID NO: 127 and a first and second light chain polypeptide comprising the amino acid sequence of SEQ ID NO: 141;
(b) the bi-specific binding protein comprises a first and second heavy chain polypeptide comprising the amino acid sequence of SEQ ID NO: 128 and a first and second light chain polypeptide comprising the amino acid sequence of SEQ ID NO: 141;
(c) the bi-specific binding protein comprises a first and second heavy chain polypeptide comprising the amino acid sequence of SEQ ID NO: 129 and a first and second light chain polypeptide comprising the amino acid sequence of SEQ ID NO: 142;
(d) the bi-specific binding protein comprises a first and second heavy chain polypeptide comprising the amino acid sequence of SEQ ID NO: 130 and a first and second light chain polypeptide comprising the amino acid sequence of SEQ ID NO: 142;
(e) the bi-specific binding protein comprises a first and second heavy chain polypeptide comprising the amino acid sequence of SEQ ID NO: 131 and a first and second light chain polypeptide comprising the amino acid sequence of SEQ ID NO: 142; or
(f) the bi-specific binding protein comprises a first and second heavy chain polypeptide comprising the amino acid sequence of SEQ ID NO: 132 and a first and second light chain polypeptide comprising the amino acid sequence of SEQ ID NO: 142.

72. A bi-specific binding protein comprising a first and a second heavy chain polypeptide and a first and a second light chain polypeptide, wherein the first and second heavy chain polypeptides comprise, from N-terminus to C-terminus, an anti-IL-17A antigen-binding domain, a linker, and an antigen-binding domain.

73. The bi-specific binding protein of claim 72, wherein the first and second heavy chain polypeptides comprise, from N-terminus to C-terminus, an anti-IL-17A antibody heavy chain, a linker, and an antigen-binding domain.

74. The bi-specific binding protein of claim 72 or 73, wherein the antigen-binding domain is a VEGF-binding domain or a TNFα binding domain.

75. The bi-specific binding protein of claim 74, wherein the first and second heavy chain polypeptides comprise, from N-terminus to C-terminus, an anti-IL-17A antibody heavy chain comprising an amino acid sequence selected from SEQ ID NO: 169, 170, 175, and 176, a linker, and a VEGF antigen-binding domain comprising SEQ ID NO: 167 or 168.

76. The bi-specific binding protein of claim 75, wherein the first and second heavy chain polypeptides comprise an amino acid sequence selected from SEQ ID NO: 109-116.

77. The bi-specific binding protein of any one of claims 72-76, wherein the first and second light chain polypeptides comprise an anti-IL-17A antibody light chain.

78. The bi-specific binding protein of claim 77, wherein the first and second light chain polypeptides comprise an anti-IL-17A antibody light chain comprising the amino acid sequence of SEQ ID NO: 136 or 137.

79. The bi-specific binding protein of any one of claims 76-78, wherein the first and second heavy chain polypeptide comprise an amino acid sequence selected from SEQ ID NO: 109-116 and the first and second light chain polypeptide comprise the amino acid sequence of SEQ ID NO: 136 or 137.

80. The bi-specific binding protein of claim 79, wherein:
(a) the bi-specific binding protein comprises a first and second heavy chain polypeptide comprising the amino acid sequence of SEQ ID NO: 109 and a first and second light chain polypeptide comprising the amino acid sequence of SEQ ID NO: 136;
(b) the bi-specific binding protein comprises a first and second heavy chain polypeptide comprising the amino acid sequence of SEQ ID NO: 110 and a first and second light chain polypeptide comprising the amino acid sequence of SEQ ID NO: 136;
(c) the bi-specific binding protein comprises a first and second heavy chain polypeptide comprising the amino acid sequence of SEQ ID NO: 111 and a first and second light chain polypeptide comprising the amino acid sequence of SEQ ID NO: 136;
(d) the bi-specific binding protein comprises a first and second heavy chain polypeptide comprising the amino acid sequence of SEQ ID NO: 112 and a first and second light chain polypeptide comprising the amino acid sequence of SEQ ID NO: 136;
(e) the bi-specific binding protein comprises a first and second heavy chain polypeptide comprising the amino acid sequence of SEQ ID NO: 113 and a first and second light chain polypeptide comprising the amino acid sequence of SEQ ID NO: 137;
(f) the bi-specific binding protein comprises a first and second heavy chain polypeptide comprising the amino acid sequence of SEQ ID NO: 114 and a first and second light chain polypeptide comprising the amino acid sequence of SEQ ID NO: 137;
(g) the bi-specific binding protein comprises a first and second heavy chain polypeptide comprising the amino acid sequence of SEQ ID NO: 115 and a first and second light chain polypeptide comprising the amino acid sequence of SEQ ID NO: 137; or
(h) the bi-specific binding protein comprises a first and second heavy chain polypeptide comprising the amino acid sequence of SEQ ID NO: 116 and a first and second light chain polypeptide comprising the amino acid sequence of SEQ ID NO: 137.

81. A bi-specific binding protein comprising a first and a second heavy chain polypeptide and a first and a second light chain polypeptide, wherein the first and second light chains comprise, from N-terminus to C-terminus, an antigen binding domain, a linker, and an anti-IL-17A antigen binding domain.

82. The bi-specific binding protein of claim 81, wherein the first and second heavy chain polypeptides comprise an anti-IL-17A antibody heavy chain.

83. The bi-specific binding protein of claim 82, wherein the first and second heavy chain polypeptides comprise an anti-IL-17A antibody heavy chain comprising an amino acid sequence selected from SEQ ID NO: 117-120.

84. The bi-specific binding protein of claim 81 or 82, wherein the anti-IL-17A antigen binding domain is an anti-IL-17A antibody light chain.

85. The bi-specific binding protein of any one of claims 81-84, wherein the antigen-binding domain is a VEGF-binding domain or a TNFα binding domain.

86. The bi-specific binding protein of claim 85, wherein the first and second light chains comprise, from N-terminus to C-terminus, a VEGF binding domain comprising SEQ ID NO: 167, a linker, and an anti-IL-17A antibody light chain comprising an amino acid sequence selected from SEQ ID NO: 136 and 137.

87. The bi-specific binding protein of claim 86, wherein the first and second light chain polypeptides comprise an amino acid sequence selected from SEQ ID NO: 138 and 139.

88. The bi-specific binding protein of any one of claims 81-87, wherein the first and second heavy chain polypeptides comprise an amino acid sequence selected from SEQ ID NO: 117-120 and the first and second light chain polypeptides comprise an amino acid sequence selected from SEQ ID NO: 138 and 139.

89. The bi-specific binding protein of claim 88, wherein
(a) the first and second heavy chain polypeptides comprise an amino acid sequence selected from SEQ ID NO: 117 and the first and second light chain polypeptides comprise an amino acid sequence selected from SEQ ID NO: 138;
(b) the first and second heavy chain polypeptides comprise an amino acid sequence selected from SEQ ID NO: 118 and the first and second light chain polypeptides comprise an amino acid sequence selected from SEQ ID NO: 138;
(c) the first and second heavy chain polypeptides comprise an amino acid sequence selected from SEQ ID NO: 119 and the first and second light chain polypeptides comprise an amino acid sequence selected from SEQ ID NO: 139; or
(d) the first and second heavy chain polypeptides comprise an amino acid sequence selected from SEQ ID NO: 120 and the first and second light chain polypeptides comprise an amino acid sequence selected from SEQ ID NO: 139.

90. A bi-specific binding protein comprising a first and a second protein monomer, wherein each monomer comprises, from N-terminus to C-terminus, an antigen binding domain, a linker, and an IL-17A binding domain.

91. The bi-specific binding protein of claim 90, wherein the antigen-binding domain is a VEGF-binding domain or a TNFα binding domain.

92. The bi-specific binding protein of claim 91, wherein the first and second monomers comprise, from N-terminus to C-terminus, a VEGF binding domain comprising SEQ ID NO: 100, a linker, and an anti-IL-17A scFv binding domain comprising an amino acid sequence selected from SEQ ID NO: 177-179.

93. The bi-specific binding protein of claim 92 wherein each monomer comprises an amino acid sequence that is at least 90%, 95%, 96%, 97%, 98%, or 99% identical to an amino acid sequence selected from SEQ ID NO: 123-125.

94. The bi-specific binding protein of claim 92, wherein each monomer comprises or consists of an amino acid sequence selected from SEQ ID NO: 123-125.

95. The bi-specific binding protein of claim 92, wherein each monomer comprises an amino acid sequence that is at least 90%, 95%, 96%, 97%, 98%, or 99% identical to SEQ ID NO: 124.

96. The bi-specific binding protein of claim 92, wherein each monomer comprises or consists of SEQ ID NO: 124.

97. The bi-specific binding protein of claim 92, wherein each monomer comprises an amino acid sequence that is at least 90%, 95%, 96%, 97%, 98%, or 99% identical to SEQ ID NO: 125.

98. The bi-specific binding protein of claim 92, wherein each monomer comprises or consists of SEQ ID NO: 125.

99. The bi-specific binding protein of any one of claims 59, 85, or 91, wherein the VEGF antigen-binding domain is a soluble VEGF receptor or an anti-VEGF antibody or antigen-binding fragment thereof.

100. The bi-specific binding protein of claim 99, wherein the soluble VEGF receptor is aflibercept.

101. The bi-specific binding protein of claim 99, wherein the anti-VEGF antibody is selected from bevacizumab, ranibizumab, brolucizumab, and ramucirumab.

102. The bi-specific binding protein of any one of claims 59, 85, or 91, wherein the TNFα antigen-binding domain is an anti-TNFα antibody or antigen binding fragment thereof.

103. The bi-specific binding protein of claim 102, wherein the anti-TNFα antibody is selected from adalimumab, infliximab, etanercept, golimumab, and certolizumab.

104. A polynucleotide encoding the bi-specific binding protein of any one of claims 57-103.

105. An expression vector comprising the polynucleotide of claim 104.

106. A host cell comprising the polynucleotide of claim 104 or the expression vector of claim 105.

107. A method of manufacturing the bi-specific binding protein of any one of claims 57-103, comprising introducing the expression vector of claim 105 into a host cell.

108. A vector comprising a polynucleotide encoding the a the bi-specific binding protein of any one of claims 57-103.

109. The vector of claim 108, wherein the vector is an AAV8 or AAV9 vector.

110. A method of treating an inflammatory condition in a subject in need thereof, comprising administering the anti-IL-17A antibody or antigen-binding fragment thereof of any one of claims 1-28, the humanized anti-IL-17A antibody or antigen-binding fragment thereof of any one of claims 29-44, the anti-IL-17A scFv of any one of claims 48-49, the bi-specific protein of any one of claims 57-103, or the vector of any one of claims 54, 55, 108, or 109, to a subject in need thereof.

111. Use of the anti-IL-17A antibody or antigen-binding fragment thereof of any one of claims 1-28, the humanized anti-IL-17A antibody or antigen-binding fragment thereof of any one of claims 29-44, the anti-IL-17A scFv of any one of claims 48-49, the bi-specific protein of any one of claims 57-103, or the vector of any one of claims 54, 55, 108, or 109 for the manufacture of a medicament for treatment of an inflammatory condition in a subject.

112. The method of claim 110 or use of claim 111, wherein the inflammatory condition is selected from airway inflammation, rheumatoid arthritis, osteoarthritis, bone erosion, intraperitoneal abscesses and adhesions, infectious disease, inflammatory bowel disorder, macular degeneration, allograft rejection, psoriasis, cancer, angiogenesis, atherosclerosis, and multiple sclerosis.

113. A method of treating an ocular disease in a subject in need thereof, comprising administering the anti-IL-17A antibody or antigen-binding fragment thereof of any one of claims 1-28, the humanized anti-IL-17A antibody or antigen-binding fragment thereof of any one of claims 29-44, the anti-IL-17A scFv of any one of claims 48-49, the bi-specific protein of any one of claims 57-103, or the vector of any one of claims 54, 55, 108, or 109, to a subject in need thereof.

114. Use of the anti-IL-17A antibody or antigen-binding fragment thereof of any one of claims 1-28, the humanized anti-IL-17A antibody or antigen-binding fragment thereof of any one of claims 29-44, the anti-IL-17A scFv of any one of claims 48-49, the bi-specific protein of any one of claims 57-103, or the vector of any one of claims 54, 55, 108, or 109 for the manufacture of a medicament for treatment of an ocular disease in a subject.

115. The method of claim 113 or use of claim 114, wherein the ocular disease is selected from macular degeneration, retinitis pigmentosa, and diabetic retinopathy.

116. The method or use of claim 115, wherein the macular degeneration is age-related macular degeneration.

117. The method or use of claim 115 or 116, wherein the macular degeneration is wet or dry.

118. A method of decreasing vascular leakage in the eye in a subject in need thereof, comprising administering the anti-IL-17A antibody or antigen-binding fragment thereof of any one of claims 1-28, the humanized anti-IL-17A antibody or antigen-binding fragment thereof of any one of claims 29-44, the anti-IL-17A scFv of any one of claims 48-49, the bi-specific protein of any one of claims 57-103, or the vector of any one of claims 54, 55, 108, or 109.

119. Use of the anti-IL-17A antibody or antigen-binding fragment thereof of any one of claims 1-28, the humanized anti-IL-17A antibody or antigen-binding fragment thereof of any one of claims 29-44, the anti-IL-17A scFv of any one of claims 48-49, the bi-specific protein of any one of claims 57-103, or the vector of any one of claims 54, 55, 108, or 109 for the manufacture of a medicament for decreasing vascular leakage in the eye in a subject in need thereof.

120. The method or use of any one of claims 110-119, wherein the antibody, antigen-binding fragment thereof, scFv, bi-specific protein, or vector are administered intravenously, intravitreally, subcutaneously, or intramuscularly.

121. The method or use of any one of claims 110-120, wherein the subject is human.
